(19) United States
(12) Patent Application Publication
(10) Pub. No.: US 2023/0247899 A1
(43) Pub. Date: Aug. 3, 2023

(54) ORGANIC ELECTRONIC ELEMENT CONTAINING COMPOUND FOR ORGANIC ELECTRONIC ELEMENT, AND ELECTRONIC DEVICE THEREFOR

(71) Applicant: DUK SAN NEOLUX CO., LTD., Cheonan-si, Chungcheongnam-do (KR)

(72) Inventors: Jong Gwang PARK, Cheonan-si, Chungcheongnam-do (KR); Nam Geol LEE, Cheonan-si, Chungcheongnam-do (KR); Sun Hee LEE, Cheonan-si, Chungcheongnam-do (KR); Soung Yun LEE, Cheonan-si, Chungcheongnam-do (KR); Guang Ming LI, Cheonan-si, Chungcheongnam-do (KR)

(73) Assignee: DUK SAN NEOLUX CO., LTD., Cheonan-si, Chungcheongnam-do (KR)

(21) Appl. No.: 18/004,542

(22) PCT Filed: Jul. 9, 2021
(86) PCT No.: PCT/KR2021/008779

§ 371 Date: Jan. 6, 2023

(30) Foreign Application Priority Data
Jul. 9, 2020 (KR) 10-2020-0084933

(51) Int. Cl. H10K 85/60; C07D 311/96; C09K 11/06; C07D 409/12; C07D 339/08; C07D 221/20; C07D 405/04; C07D 405/14; C07D 409/14

(52) U.S. Cl. CPC H10K 85/636; C07D 311/96; C09K 11/06; H10K 85/633; C07D 409/12; C07D 339/08; C07D 221/20; C07D 405/04; H10K 85/654; H10K 85/6574; C07D 405/14; C07D 409/14; H10K 85/6576; H10K 50/15

(57) ABSTRACT

Provides is an organic electronic element comprising an anode, a cathode, and an organic material layer between the anode and the cathode; and an electronic device comprising the organic electronic element, wherein the organic material layer comprises compounds represented by Formula 1 and Formula 2, respectively, and thus can lower the driving voltage of the organic electronic element and improve the luminosity and lifespan thereof.

### BACKGROUND

### Technical Field

The present invention relates to organic electronic element using compound for organic electronic element and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electronic element may be classified into a light emitting material and a charge transport material, such as a hole injection material, a hole transport material, an electron transport material, an electron injection material and the like depending on its function.

In the organic light emitting diode, the most problematic is the lifespan and the efficiency. As the display becomes large, the efficiency and the lifespan problem must be solved. Efficiency, lifespan, driving voltage and the like are related to each other, as the efficiency is increased, the driving voltage is relatively decreased, and as the driving voltage drops, the crystallization of the organic material due to Joule heating generated during driving is reduced, and as a result, the lifespan tends to increase.

However, simply improving the organic material layer cannot maximize the efficiency. This is because, when the optimal combination of the energy level and T1 value between each organic material layer, and the intrinsic properties (mobility, interface characteristics, etc.) of the material are achieved, long life and high efficiency can be achieved at the same time.

Further, recently, in organic electroluminescent devices, in order to solve the emitting problem in the hole transport layer, an emitting-auxiliary layer must be present between the hole transport layer and the emitting layer, and it is necessary to develop different emitting-auxiliary layers according to the respective emitting layers (R, G, B).

In general, electrons are transferred from the electron transport layer to the emitting layer, and holes are transferred from the hole transport layer to the emitting layer to generate excitons by recombination.

However, the material used for the hole transport layer has a low HOMO value and therefore has mostly low T1 value. As a result, the exciton generated in the emitting layer is transferred to the hole transport layer, resulting in charge unbalance in the emitting layer, and light is emitted at the interface of the hole transport layer.

When light is emitted at the interface of the hole transport layer, the color purity and efficiency of the organic electronic element are lowered and the lifespan is shortened. Therefore, it is urgently required to develop an emitting-auxiliary layer having a high T1 value and a HOMO level between the HOMO energy level of the hole transport layer and the HOMO energy level of the emitting layer.

Meanwhile, it is necessary to develop a hole injection layer material having stable characteristics, that is, a high glass transition temperature, against joule heating generated when the element is driven, while delaying penetration of the metal oxide from the anode electrode (ITO), which is one of the causes of shortening the lifespan of the organic electronic element, into the organic layer. The low glass transition temperature of the hole transport layer material has a characteristic that when the device is driven, the uniformity of the surface of the thin film is lowered, which has been reported to have a great influence on the lifespan of the element. In addition, OLED devices are mainly formed by a deposition method, and it is necessary to develop a material that can withstand long time in deposition, that is, a material having high heat resistance characteristics.

That is, in order to sufficiently exhibit the excellent characteristics of the organic electronic element, a material for forming an organic material layer in an element such as a hole injection material, a hole transport material, a light emitting material, an electron transport material, an electron injection material, an emitting-auxiliary layer material should be supported by stable and efficient materials. However, such a stable and efficient organic material layer material for an organic electronic element has not been sufficiently developed yet. Therefore, development of new materials is continuously required, and development of materials for the hole transport layer or the emitting-auxiliary layer is urgently required.

As a reference prior art document, KR020190038246 A was used.

### BRIEF DESCRIPTION OF THE INVENTION

### Summary

An object of the present invention is to provide an organic electronic element and an electronic device thereof comprising a compound capable of lowering the driving voltage of the element and improving the light emitting efficiency, color purity, stability and lifespan of the element.

### Technical Solution

In one aspect, the present invention provides an organic electronic element comprising an anode, a cathode, and an organic material layer formed between the anode and the cathode, wherein the organic material layer comprises an emitting layer and a hole transport band layer formed between the emitting layer and the anode, wherein the hole transport band layer comprises a compound represented by Formula 1, wherein the emitting layer provides an organic electronic element comprising a compound represented by Formula 2.

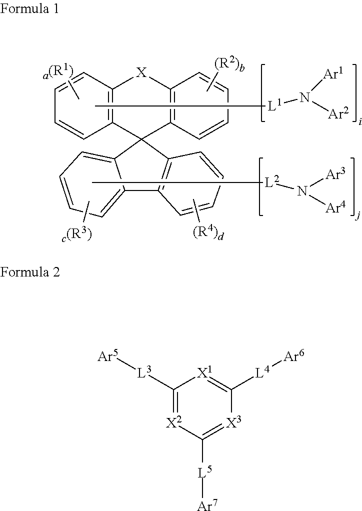

In another aspect, the present invention provides an electronic device including the organic electronic element.

### Effects of the Invention

By using the compound according to the present invention, it is possible to achieve a high luminous efficiency, a low driving voltage, and a high heat resistance of the element, and can greatly improve the color purity and lifespan of the element.

### BRIEF DESCRIPTION OF THE DRAWINGS

FIG. 1 to FIG. 3 illustrate an example of an organic electronic element according to the present invention.

100, 200, 300: organic electronic element
110: the first electrode
120: hole injection layer
130: hole transport layer
140: emitting layer
150: electron transport layer
160: electron injection layer
170: second electrode
180: light efficiency enhancing Layer
210: buffer layer
220: emitting auxiliary layer
320: first hole injection layer
330: first hole transport layer
340: first emitting layer
350: first electron transport layer
360: first charge generation layer
361: second charge generation layer
420: second hole injection layer
430: second hole transport layer
440: second emitting layer
450: second electron transport layer
CGL: charge generation layer
ST1: first stack
ST2: second stack

### DETAILED DESCRIPTION OF THE INVENTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected”,“coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

Unless otherwise stated, the term “aryl group” or “arylene group”, as used herein, has 6 to 60 carbon atoms, but is not limited thereto. Herein, the aryl group or arylene group means a monocyclic and polycyclic aromatic group, and may also be formed in conjunction with an adjacent group. Examples of “aryl group” may include a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of monocyclic and polycyclic rings, and may include heteroaliphatic ring and/or heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes compound below.

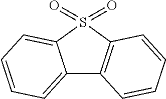 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

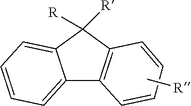

The term “spiro compound”, as used herein, has a ‘spiro union’, and a spiro union means a connection in which two rings share only one atom. At this time, atoms shared in the two rings are called ‘spiro atoms’, and these compounds are called ‘monospiro’, ‘di-spiro’ and ‘tri-spiro’, respectively, depending on the number of atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include one or more heteroatoms, but are not limited thereto.

Unless otherwise stated, the term “substituted or unsubstituted”, as used herein, means that substitution is substituted by at least one substituent selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiophen group, a C₆-C₂₀ arylthiophen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited thereto.

Unless otherwise expressly stated, the Formula used in the present invention, as used herein, is applied in the same manner as the substituent definition according to the definition of the exponent of the following Formula.

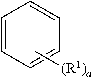

wherein, when a is an integer of zero, the substituent R¹ is absent, when a is an integer of 1, the sole substituent R¹ is linked to any one of the carbon constituting the benzene ring, when a is an integer of 2 or 3, each substituent R¹s may be the same and different, when a is an integer of 4 to 6, and is linked to the benzene ring in a similar manner, whereas the indication of hydrogen bound to the carbon forming the benzene ring is omitted.

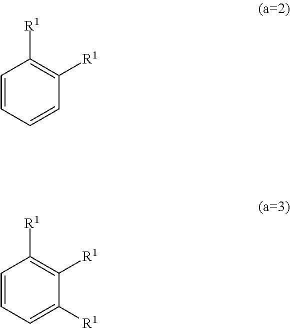

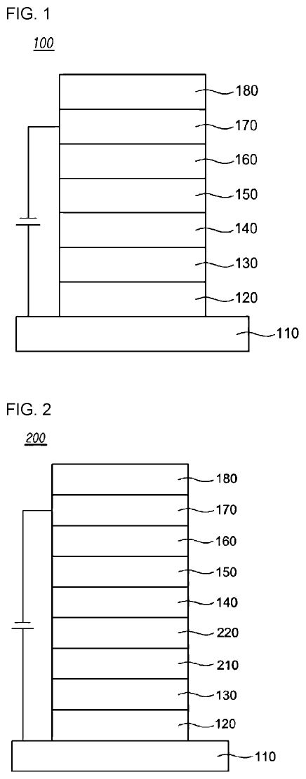

Hereinafter, a layered structure of an organic electronic element comprising the compound of the present invention will be described with reference to FIGS. 1 to 3.

In adding reference numerals to the components of each drawing, it should be noted that the same components have the same numerals as much as possible even if they are displayed on different drawings. Also, in describing the present invention, if it is determined that a detailed description of a related known configuration or function may obscure the gist of the present invention, the detailed description will be omitted.

FIGS. 1 to 3 are exemplary views of an organic electronic element according to an embodiment of the present invention.

Referring to FIG. 1, an organic electronic element (100) according to an embodiment of the present invention comprises a first electrode (110), a second electrode (170), and an organic material layer formed between the first electrode (110) and the second electrode (170) formed on a substrate (not shown).

The first electrode (110) may be an anode (anode), the second electrode (170) may be a cathode (cathode), and in the case of an inverted type, the first electrode may be a cathode and the second electrode may be an anode.

The organic material layer may include a hole injection layer (120), a hole transport layer (130), an emitting layer (140), an electron transport layer (150), and an electron injection layer (160). Specifically, the hole injection layer (120), the hole transport layer (130), the emitting layer (140), the electron transport layer (150), and the electron injection layer (160) may be sequentially formed on the first electrode (110).

Preferably, the light efficiency enhancing layer (180) may be formed on one surface of both surfaces of the first electrode (110) or the second electrode (170), not being contacted to the organic material layer, and when the light efficiency enhancing layer (180) is formed, the light efficiency of the organic electronic element may be improved.

For example, the light efficiency enhancing layer (180) may be formed on the second electrode (170), in the case of a top emission organic light emitting device, it is possible to reduce optical energy loss due to surface plasmon polaritons (SPPs) in the second electrode (170) by forming the light efficiency enhancing layer (180), and in the case of a bottom emission organic light emitting device, the light efficiency improving layer (180) may serve as a buffer for the second electrode (170).

A buffer layer (210) or an emitting auxiliary layer (220) may be further formed between the hole transport layer (130) and the emitting layer (140), which will be described with reference to FIG. 2.

Referring to FIG. 2, the organic electronic element (200) according to another embodiment of the present invention comprises a hole injection layer (120), a hole transport layer (130), a buffer layer (210), and an emitting auxiliary layer (220), an emitting layer (140), an electron transport layer (150), an electron injection layer (160) and a second electrode (170), sequentially formed on the first electrode (110), and a light efficiency enhancing layer (180) may be formed on the second electrode.

Although not shown in FIG. 2, an electron transport auxiliary layer may be further formed between the emitting layer (140) and the electron transport layer (150).

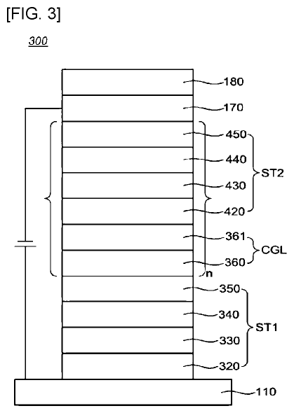

In addition, according to another embodiment of the present invention, the organic material layer may have a form in which a plurality of stacks including a hole transport layer, an emitting layer, and an electron transport layer are formed. This will be described with reference to FIG. 3.

Referring to FIG. 3, in the organic electronic element (300) according to another embodiment of the present invention, 2 or more sets of stacks (ST1, ST2) of an organic material layer comprising a multi-layered structure may be formed between the first electrode (110) and the second electrode (170), and a charge generation layer (CGL) may be formed between the stacks of the organic material layers.

Specifically, the organic electronic element according to an embodiment of the present invention may comprise a first electrode (110), a first stack (ST1), a charge generation layer (CGL), a second stack (ST2), and a second electrode (170) and the light efficiency enhancing layer (180).

The first stack (ST1), which is an organic material layer formed on the first electrode (110), may comprise a first hole injection layer (320), a first hole transport layer (330), a first emitting layer (340), and a first electron transport layer (350), and the second stack (ST2) may comprise a second hole injection layer (420), a second hole transport layer (430), a second emitting layer (440), and a second electron transport layer (450). As such, the first stack and the second stack may be organic material layers having the same stacked structure or organic material layers having different stacked structures.

A charge generation layer (CGL) may be formed between the first stack (ST1) and the second stack (ST2). The charge generation layer (CGL) may comprise a first charge generation layer (360) and a second charge generation layer (361). The charge generation layer (CGL) is formed between the first emitting layer (340) and the second emitting layer (440) to increase the current efficiency generated in each emitting layer, and to smoothly distribute charges.

As shown in FIG. 3, when a plurality of emitting layers are formed by a multi-layer stack structure method, an organic light emitting device emitting white light by a mixing effect of light emitted from each emitting layer may be manufactured, as well as an organic light emitting device emitting light of various colors.

The compound represented by Formula 1 of the present invention may be used as a material of the hole injection layer (120, 320, 420), the hole transport layer (130, 330, 430), the buffer layer (210), the emitting auxiliary layer (220), the electron transport layer (150, 350, 450), the electron injection layer (160), the emitting layer (140, 340, 440), or the light efficiency enhancing layer (180), but preferably, the compound represented by Formula 1 of the present invention may be used as a material for the emitting auxiliary layer (220), and the compound represented by Formula 2 of the present invention may be used as a host of the emitting layers (140, 340, 440).

Even with the same and similar core, the band gap, electrical properties, interface properties, etc. may vary depending on which position the substituent is bonded to, therefore it is necessary to study the selection of the core and the combination of sub-substituents bound thereto, and in particular, when the energy level and T1 value between each organic material layer, and the intrinsic properties (mobility, interfacial properties, etc.) of materials are optimally combined, long lifespan and high efficiency can be achieved at the same time.

The organic electroluminescent device according to an embodiment of the present invention may be manufactured using various deposition methods. It can be manufactured using a deposition method such as PVD or CVD, for example, by depositing a metal or a metal oxide having conductivity or an alloy thereof on a substrate to form the anode (110), and thereon, after forming an organic material layer including the hole injection layer (120), the hole transport layer (130), the emitting layer (140), the electron the transport layer (150) and the electron injection layer (160), it may be manufactured by depositing a material that can be used as the cathode (170) thereon. In addition, an emitting auxiliary layer (220) may be further formed between the hole transport layer (130) and the emitting layer (140), and an electron transport auxiliary layer (not shown) may be furtherformed between the emitting layer (140) and the electron transport layer (150), it can also be formed in a stack structure as shown.

Furthermore, the organic material layer may be manufactured in a smaller number of layers by a method such as a solution process or a solvent process, for example, a spin coating process, a nozzle printing process, an inkjet printing process, a slot coating process, a dip coating process, and a roll-to-roll process, doctor blading process, screen printing process, or a thermal transfer method, rather than a vapor deposition method, using various polymer materials. Since the organic material layer according to the present invention can be formed by various methods, the scope of the present invention is not limited by the formation method.

Also, the organic electronic element according to an embodiment of the present invention may be selected from the group consisting of an organic electroluminescent device, an organic solar cell, an organic photoreceptor, an organic transistor, a monochromatic lighting device, and a quantum dot display device.

Another embodiment of the present invention may include a display device including the organic electronic element of the present invention described above, and an electronic device including a control unit for driving the display device. In this case, the electronic device may be a current or future wired/wireless communication terminal, and includes all electronic devices such as a mobile communication terminal such as a mobile phone, a PDA, an electronic dictionary, a PMP, a remote control, a navigation system, a game machine, various TVs, and various computers.

Hereinafter, an organic electric device according to an aspect of the present invention will be described.

An organic electronic element according to an embodiment of the present invention comprises an anode, a cathode, and an organic material layer formed between the anode and the cathode, wherein the organic material layer comprises an emitting layer, and a hole transport band layer formed between the emitting layer and the anode, wherein the hole transport band layer comprises a compound represented by Formula 1, and the emitting layer comprises a compound represented by Formula 2:

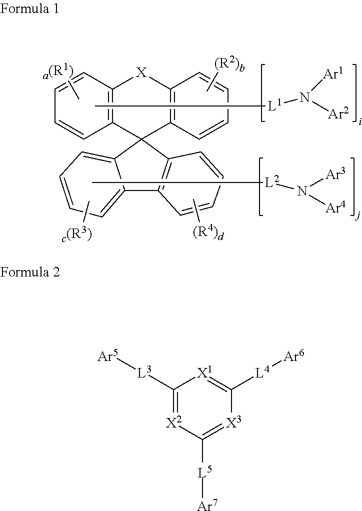

In Formula 1 and Formula 2, each symbol may be defined as follows.

1) X is O, S or NR, except when X is NR, i is 0 and j is 1,

2) X¹, X² and X³ are each independently CR′ or N, provided that at least two of X¹, X² and X³ are N,

3) R¹, R², R³, R⁴, R and R′ are each independently the same as or different from each other, and are each independently selected from the group consisting of hydrogen; deuterium; halogen; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and -L′-NR^{a}R^{b}; or in case a, b, c and d are 2 or more, a plurality of adjacent R¹s, or a plurality of R²s, or a plurality of R³s, or a plurality of R⁴s may be bonded to each other to form a ring,

wherein in case R¹, R², R³, R⁴, R and R′ are an alkyl group, it may be preferably a C₁-C₃₀ alkyl group, and more preferably a C₁-C₂₄ alkyl group,

wherein in case R¹, R², R³, R⁴, R and R′ are an alkenyl group, it may be preferably a C₂-C₃₀ alkenyl group, and more preferably a C₂-C₂₄ alkenyl group,

wherein in case R¹, R², R³, R⁴, R and R′ are an alkynyl group, it may be preferably a C₂-C₃₀ alkynyl group, and more preferably a C₂-C₂₄ alkynyl group,

wherein in case R¹, R², R³, R⁴, R and R′ are an alkoxyl group, it may be preferably an C₁˜C₃₀ alkoxyl group, and more preferably an C₁˜C₂₄ alkoxyl group,

wherein in case R¹, R², R³, R⁴, R and R′ are an aryloxy group, it may be preferably an C₆˜C₃₀ aryloxy group, and more preferably an C₆˜C₂₄ aryloxy group,

wherein in case R¹, R², R³, R⁴, R and R′ are an aryl group, it may be preferably a C₆-C₃₀ aryl group, and more preferably a C₆-C₂₄ aryl group, for example, it may be phenylene, biphenyl, naphthalene, terphenyl, etc,

wherein in case R¹, R², R³, R⁴, R and R′ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc., wherein in case R¹, R², R³, R⁴, R and R′ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

4) L′, L¹, L², L³, L⁴ and L⁵ are each independently selected from the group consisting of a single bond; a C₆-C₆₀ arylene group; fluorenylene group; a fused ring group of a C₃-C₆₀ aliphatic ring and a C₆-C₆₀ aromatic ring; a C₂-C₆₀ heterocyclic group;

wherein in case L′, L¹, L², L³, L⁴ and L⁵ are an arylene group, it may be preferably a C₆-C₃₀ arylene group, more preferably a C₆-C₂₄ arylene group, for example, phenylene, biphenyl, naphthalene, terphenyl, etc.,

wherein in case L′, L¹, L², L³, L⁴ and L⁵ are a fused ring group, it may be preferably a fused ring group of a C₃-C₃₀ aliphatic ring and a C₆-030 aromatic ring, more preferably a fused ring group of a C₃-C₂₄ aliphatic ring and a C₆-C₂₄ aromatic ring,

wherein in case L′, L¹, L², L³, L⁴ and L⁵ are a heterocyclic group, it may be preferably a C₂˜C₃₀ heterocyclic group, and more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

5) wherein R^{a} and R^{b} are each independently selected from the group consisting of a C₆˜C₆₀ aryl group; fluorenyl group; a fused ring group of a C₃-C₆₀ aliphatic ring and a C₆-C₆₀ aromatic ring; a C₂-C₆₀ heterocyclic group including at least one heteroatom of O, N, S, Si or P;

wherein in case R^{a} and R^{b} are an aryl group, it may be preferably a C₆-C₃₀ aryl group, more preferably a C₆-C₂₄ aryl group, for example, phenylene, biphenyl, naphthalene, terphenyl, etc.,

wherein in case R^{a} and R^{b} are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring,

wherein in case R^{a} and R^{b} are a heterocyclic group, it may be preferably a C₂˜C₃₀ heterocyclic group, and more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, triazine, quinoxaline, etc.,

6) a, b, c and d are each independently an integer of 0 to 4,

7) i and j are independently integers from 0 to 2, provided that i+j is an integer of 1 or more;

8) Ar¹, Ar², Ar³, Ar⁴, Ar⁵, Ar⁶ and Ar⁷ are each independently selected from the group consisting of a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a C₂-C₆₀ heterocyclic group including at least one heteroatom of O, N, S, Si or P; and a fused ring group of a C₃-C₆₀ aliphatic ring and a C₆-C₆₀ aromatic ring; alternatively, Ar¹ and Ar² or Ar³ and Ar⁴ may be bonded to each other to form a ring.

wherein in case Ar¹, Ar², Ar³, Ar⁴, Ar⁵, Ar⁶ and Ar⁷ are an alkyl group, it may be preferably a C₁-C₃₀ alkyl group, and more preferably a C₁-C₂₄ alkyl group.

wherein in case Ar¹, Ar², Ar³, Ar⁴, Ar⁵, Ar⁶ and Ar⁷ are an alkenyl group, it may be preferably a C₂-C₃₀ alkenyl group, and more preferably a C₂-C₂₄ alkenyl group,

wherein in case Ar¹, Ar², Ar³, Ar⁴, Ar⁵, Ar⁶ and Ar⁷ are an alkynyl group, it may be preferably a C₂-C₃₀ alkynyl group, and more preferably a C₂-C₂₄ alkynyl group,

wherein in case Ar¹, Ar², Ar³, Ar⁴, Ar⁵, Ar⁶ and Ar⁷ are an alkoxyl group, it may be preferably an C₁˜C₃₀ alkoxyl group, and more preferably an C₁˜C₂₄ alkoxyl group,

wherein in case Ar¹, Ar², Ar³, Ar⁴, Ar⁵, Ar⁶ and Ar⁷ are an aryloxy group, it may be preferably an C₆˜C₃₀ aryloxy group, and more preferably an C₁˜C₂₄ aryloxy group, wherein in case Ar¹, Ar², Ar³, Ar⁴, Ar⁵, Ar⁶ and Ar⁷ are an aryl group, it may be preferably a C₆-C₃₀ aryl group, and more preferably a C₆-C₂₄ aryl group, for example, it may be phenylene, biphenyl, naphthalene, terphenyl, etc,

wherein in case Ar¹, Ar², Ar³, Ar⁴, Ar⁵, Ar⁶ and Ar⁷ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, di benzoqui nazoline, dibenzofuran, benzothienopyrimidine, benzofuropyrimidine, phenothiazine, phenylphenothiazine, etc.,

wherein in case Ar¹, Ar², Ar³, Ar⁴, Ar⁵, Ar⁶ and Ar⁷ are a fused ring group, it may be preferably a fused ring group of a C₃-C₃₀ aliphatic ring and a C₆-030 aromatic ring, more preferably a fused ring group of a C₃-C₂₄ aliphatic ring and a C₆-C₂₄ aromatic ring.

9) wherein the aryl group, arylene group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and -L′-NR^{a}R^{b}, and also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, the compound represented by Formula 1 is represented by any one of Formulas 1-1 to 1-7.

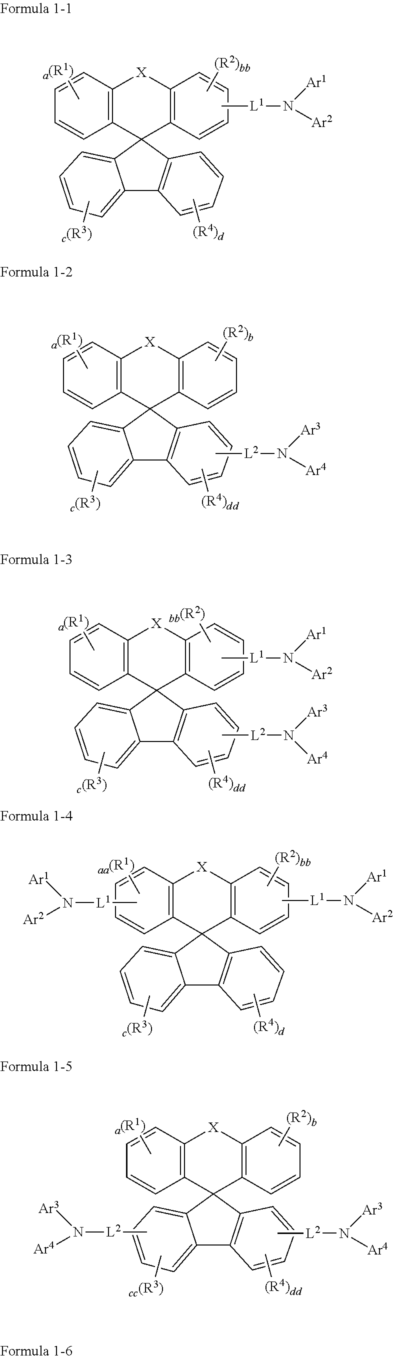

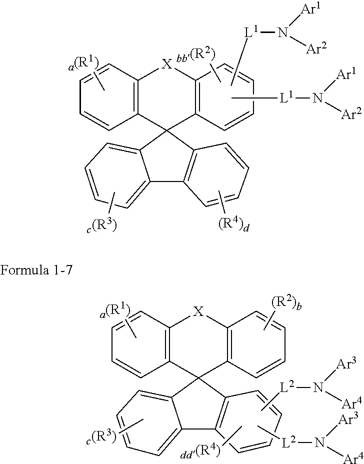

wherein:

1) X, R¹, R², R³, R⁴, a, b, c, d, L¹, L², Ar¹, Ar², Ar³ and Ar⁴ are the same as defined in Formula 1,

2) aa, bb, cc and dd are each independently an integer of 0 to 3,

3) bb′ and dd′ are each independently an integer of 0 to 2.

Also, the compound represented by Formula 1 is represented by any one of Formulas 1-8 to 1-9.

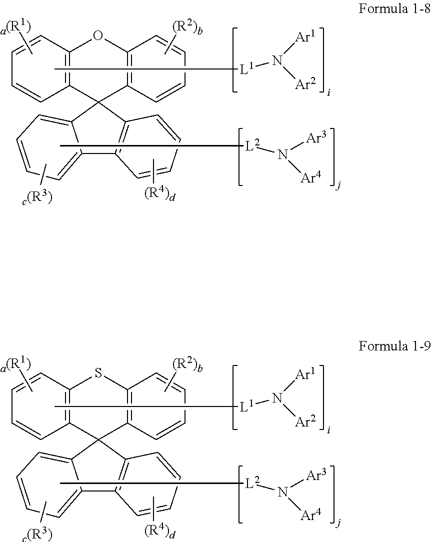

wherein R¹, R², R³, R⁴, a, b, c, d, L¹, L², Ar¹, Ar², Ar³, Ar⁴, i and j are the same as defined in Formula 1.

Also, at least one of Ar¹ to Ar⁴ in Formula 1 is represented by Formula B-1.

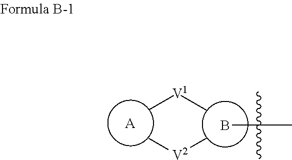

wherein

1) V¹ and V² are each independently a single bond, NR⁵, CR⁶R⁷, O or S,

2) R⁵, R⁶ and R⁷ are the same as the definition of R¹ in Formula 1, provided that, R⁶ and R⁷ may be bonded to each other to form a ring,

3) Ring A and ring B are each independently a substituted or unsubstituted C₆-C₂₀ aryl group; or a substituted or unsubstituted C₄˜C₂₀ heterocyclic group;

Specifically, the compound represented by Formula 1 may be any one of the following compounds:

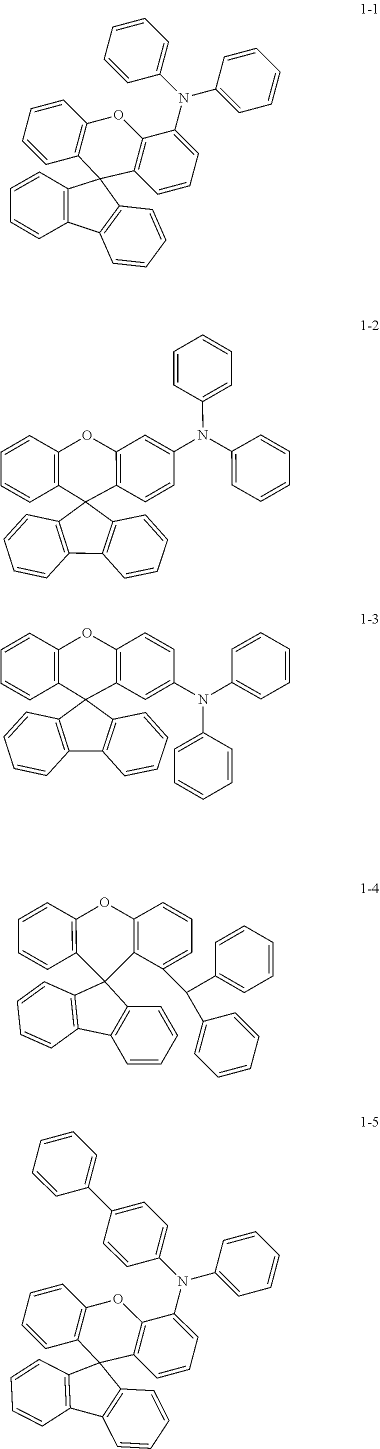 [SMILES: c1ccc(N(c2ccccc2)c2cccc3c2Oc2ccccc2C32c3ccccc3-c3ccccc32)cc1 ; c1ccc(N(c2ccccc2)c2ccc3c(c2)Oc2ccccc2C32c3ccccc3-c3ccccc32)cc1 ; c1ccc(N(c2ccccc2)c2ccc3c(c2)C2(c4ccccc4O3)c3ccccc3-c3ccccc32)cc1 ; c1ccc(C(c2ccccc2)c2cccc3c2C2(c4ccccc4O3)c3ccccc3-c3ccccc32)cc1 ; c1ccc(-c2ccc(N(c3ccccc3)c3cccc4c3Oc3ccccc3C43c4ccccc4-c4ccccc43)cc2)cc1]

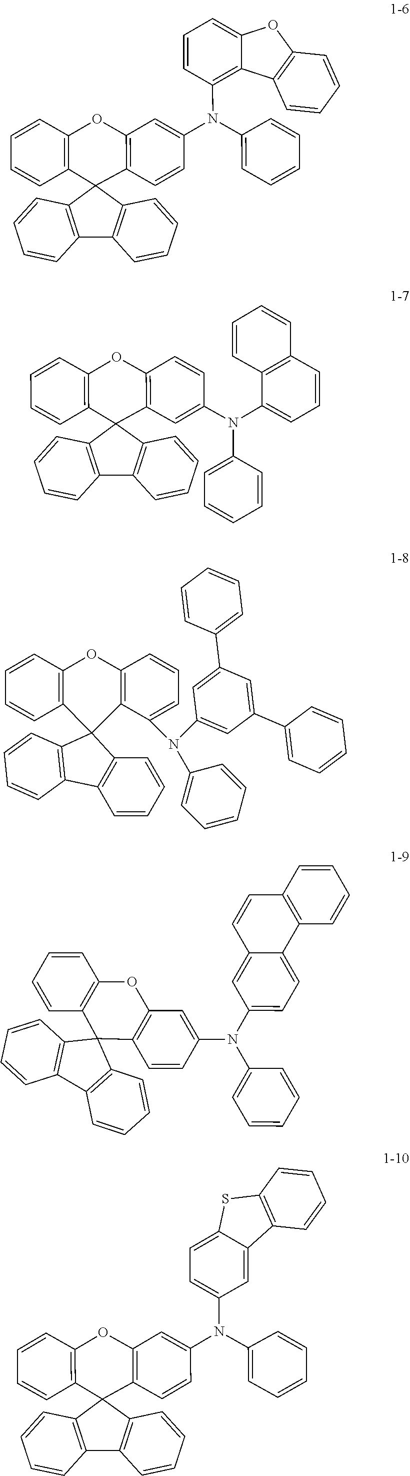

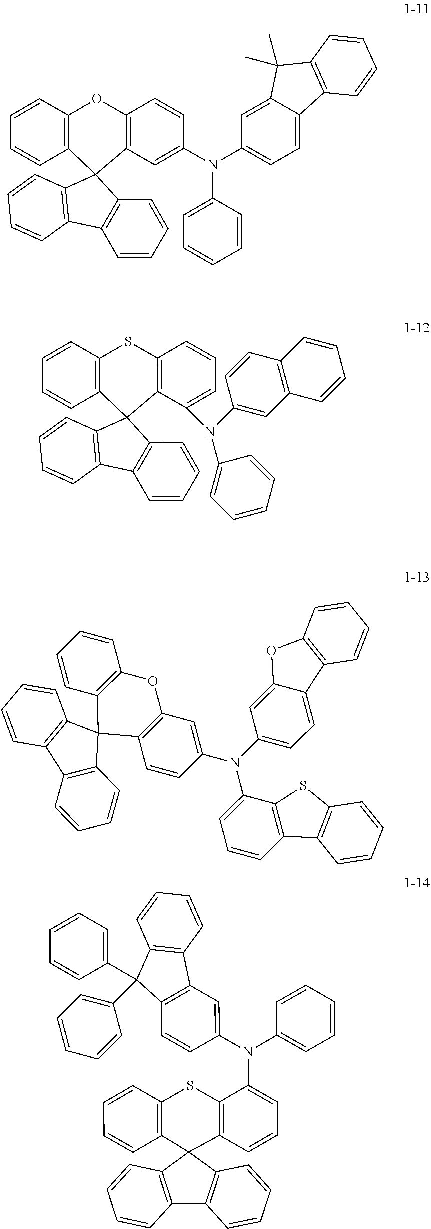 [SMILES: CC1(C)c2ccccc2-c2ccc(N(c3ccccc3)c3ccc4c(c3)C3(c5ccccc5O4)c4ccccc4-c4ccccc43)cc21 ; c1ccc(N(c2ccc3ccccc3c2)c2cccc3c2C2(c4ccccc4S3)c3ccccc3-c3ccccc32)cc1 ; c1ccc2c(c1)Oc1cc(N(c3ccc4c(c3)oc3ccccc34)c3cccc4c3sc3ccccc34)ccc1C21c2ccccc2-c2ccccc21 ; c1ccc(N(c2ccc3c(c2)-c2ccccc2C3(c2ccccc2)c2ccccc2)c2cccc3c2Sc2ccccc2C32c3ccccc3-c3ccccc32)cc1]

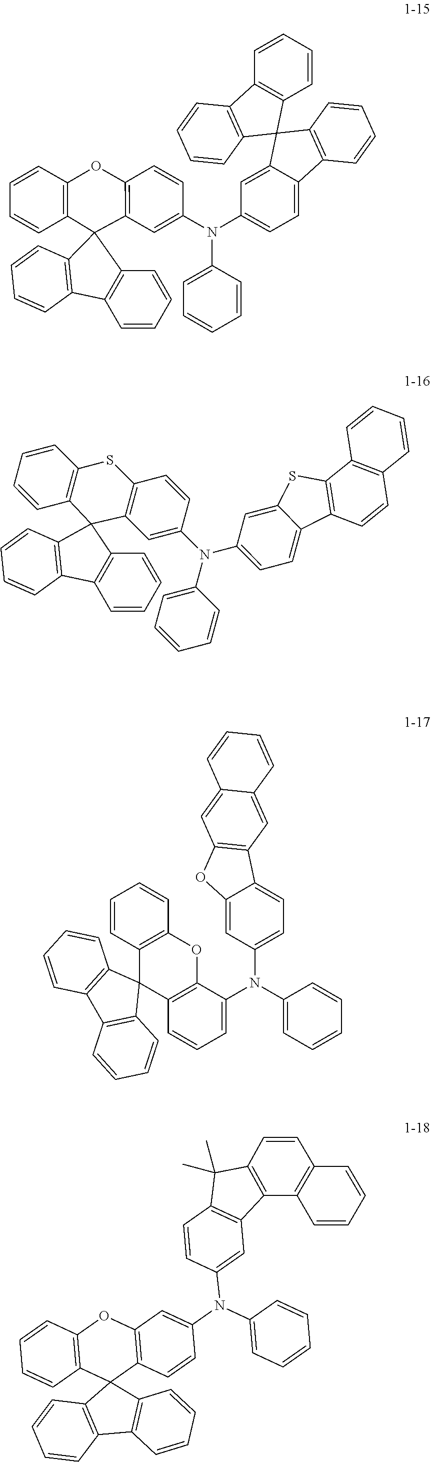 [SMILES: c1ccc(N(c2ccc3c(c2)C2(c4ccccc4O3)c3ccccc3-c3ccccc32)c2ccc3c(c2)C2(c4ccccc4-c4ccccc42)c2ccccc2-3)cc1 ; c1ccc(N(c2ccc3c(c2)C2(c4ccccc4S3)c3ccccc3-c3ccccc32)c2ccc3c(c2)sc2c4ccccc4ccc32)cc1 ; c1ccc(N(c2ccc3c(c2)oc2cc4ccccc4cc23)c2cccc3c2Oc2ccccc2C32c3ccccc3-c3ccccc32)cc1 ; CC1(C)c2ccc(N(c3ccccc3)c3ccc4c(c3)Oc3ccccc3C43c4ccccc4-c4ccccc43)cc2-c2c1ccc1ccccc21]

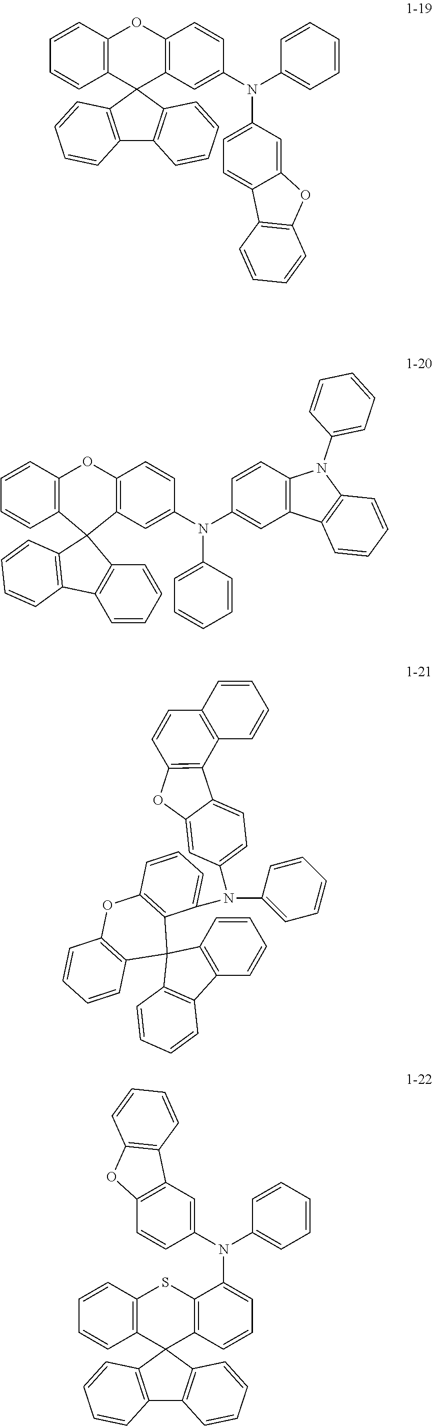

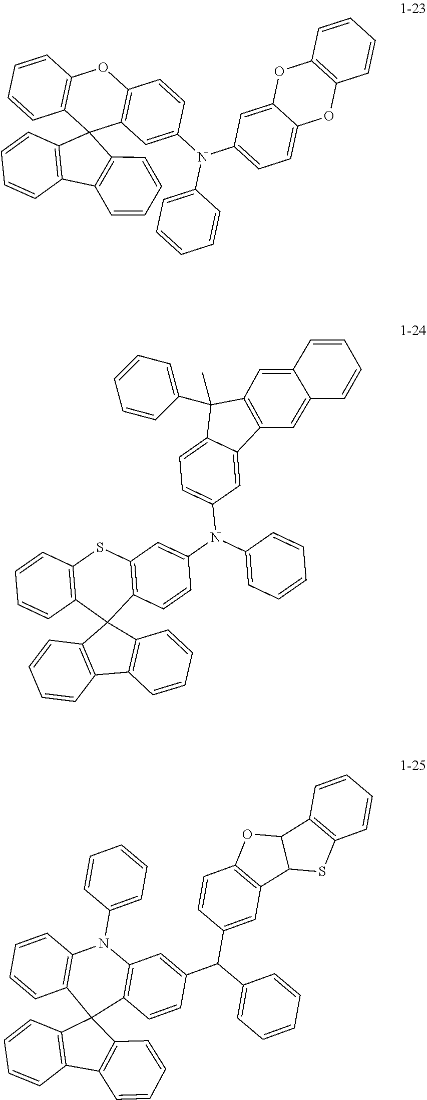 [SMILES: c1ccc(N(c2ccc3c(c2)Oc2ccccc2O3)c2ccc3c(c2)C2(c4ccccc4O3)c3ccccc3-c3ccccc32)cc1 ; CC1(c2ccccc2)c2ccc(N(c3ccccc3)c3ccc4c(c3)Sc3ccccc3C43c4ccccc4-c4ccccc43)cc2-c2cc3ccccc3cc21 ; c1ccc(C(c2ccc3c(c2)C2Sc4ccccc4C2O3)c2ccc3c(c2)N(c2ccccc2)c2ccccc2C32c3ccccc3-c3ccccc32)cc1]

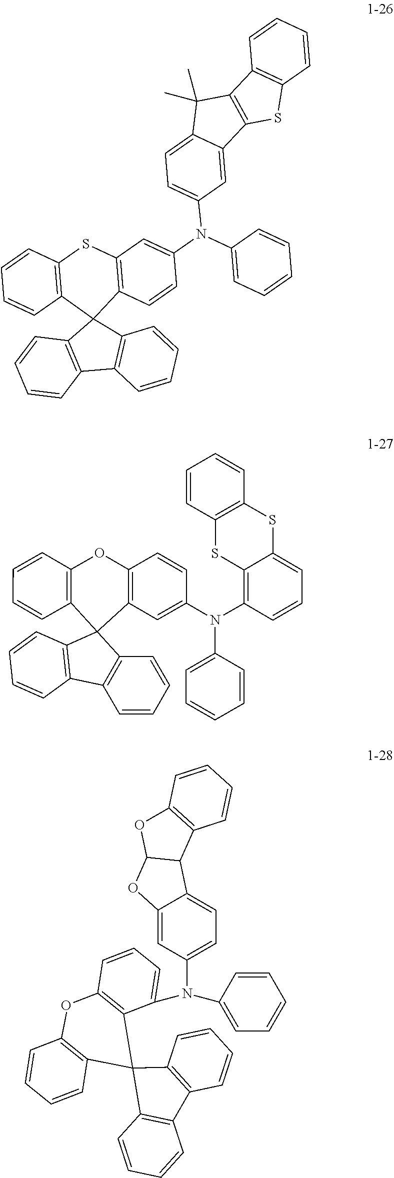

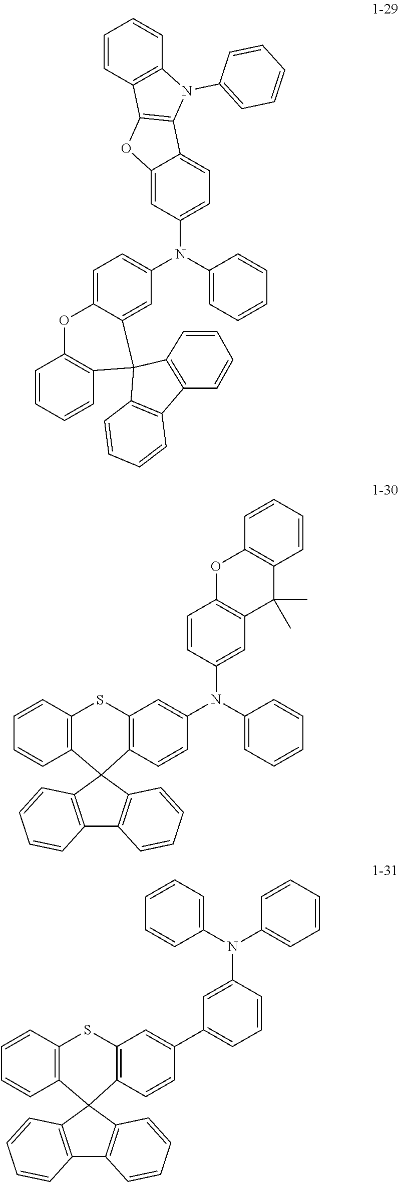

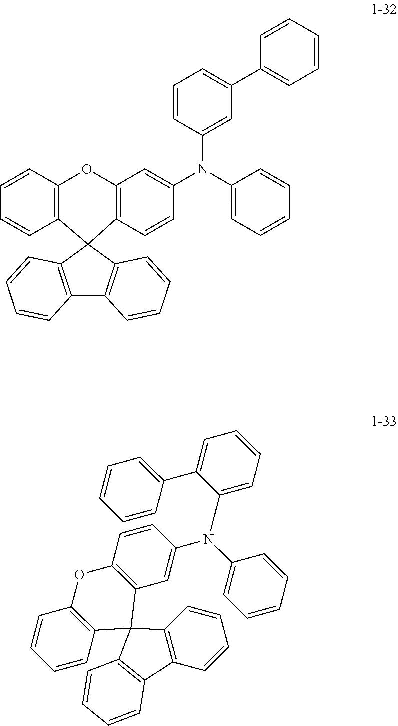 [SMILES: c1ccc(-c2cccc(N(c3ccccc3)c3ccc4c(c3)Oc3ccccc3C43c4ccccc4-c4ccccc43)c2)cc1 ; c1ccc(-c2ccccc2N(c2ccccc2)c2ccc3c(c2)C2(c4ccccc4O3)c3ccccc3-c3ccccc32)cc1]

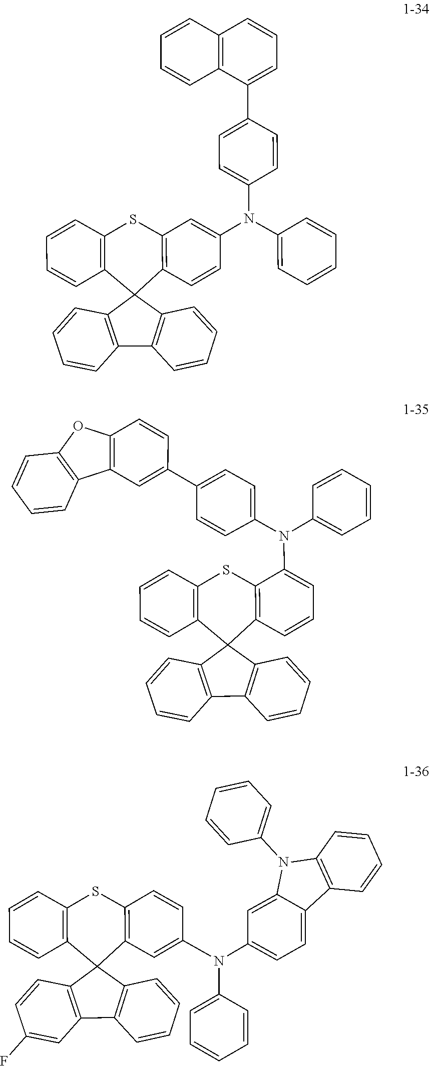 [SMILES: c1ccc(N(c2ccc(-c3cccc4ccccc34)cc2)c2ccc3c(c2)Sc2ccccc2C32c3ccccc3-c3ccccc32)cc1 ; c1ccc(N(c2ccc(-c3ccc4oc5ccccc5c4c3)cc2)c2cccc3c2Sc2ccccc2C32c3ccccc3-c3ccccc32)cc1 ; Fc1ccc2c(c1)-c1ccccc1C21c2ccccc2Sc2ccc(N(c3ccccc3)c3ccc4c5ccccc5n(-c5ccccc5)c4c3)cc21]

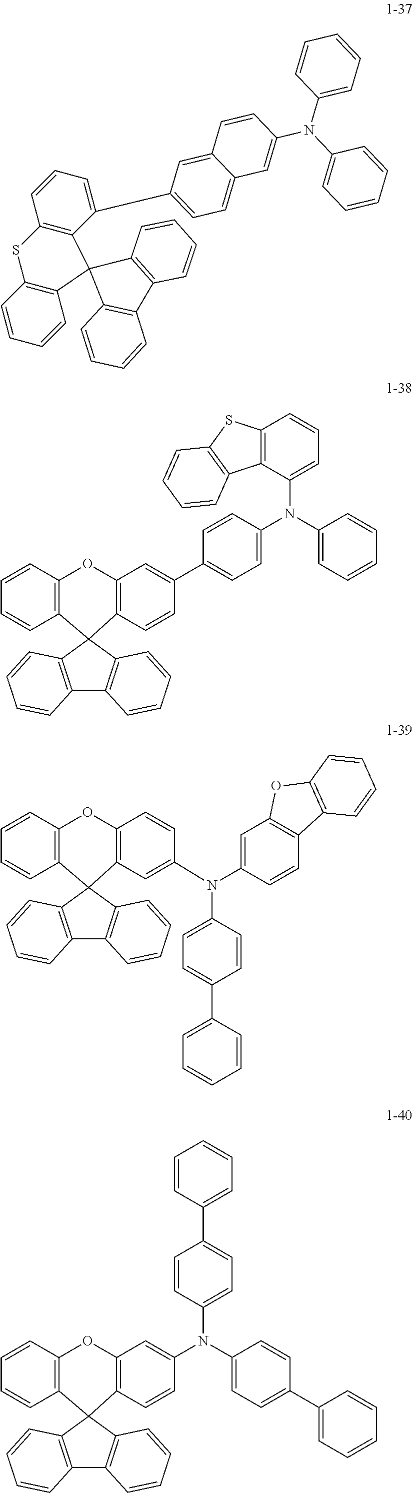 [SMILES: c1ccc(N(c2ccccc2)c2ccc3cc(-c4cccc5c4C4(c6ccccc6S5)c5ccccc5-c5ccccc54)ccc3c2)cc1 ; c1ccc(N(c2ccc(-c3ccc4c(c3)Oc3ccccc3C43c4ccccc4-c4ccccc43)cc2)c2cccc3sc4ccccc4c23)cc1 ; c1ccc(-c2ccc(N(c3ccc4c(c3)C3(c5ccccc5O4)c4ccccc4-c4ccccc43)c3ccc4c(c3)oc3ccccc34)cc2)cc1 ; c1ccc(-c2ccc(N(c3ccc(-c4ccccc4)cc3)c3ccc4c(c3)Oc3ccccc3C43c4ccccc4-c4ccccc43)cc2)cc1]

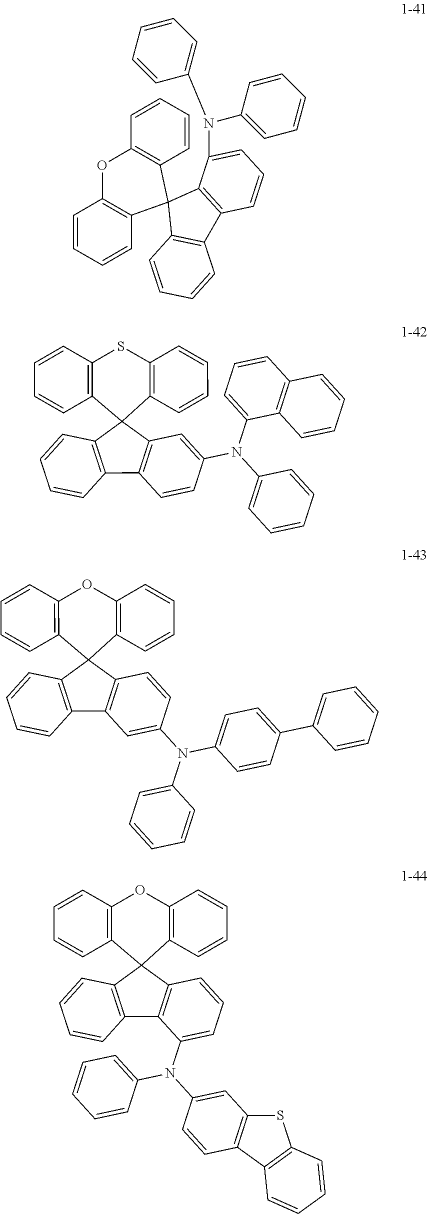

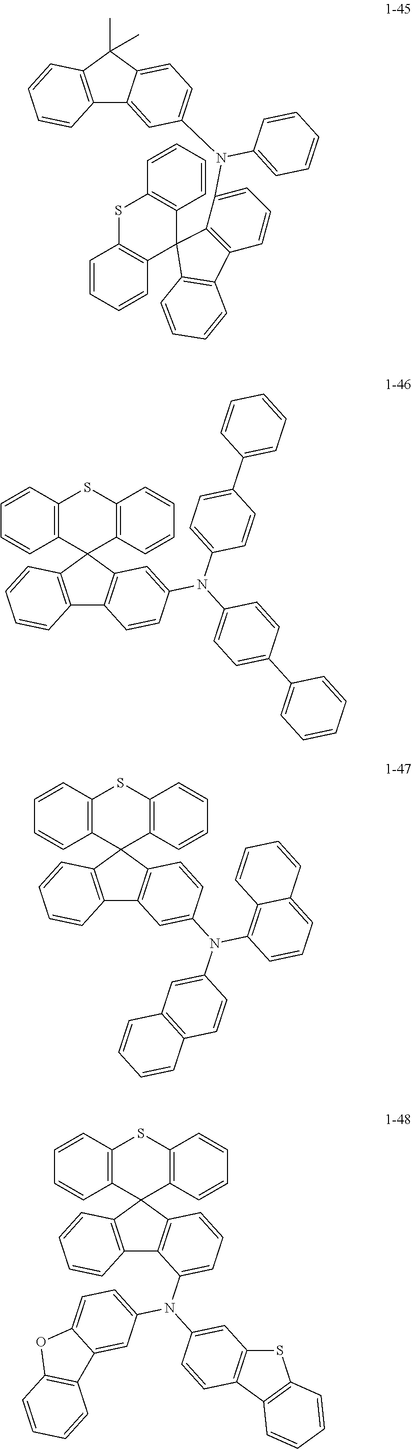 [SMILES: CC1(C)c2ccccc2-c2cc(N(c3ccccc3)c3cccc4c3C3(c5ccccc5Sc5ccccc53)c3ccccc3-4)ccc21 ; c1ccc(-c2ccc(N(c3ccc(-c4ccccc4)cc3)c3ccc4c(c3)C3(c5ccccc5Sc5ccccc53)c3ccccc3-4)cc2)cc1 ; c1ccc2c(c1)Sc1ccccc1C21c2ccccc2-c2cc(N(c3ccc4ccccc4c3)c3cccc4ccccc34)ccc21 ; c1ccc2c(c1)Sc1ccccc1C21c2ccccc2-c2c(N(c3ccc4c(c3)sc3ccccc34)c3ccc4oc5ccccc5c4c3)cccc21]

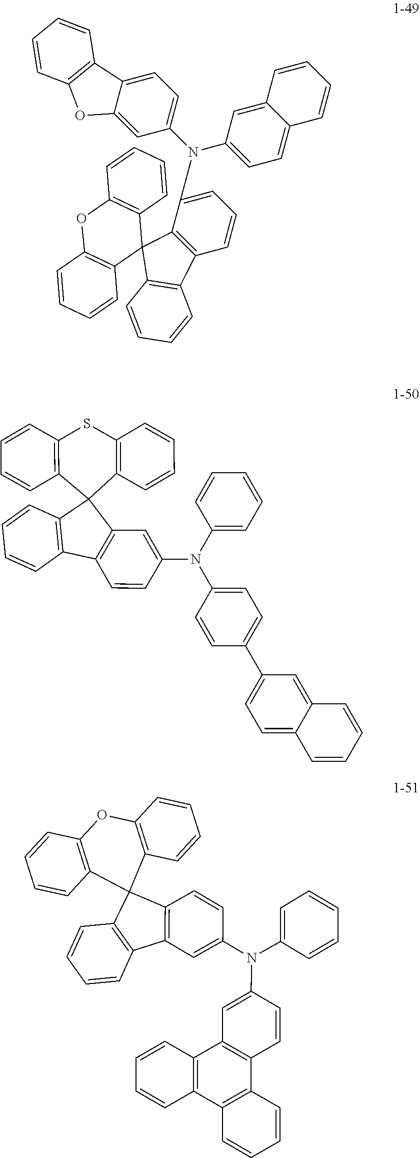 [SMILES: c1ccc2c(c1)Oc1ccccc1C21c2ccccc2-c2cccc(N(c3ccc4ccccc4c3)c3ccc4c(c3)oc3ccccc34)c21 ; c1ccc(N(c2ccc(-c3ccc4ccccc4c3)cc2)c2ccc3c(c2)C2(c4ccccc4Sc4ccccc42)c2ccccc2-3)cc1 ; c1ccc(N(c2ccc3c(c2)-c2ccccc2C32c3ccccc3Oc3ccccc32)c2ccc3c4ccccc4c4ccccc4c3c2)cc1]

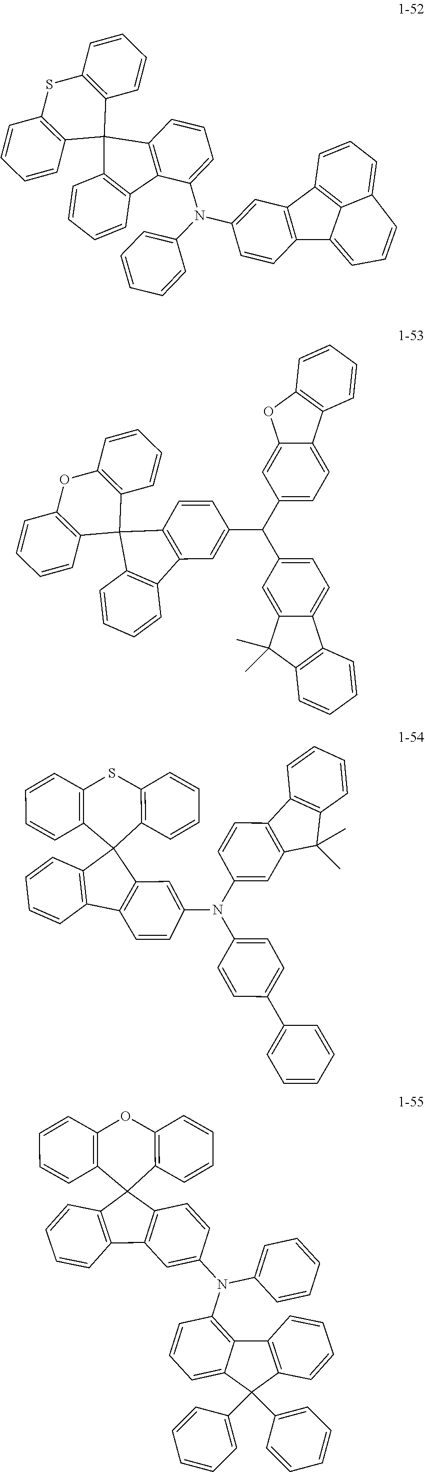

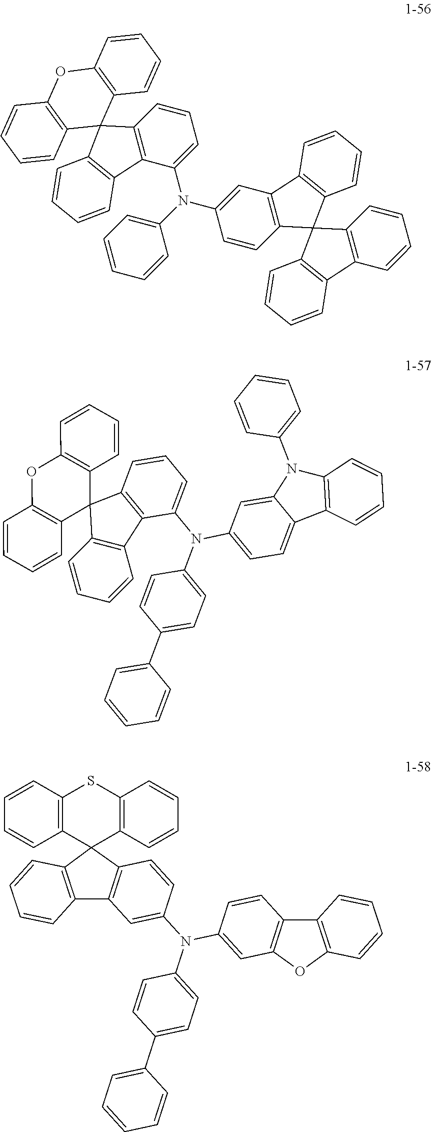 [SMILES: c1ccc(N(c2ccc3c(c2)-c2ccccc2C32c3ccccc3-c3ccccc32)c2cccc3c2-c2ccccc2C32c3ccccc3Oc3ccccc32)cc1 ; c1ccc(-c2ccc(N(c3ccc4c5ccccc5n(-c5ccccc5)c4c3)c3cccc4c3-c3ccccc3C43c4ccccc4Oc4ccccc43)cc2)cc1 ; c1ccc(-c2ccc(N(c3ccc4c(c3)-c3ccccc3C43c4ccccc4Sc4ccccc43)c3ccc4c(c3)oc3ccccc34)cc2)cc1]

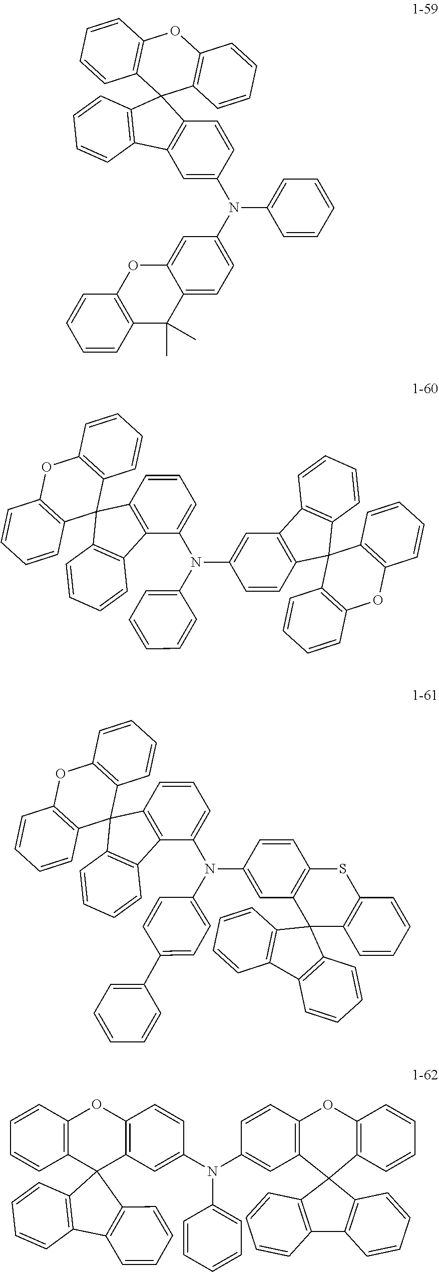 [SMILES: CC1(C)c2ccccc2Oc2cc(N(c3ccccc3)c3ccc4c(c3)-c3ccccc3C43c4ccccc4Oc4ccccc43)ccc21 ; c1ccc(N(c2ccc3c(c2)-c2ccccc2C32c3ccccc3Oc3ccccc32)c2cccc3c2-c2ccccc2C32c3ccccc3Oc3ccccc32)cc1 ; c1ccc(-c2ccc(N(c3ccc4c(c3)C3(c5ccccc5S4)c4ccccc4-c4ccccc43)c3cccc4c3-c3ccccc3C43c4ccccc4Oc4ccccc43)cc2)cc1 ; c1ccc(N(c2ccc3c(c2)C2(c4ccccc4O3)c3ccccc3-c3ccccc32)c2ccc3c(c2)C2(c4ccccc4O3)c3ccccc3-c3ccccc32)cc1]

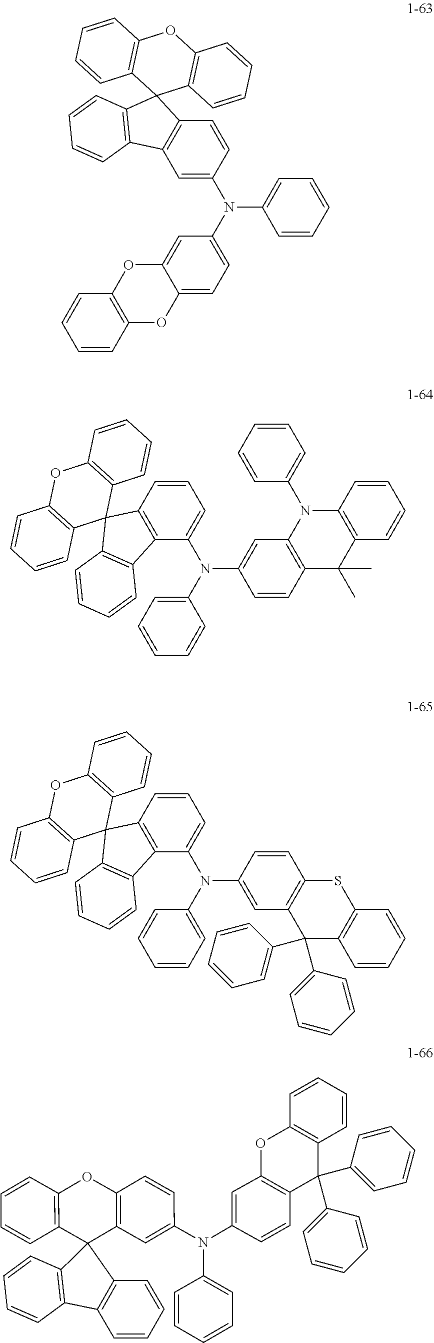

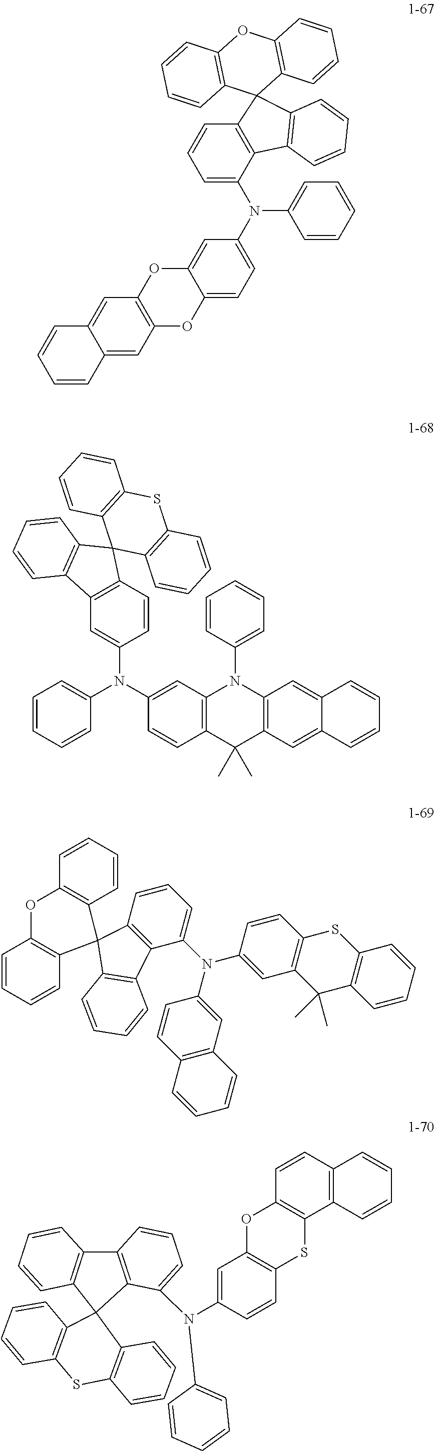 [SMILES: c1ccc(N(c2ccc3c(c2)Oc2cc4ccccc4cc2O3)c2cccc3c2-c2ccccc2C32c3ccccc3Oc3ccccc32)cc1 ; CC1(C)c2ccc(N(c3ccccc3)c3ccc4c(c3)-c3ccccc3C43c4ccccc4Sc4ccccc43)cc2N(c2ccccc2)c2cc3ccccc3cc21 ; CC1(C)c2ccccc2Sc2ccc(N(c3ccc4ccccc4c3)c3cccc4c3-c3ccccc3C43c4ccccc4Oc4ccccc43)cc21 ; c1ccc(N(c2ccc3c(c2)Oc2ccc4ccccc4c2S3)c2cccc3c2C2(c4ccccc4Sc4ccccc42)c2ccccc2-3)cc1]

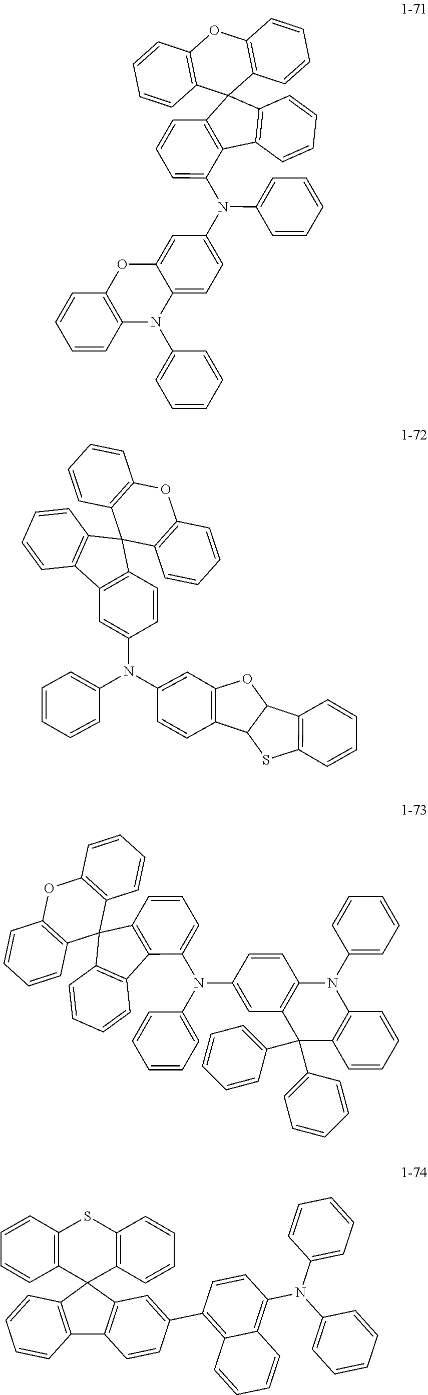 [SMILES: c1ccc(N(c2ccc3c(c2)Oc2ccccc2N3c2ccccc2)c2cccc3c2-c2ccccc2C32c3ccccc3Oc3ccccc32)cc1 ; c1ccc(N(c2ccc3c(c2)OC2c4ccccc4SC32)c2ccc3c(c2)-c2ccccc2C32c3ccccc3Oc3ccccc32)cc1 ; c1ccc(N(c2ccc3c(c2)C(c2ccccc2)(c2ccccc2)c2ccccc2N3c2ccccc2)c2cccc3c2-c2ccccc2C32c3ccccc3Oc3ccccc32)cc1 ; c1ccc(N(c2ccccc2)c2ccc(-c3ccc4c(c3)C3(c5ccccc5Sc5ccccc53)c3ccccc3-4)c3ccccc23)cc1]

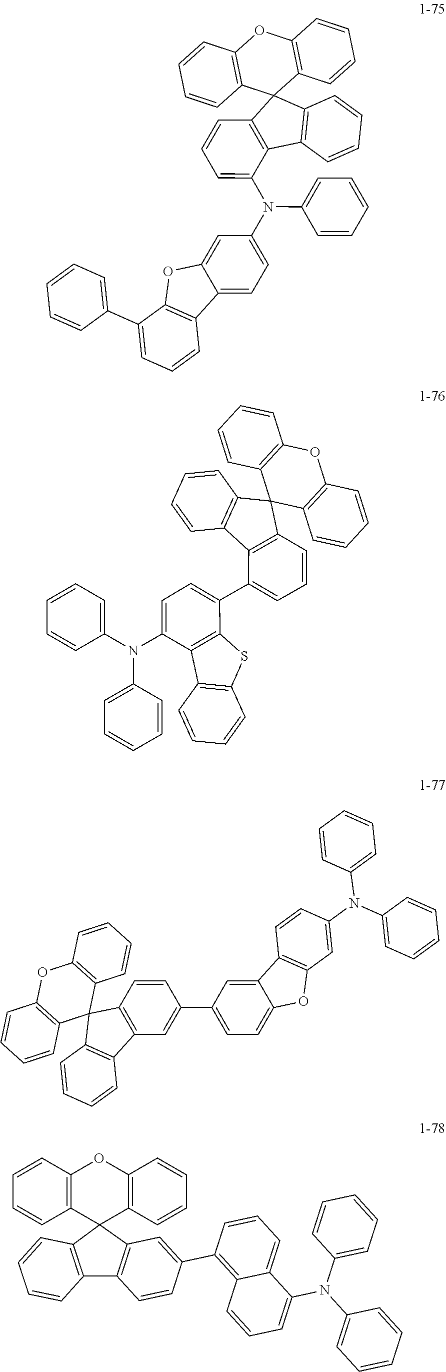 [SMILES: c1ccc(-c2cccc3c2oc2cc(N(c4ccccc4)c4cccc5c4-c4ccccc4C54c5ccccc5Oc5ccccc54)ccc23)cc1 ; c1ccc(N(c2ccccc2)c2ccc(-c3cccc4c3-c3ccccc3C43c4ccccc4Oc4ccccc43)c3sc4ccccc4c23)cc1 ; c1ccc(N(c2ccccc2)c2ccc3c(c2)oc2ccc(-c4ccc5c(c4)-c4ccccc4C54c5ccccc5Oc5ccccc54)cc23)cc1 ; c1ccc(N(c2ccccc2)c2cccc3c(-c4ccc5c(c4)C4(c6ccccc6Oc6ccccc64)c4ccccc4-5)cccc23)cc1]

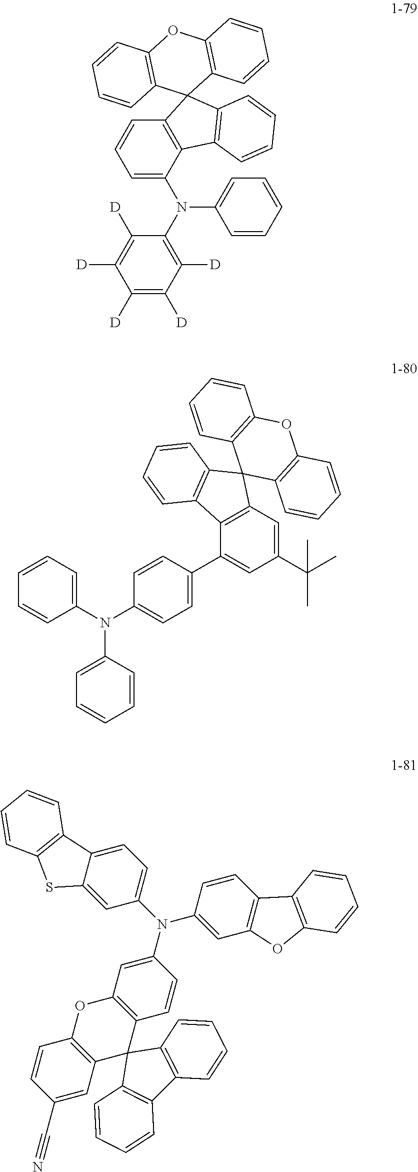 [SMILES: [2H]c1c([2H])c([2H])c(N(c2ccccc2)c2cccc3c2-c2ccccc2C32c3ccccc3Oc3ccccc32)c([2H])c1[2H] ; CC(C)(C)c1cc(-c2ccc(N(c3ccccc3)c3ccccc3)cc2)c2c(c1)C1(c3ccccc3Oc3ccccc31)c1ccccc1-2 ; N#Cc1ccc2c(c1)C1(c3ccc(N(c4ccc5c(c4)oc4ccccc45)c4ccc5c(c4)sc4ccccc45)cc3O2)c2ccccc2-c2ccccc21]

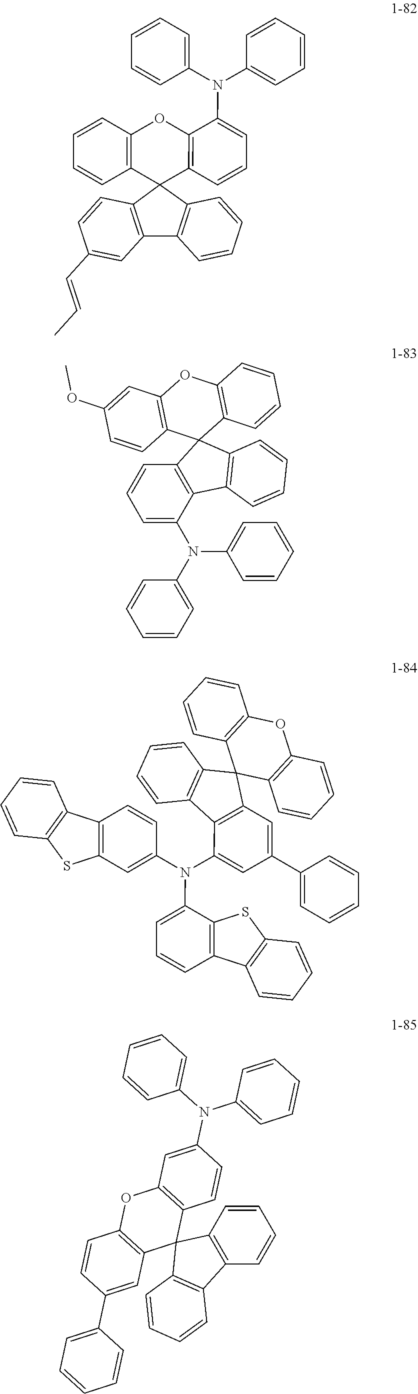

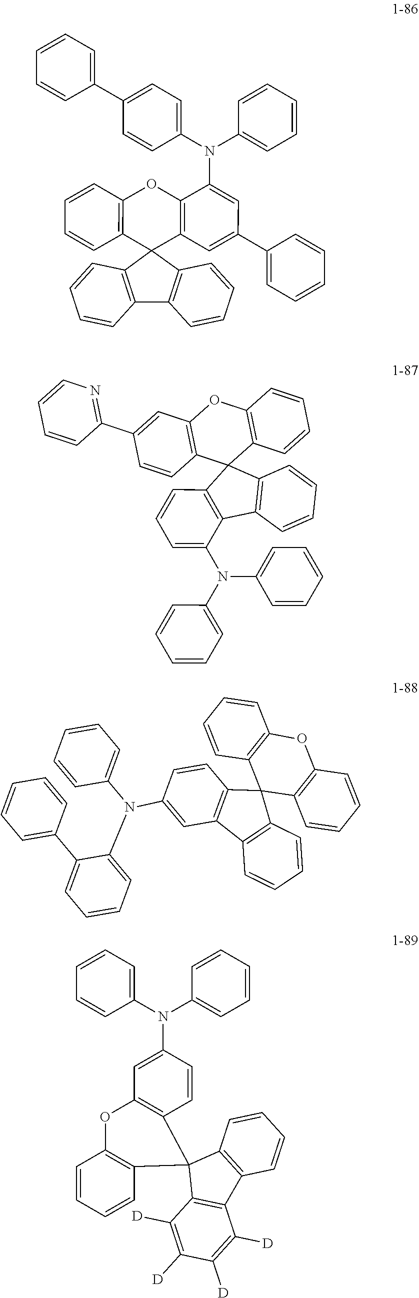

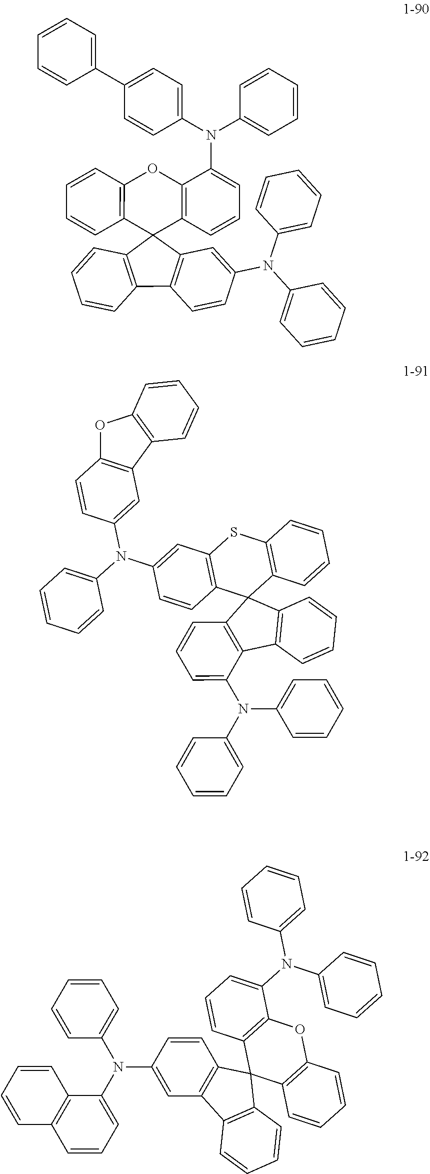 [SMILES: c1ccc(-c2ccc(N(c3ccccc3)c3cccc4c3Oc3ccccc3C43c4ccccc4-c4ccc(N(c5ccccc5)c5ccccc5)cc43)cc2)cc1 ; c1ccc(N(c2ccc3c(c2)Sc2ccccc2C32c3ccccc3-c3c(N(c4ccccc4)c4ccccc4)cccc32)c2ccc3oc4ccccc4c3c2)cc1 ; c1ccc(N(c2ccccc2)c2cccc3c2Oc2ccccc2C32c3ccccc3-c3cc(N(c4ccccc4)c4cccc5ccccc45)ccc32)cc1]

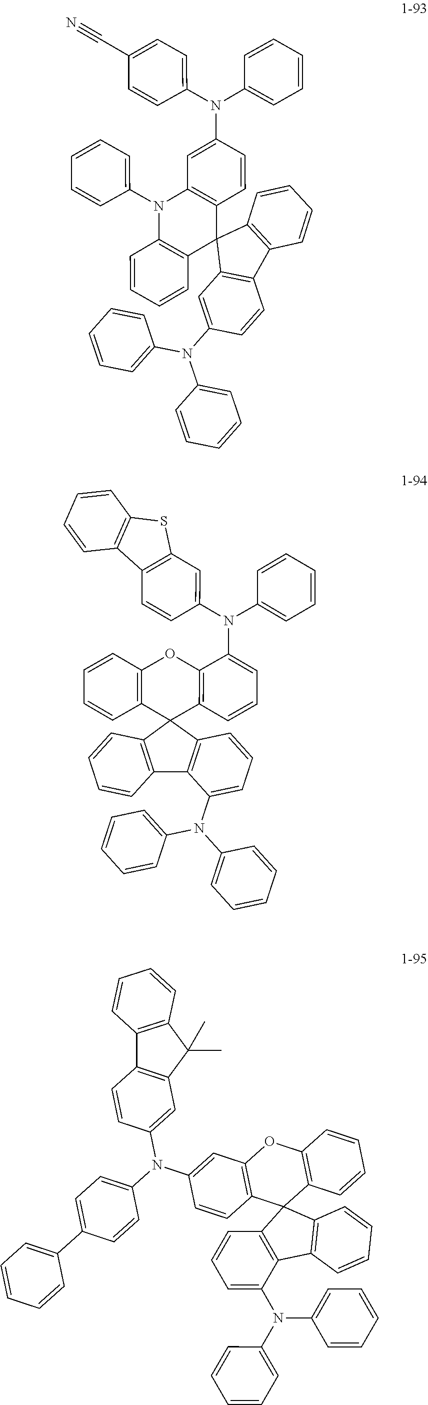 [SMILES: N#Cc1ccc(N(c2ccccc2)c2ccc3c(c2)N(c2ccccc2)c2ccccc2C32c3ccccc3-c3ccc(N(c4ccccc4)c4ccccc4)cc32)cc1 ; c1ccc(N(c2ccc3c(c2)sc2ccccc23)c2cccc3c2Oc2ccccc2C32c3ccccc3-c3c(N(c4ccccc4)c4ccccc4)cccc32)cc1 ; CC1(C)c2ccccc2-c2ccc(N(c3ccc(-c4ccccc4)cc3)c3ccc4c(c3)Oc3ccccc3C43c4ccccc4-c4c(N(c5ccccc5)c5ccccc5)cccc43)cc21]

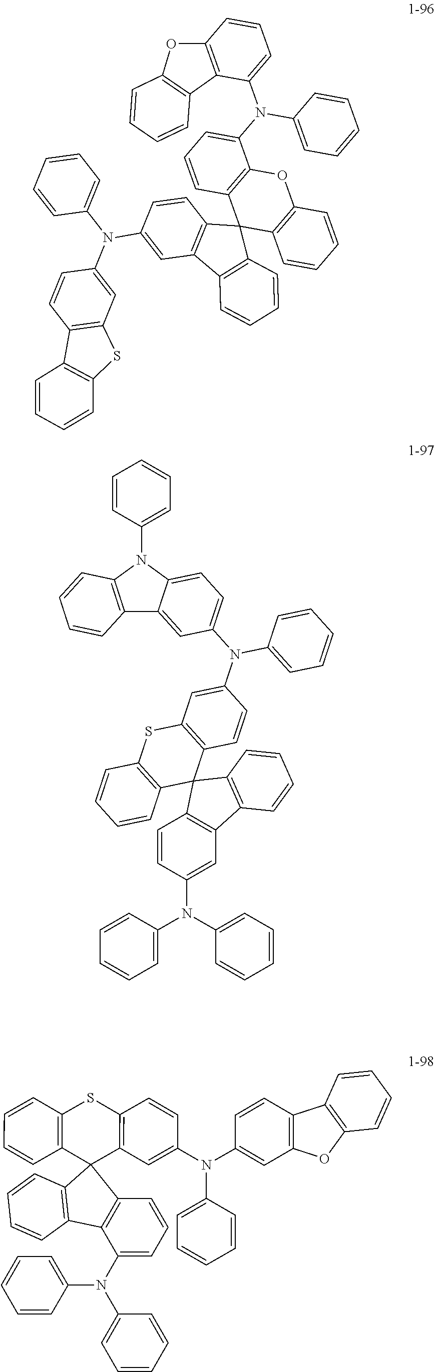 [SMILES: c1ccc(N(c2ccc3c(c2)-c2ccccc2C32c3ccccc3Oc3c(N(c4ccccc4)c4cccc5oc6ccccc6c45)cccc32)c2ccc3c(c2)sc2ccccc23)cc1 ; c1ccc(N(c2ccccc2)c2ccc3c(c2)-c2ccccc2C32c3ccccc3Sc3cc(N(c4ccccc4)c4ccc5c(c4)c4ccccc4n5-c4ccccc4)ccc32)cc1 ; c1ccc(N(c2ccc3c(c2)C2(c4ccccc4S3)c3ccccc3-c3c(N(c4ccccc4)c4ccccc4)cccc32)c2ccc3c(c2)oc2ccccc23)cc1]

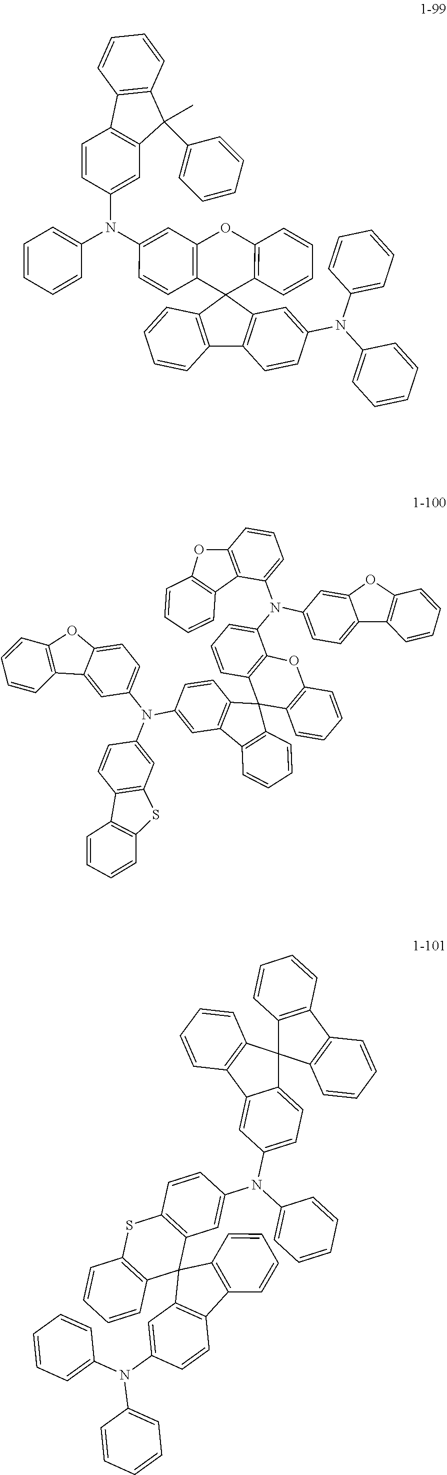 [SMILES: CC1(c2ccccc2)c2ccccc2-c2ccc(N(c3ccccc3)c3ccc4c(c3)Oc3ccccc3C43c4ccccc4-c4ccc(N(c5ccccc5)c5ccccc5)cc43)cc21 ; c1ccc2c(c1)Oc1c(N(c3ccc4c(c3)oc3ccccc34)c3cccc4oc5ccccc5c34)cccc1C21c2ccccc2-c2cc(N(c3ccc4c(c3)sc3ccccc34)c3ccc4oc5ccccc5c4c3)ccc21 ; c1ccc(N(c2ccc3c(c2)-c2ccccc2C32c3ccccc3-c3ccccc32)c2ccc3c(c2)C2(c4ccccc4S3)c3ccccc3-c3ccc(N(c4ccccc4)c4ccccc4)cc32)cc1]

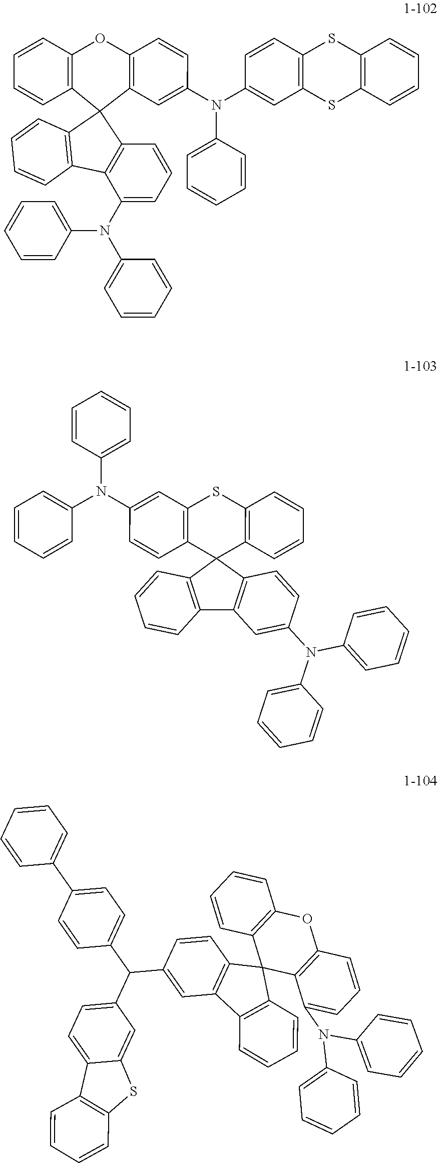 [SMILES: c1ccc(N(c2ccc3c(c2)Sc2ccccc2S3)c2ccc3c(c2)C2(c4ccccc4O3)c3ccccc3-c3c(N(c4ccccc4)c4ccccc4)cccc32)cc1 ; c1ccc(N(c2ccccc2)c2ccc3c(c2)Sc2ccccc2C32c3ccccc3-c3cc(N(c4ccccc4)c4ccccc4)ccc32)cc1 ; c1ccc(-c2ccc(C(c3ccc4c(c3)-c3ccccc3C43c4ccccc4Oc4cccc(N(c5ccccc5)c5ccccc5)c43)c3ccc4c(c3)sc3ccccc34)cc2)cc1]

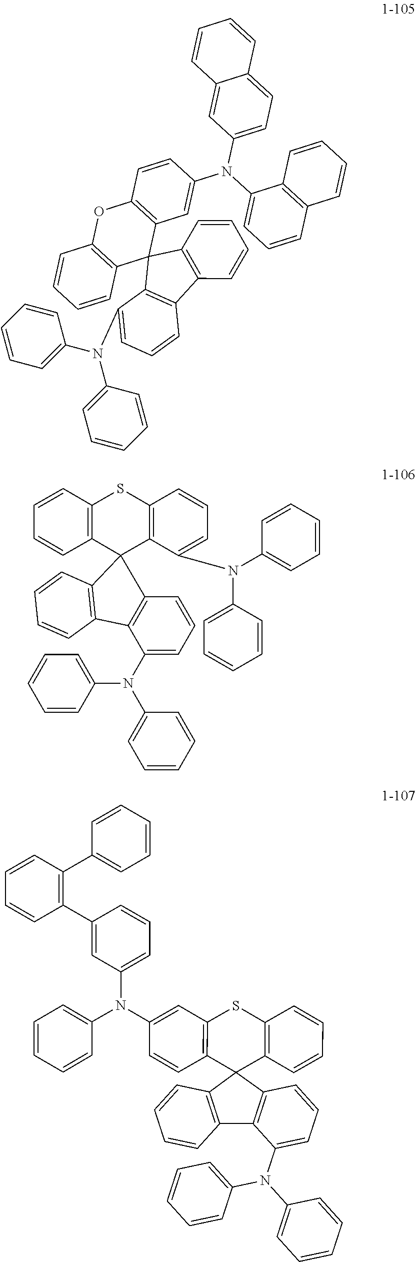

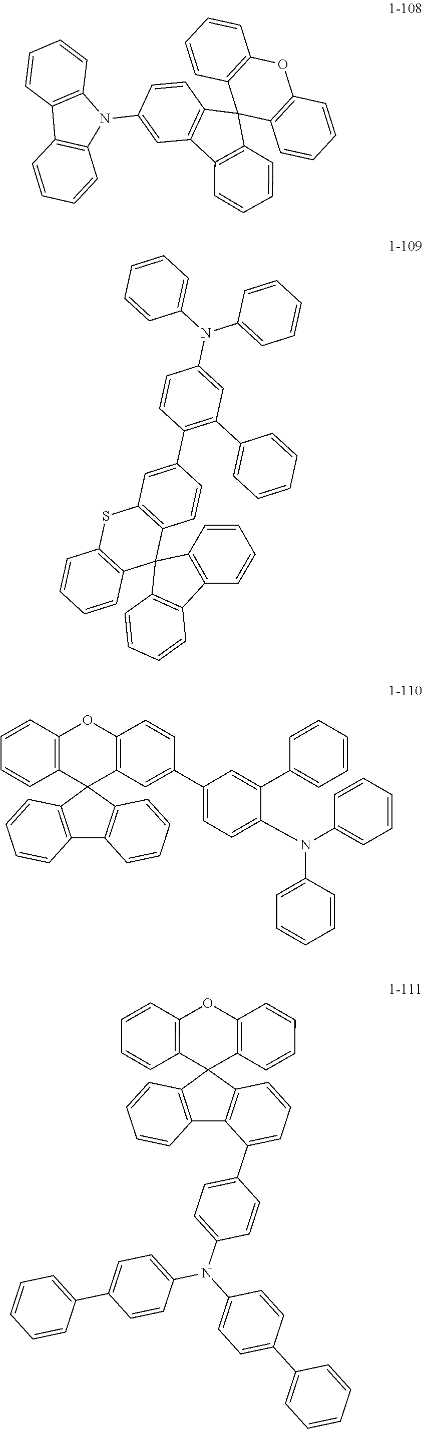 [SMILES: c1ccc2c(c1)Oc1ccccc1C21c2ccccc2-c2cc(-n3c4ccccc4c4ccccc43)ccc21 ; c1ccc(-c2cc(N(c3ccccc3)c3ccccc3)ccc2-c2ccc3c(c2)Sc2ccccc2C32c3ccccc3-c3ccccc32)cc1 ; c1ccc(-c2cc(-c3ccc4c(c3)C3(c5ccccc5O4)c4ccccc4-c4ccccc43)ccc2N(c2ccccc2)c2ccccc2)cc1 ; c1ccc(-c2ccc(N(c3ccc(-c4ccccc4)cc3)c3ccc(-c4cccc5c4-c4ccccc4C54c5ccccc5Oc5ccccc54)cc3)cc2)cc1]

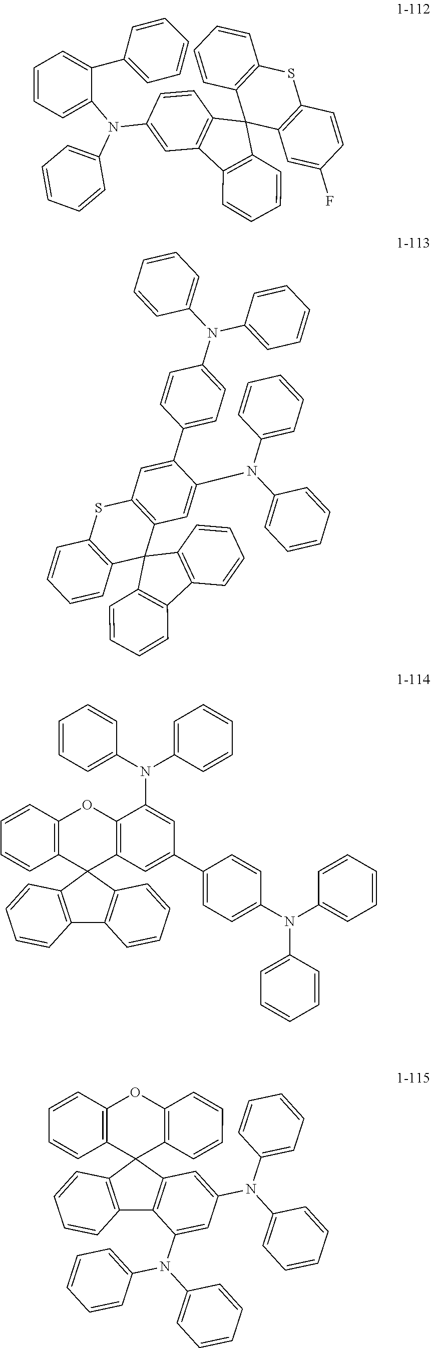 [SMILES: Fc1ccc2c(c1)C1(c3ccccc3S2)c2ccccc2-c2cc(N(c3ccccc3)c3ccccc3-c3ccccc3)ccc21 ; c1ccc(N(c2ccccc2)c2ccc(-c3cc4c(cc3N(c3ccccc3)c3ccccc3)C3(c5ccccc5S4)c4ccccc4-c4ccccc43)cc2)cc1 ; c1ccc(N(c2ccccc2)c2ccc(-c3cc(N(c4ccccc4)c4ccccc4)c4c(c3)C3(c5ccccc5O4)c4ccccc4-c4ccccc43)cc2)cc1 ; c1ccc(N(c2ccccc2)c2cc(N(c3ccccc3)c3ccccc3)c3c(c2)C2(c4ccccc4Oc4ccccc42)c2ccccc2-3)cc1]

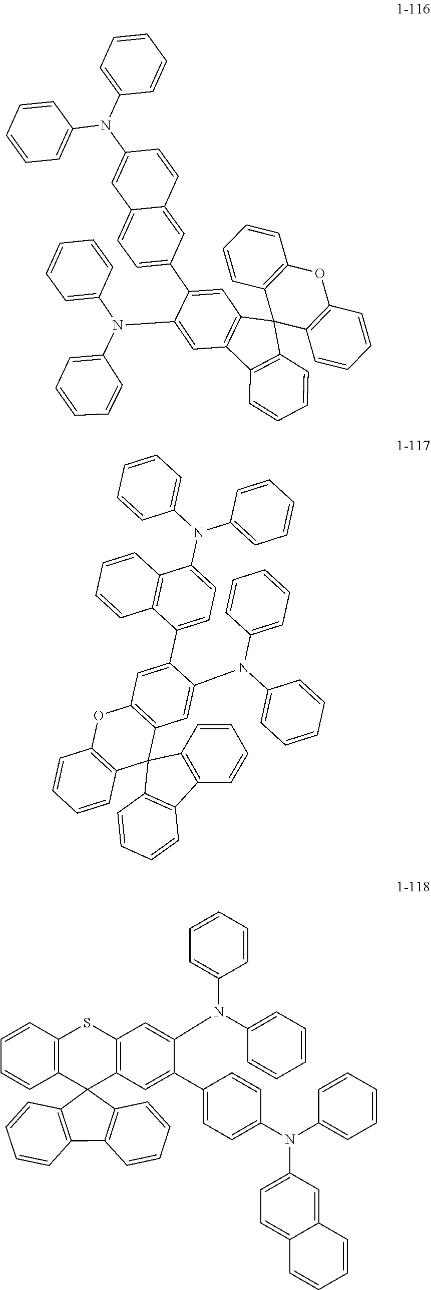 [SMILES: c1ccc(N(c2ccccc2)c2ccc3cc(-c4cc5c(cc4N(c4ccccc4)c4ccccc4)-c4ccccc4C54c5ccccc5Oc5ccccc54)ccc3c2)cc1 ; c1ccc(N(c2ccccc2)c2cc3c(cc2-c2ccc(N(c4ccccc4)c4ccccc4)c4ccccc24)Oc2ccccc2C32c3ccccc3-c3ccccc32)cc1 ; c1ccc(N(c2ccc(-c3cc4c(cc3N(c3ccccc3)c3ccccc3)Sc3ccccc3C43c4ccccc4-c4ccccc43)cc2)c2ccc3ccccc3c2)cc1]

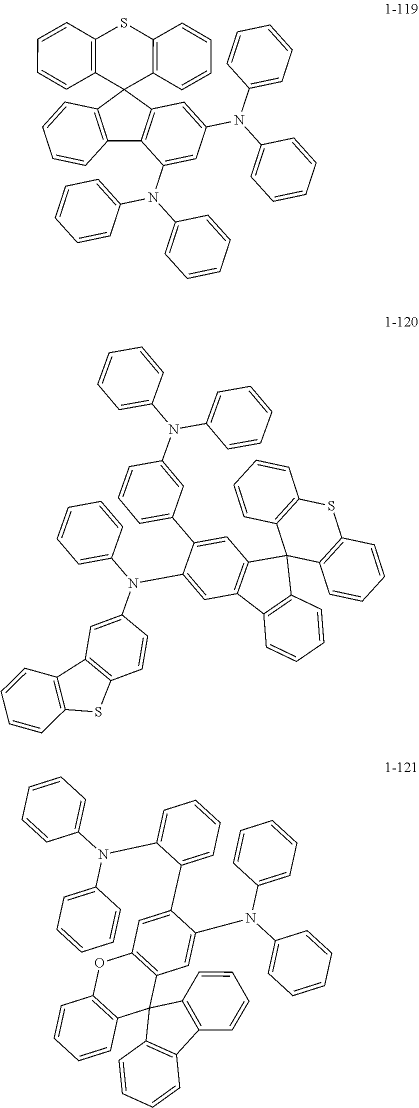 [SMILES: c1ccc(N(c2ccccc2)c2cc(N(c3ccccc3)c3ccccc3)c3c(c2)C2(c4ccccc4Sc4ccccc42)c2ccccc2-3)cc1 ; c1ccc(N(c2ccccc2)c2cccc(-c3cc4c(cc3N(c3ccccc3)c3ccc5sc6ccccc6c5c3)-c3ccccc3C43c4ccccc4Sc4ccccc43)c2)cc1 ; c1ccc(N(c2ccccc2)c2ccccc2-c2cc3c(cc2N(c2ccccc2)c2ccccc2)C2(c4ccccc4O3)c3ccccc3-c3ccccc32)cc1]

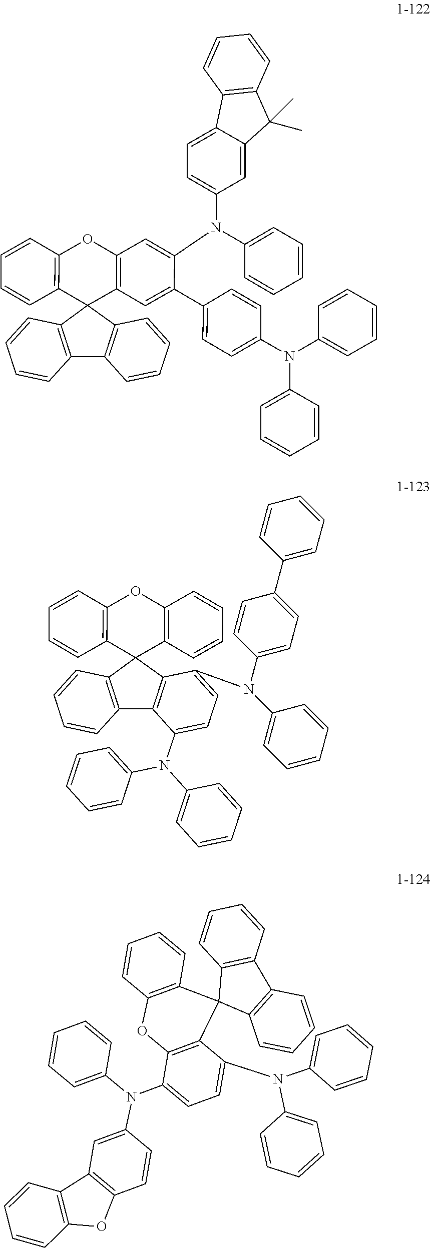

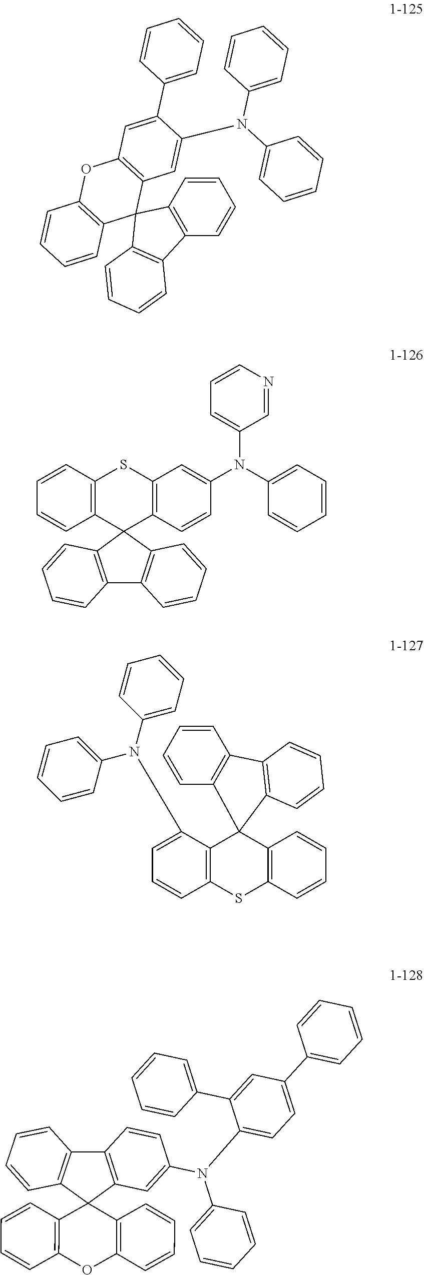 [SMILES: c1ccc(-c2cc3c(cc2N(c2ccccc2)c2ccccc2)C2(c4ccccc4O3)c3ccccc3-c3ccccc32)cc1 ; c1ccc(N(c2cccnc2)c2ccc3c(c2)Sc2ccccc2C32c3ccccc3-c3ccccc32)cc1 ; c1ccc(N(c2ccccc2)c2cccc3c2C2(c4ccccc4S3)c3ccccc3-c3ccccc32)cc1 ; c1ccc(-c2ccc(N(c3ccccc3)c3ccc4c(c3)C3(c5ccccc5Oc5ccccc53)c3ccccc3-4)c(-c3ccccc3)c2)cc1]

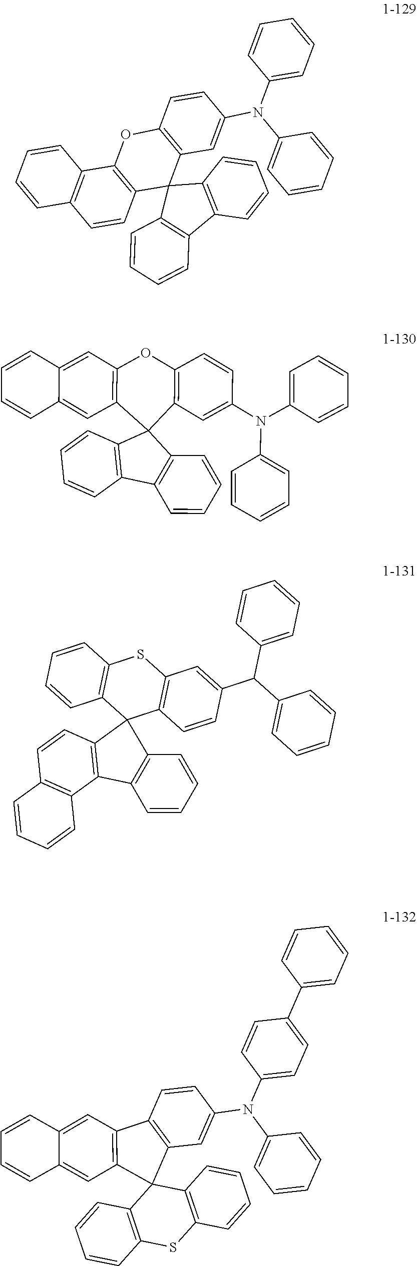 [SMILES: c1ccc(N(c2ccccc2)c2ccc3c(c2)C2(c4ccccc4-c4ccccc42)c2ccc4ccccc4c2O3)cc1 ; c1ccc(N(c2ccccc2)c2ccc3c(c2)C2(c4cc5ccccc5cc4O3)c3ccccc3-c3ccccc32)cc1 ; c1ccc(C(c2ccccc2)c2ccc3c(c2)Sc2ccccc2C32c3ccccc3-c3c2ccc2ccccc32)cc1 ; c1ccc(-c2ccc(N(c3ccccc3)c3ccc4c(c3)C3(c5ccccc5Sc5ccccc53)c3cc5ccccc5cc3-4)cc2)cc1]

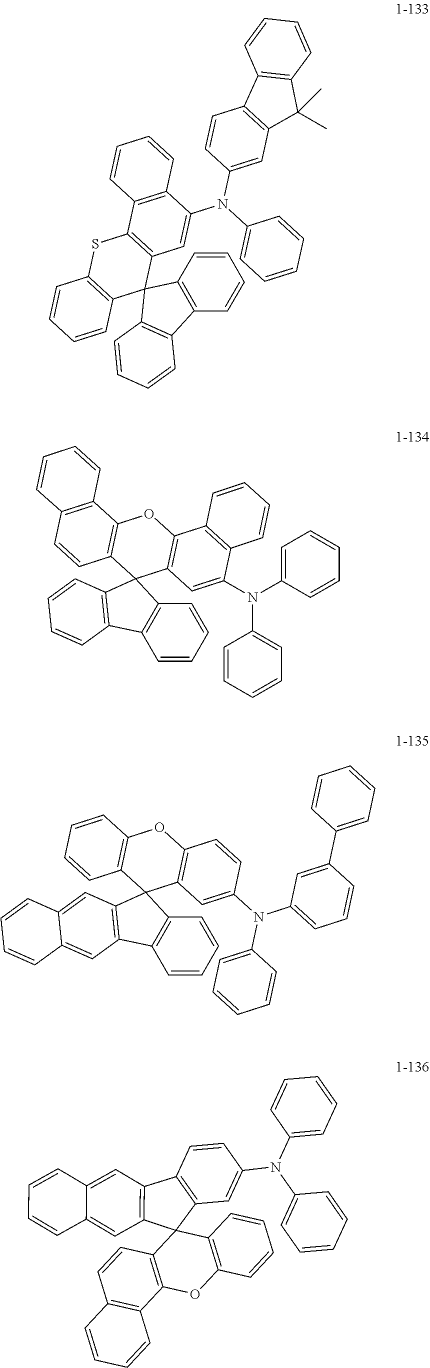 [SMILES: CC1(C)c2ccccc2-c2ccc(N(c3ccccc3)c3cc4c(c5ccccc35)Sc3ccccc3C43c4ccccc4-c4ccccc43)cc21 ; c1ccc(N(c2ccccc2)c2cc3c(c4ccccc24)Oc2c(ccc4ccccc24)C32c3ccccc3-c3ccccc32)cc1 ; c1ccc(-c2cccc(N(c3ccccc3)c3ccc4c(c3)C3(c5ccccc5O4)c4ccccc4-c4cc5ccccc5cc43)c2)cc1 ; c1ccc(N(c2ccccc2)c2ccc3c(c2)C2(c4ccccc4Oc4c2ccc2ccccc42)c2cc4ccccc4cc2-3)cc1]

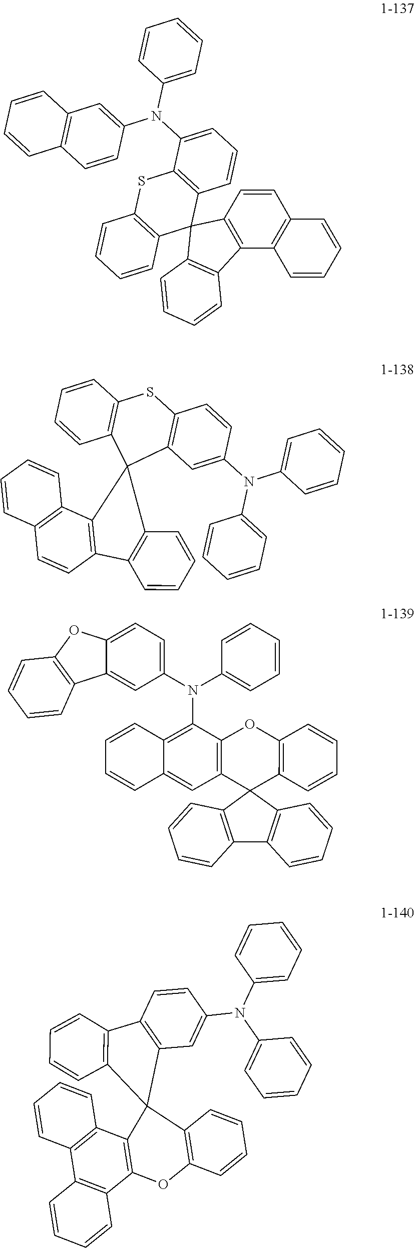

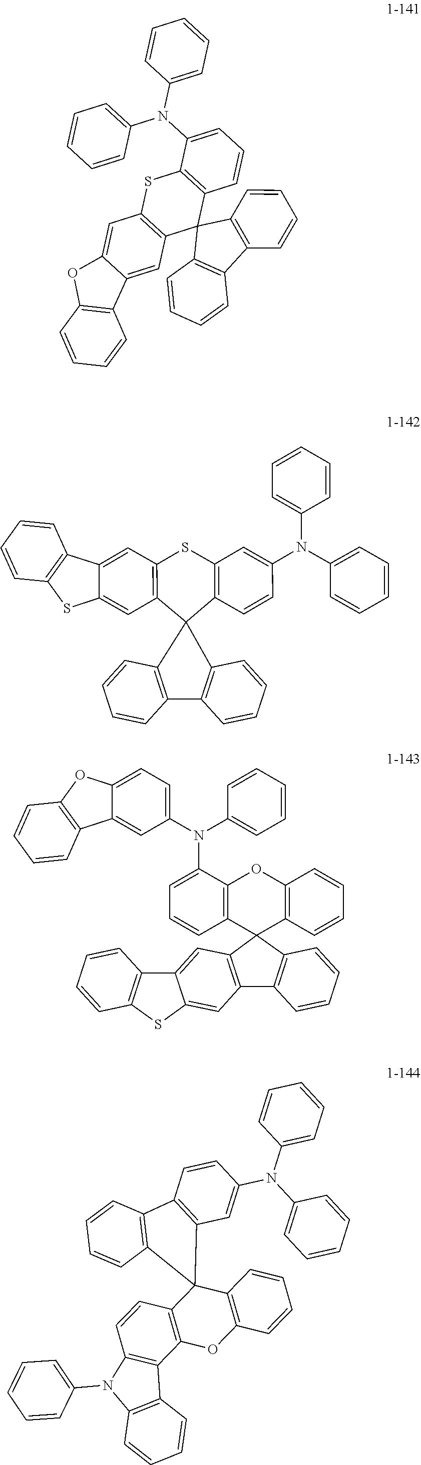

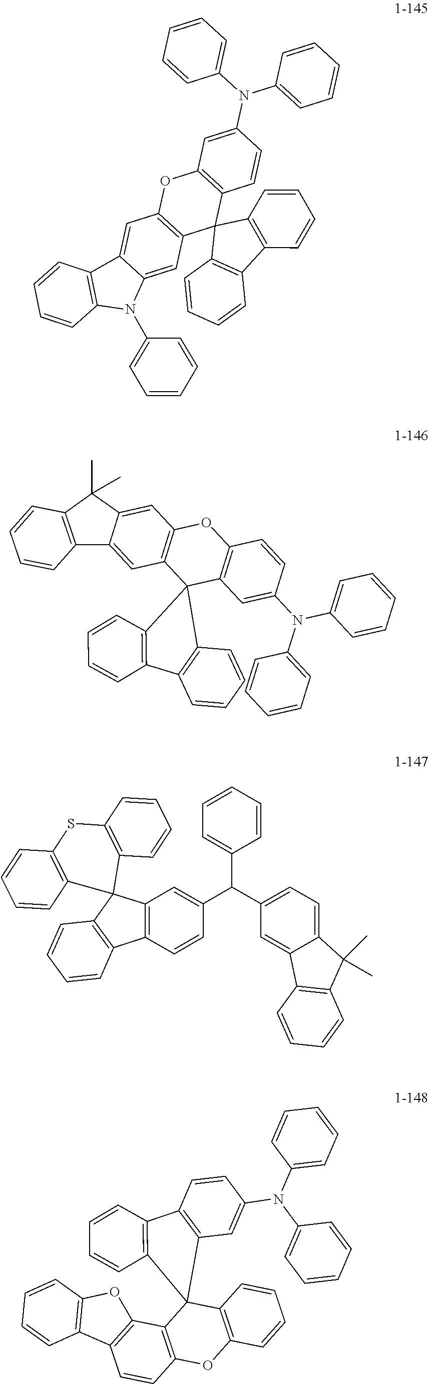 [SMILES: c1ccc(N(c2ccccc2)c2ccc3c(c2)Oc2cc4c5ccccc5n(-c5ccccc5)c4cc2C32c3ccccc3-c3ccccc32)cc1 ; CC1(C)c2ccccc2-c2cc3c(cc21)Oc1ccc(N(c2ccccc2)c2ccccc2)cc1C31c2ccccc2-c2ccccc21 ; CC1(C)c2ccccc2-c2cc(C(c3ccccc3)c3ccc4c(c3)C3(c5ccccc5Sc5ccccc53)c3ccccc3-4)ccc21 ; c1ccc(N(c2ccccc2)c2ccc3c(c2)C2(c4ccccc4Oc4ccc5c(oc6ccccc65)c42)c2ccccc2-3)cc1]

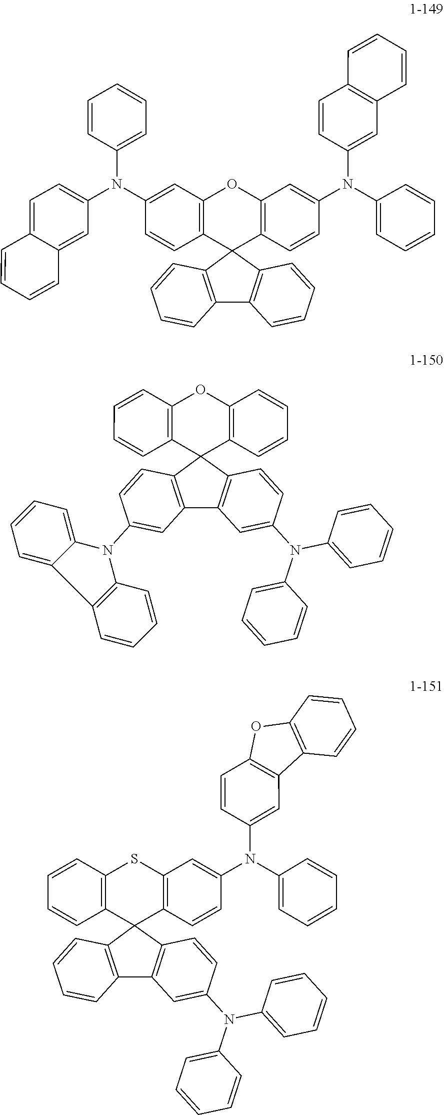 [SMILES: c1ccc(N(c2ccc3c(c2)Oc2cc(N(c4ccccc4)c4ccc5ccccc5c4)ccc2C32c3ccccc3-c3ccccc32)c2ccc3ccccc3c2)cc1 ; c1ccc(N(c2ccccc2)c2ccc3c(c2)-c2cc(-n4c5ccccc5c5ccccc54)ccc2C32c3ccccc3Oc3ccccc32)cc1 ; c1ccc(N(c2ccccc2)c2ccc3c(c2)-c2ccccc2C32c3ccccc3Sc3cc(N(c4ccccc4)c4ccc5oc6ccccc6c5c4)ccc32)cc1]

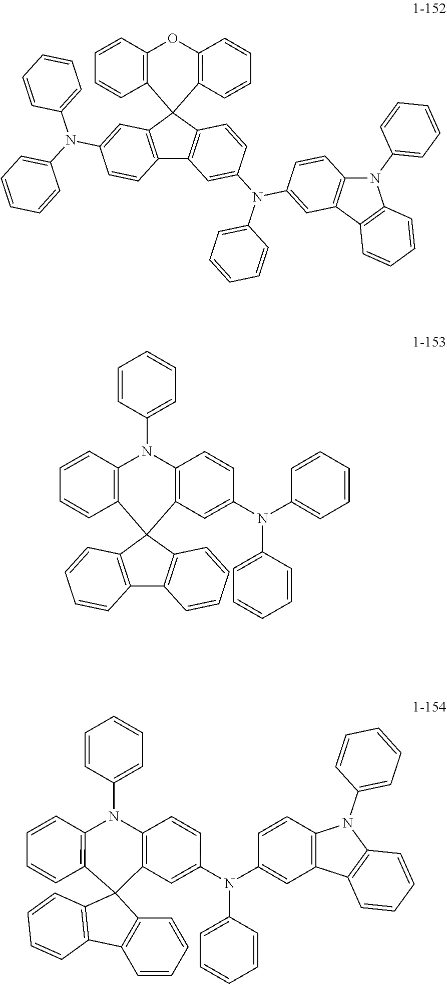

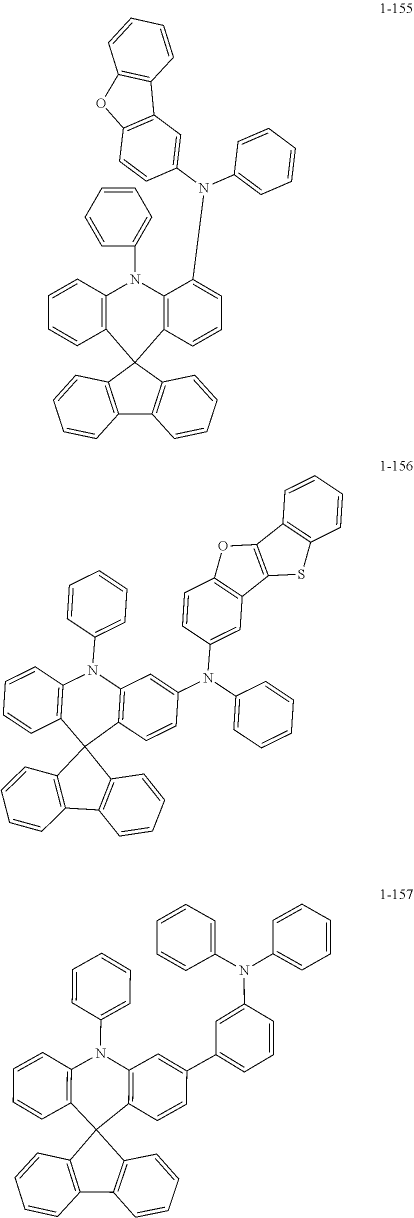 [SMILES: c1ccc(N(c2ccc3oc4ccccc4c3c2)c2cccc3c2N(c2ccccc2)c2ccccc2C32c3ccccc3-c3ccccc32)cc1 ; c1ccc(N(c2ccc3c(c2)N(c2ccccc2)c2ccccc2C32c3ccccc3-c3ccccc32)c2ccc3oc4c5ccccc5sc4c3c2)cc1 ; c1ccc(N(c2ccccc2)c2cccc(-c3ccc4c(c3)N(c3ccccc3)c3ccccc3C43c4ccccc4-c4ccccc43)c2)cc1]

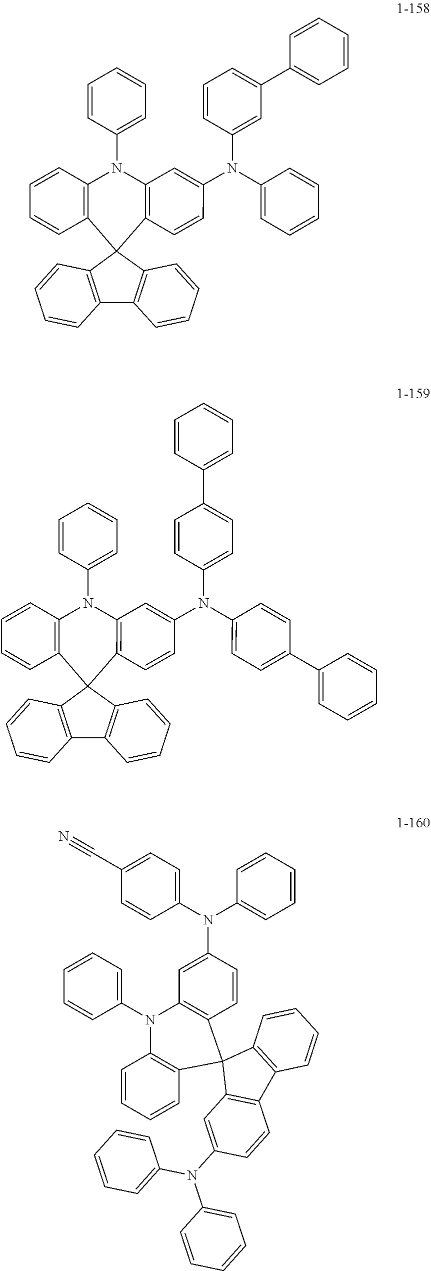 [SMILES: c1ccc(-c2cccc(N(c3ccccc3)c3ccc4c(c3)N(c3ccccc3)c3ccccc3C43c4ccccc4-c4ccccc43)c2)cc1 ; c1ccc(-c2ccc(N(c3ccc(-c4ccccc4)cc3)c3ccc4c(c3)N(c3ccccc3)c3ccccc3C43c4ccccc4-c4ccccc43)cc2)cc1 ; N#Cc1ccc(N(c2ccccc2)c2ccc3c(c2)N(c2ccccc2)c2ccccc2C32c3ccccc3-c3ccc(N(c4ccccc4)c4ccccc4)cc32)cc1]

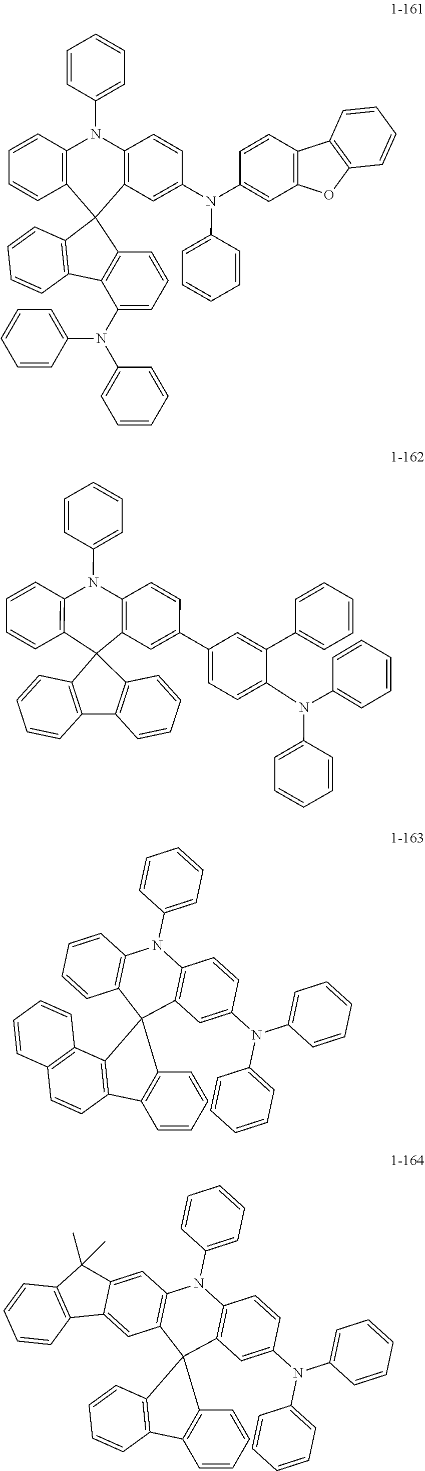 [SMILES: c1ccc(N(c2ccc3c(c2)C2(c4ccccc4-c4c(N(c5ccccc5)c5ccccc5)cccc42)c2ccccc2N3c2ccccc2)c2ccc3c(c2)oc2ccccc23)cc1 ; c1ccc(-c2cc(-c3ccc4c(c3)C3(c5ccccc5-c5ccccc53)c3ccccc3N4c3ccccc3)ccc2N(c2ccccc2)c2ccccc2)cc1 ; c1ccc(N(c2ccccc2)c2ccc3c(c2)C2(c4ccccc4-c4ccc5ccccc5c42)c2ccccc2N3c2ccccc2)cc1 ; CC1(C)c2ccccc2-c2cc3c(cc21)N(c1ccccc1)c1ccc(N(c2ccccc2)c2ccccc2)cc1C31c2ccccc2-c2ccccc21]

Also, at least one of Ar⁵ to Ar⁷ in Formula 2 is represented by any one of Formulas 2-1 to 2-6:

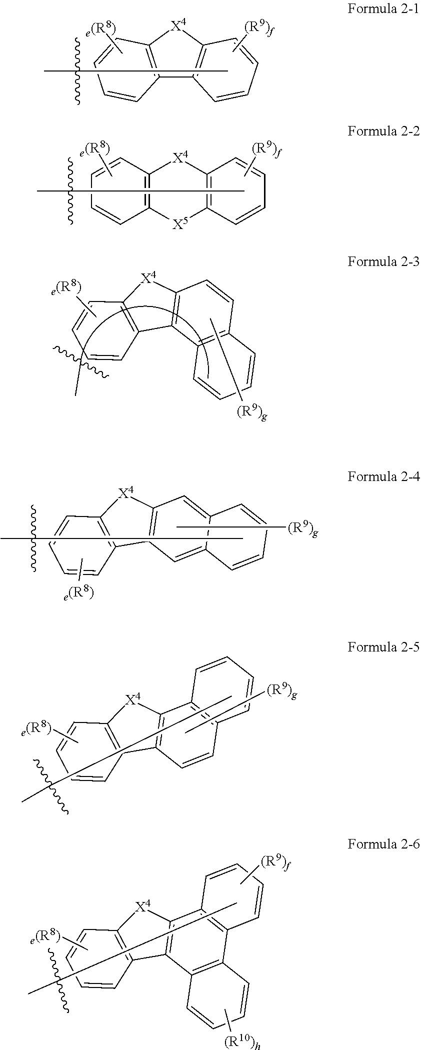

wherein

1) X⁴ and X⁵ are each independently NAr⁸, O, S or CR^{c}R^{d},

2) Ar⁸ is the same as the definition of Ar¹ in Formula 1,

3) R⁸, R⁹, R¹⁰, R^{c} and R^{d} are each independently selected from the group consisting of hydrogen; deuterium; halogen; a silane group unsubstituted or substituted with a C₁-C₂₀ alkyl group or a C₆-C₂₀ aryl group; cyano group; nitro group; C₁-C₂₀ alkoxy group; C₆-C₂₀ aryloxy group; C₁-C₂₀ alkyl group; C₂-C₂₀ alkenyl group; C₂-C₂₀ alkynyl group; C₆-C₂₀ aryl group; fluorenyl group; a C₂-C₂₀ heterocyclic group including at least one heteroatom of O, N, S, Si or P; and a C₃-C₂₀ aliphatic ring; alternatively, adjacent groups may be bonded to each other to form a ring, and adjacent substituents may be bonded to form a ring,

4) e, f and h are integers from 0 to 4, and g is an integer from 0 to 6.

Also, the compound represented by Formula 2 is represented by any one of Formulas 2-7 to 2-9.

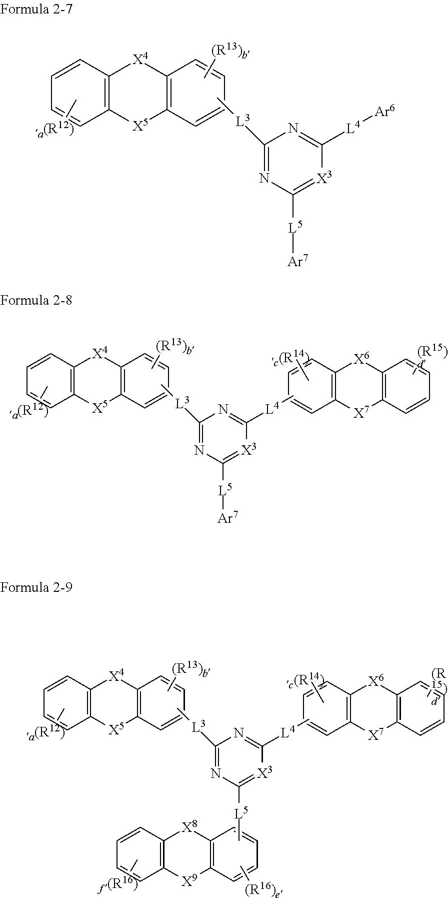

wherein

1) X³, L³, L⁴, L⁵, Ar⁶ and Ar⁷ are the same as defined in Formula 2,

2) X⁴, X⁶ and X⁸ are each independently O, S, NAr⁹ or CR^{c}R^{d},

3) X⁵, X⁷ and X⁹ are each independently O, S, NAr¹⁰, CR^{e}R^{f} or single bond,

4) a′, d′ and f′ are an integer of 0 to 4, b′, c′ and e′ are an integer of 0 to 3,

5) Arg and Ar¹⁰ are the same as the definition of Ar¹ in Formula 1,

6) R_{c}, R_{d}, Rₑ, R_{f}, R¹², R¹³, R¹⁴, R¹⁵, R¹⁶ and R¹⁷ the same as the definition of R⁸ in Formula 2-1.

Also, at least one of L¹ to L⁵ in Formulas 1 to 2 is represented by one of the following Formulas b-1 to Formula b-13.

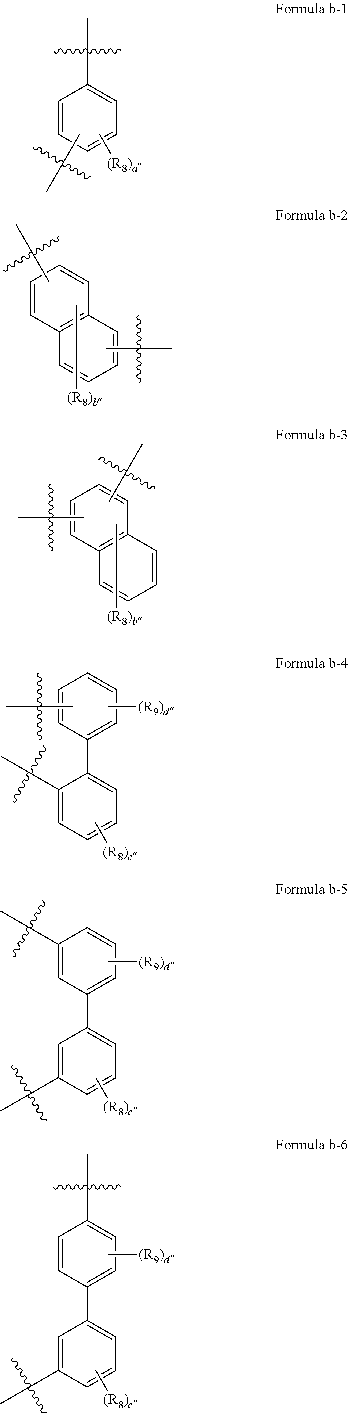

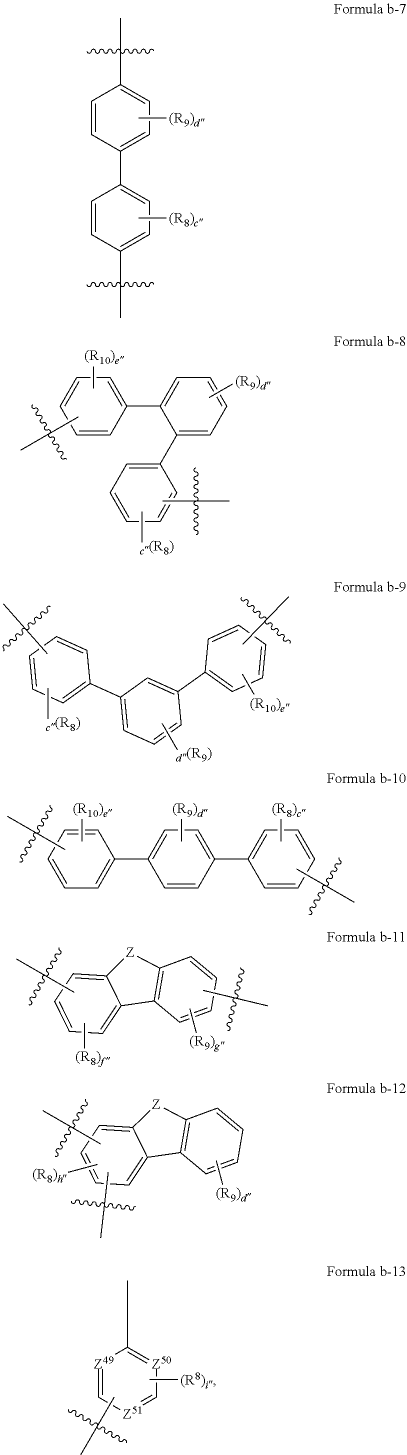

wherein

1) Z is O, S, N-L⁶-Ar¹¹ or CR₆R₇,

2) L⁶ is the same as the definition of L¹ in Formula 1,

3) Ar¹¹ is the same as the definition of Ar¹ in Formula 1,

4) R⁶, R⁷, R⁸, R⁹ and R¹⁰ are the same or different from each other, and each independently selected from the group consisting of hydrogen; deuterium; C₆-C₂₀ aryl group; fluorenyl group; a C₂-C₂₀ heterocyclic group including at least one heteroatom of O, N, S, Si or P; and a C₃-C₂₀ aliphatic ring; and adjacent groups can be bonded to each other to form a ring,

5) a″, c″, d″ and e″ are each independently an integer of 0 to 4, b″ is an integer of 0 to 6, f″ and g″ are each independently an integer of 0 to 3, h″ is an integer of 0 to 2, i″ is 0 or 1,

6) Z⁴⁹, Z⁵⁰ and Z⁵¹ are each independently CR^{g} or N, and at least one of Z⁴⁹, Z⁵⁰ and Z⁵¹ is N,

7) R^{g} is are selected from the group consisting of hydrogen; deuterium; halogen; a silane group unsubstituted or substituted with a C₁-C₂₀ alkyl group or a C₆-C₂₀ aryl group; cyano group; nitro group; C₁˜C₂₀ alkylthio group; C₁-C₂₀ alkoxy group; C₆-C₂₀ aryloxy group; C₁-C₂₀ alkyl group; C₂-C₂₀ alkenyl group; C₂-C₂₀ alkynyl group; C₆-020 aryl group; fluorenyl group; a C₂-C₂₀ heterocyclic group including at least one heteroatom of O, N, S, Si or P; and a C₃-C₂₀ aliphatic ring; C₇˜C₂₀ arylalkyl group; and a C₈˜C₂₀ arylalkenyl group;

Specifically, the compound represented by Formula 2 may be any one of the following compounds:

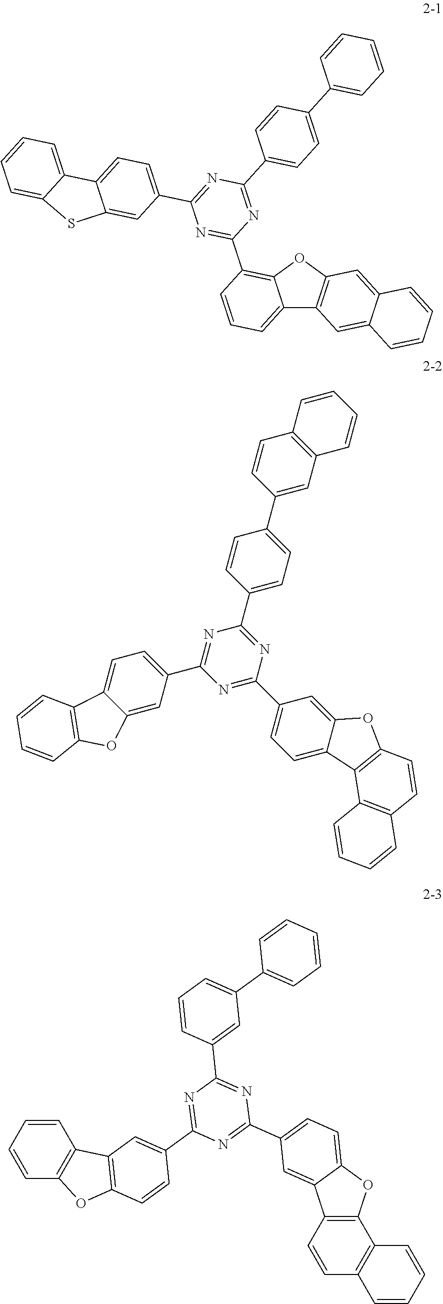 [SMILES: c1ccc(-c2ccc(-c3nc(-c4ccc5c(c4)sc4ccccc45)nc(-c4cccc5c4oc4cc6ccccc6cc45)n3)cc2)cc1 ; c1ccc2cc(-c3ccc(-c4nc(-c5ccc6c(c5)oc5ccccc56)nc(-c5ccc6c(c5)oc5ccc7ccccc7c56)n4)cc3)ccc2c1 ; c1ccc(-c2cccc(-c3nc(-c4ccc5oc6ccccc6c5c4)nc(-c4ccc5oc6c7ccccc7ccc6c5c4)n3)c2)cc1]

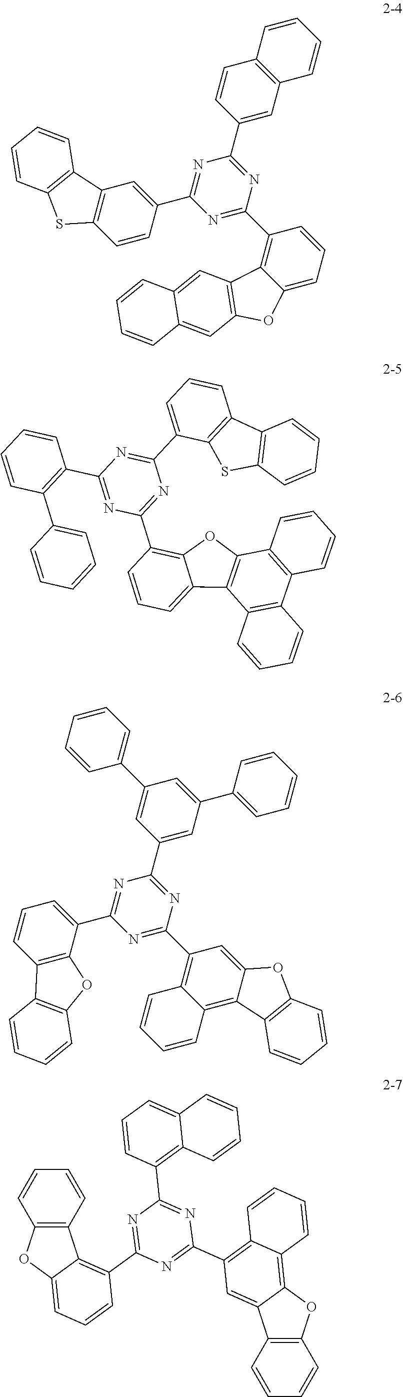 [SMILES: c1ccc2cc(-c3nc(-c4ccc5sc6ccccc6c5c4)nc(-c4cccc5oc6cc7ccccc7cc6c45)n3)ccc2c1 ; c1ccc(-c2ccccc2-c2nc(-c3cccc4c3oc3c5ccccc5c5ccccc5c43)nc(-c3cccc4c3sc3ccccc34)n2)cc1 ; c1ccc(-c2cc(-c3ccccc3)cc(-c3nc(-c4cc5oc6ccccc6c5c5ccccc45)nc(-c4cccc5c4oc4ccccc45)n3)c2)cc1 ; c1ccc2c(-c3nc(-c4cc5c6ccccc6oc5c5ccccc45)nc(-c4cccc5oc6ccccc6c45)n3)cccc2c1]

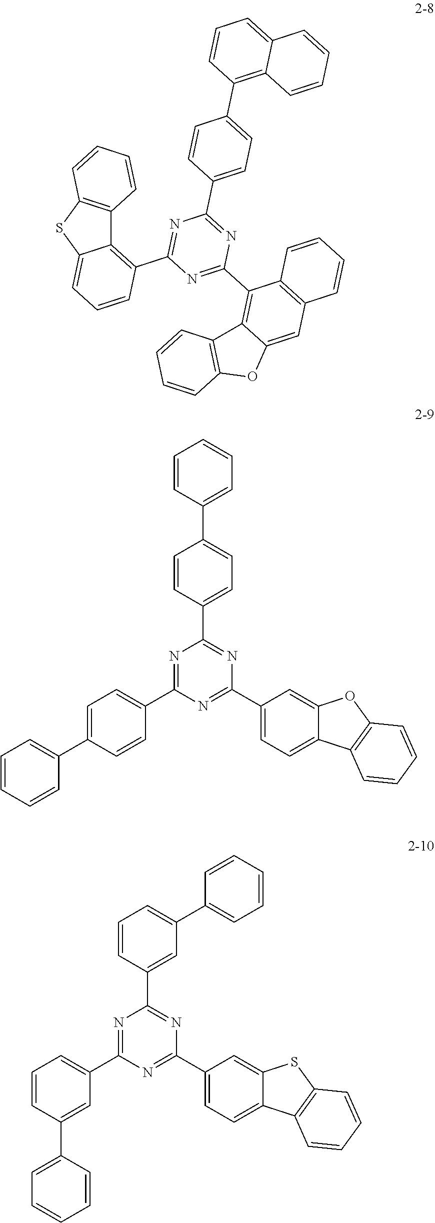

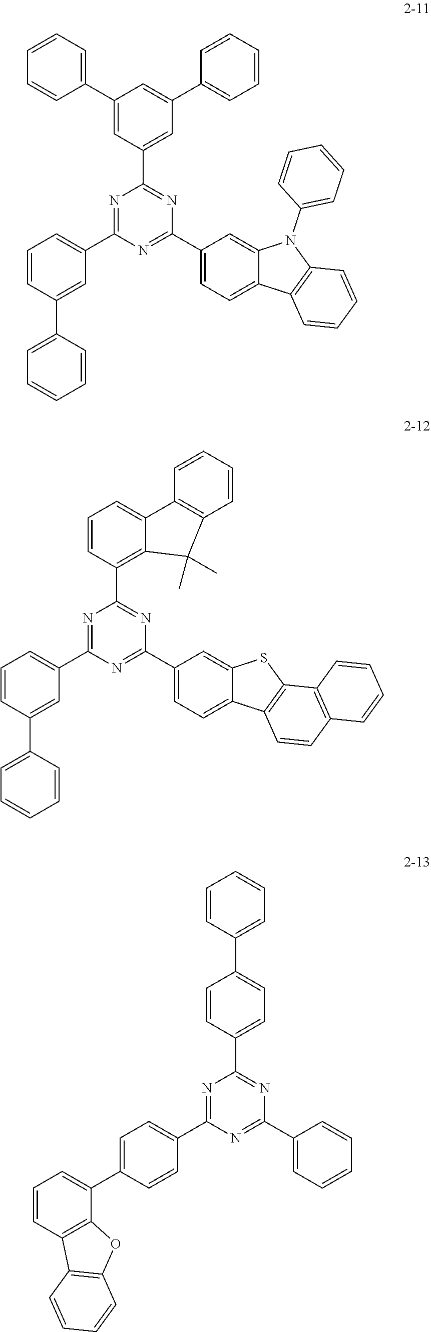 [SMILES: c1ccc(-c2cccc(-c3nc(-c4cc(-c5ccccc5)cc(-c5ccccc5)c4)nc(-c4ccc5c6ccccc6n(-c6ccccc6)c5c4)n3)c2)cc1 ; CC1(C)c2ccccc2-c2cccc(-c3nc(-c4cccc(-c5ccccc5)c4)nc(-c4ccc5c(c4)sc4c6ccccc6ccc54)n3)c21 ; c1ccc(-c2ccc(-c3nc(-c4ccccc4)nc(-c4ccc(-c5cccc6c5oc5ccccc56)cc4)n3)cc2)cc1]

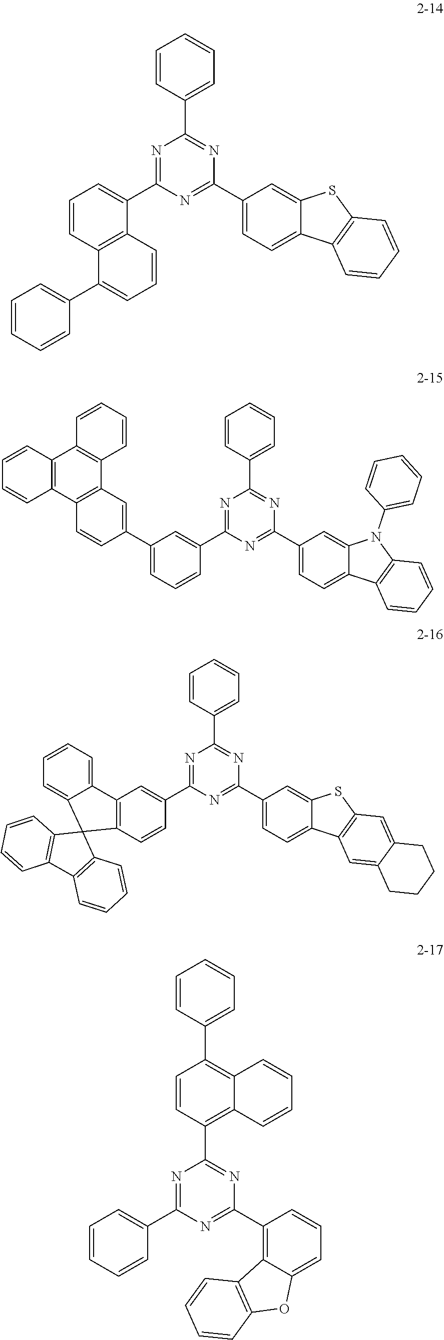 [SMILES: c1ccc(-c2nc(-c3ccc4c(c3)sc3ccccc34)nc(-c3cccc4c(-c5ccccc5)cccc34)n2)cc1 ; c1ccc(-c2nc(-c3cccc(-c4ccc5c6ccccc6c6ccccc6c5c4)c3)nc(-c3ccc4c5ccccc5n(-c5ccccc5)c4c3)n2)cc1 ; c1ccc(-c2nc(-c3ccc4c(c3)-c3ccccc3C43c4ccccc4-c4ccccc43)nc(-c3ccc4c(c3)sc3cc5c(cc34)CCCC5)n2)cc1 ; c1ccc(-c2nc(-c3ccc(-c4ccccc4)c4ccccc34)nc(-c3cccc4oc5ccccc5c34)n2)cc1]

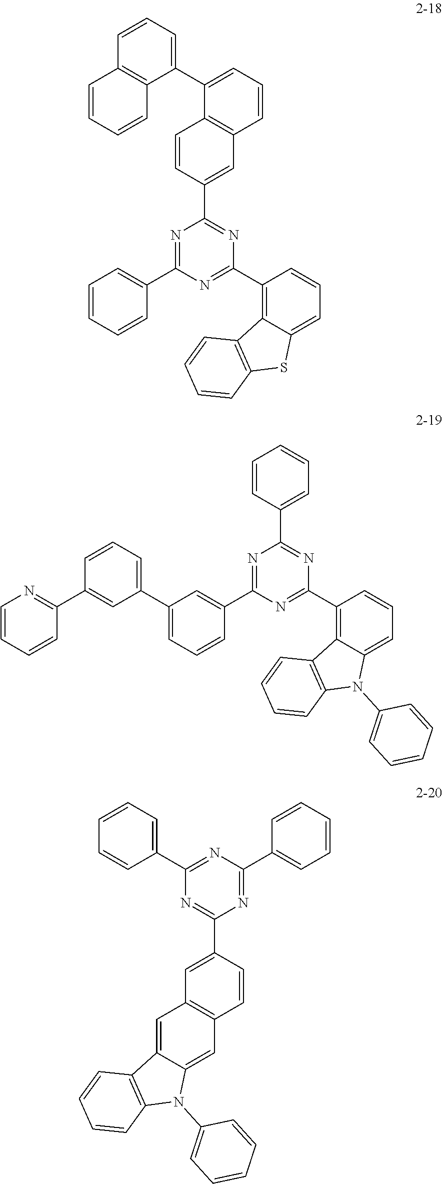 [SMILES: c1ccc(-c2nc(-c3ccc4c(-c5cccc6ccccc56)cccc4c3)nc(-c3cccc4sc5ccccc5c34)n2)cc1 ; c1ccc(-c2nc(-c3cccc(-c4cccc(-c5ccccn5)c4)c3)nc(-c3cccc4c3c3ccccc3n4-c3ccccc3)n2)cc1 ; c1ccc(-c2nc(-c3ccccc3)nc(-c3ccc4cc5c(cc4c3)c3ccccc3n5-c3ccccc3)n2)cc1]

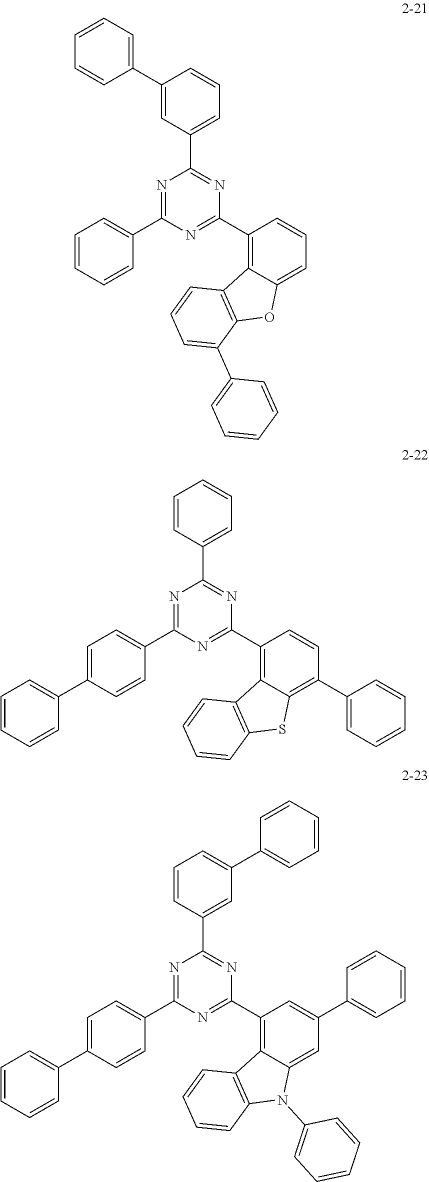 [SMILES: c1ccc(-c2cccc(-c3nc(-c4ccccc4)nc(-c4cccc5oc6c(-c7ccccc7)cccc6c45)n3)c2)cc1 ; c1ccc(-c2ccc(-c3nc(-c4ccccc4)nc(-c4ccc(-c5ccccc5)c5sc6ccccc6c45)n3)cc2)cc1 ; c1ccc(-c2ccc(-c3nc(-c4cccc(-c5ccccc5)c4)nc(-c4cc(-c5ccccc5)cc5c4c4ccccc4n5-c4ccccc4)n3)cc2)cc1]

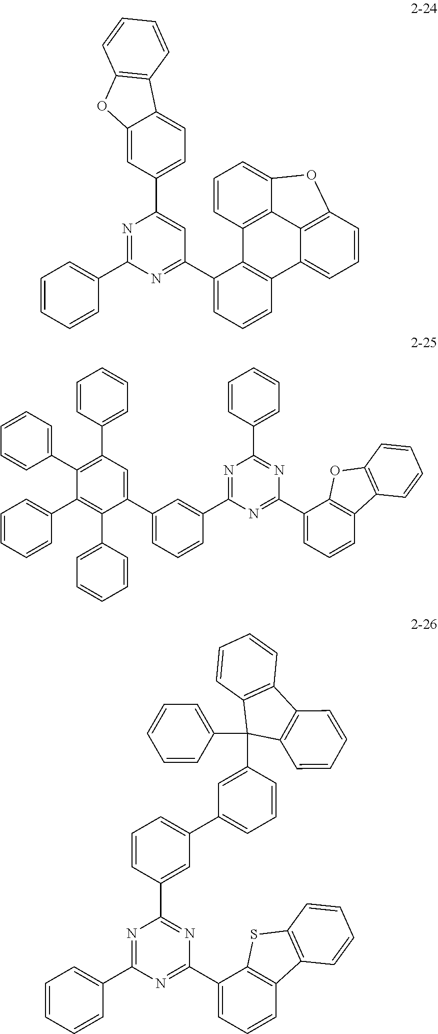

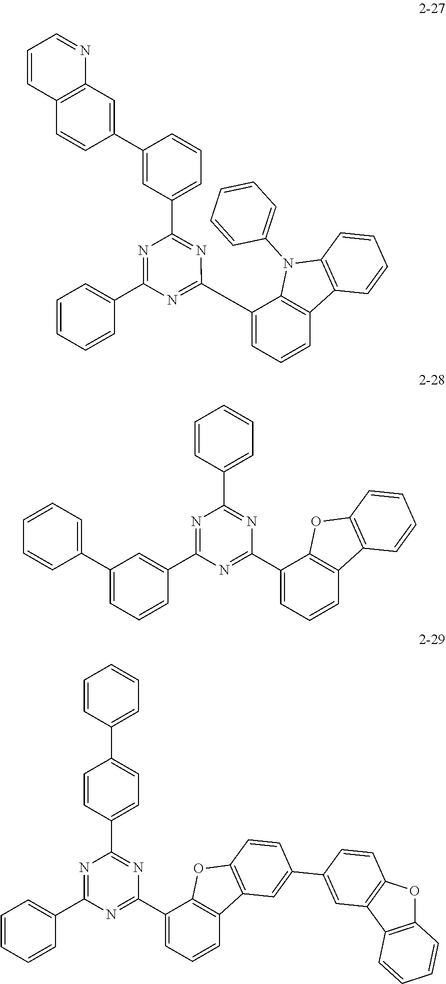 [SMILES: c1ccc(-c2nc(-c3cccc(-c4ccc5cccnc5c4)c3)nc(-c3cccc4c5ccccc5n(-c5ccccc5)c34)n2)cc1 ; c1ccc(-c2cccc(-c3nc(-c4ccccc4)nc(-c4cccc5c4oc4ccccc45)n3)c2)cc1 ; c1ccc(-c2ccc(-c3nc(-c4ccccc4)nc(-c4cccc5c4oc4ccc(-c6ccc7oc8ccccc8c7c6)cc45)n3)cc2)cc1]

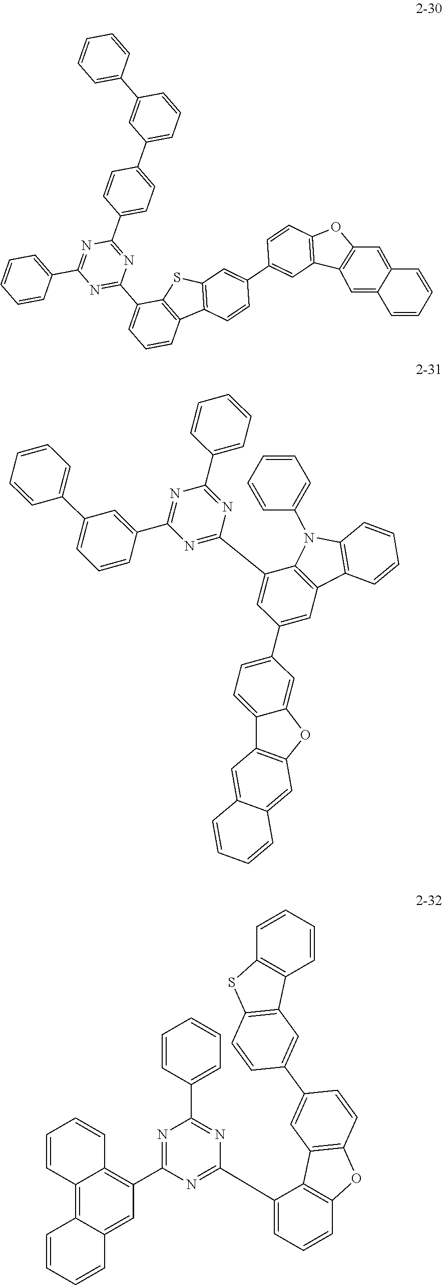 [SMILES: c1ccc(-c2cccc(-c3ccc(-c4nc(-c5ccccc5)nc(-c5cccc6c5sc5cc(-c7ccc8oc9cc%10ccccc%10cc9c8c7)ccc56)n4)cc3)c2)cc1 ; c1ccc(-c2cccc(-c3nc(-c4ccccc4)nc(-c4cc(-c5ccc6c(c5)oc5cc7ccccc7cc56)cc5c6ccccc6n(-c6ccccc6)c45)n3)c2)cc1 ; c1ccc(-c2nc(-c3cc4ccccc4c4ccccc34)nc(-c3cccc4oc5ccc(-c6ccc7sc8ccccc8c7c6)cc5c34)n2)cc1]

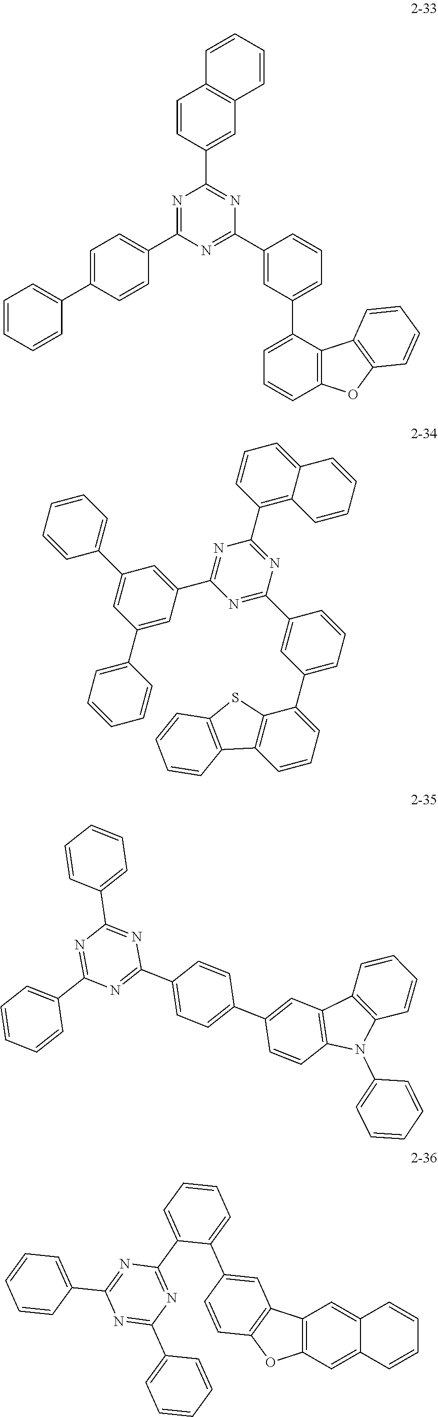

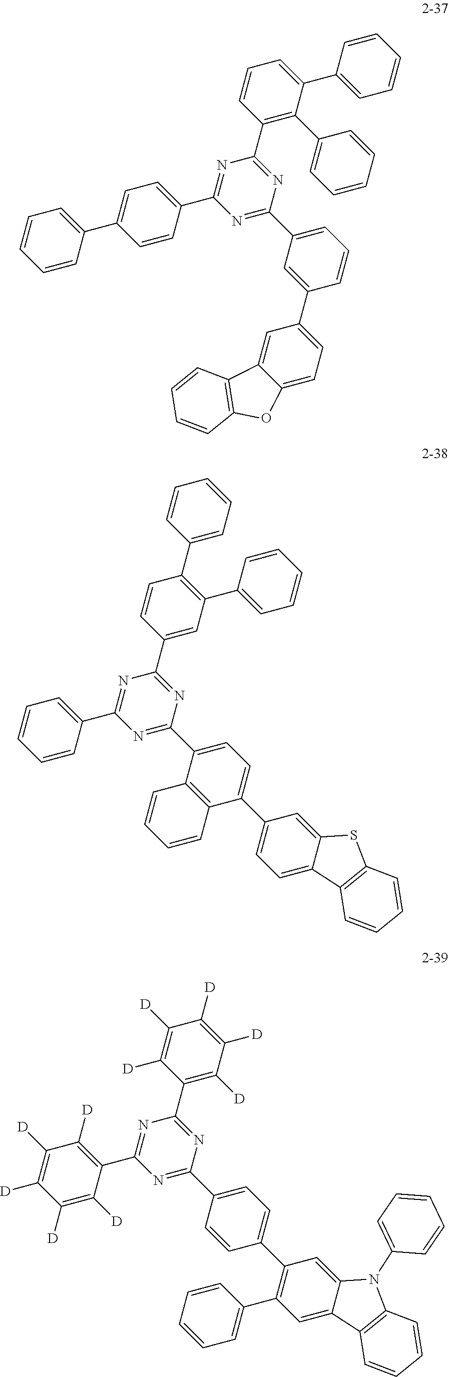

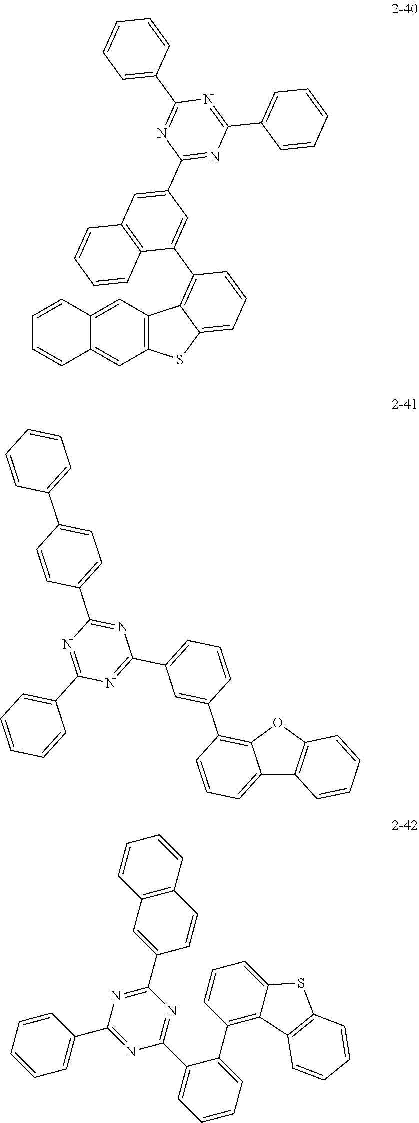 [SMILES: c1ccc(-c2nc(-c3ccccc3)nc(-c3cc(-c4cccc5sc6cc7ccccc7cc6c45)c4ccccc4c3)n2)cc1 ; c1ccc(-c2ccc(-c3nc(-c4ccccc4)nc(-c4cccc(-c5cccc6c5oc5ccccc56)c4)n3)cc2)cc1 ; c1ccc(-c2nc(-c3ccc4ccccc4c3)nc(-c3ccccc3-c3cccc4sc5ccccc5c34)n2)cc1]

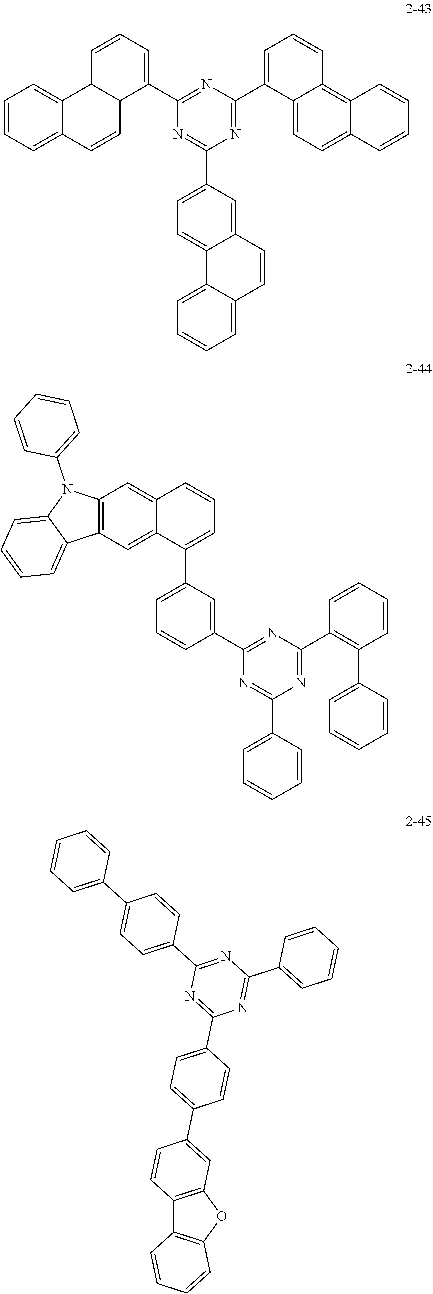 [SMILES: C1=CC2c3ccccc3C=CC2C(c2nc(-c3ccc4c(ccc5ccccc54)c3)nc(-c3cccc4c3ccc3ccccc34)n2)=C1 ; c1ccc(-c2nc(-c3cccc(-c4cccc5cc6c(cc45)c4ccccc4n6-c4ccccc4)c3)nc(-c3ccccc3-c3ccccc3)n2)cc1 ; c1ccc(-c2ccc(-c3nc(-c4ccccc4)nc(-c4ccc(-c5ccc6c(c5)oc5ccccc56)cc4)n3)cc2)cc1]

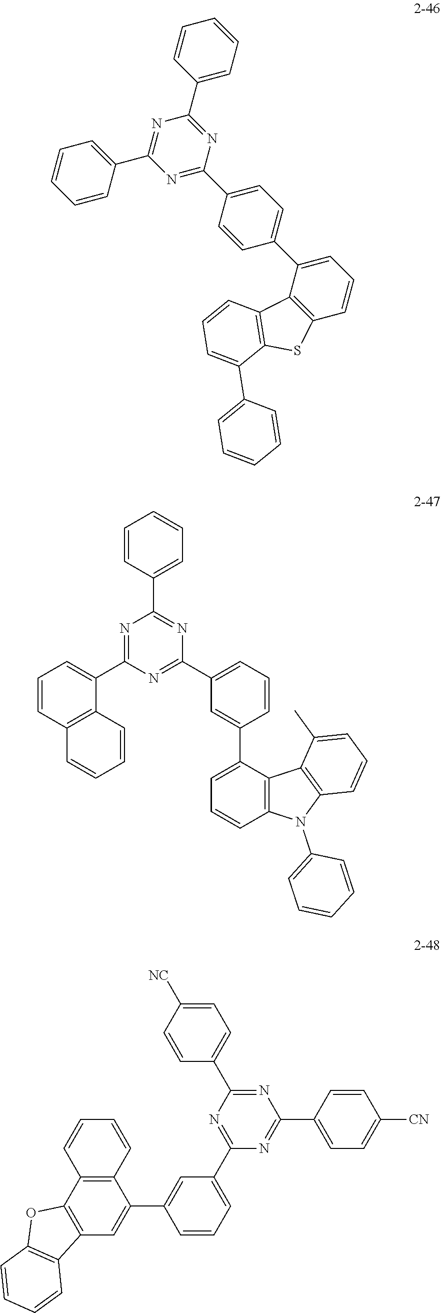 [SMILES: c1ccc(-c2nc(-c3ccccc3)nc(-c3ccc(-c4cccc5sc6c(-c7ccccc7)cccc6c45)cc3)n2)cc1 ; Cc1cccc2c1c1c(-c3cccc(-c4nc(-c5ccccc5)nc(-c5cccc6ccccc56)n4)c3)cccc1n2-c1ccccc1 ; N#Cc1ccc(-c2nc(-c3ccc(C#N)cc3)nc(-c3cccc(-c4cc5c6ccccc6oc5c5ccccc45)c3)n2)cc1]

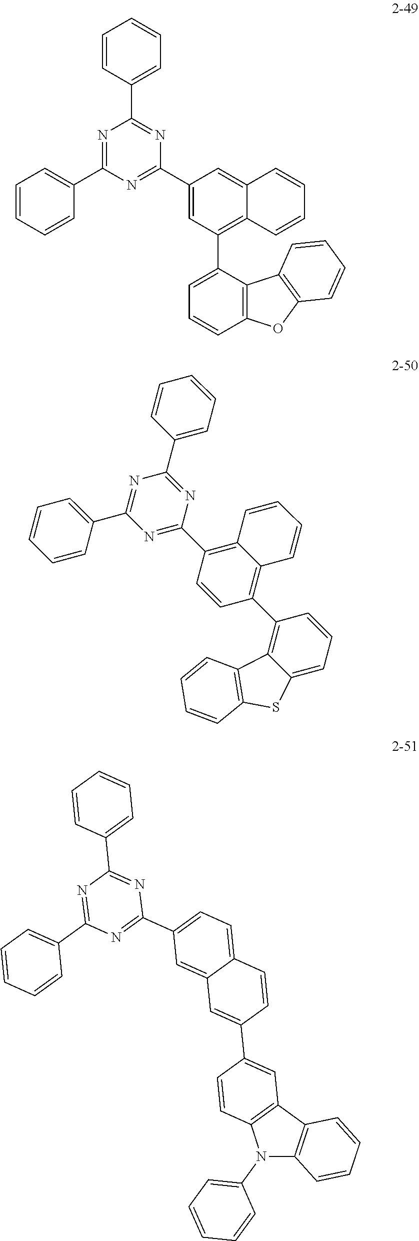 [SMILES: c1ccc(-c2nc(-c3ccccc3)nc(-c3cc(-c4cccc5oc6ccccc6c45)c4ccccc4c3)n2)cc1 ; c1ccc(-c2nc(-c3ccccc3)nc(-c3ccc(-c4cccc5sc6ccccc6c45)c4ccccc34)n2)cc1 ; c1ccc(-c2nc(-c3ccccc3)nc(-c3ccc4ccc(-c5ccc6c(c5)c5ccccc5n6-c5ccccc5)cc4c3)n2)cc1]

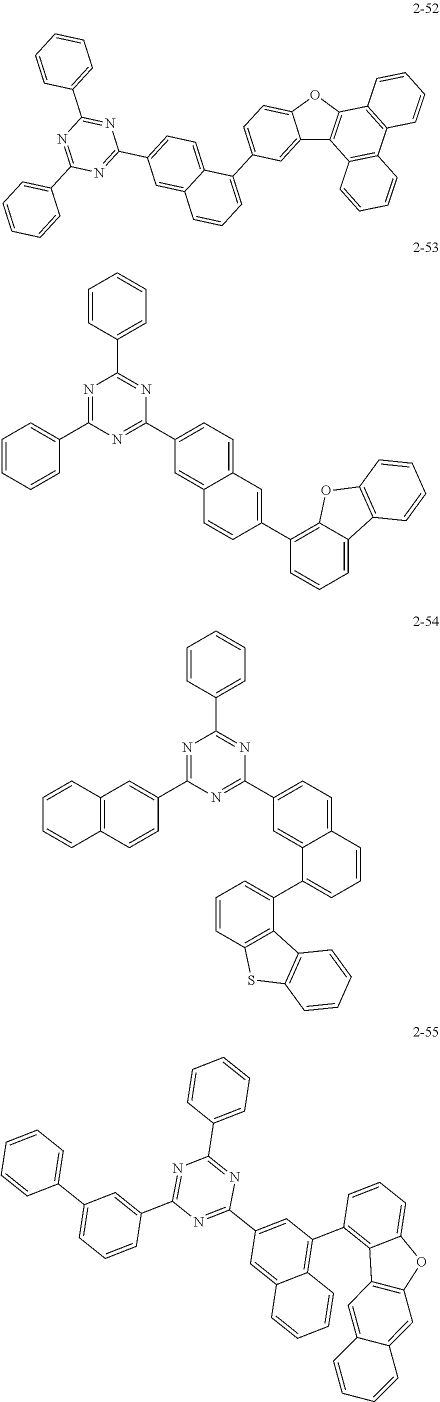 [SMILES: c1ccc(-c2nc(-c3ccccc3)nc(-c3ccc4c(-c5ccc6oc7c8ccccc8c8ccccc8c7c6c5)cccc4c3)n2)cc1 ; c1ccc(-c2nc(-c3ccccc3)nc(-c3ccc4cc(-c5cccc6c5oc5ccccc56)ccc4c3)n2)cc1 ; c1ccc(-c2nc(-c3ccc4ccccc4c3)nc(-c3ccc4cccc(-c5cccc6sc7ccccc7c56)c4c3)n2)cc1 ; c1ccc(-c2cccc(-c3nc(-c4ccccc4)nc(-c4cc(-c5cccc6oc7cc8ccccc8cc7c56)c5ccccc5c4)n3)c2)cc1]

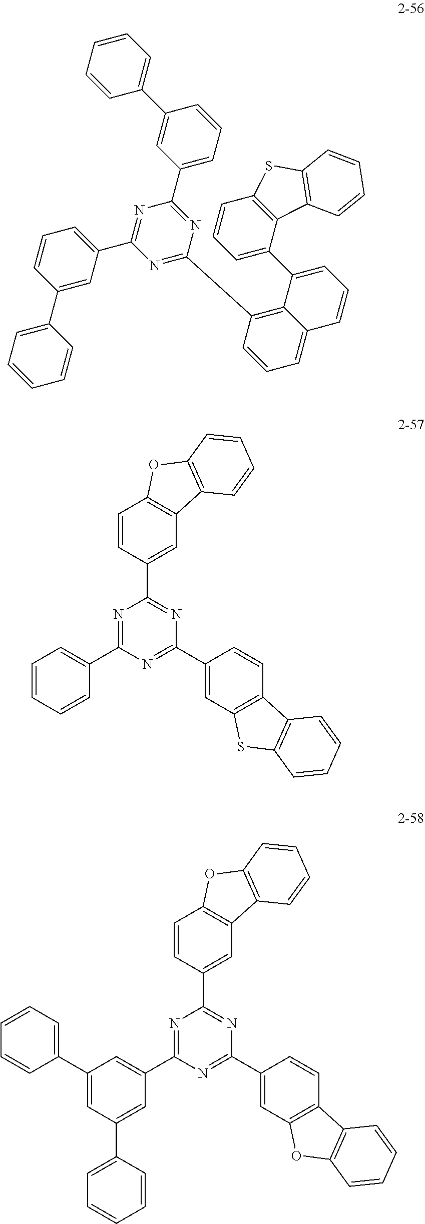 [SMILES: c1ccc(-c2cccc(-c3nc(-c4cccc(-c5ccccc5)c4)nc(-c4cccc5cccc(-c6cccc7sc8ccccc8c67)c45)n3)c2)cc1 ; c1ccc(-c2nc(-c3ccc4c(c3)sc3ccccc34)nc(-c3ccc4oc5ccccc5c4c3)n2)cc1 ; c1ccc(-c2cc(-c3ccccc3)cc(-c3nc(-c4ccc5c(c4)oc4ccccc45)nc(-c4ccc5oc6ccccc6c5c4)n3)c2)cc1]

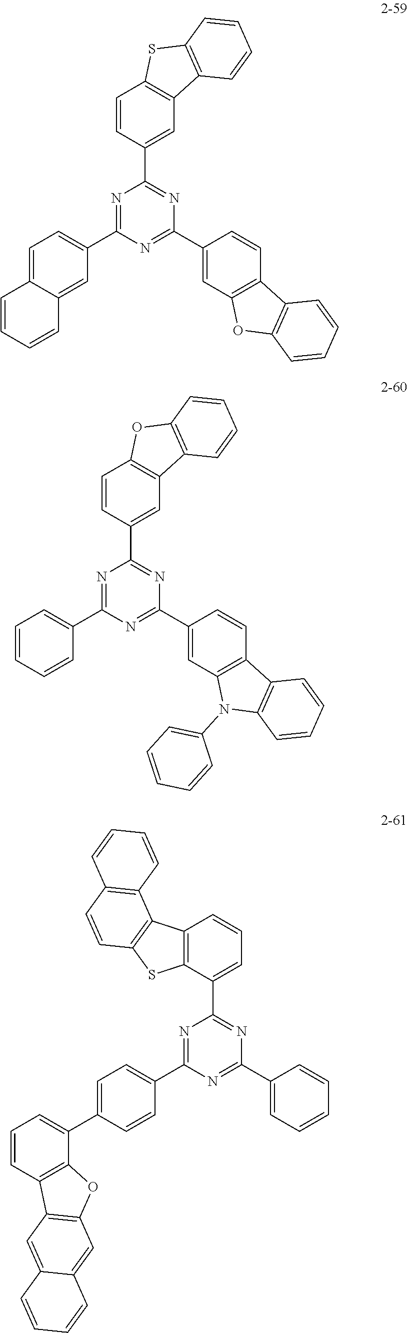 [SMILES: c1ccc2cc(-c3nc(-c4ccc5c(c4)oc4ccccc45)nc(-c4ccc5sc6ccccc6c5c4)n3)ccc2c1 ; c1ccc(-c2nc(-c3ccc4oc5ccccc5c4c3)nc(-c3ccc4c5ccccc5n(-c5ccccc5)c4c3)n2)cc1 ; c1ccc(-c2nc(-c3ccc(-c4cccc5c4oc4cc6ccccc6cc45)cc3)nc(-c3cccc4c3sc3ccc5ccccc5c34)n2)cc1]

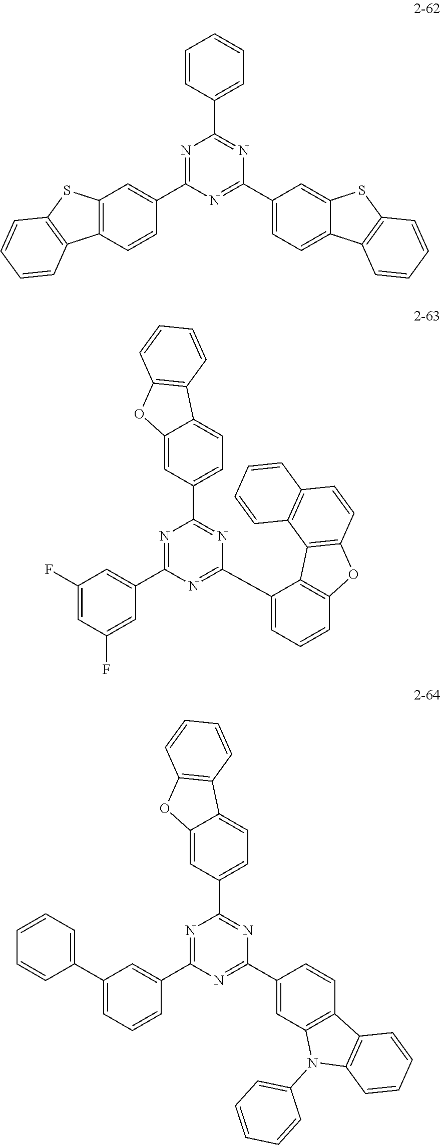 [SMILES: c1ccc(-c2nc(-c3ccc4c(c3)sc3ccccc34)nc(-c3ccc4c(c3)sc3ccccc34)n2)cc1 ; Fc1cc(F)cc(-c2nc(-c3ccc4c(c3)oc3ccccc34)nc(-c3cccc4oc5ccc6ccccc6c5c34)n2)c1 ; c1ccc(-c2cccc(-c3nc(-c4ccc5c(c4)oc4ccccc45)nc(-c4ccc5c6ccccc6n(-c6ccccc6)c5c4)n3)c2)cc1]

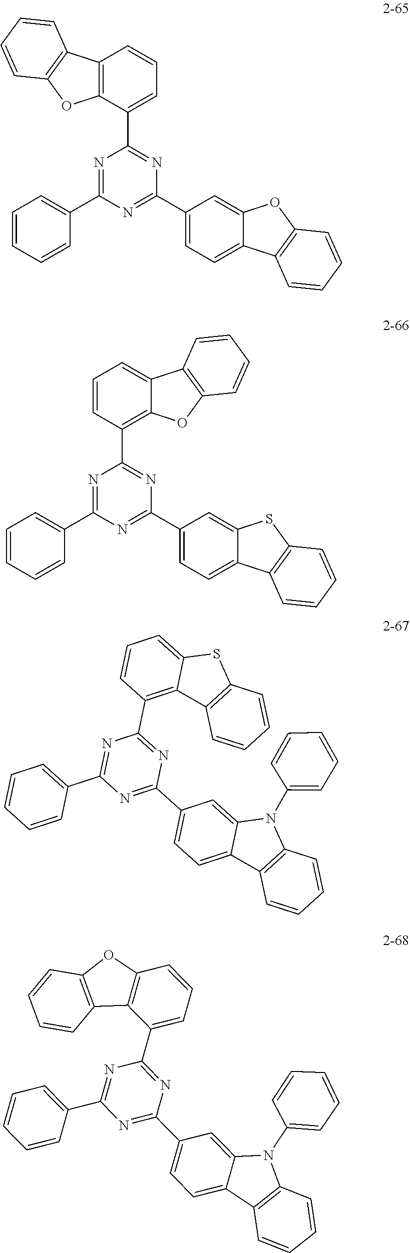 [SMILES: c1ccc(-c2nc(-c3ccc4c(c3)oc3ccccc34)nc(-c3cccc4c3oc3ccccc34)n2)cc1 ; c1ccc(-c2nc(-c3ccc4c(c3)sc3ccccc34)nc(-c3cccc4c3oc3ccccc34)n2)cc1 ; c1ccc(-c2nc(-c3ccc4c5ccccc5n(-c5ccccc5)c4c3)nc(-c3cccc4sc5ccccc5c34)n2)cc1 ; c1ccc(-c2nc(-c3ccc4c5ccccc5n(-c5ccccc5)c4c3)nc(-c3cccc4oc5ccccc5c34)n2)cc1]

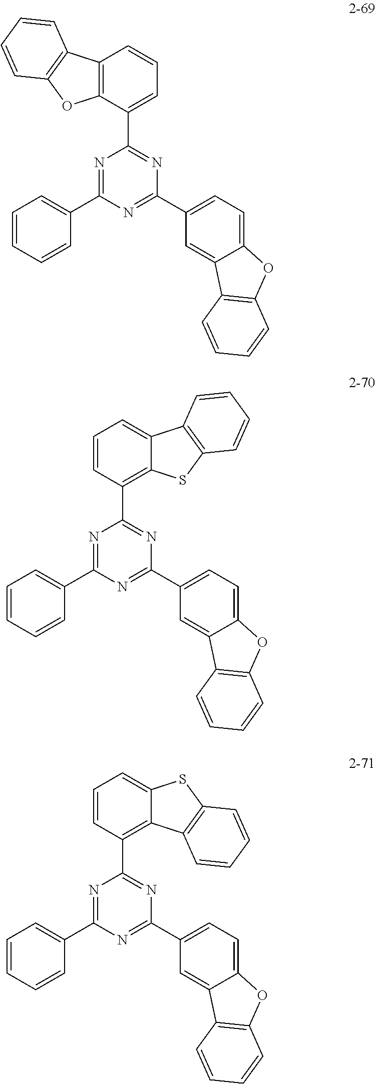

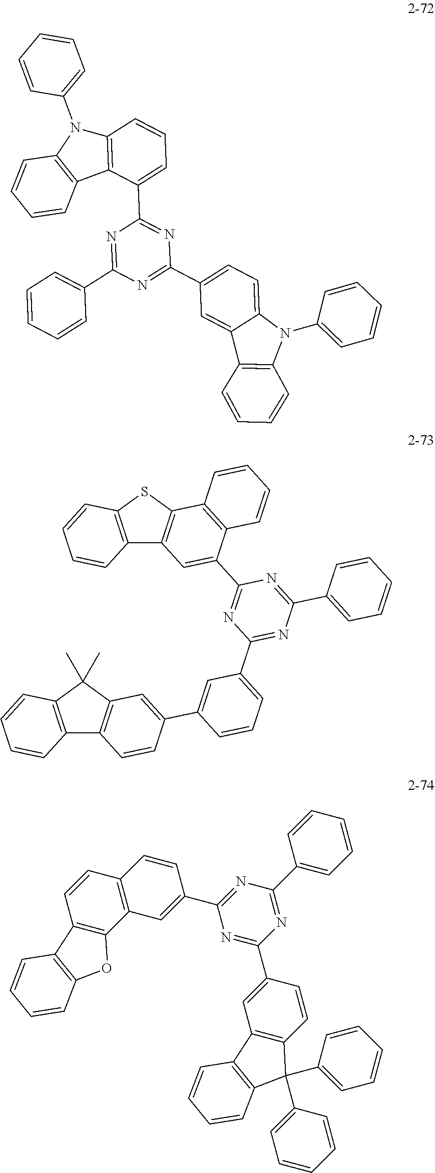

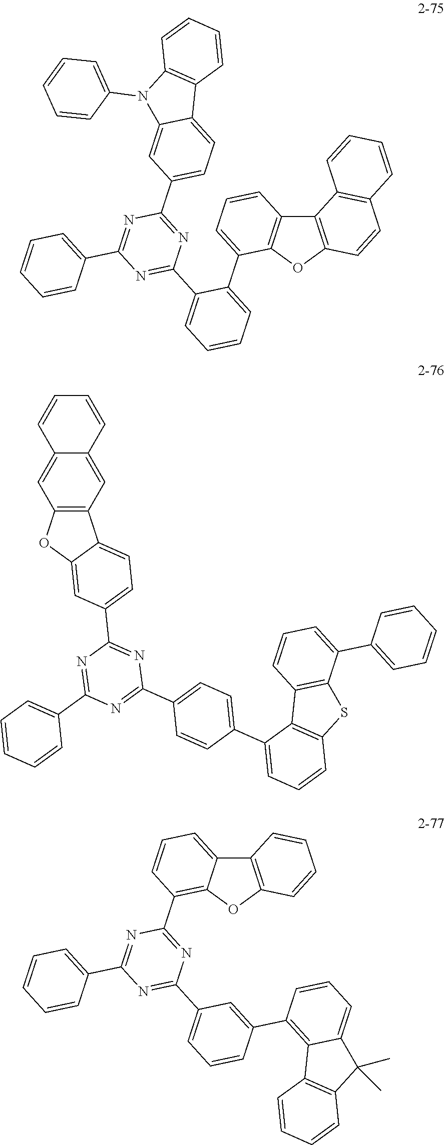 [SMILES: c1ccc(-c2nc(-c3ccc4c5ccccc5n(-c5ccccc5)c4c3)nc(-c3ccccc3-c3cccc4c3oc3ccc5ccccc5c34)n2)cc1 ; c1ccc(-c2nc(-c3ccc(-c4cccc5sc6c(-c7ccccc7)cccc6c45)cc3)nc(-c3ccc4c(c3)oc3cc5ccccc5cc34)n2)cc1 ; CC1(C)c2ccccc2-c2c(-c3cccc(-c4nc(-c5ccccc5)nc(-c5cccc6c5oc5ccccc56)n4)c3)cccc21]

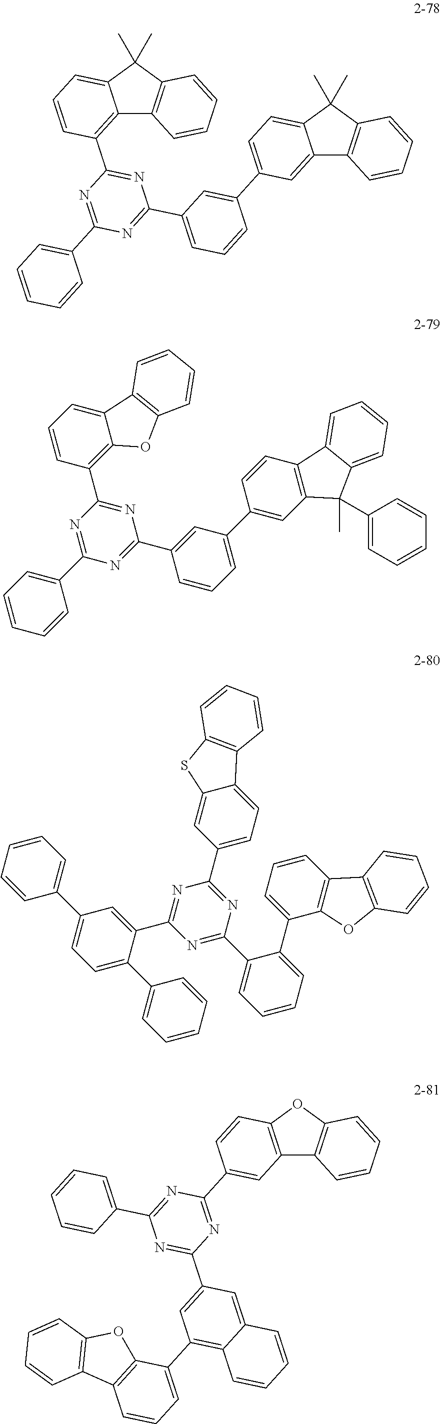 [SMILES: CC1(C)c2ccccc2-c2cc(-c3cccc(-c4nc(-c5ccccc5)nc(-c5cccc6c5-c5ccccc5C6(C)C)n4)c3)ccc21 ; CC1(c2ccccc2)c2ccccc2-c2ccc(-c3cccc(-c4nc(-c5ccccc5)nc(-c5cccc6c5oc5ccccc56)n4)c3)cc21 ; c1ccc(-c2ccc(-c3ccccc3)c(-c3nc(-c4ccc5c(c4)sc4ccccc45)nc(-c4ccccc4-c4cccc5c4oc4ccccc45)n3)c2)cc1 ; c1ccc(-c2nc(-c3cc(-c4cccc5c4oc4ccccc45)c4ccccc4c3)nc(-c3ccc4oc5ccccc5c4c3)n2)cc1]

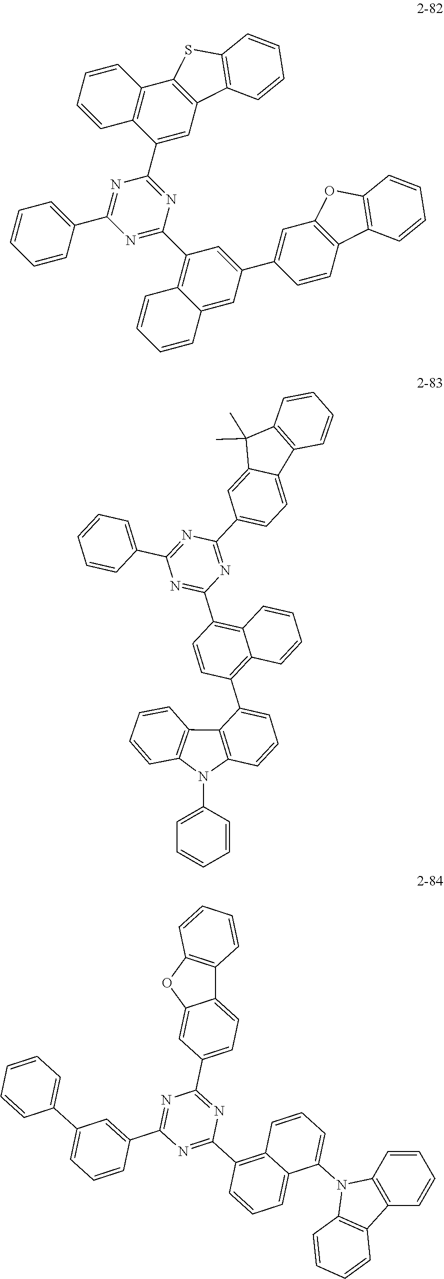

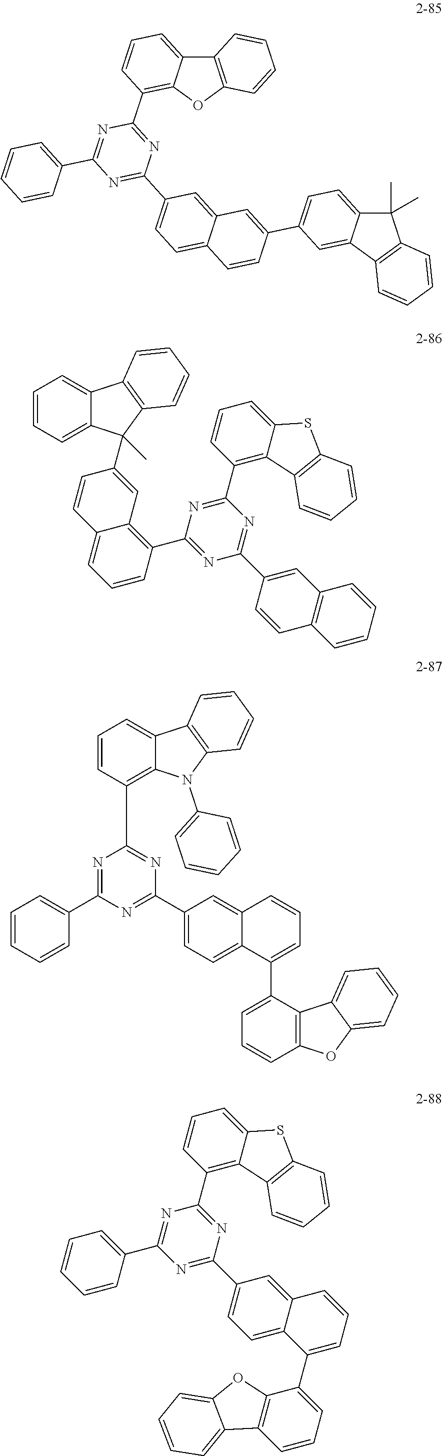 [SMILES: CC1(C)c2ccccc2-c2cc(-c3ccc4ccc(-c5nc(-c6ccccc6)nc(-c6cccc7c6oc6ccccc67)n5)cc4c3)ccc21 ; CC1(c2ccc3cccc(-c4nc(-c5ccc6ccccc6c5)nc(-c5cccc6sc7ccccc7c56)n4)c3c2)c2ccccc2-c2ccccc21 ; c1ccc(-c2nc(-c3ccc4c(-c5cccc6oc7ccccc7c56)cccc4c3)nc(-c3cccc4c5ccccc5n(-c5ccccc5)c34)n2)cc1 ; c1ccc(-c2nc(-c3ccc4c(-c5cccc6c5oc5ccccc56)cccc4c3)nc(-c3cccc4sc5ccccc5c34)n2)cc1]

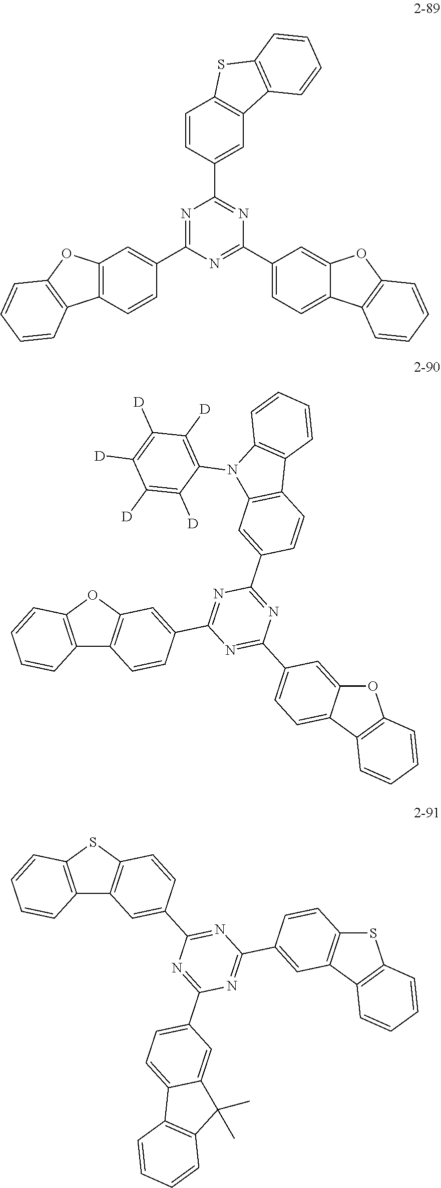 [SMILES: c1ccc2c(c1)oc1cc(-c3nc(-c4ccc5c(c4)oc4ccccc45)nc(-c4ccc5sc6ccccc6c5c4)n3)ccc12 ; [2H]c1c([2H])c([2H])c(-n2c3ccccc3c3ccc(-c4nc(-c5ccc6c(c5)oc5ccccc56)nc(-c5ccc6c(c5)oc5ccccc56)n4)cc32)c([2H])c1[2H] ; CC1(C)c2ccccc2-c2ccc(-c3nc(-c4ccc5sc6ccccc6c5c4)nc(-c4ccc5sc6ccccc6c5c4)n3)cc21]

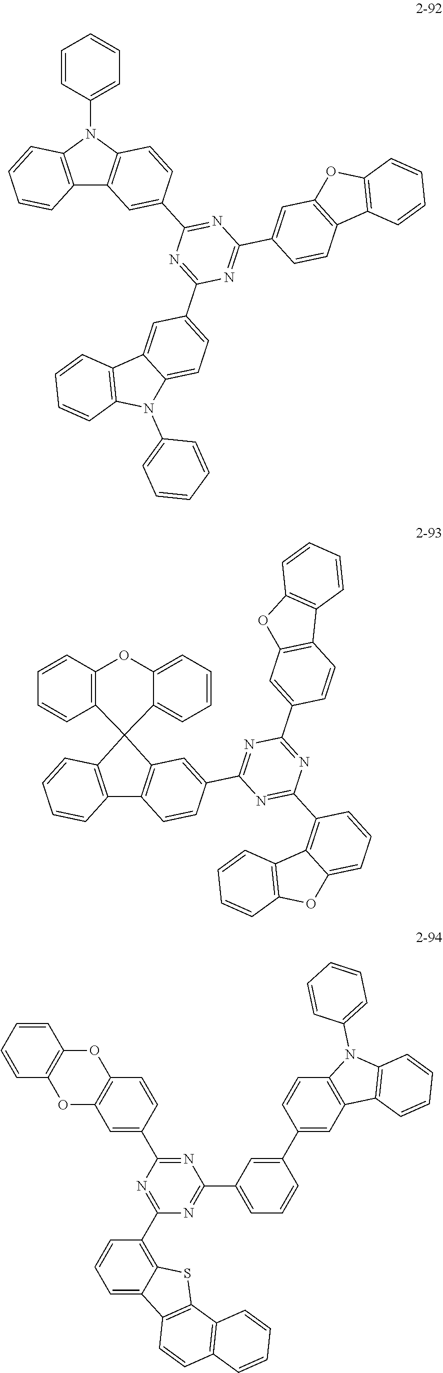 [SMILES: c1ccc(-n2c3ccccc3c3cc(-c4nc(-c5ccc6c(c5)oc5ccccc56)nc(-c5ccc6c(c5)c5ccccc5n6-c5ccccc5)n4)ccc32)cc1 ; c1ccc2c(c1)Oc1ccccc1C21c2ccccc2-c2ccc(-c3nc(-c4ccc5c(c4)oc4ccccc45)nc(-c4cccc5oc6ccccc6c45)n3)cc21 ; c1ccc(-n2c3ccccc3c3cc(-c4cccc(-c5nc(-c6ccc7c(c6)Oc6ccccc6O7)nc(-c6cccc7c6sc6c8ccccc8ccc76)n5)c4)ccc32)cc1]

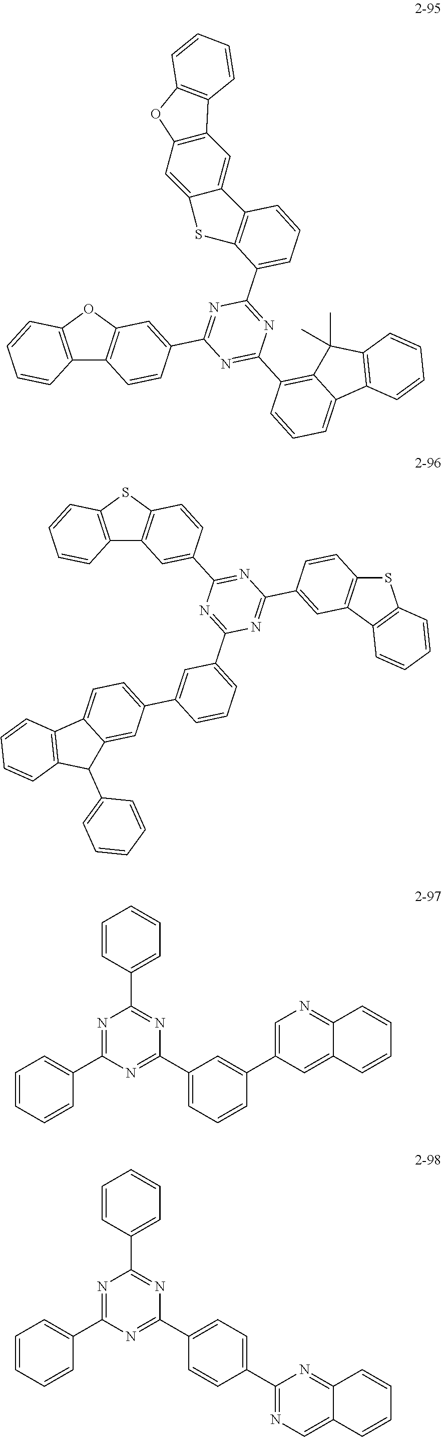 [SMILES: CC1(C)c2ccccc2-c2cccc(-c3nc(-c4ccc5c(c4)oc4ccccc45)nc(-c4cccc5c4sc4cc6oc7ccccc7c6cc45)n3)c21 ; c1ccc(C2c3ccccc3-c3ccc(-c4cccc(-c5nc(-c6ccc7sc8ccccc8c7c6)nc(-c6ccc7sc8ccccc8c7c6)n5)c4)cc32)cc1 ; c1ccc(-c2nc(-c3ccccc3)nc(-c3cccc(-c4cnc5ccccc5c4)c3)n2)cc1 ; c1ccc(-c2nc(-c3ccccc3)nc(-c3ccc(-c4ncc5ccccc5n4)cc3)n2)cc1]

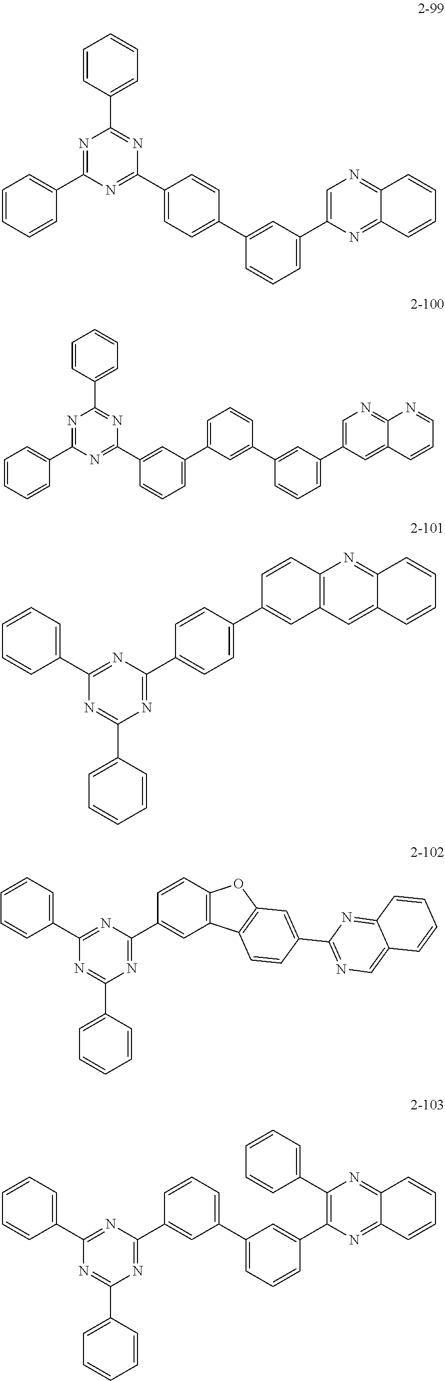 [SMILES: c1ccc(-c2nc(-c3ccccc3)nc(-c3ccc(-c4cccc(-c5cnc6ccccc6n5)c4)cc3)n2)cc1 ; c1ccc(-c2nc(-c3ccccc3)nc(-c3cccc(-c4cccc(-c5cccc(-c6cnc7ncccc7c6)c5)c4)c3)n2)cc1 ; c1ccc(-c2nc(-c3ccccc3)nc(-c3ccc(-c4ccc5nc6ccccc6cc5c4)cc3)n2)cc1 ; c1ccc(-c2nc(-c3ccccc3)nc(-c3ccc4oc5cc(-c6ncc7ccccc7n6)ccc5c4c3)n2)cc1 ; c1ccc(-c2nc(-c3ccccc3)nc(-c3cccc(-c4cccc(-c5nc6ccccc6nc5-c5ccccc5)c4)c3)n2)cc1]

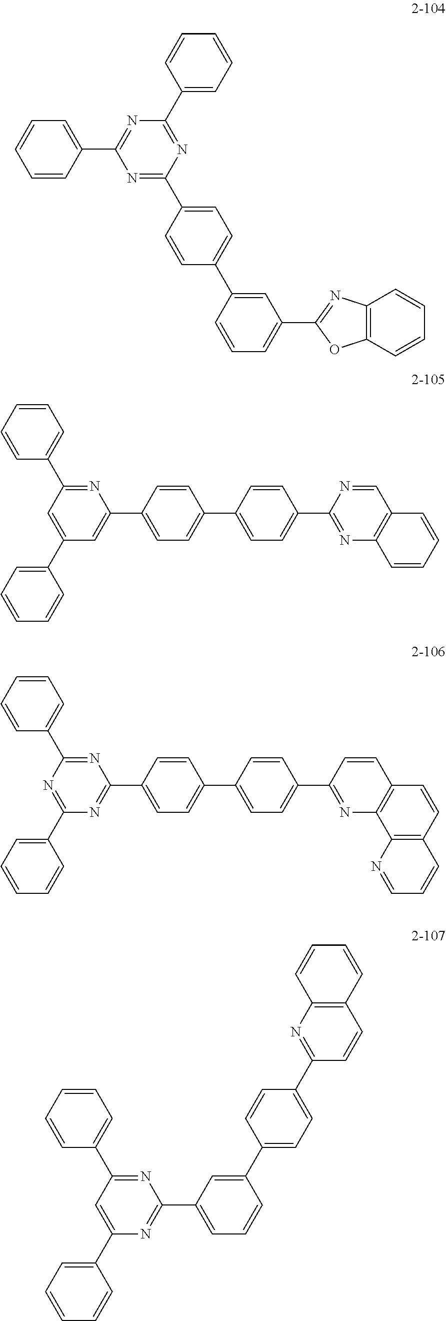 [SMILES: c1ccc(-c2nc(-c3ccccc3)nc(-c3ccc(-c4cccc(-c5nc6ccccc6o5)c4)cc3)n2)cc1 ; c1ccc(-c2cc(-c3ccccc3)nc(-c3ccc(-c4ccc(-c5ncc6ccccc6n5)cc4)cc3)c2)cc1 ; c1ccc(-c2nc(-c3ccccc3)nc(-c3ccc(-c4ccc(-c5ccc6ccc7cccnc7c6n5)cc4)cc3)n2)cc1 ; c1ccc(-c2cc(-c3ccccc3)nc(-c3cccc(-c4ccc(-c5ccc6ccccc6n5)cc4)c3)n2)cc1]

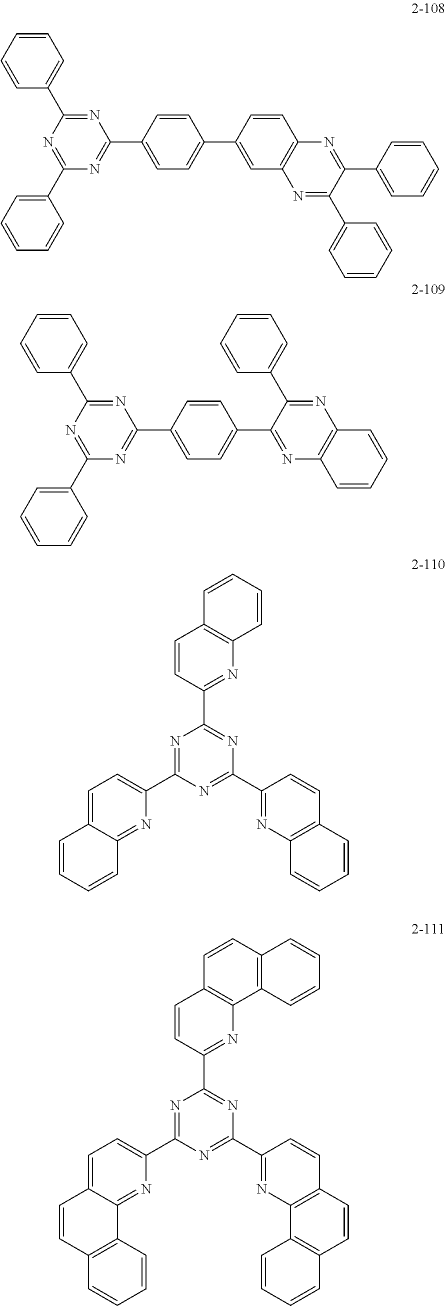 [SMILES: c1ccc(-c2nc(-c3ccccc3)nc(-c3ccc(-c4ccc5nc(-c6ccccc6)c(-c6ccccc6)nc5c4)cc3)n2)cc1 ; c1ccc(-c2nc(-c3ccccc3)nc(-c3ccc(-c4nc5ccccc5nc4-c4ccccc4)cc3)n2)cc1 ; c1ccc2nc(-c3nc(-c4ccc5ccccc5n4)nc(-c4ccc5ccccc5n4)n3)ccc2c1 ; c1ccc2c(c1)ccc1ccc(-c3nc(-c4ccc5ccc6ccccc6c5n4)nc(-c4ccc5ccc6ccccc6c5n4)n3)nc12]

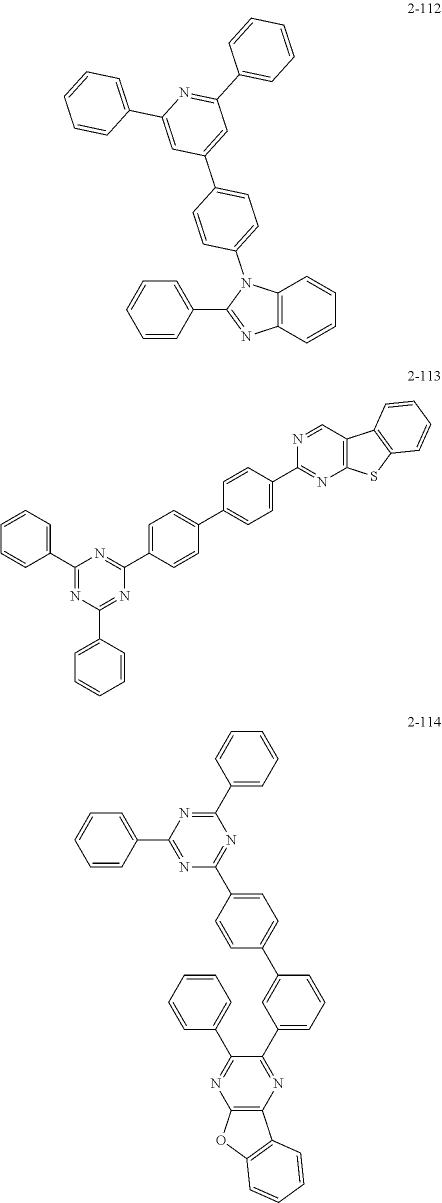 [SMILES: c1ccc(-c2cc(-c3ccc(-n4c(-c5ccccc5)nc5ccccc54)cc3)cc(-c3ccccc3)n2)cc1 ; c1ccc(-c2nc(-c3ccccc3)nc(-c3ccc(-c4ccc(-c5ncc6c(n5)sc5ccccc56)cc4)cc3)n2)cc1 ; c1ccc(-c2nc(-c3ccccc3)nc(-c3ccc(-c4cccc(-c5nc6c(nc5-c5ccccc5)oc5ccccc56)c4)cc3)n2)cc1]

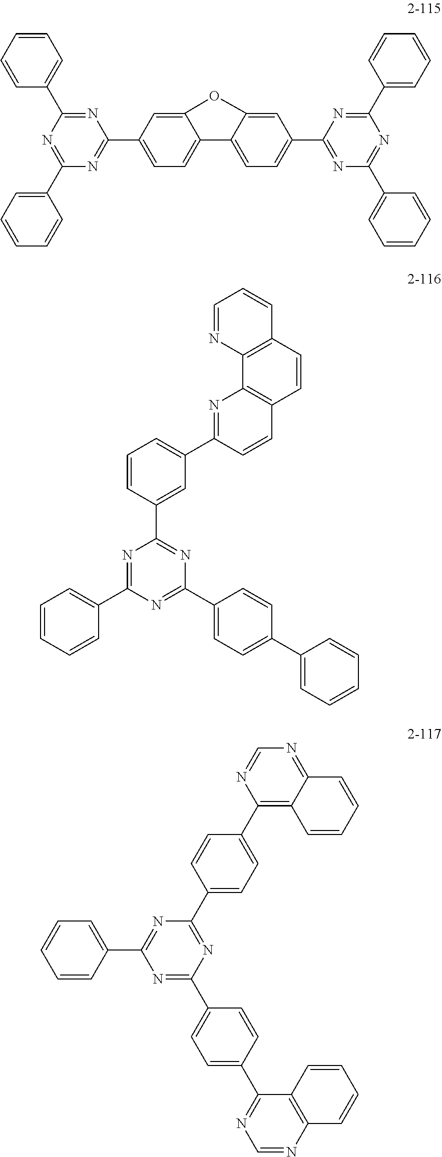 [SMILES: c1ccc(-c2nc(-c3ccccc3)nc(-c3ccc4c(c3)oc3cc(-c5nc(-c6ccccc6)nc(-c6ccccc6)n5)ccc34)n2)cc1 ; c1ccc(-c2ccc(-c3nc(-c4ccccc4)nc(-c4cccc(-c5ccc6ccc7cccnc7c6n5)c4)n3)cc2)cc1 ; c1ccc(-c2nc(-c3ccc(-c4ncnc5ccccc45)cc3)nc(-c3ccc(-c4ncnc5ccccc45)cc3)n2)cc1]

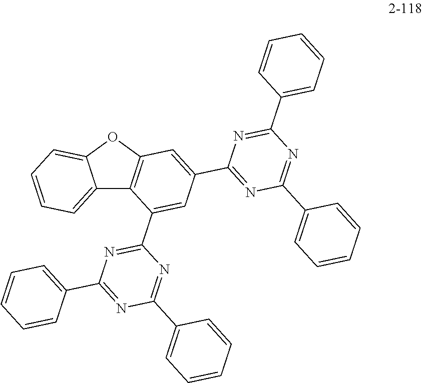 [SMILES: c1ccc(-c2nc(-c3ccccc3)nc(-c3cc(-c4nc(-c5ccccc5)nc(-c5ccccc5)n4)c4c(c3)oc3ccccc34)n2)cc1]

In addition, the present invention provides a compound comprising one or more hole transport band layers between the anode and the emitting layer, wherein the hole transport band layer comprises a hole transport layer, an emitting auxiliary layer, or both, wherein the hole transport band layer comprises the compound represented by Formula 1, wherein the emitting layer comprises the compound represented by Formula 2.

The emitting layer may comprise the compound represented by Formula 2 as a first host, and may further comprise a second host different from the first host.

For example, the second host may be any one selected from Formula 3-1, Formula 3-2, and Compounds 3-1 to 3-135 and 4-1 to 4-65, but are not limited thereto.

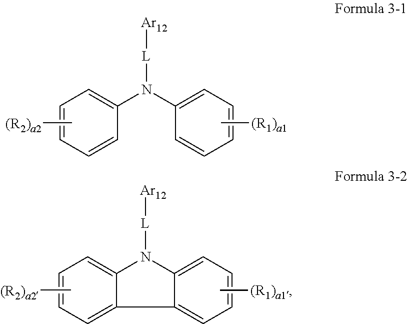

wherein

1) R₁ and R₂ are the same as the definition of R¹ in Formula 1,

2) a1 and a2 are each independently an integer of 0 to 5, a1′ and a2′ are each independently an integer of 0 to 4,

3) L is the same as definition of L¹ in Formula 1,

4) Ar₁₂ is the same as definition of Ar¹ in Formula 1.

However, the first host compound may be excluding Formula C:

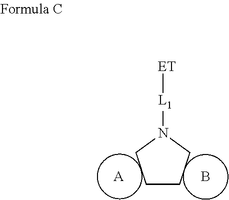

wherein:

1) Ring A and Ring B are each independently a C₆-C₁₄ aryl group,

2) L₁ is selected from the group consisting of a single bond; a C₆-C₆₀ arylene group; fluorenylene group; a fused ring group of a C₃-C₆₀ aliphatic ring and a C₆-C₆₀ aromatic ring; a C₂-C₆₀ heterocyclic group;

3) ET is a C₂-C₆₀ heterocyclic group containing one or more N.

The compound represented by Formula C may be any one of the following compounds:

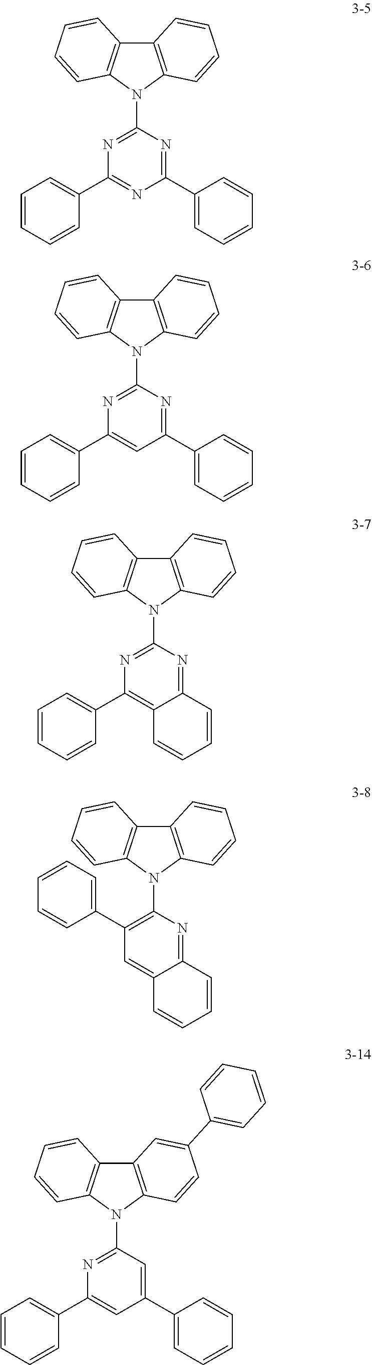

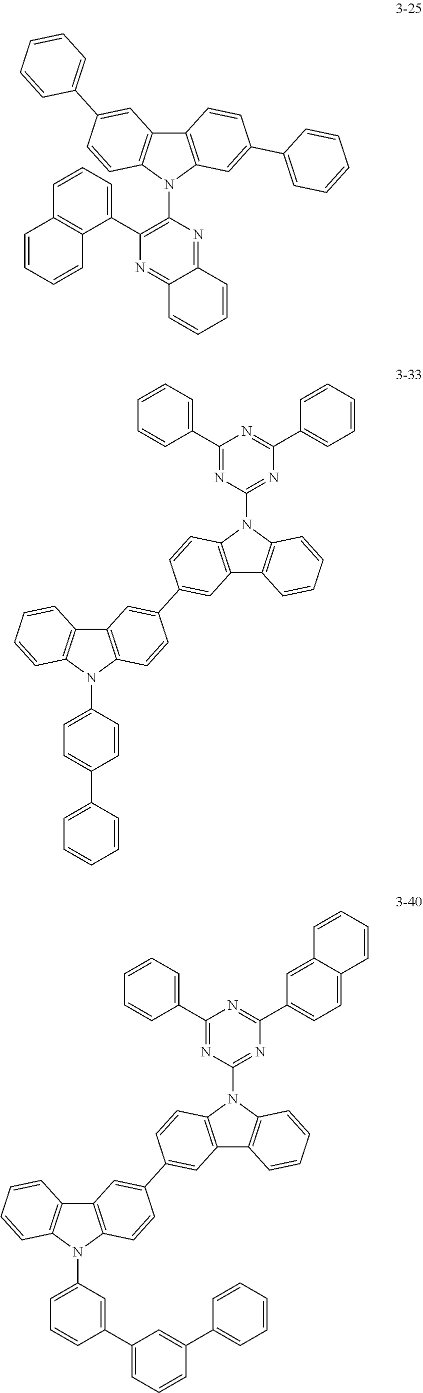 [SMILES: c1ccc(-c2ccc3c(c2)c2ccc(-c4ccccc4)cc2n3-c2nc3ccccc3nc2-c2cccc3ccccc23)cc1 ; c1ccc(-c2ccc(-n3c4ccccc4c4cc(-c5ccc6c(c5)c5ccccc5n6-c5nc(-c6ccccc6)nc(-c6ccccc6)n5)ccc43)cc2)cc1 ; c1ccc(-c2cccc(-c3cccc(-n4c5ccccc5c5cc(-c6ccc7c(c6)c6ccccc6n7-c6nc(-c7ccccc7)nc(-c7ccc8ccccc8c7)n6)ccc54)c3)c2)cc1]

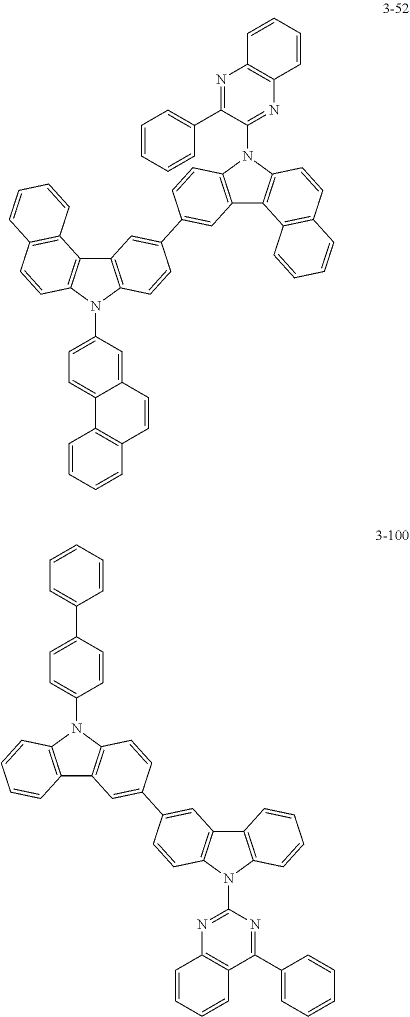 [SMILES: c1ccc(-c2nc3ccccc3nc2-n2c3ccc(-c4ccc5c(c4)c4c6ccccc6ccc4n5-c4ccc5c(ccc6ccccc65)c4)cc3c3c4ccccc4ccc32)cc1 ; c1ccc(-c2ccc(-n3c4ccccc4c4cc(-c5ccc6c(c5)c5ccccc5n6-c5nc(-c6ccccc6)c6ccccc6n5)ccc43)cc2)cc1]

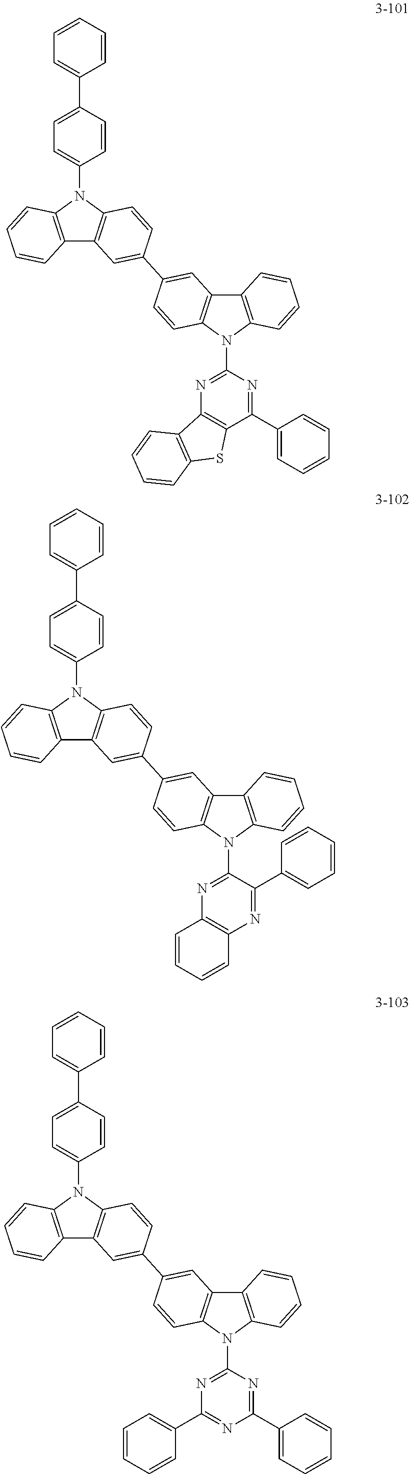 [SMILES: c1ccc(-c2ccc(-n3c4ccccc4c4cc(-c5ccc6c(c5)c5ccccc5n6-c5nc(-c6ccccc6)c6sc7ccccc7c6n5)ccc43)cc2)cc1 ; c1ccc(-c2ccc(-n3c4ccccc4c4cc(-c5ccc6c(c5)c5ccccc5n6-c5nc6ccccc6nc5-c5ccccc5)ccc43)cc2)cc1 ; c1ccc(-c2ccc(-n3c4ccccc4c4cc(-c5ccc6c(c5)c5ccccc5n6-c5nc(-c6ccccc6)nc(-c6ccccc6)n5)ccc43)cc2)cc1]

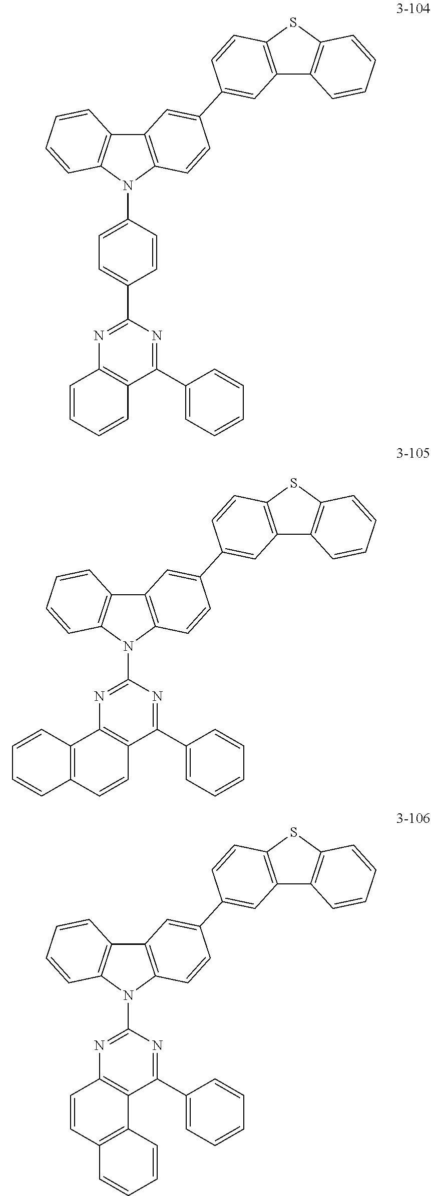

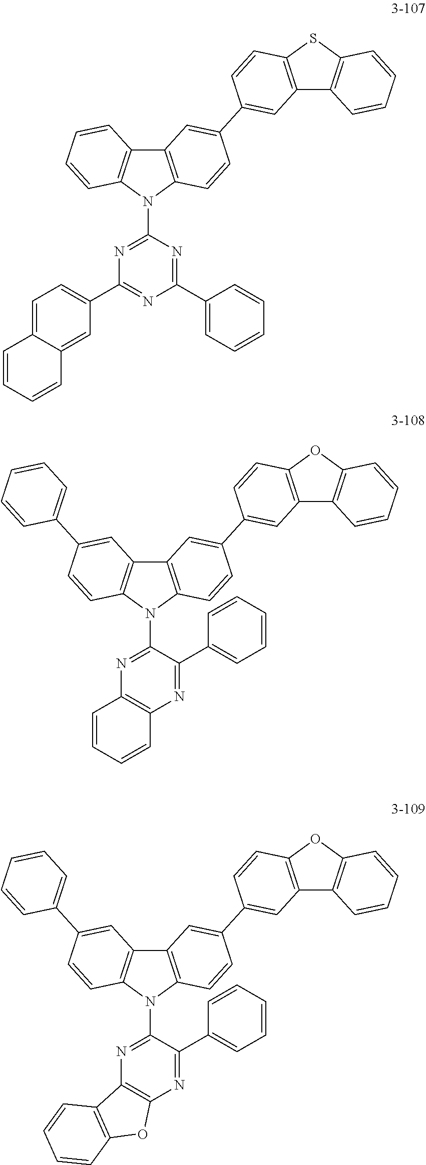

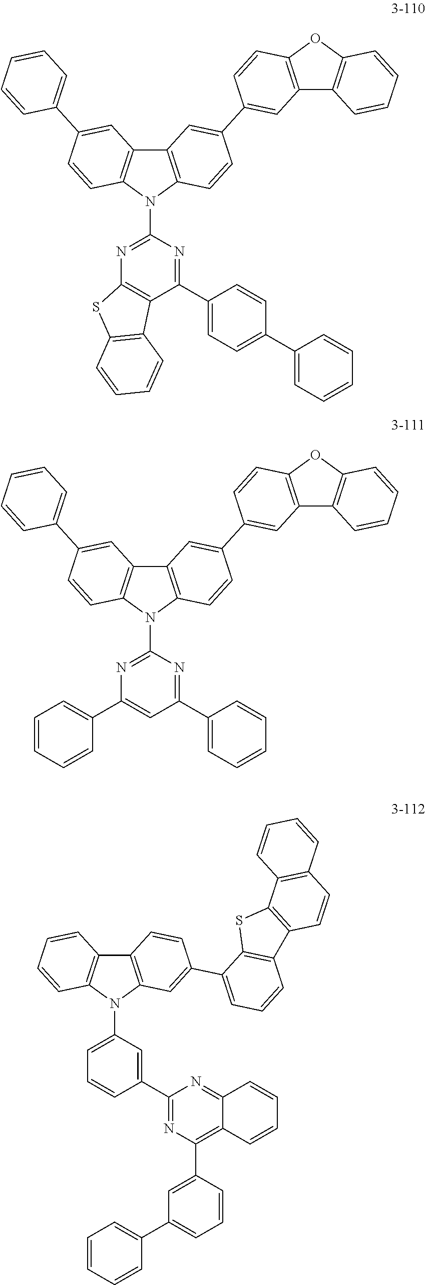 [SMILES: c1ccc(-c2ccc(-c3nc(-n4c5ccc(-c6ccccc6)cc5c5cc(-c6ccc7oc8ccccc8c7c6)ccc54)nc4sc5ccccc5c34)cc2)cc1 ; c1ccc(-c2ccc3c(c2)c2cc(-c4ccc5oc6ccccc6c5c4)ccc2n3-c2nc(-c3ccccc3)cc(-c3ccccc3)n2)cc1 ; c1ccc(-c2cccc(-c3nc(-c4cccc(-n5c6ccccc6c6ccc(-c7cccc8c7sc7c9ccccc9ccc87)cc65)c4)nc4ccccc34)c2)cc1]

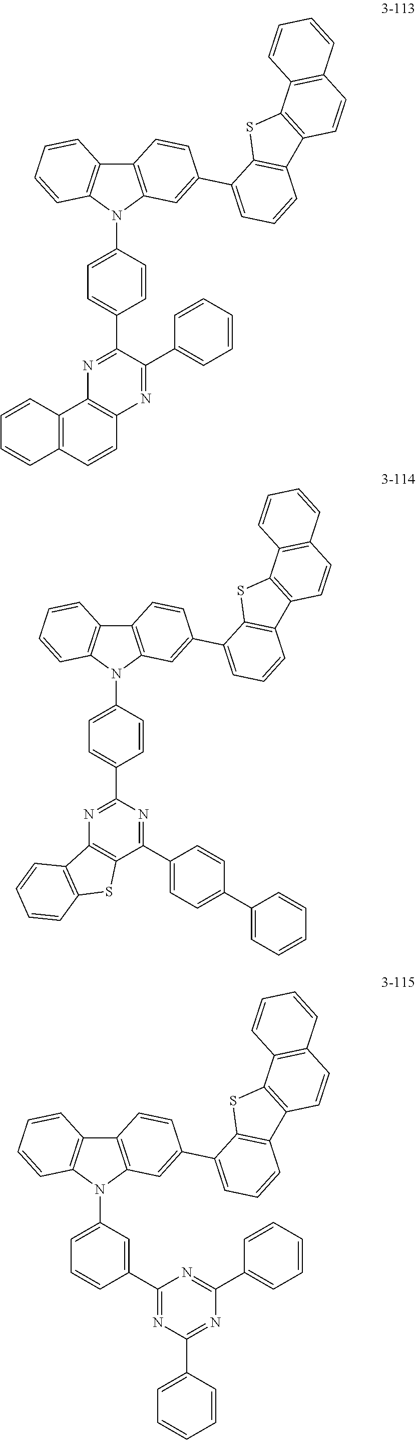

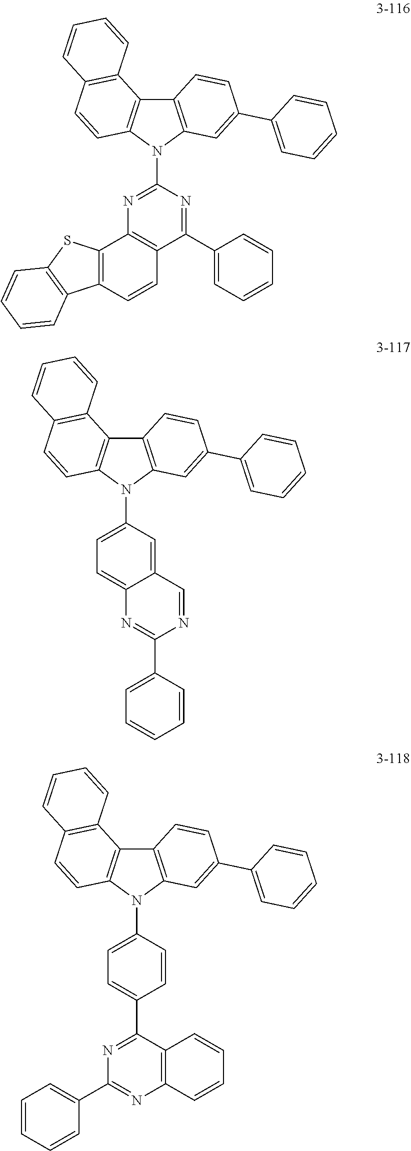

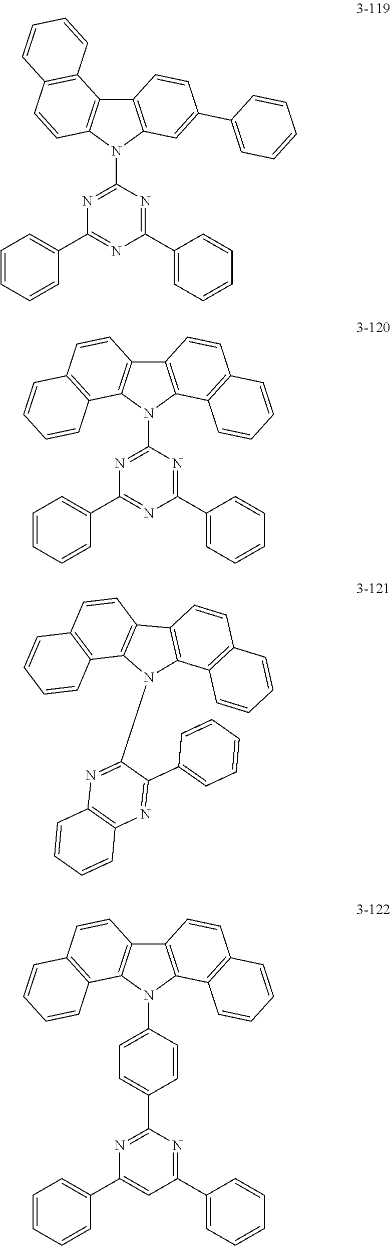

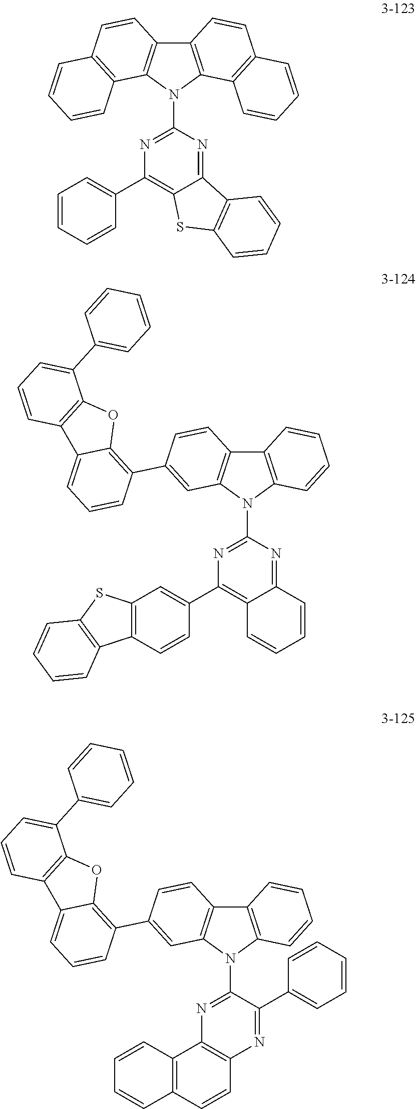 [SMILES: c1ccc(-c2nc(-n3c4c5ccccc5ccc4c4ccc5ccccc5c43)nc3c2sc2ccccc23)cc1 ; c1ccc(-c2cccc3c2oc2c(-c4ccc5c6ccccc6n(-c6nc(-c7ccc8c(c7)sc7ccccc78)c7ccccc7n6)c5c4)cccc23)cc1 ; c1ccc(-c2nc3ccc4ccccc4c3nc2-n2c3ccccc3c3ccc(-c4cccc5c4oc4c(-c6ccccc6)cccc45)cc32)cc1]

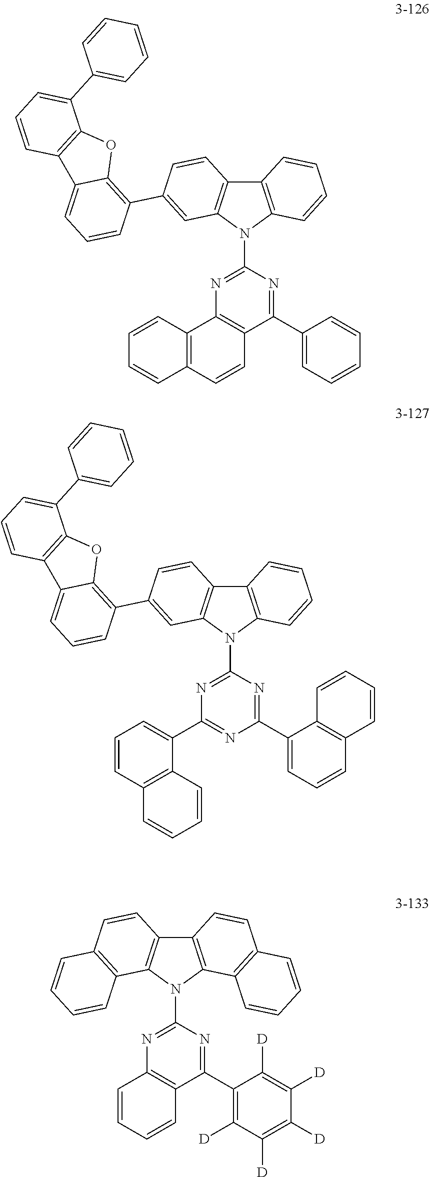 [SMILES: c1ccc(-c2nc(-n3c4ccccc4c4ccc(-c5cccc6c5oc5c(-c7ccccc7)cccc56)cc43)nc3c2ccc2ccccc23)cc1 ; c1ccc(-c2cccc3c2oc2c(-c4ccc5c6ccccc6n(-c6nc(-c7cccc8ccccc78)nc(-c7cccc8ccccc78)n6)c5c4)cccc23)cc1 ; [2H]c1c([2H])c([2H])c(-c2nc(-n3c4c5ccccc5ccc4c4ccc5ccccc5c43)nc3ccccc23)c([2H])c1[2H]]

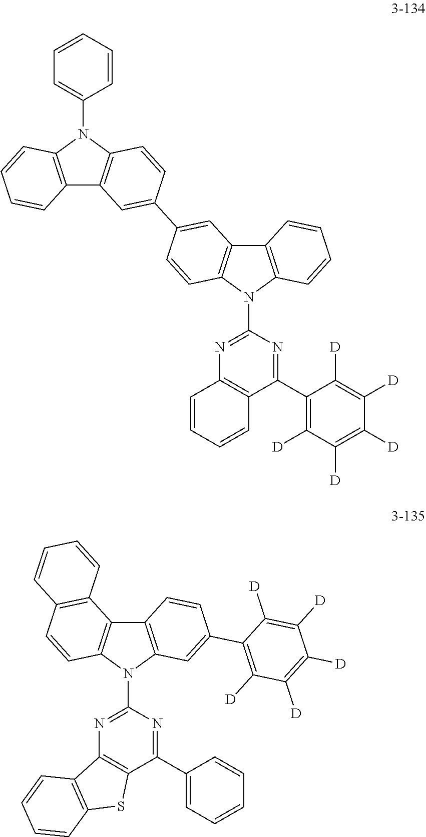 [SMILES: [2H]c1c([2H])c([2H])c(-c2nc(-n3c4ccccc4c4cc(-c5ccc6c(c5)c5ccccc5n6-c5ccccc5)ccc43)nc3ccccc23)c([2H])c1[2H] ; [2H]c1c([2H])c([2H])c(-c2ccc3c4c5ccccc5ccc4n(-c4nc(-c5ccccc5)c5sc6ccccc6c5n4)c3c2)c([2H])c1[2H]]

The organic electronic element further comprises a light efficiency enhancing layer formed on at least one surface of the anode and the cathode, the surface being opposite to the organic material layer.

Moreover, the organic material layer may comprise 2 or more stacks comprising a hole transport layer, an emitting layer, and an electron transport layer sequentially formed on the anode, and the organic material layer may further comprise a charge generating layer formed between 2 or more stacks.

In another aspect, the present invention provides an electronic device comprising a display device comprising the organic electronic element; and a control unit for driving the display device. In this case, the organic electronic element is at least one of an OLED, an organic solar cell, an organic photo conductor (OPC), an organic transistor (organic TFT), and an element for monochromic or white illumination.

Hereinafter, examples of the synthesis of the compound represented by Formula according to the present invention and the preparation of the organic electric device will be described in detail with reference to examples, but the present invention is not limited to the following examples.

### EXAMPLES

### Synthesis Example 1

The compound (Final product 1) represented by Formula 1 according to the present invention may be prepared by reacting as shown in Reaction Scheme 1, but is not limited thereto.

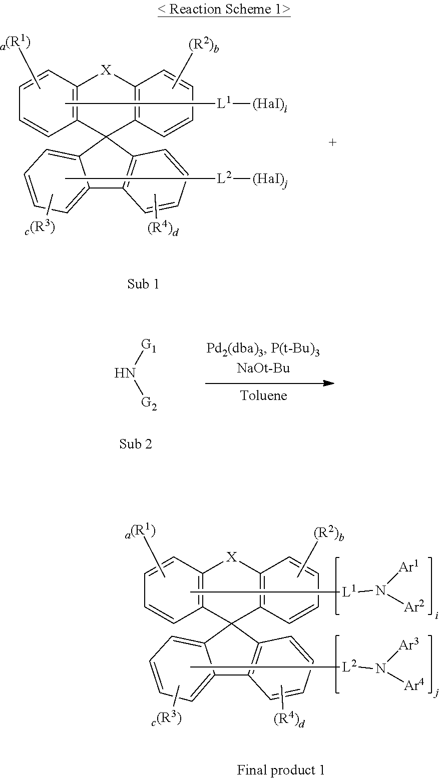

In Reaction scheme 1, Hal is Cl, Br or I, G₁ is Ar¹ or Ar³, G₂ is Ar² or Ar⁴.

### I. Synthesis Example of Sub 1

Sub 1 of Reaction Scheme 1 may be synthesized by the reaction scheme of Reaction Scheme 2, but is not limited thereto.

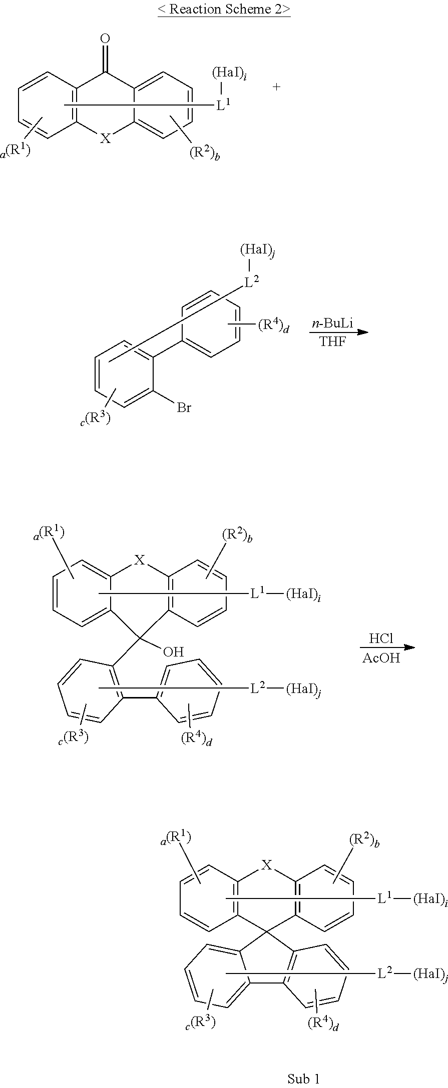

Synthesis examples of specific compounds belonging to Sub 1 are as follows.

### Synthesis Example of Sub 1-1

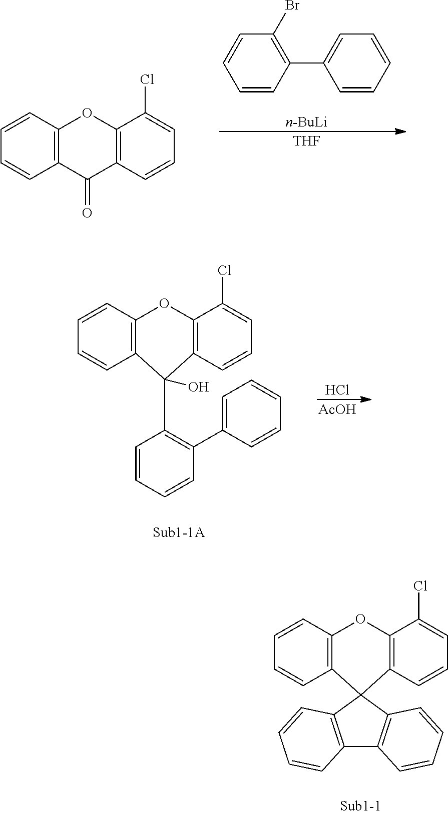

(1) Synthesis of Sub 1-1A

After dissolving 4-chloro-9H-xanthen-9-one (20 g, 86.71 mmol) and 2-bromo-1,1′-biphenyl (21.22 g, 91.05 mmol) in THF (600 ml), the temperature of the reaction was lowered to −78° C., slowly adding n-BuLi (2.5 M in hexane) (6.11 g, 95.38 mmol), the reaction mixture was stirred at room temperature for 4 hours. When the reaction was completed, the reactant was put into H₂O for quenching, and then water in the reactant was removed, filtered under reduced pressure, and the organic solvent was concentrated. The resulting product was separated using column chromatography to obtain 29.7 g of the product. (Yield: 89%)

(2) Synthesis of Sub 1-1

Sub 1-1A (20 g, 51.97 mmol), HCl (4 ml), Acetic acid (208 ml) were added and stirred at 80° C. for 1 hour. When the reaction was completed, after filtration under reduced pressure, the organic solvent was concentrated and the resulting product was separated using column chromatography to obtain 17.54 g of the product. (Yield: 92%)

### Synthesis Example of Sub 1-6

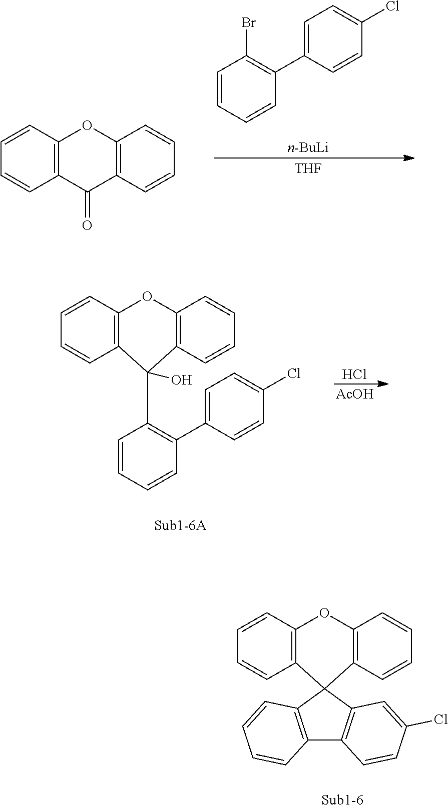

(1) Synthesis of Sub 1-6A

4-chloro-9H-xanthen-9-one (20 g, 101.93 mmol) and 2-bromo-1,1′-biphenyl (28.64 g, 107.03 mmol), THF (680 ml), n-BuLi (2.5 M in hexane) (7.18 g, 112.12 mmol) were used to obtain 33.3 g of the product by using the method for synthesizing Sub 1-1A. (Yield: 85%)

(2) Synthesis of Sub 1-6

Sub 1-6A (20 g, 51.97 mmol), HCl (4 ml), and acetic acid (208 ml) were used to obtain 16.78 g of the product using the synthesis method of Sub 1-1. (Yield: 88%)

### Synthesis Example of Sub 1-46

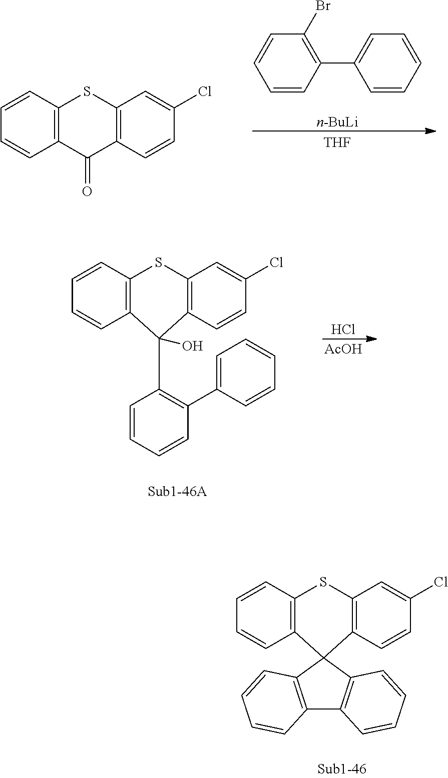

(1) Synthesis of Sub 1-46A

3-chloro-9H-thioxanthen-9-one (20 g, 81.07 mmol), 2-bromo-1,1′-biphenyl (19.84 g, 85.12 mmol), THF (600 ml), n-BuLi (2.5 M in hexane) (5.71 g, 89.17 mmol) were used to obtain 25.7 g of the product by using the method for synthesizing Sub 1-1A. (Yield: 79%)

(2) Synthesis of Sub 1-46

Sub 1-46A (20.8 g, 51.97 mmol), HCl (4 ml), Acetic acid (200 ml) were used to obtain 15.47 g of the product using the synthesis method of Sub 1-1. (Yield: 81%)

### Synthesis Example of Sub 1-55

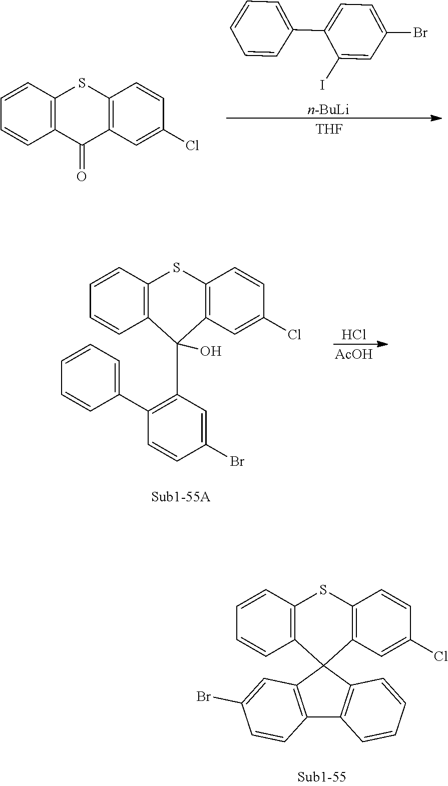

(1) Synthesis of Sub 1-55A

2-chloro-9H-thioxanthen-9-one (20 g, 81.07 mmol), 4-bromo-2-iodo-1,1′-biphenyl (30.56 g, 85.12 mmol), THF (600 ml), n-BuLi (2.5 M in hexane) (5.71 g, 89.17 mmol) were used to obtain 33.06 g of the product by using the method for synthesizing Sub 1-1A. (Yield: 85%)

(2) Synthesis of Sub 1-55

Sub 1-55A (20 g, 41.68 mmol), HCl (3.5 ml), Acetic acid (167 ml) were used to obtain 16.75 g of the product using the synthesis method of Sub 1-1. (Yield: 87%)

### Synthesis Example of Sub 1-72

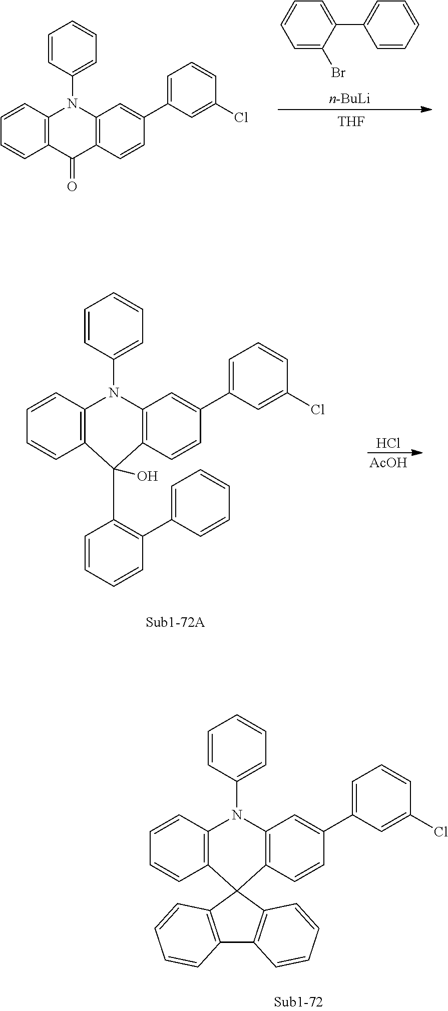

(1) Synthesis of Sub 1-72A

3-(3-chlorophenyl)-10-phenylacridin-9(10H)-one (20 g, 52.38 mmol), 2-bromo-1,1′-biphenyl (12.82 g, 54.99 mmol), THF (500 ml), n-BuLi (2.5 M in hexane) (3.7 g, 57.61 mmol) were used to obtain 25 g of the product using the synthesis method of Sub 1-1A. (Yield: 89%)

(2) Synthesis of Sub 1-72

Sub 1-72A (20 g, 37.31 mmol), HCl (3 ml), Acetic acid (150 ml) were used to obtain 17.59 g of the product using the synthesis method of Sub 1-1. (Yield: 91%)

The compound belonging to Sub 1 may be the following compounds, but is not limited thereto:

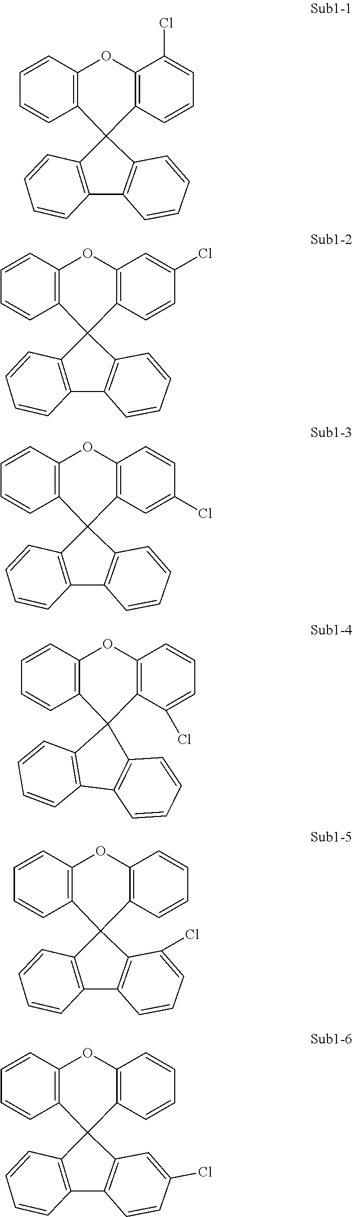

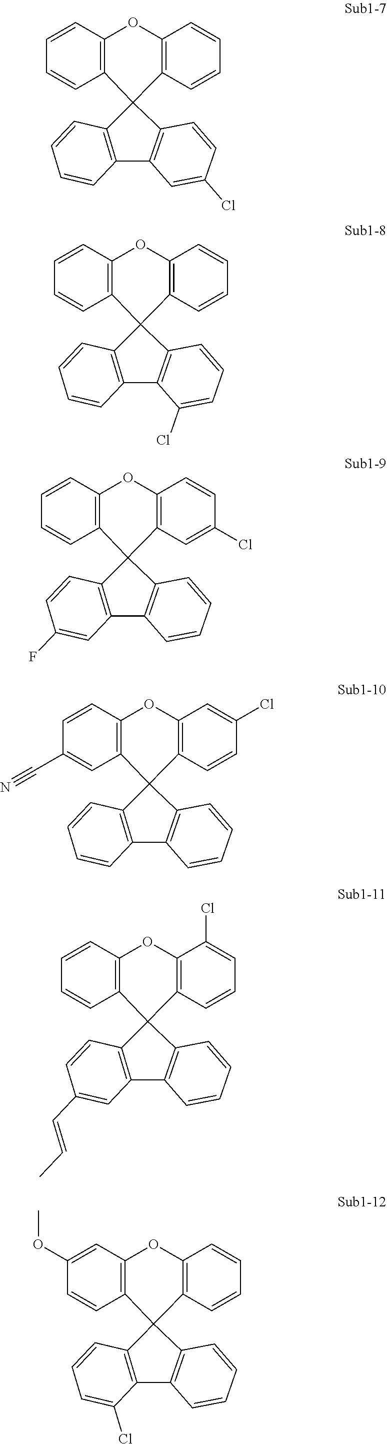

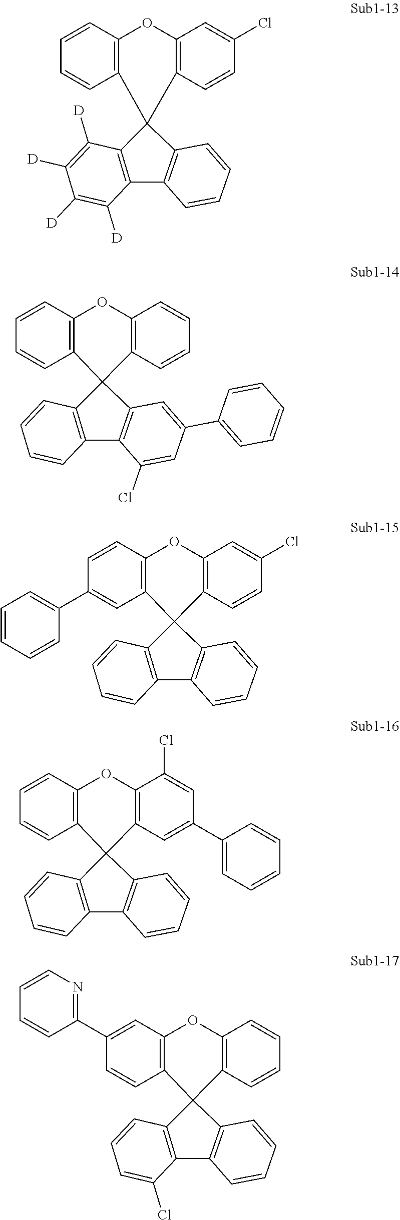

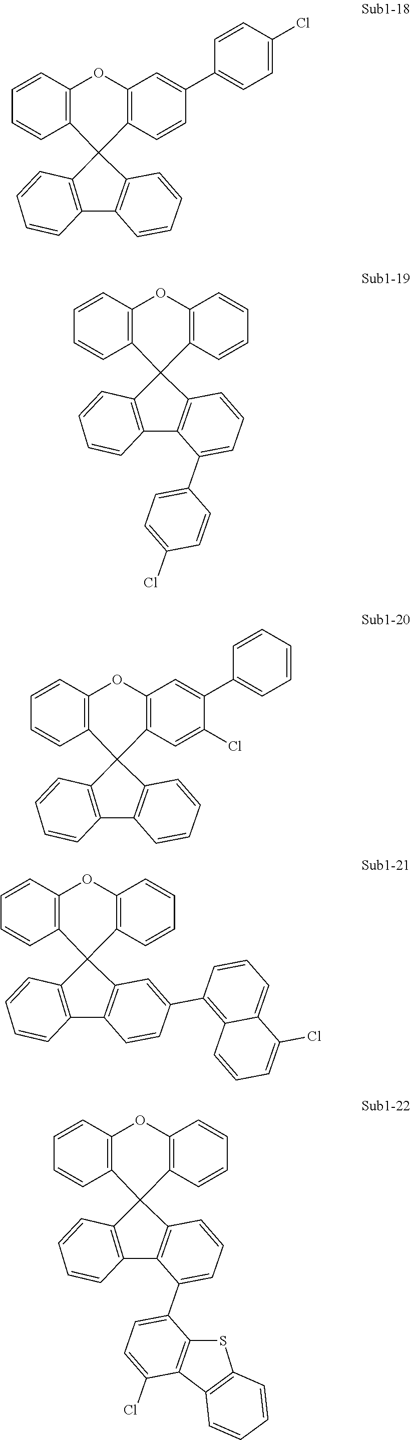

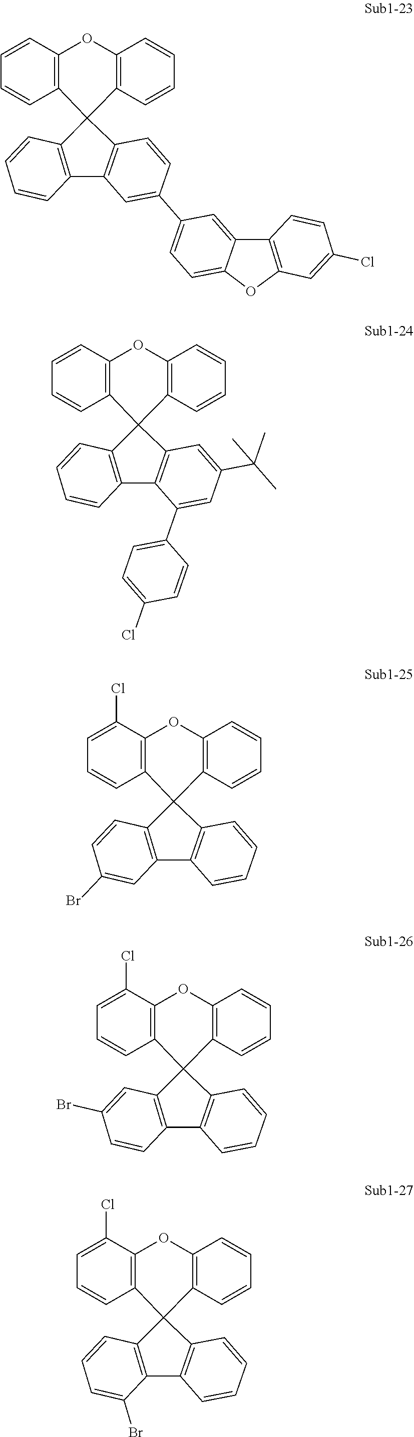 [SMILES: Clc1ccc2c(c1)oc1ccc(-c3ccc4c(c3)-c3ccccc3C43c4ccccc4Oc4ccccc43)cc12 ; CC(C)(C)c1cc(-c2ccc(Cl)cc2)c2c(c1)C1(c3ccccc3Oc3ccccc31)c1ccccc1-2 ; Clc1cccc2c1Oc1ccccc1C21c2ccccc2-c2cc(Br)ccc21 ; Clc1cccc2c1Oc1ccccc1C21c2ccccc2-c2ccc(Br)cc21 ; Clc1cccc2c1Oc1ccccc1C21c2ccccc2-c2c(Br)cccc21]

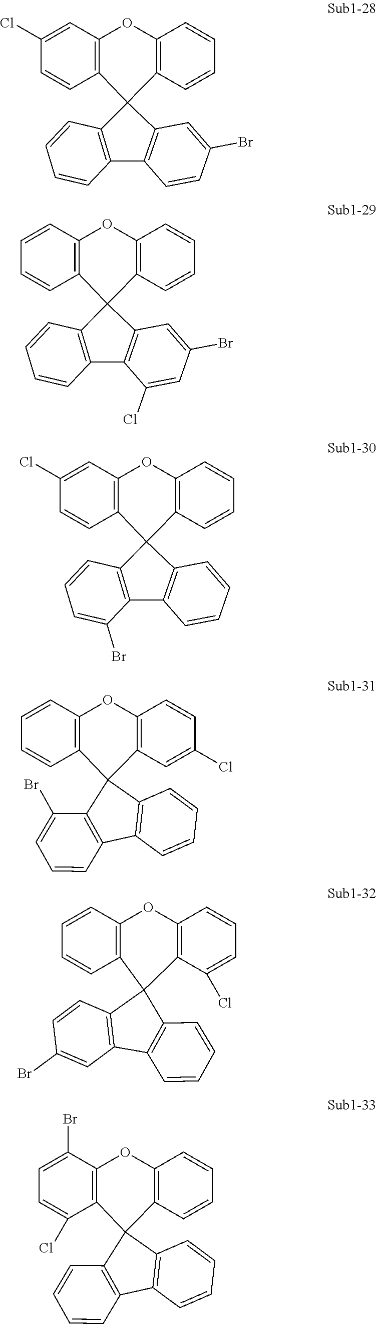

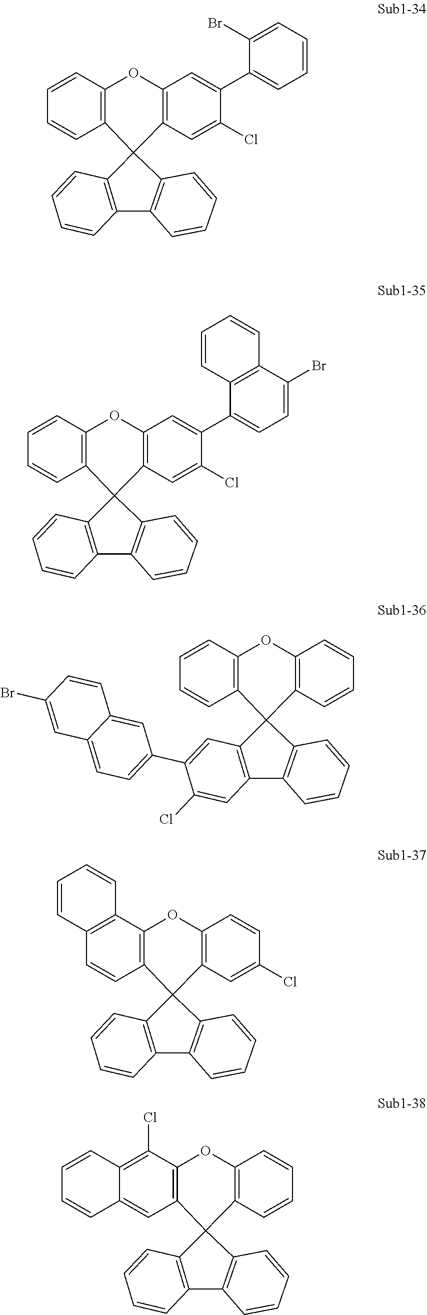

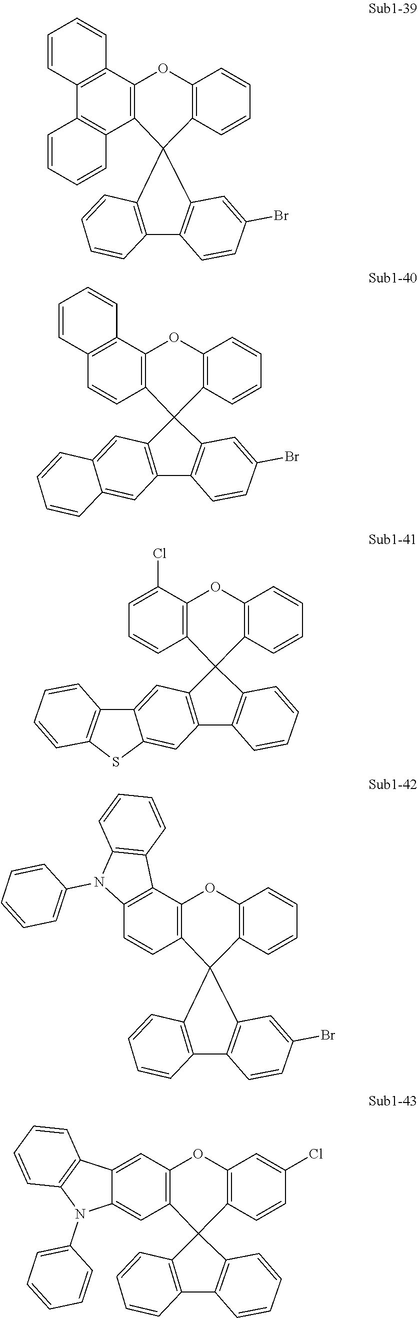

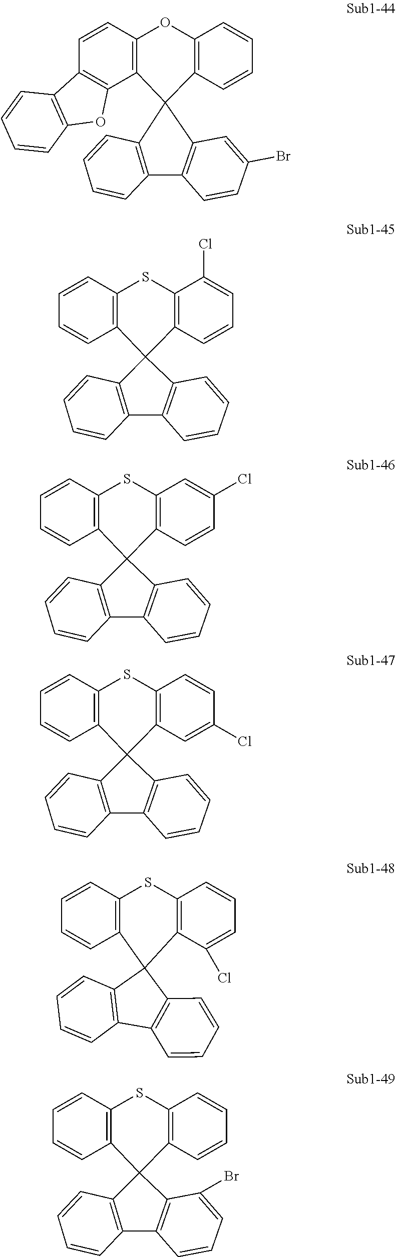

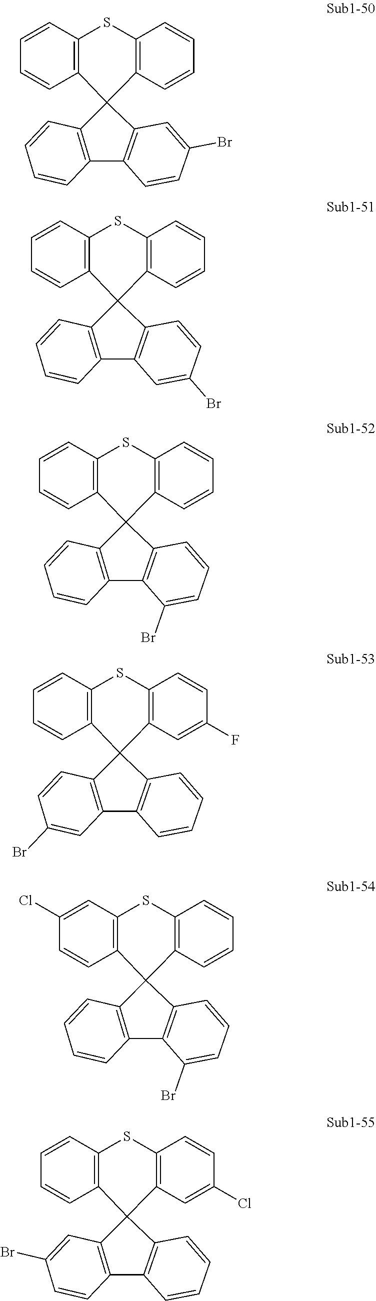

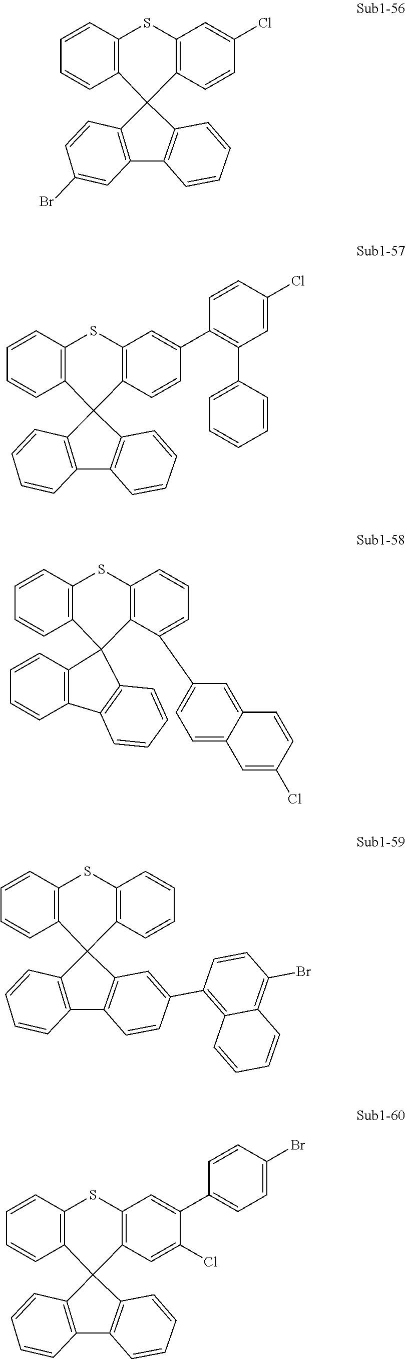 [SMILES: Clc1ccc2c(c1)Sc1ccccc1C21c2ccccc2-c2cc(Br)ccc21 ; Clc1ccc(-c2ccc3c(c2)Sc2ccccc2C32c3ccccc3-c3ccccc32)c(-c2ccccc2)c1 ; Clc1ccc2cc(-c3cccc4c3C3(c5ccccc5S4)c4ccccc4-c4ccccc43)ccc2c1 ; Brc1ccc(-c2ccc3c(c2)C2(c4ccccc4Sc4ccccc42)c2ccccc2-3)c2ccccc12 ; Clc1cc2c(cc1-c1ccc(Br)cc1)Sc1ccccc1C21c2ccccc2-c2ccccc21]

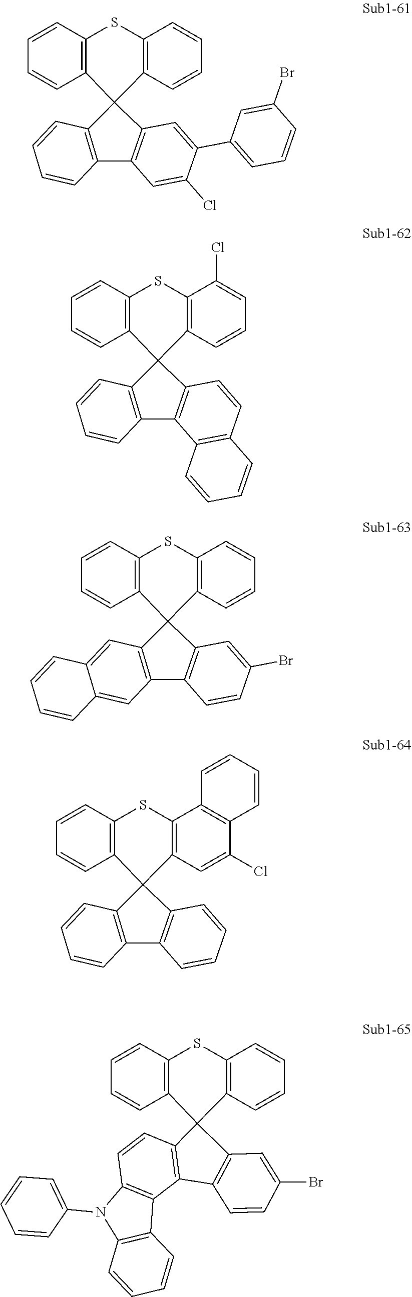

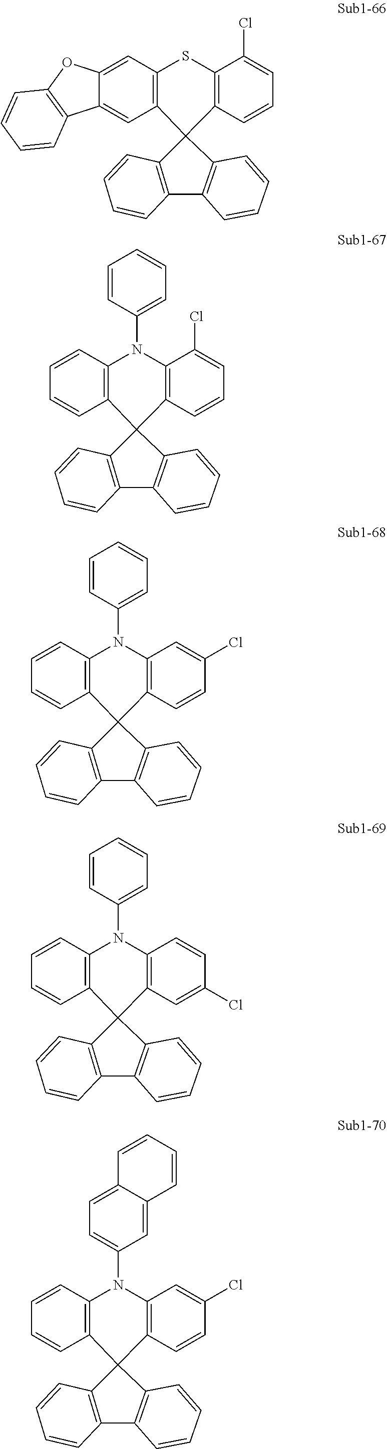

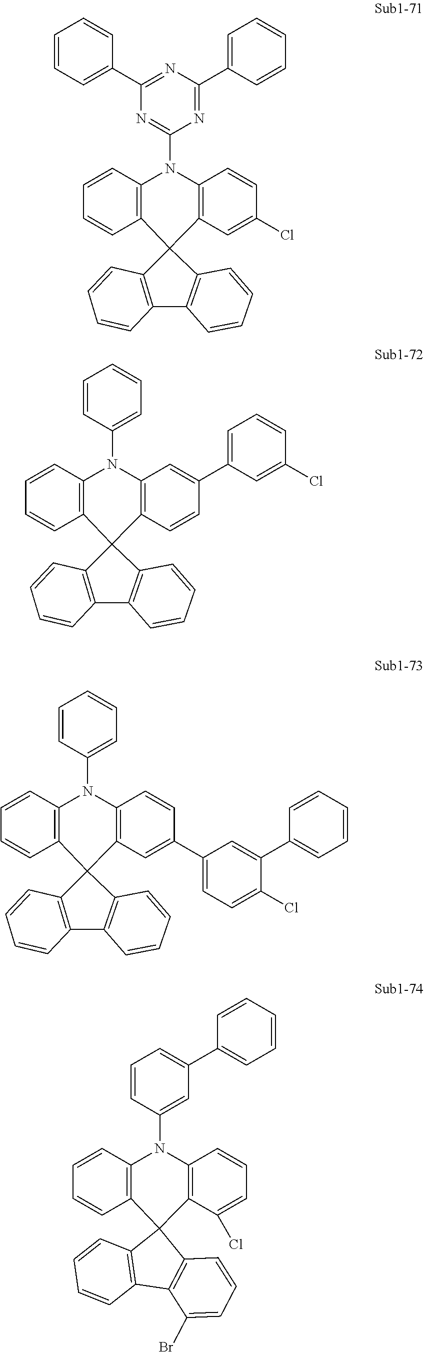

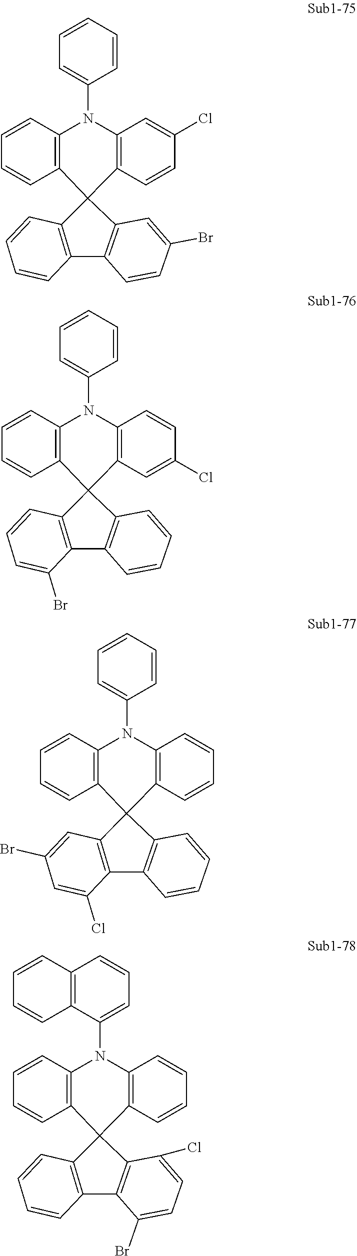

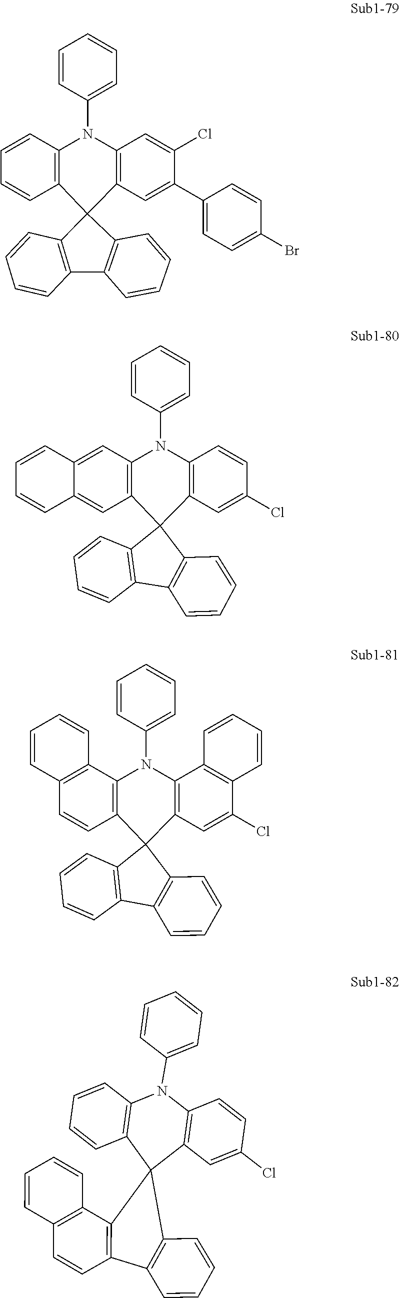 [SMILES: Clc1cc2c(cc1-c1ccc(Br)cc1)C1(c3ccccc3-c3ccccc31)c1ccccc1N2c1ccccc1 ; Clc1ccc2c(c1)C1(c3ccccc3-c3ccccc31)c1cc3ccccc3cc1N2c1ccccc1 ; Clc1cc2c(c3ccccc13)N(c1ccccc1)c1c(ccc3ccccc13)C21c2ccccc2-c2ccccc21 ; Clc1ccc2c(c1)C1(c3ccccc3-c3ccc4ccccc4c31)c1ccccc1N2c1ccccc1]

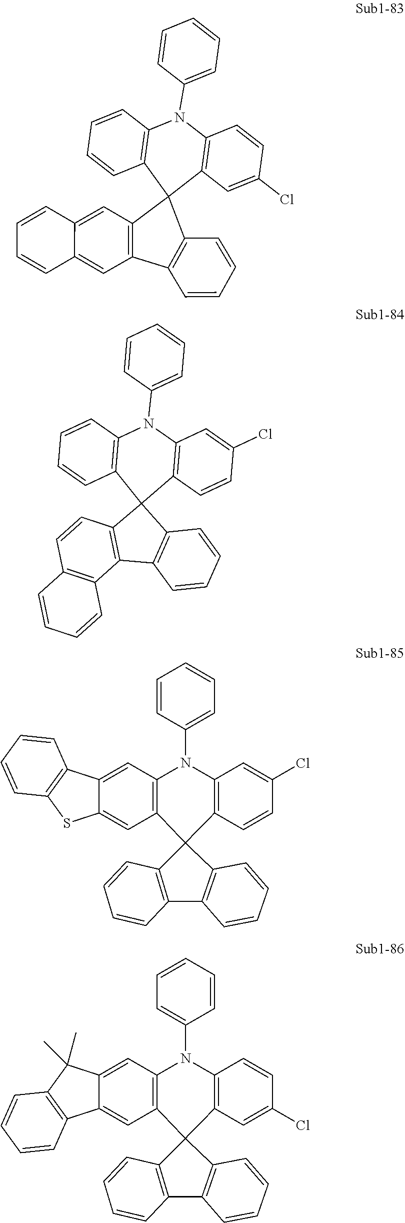

Table 1 shows FD-MS (Field Desorption-Mass Spectrometry) values of compounds belonging to Sub 1.

TABLE 1

| Com- | |
|---|---|
| pound | FD-MS |
| Sub 1-1 | m/z = 366.08 (C₂₅H₁₅ClO = 366.84) |
| Sub 1-2 | m/z = 366.08 (C₂₅H₁₅ClO = 366.84) |
| Sub 1-3 | m/z = 366.08 (C₂₅H₁₅ClO = 366.84) |
| Sub 1-4 | m/z = 366.08 (C₂₅H₁₅ClO = 366.84) |
| Sub 1-5 | m/z = 366.08 (C₂₅H₁₅ClO = 366.84) |
| Sub 1-6 | m/z = 366.08 (C₂₅H₁₅ClO = 366.84) |
| Sub 1-7 | m/z = 366.08 (C₂₅H₁₅ClO = 366.84) |
| Sub 1-8 | m/z = 366.08 (C₂₅H₁₅ClO = 366.84) |
| Sub 1-9 | m/z = 384.07 (C₂₅H₁₄ClFO = 384.83) |
| Sub 1-10 | m/z = 391.08 (C₂₆H₁₄ClNO = 391.85) |
| Sub 1-11 | m/z = 406.11 (C₂₈H₁₉ClO = 406.91) |
| Sub 1-12 | m/z = 396.09 (C₂₆H₁₇ClO₂ = 396.87) |
| Sub 1-13 | m/z = 370.11 (C₂₅H₁₁D₄ClO = 370.87) |
| Sub 1-14 | m/z = 442.11 (C₃₁H₁₉ClO = 442.94) |
| Sub 1-15 | m/z = 442.11 (C₃₁H₁₉ClO = 442.94) |
| Sub 1-16 | m/z = 442.11 (C₃₁H₁₉ClO = 442.94) |
| Sub 1-17 | m/z = 443.11 (C₃₀H₁₈ClNO = 443.93) |
| Sub 1-18 | m/z = 442.11 (C₃₁H₁₉ClO = 442.94) |
| Sub 1-19 | m/z = 442.11 (C₃₁H₁₉ClO = 442.94) |
| Sub 1-20 | m/z = 442.11 (C₃₁H₁₉ClO = 442.94) |
| Sub 1-21 | m/z = 492.13 (C₃₅H₂₁ClO = 493) |
| Sub 1-22 | m/z = 548.1 (C₃₇H₂₁ClOS = 549.08) |
| Sub 1-23 | m/z = 532.12 (C₃₇H₂₁ClO₂ = 533.02) |
| Sub 1-24 | m/z = 498.18 (C₃₅H₂₇ClO = 499.05) |
| Sub 1-25 | m/z = 443.99 (C₂₅H₁₄BrClO = 445.74) |
| Sub 1-26 | m/z = 443.99 (C₂₅H₁₄BrClO = 445.74) |
| Sub 1-27 | m/z = 443.99 (C₂₅H₁₄BrClO = 445.74) |
| Sub 1-28 | m/z = 443.99 (C₂₅H₁₄BrClO = 445.74) |
| Sub 1-29 | m/z = 443.99 (C₂₅H₁₄BrClO = 445.74) |
| Sub 1-30 | m/z = 443.99 (C₂₅H₁₄BrClO = 445.74) |
| Sub 1-31 | m/z = 443.99 (C₂₅H₁₄BrClO = 445.74) |
| Sub 1-32 | m/z = 443.99 (C₂₅H₁₄BrClO = 445.74) |
| Sub 1-33 | m/z = 443.99 (C₂₅H₁₄BrClO = 445.74) |
| Sub 1-34 | m/z = 520.02 (C₃₁H₁₈BrClO = 521.84) |
| Sub 1-35 | m/z = 570.04 (C₃₅H₂₀BrClO = 571.9) |
| Sub 1-36 | m/z = 570.04 (C₃₅H₂₀BrClO = 571.9) |
| Sub 1-37 | m/z = 416.1 (C₂₉H₁₇ClO = 416.9) |
| Sub 1-38 | m/z = 416.1 (C₂₉H₁₇ClO = 416.9) |
| Sub 1-39 | m/z = 510.06 (C₃₃H₁₉BrO = 511.42) |
| Sub 1-40 | m/z = 510.06 (C₃₃H₁₉BrO = 511.42) |
| Sub 1-41 | m/z = 472.07 (C₃₁H₁₇ClOS = 472.99) |
| Sub 1-42 | m/z = 575.09 (C₃₇H₂₂BrNO = 576.49) |
| Sub 1-43 | m/z = 531.14 (C₃₇H₂₂ClNO = 532.04) |
| Sub 1-44 | m/z = 500.04 (C₃₁H₁₇BrO₂ = 501.38) |
| Sub 1-45 | m/z = 382.06 (C₂₅H₁₅ClS = 382.91) |
| Sub 1-46 | m/z = 382.06 (C₂₅H₁₅ClS = 382.91) |
| Sub 1-47 | m/z = 382.06 (C₂₅H₁₅ClS = 382.91) |
| Sub 1-48 | m/z = 382.06 (C₂₅H₁₅ClS = 382.91) |
| Sub 1-49 | m/z = 426.01 (C₂₅H₁₅BrS = 427.36) |
| Sub 1-50 | m/z = 426.01 (C₂₅H₁₅BrS = 427.36) |
| Sub 1-51 | m/z = 426.01 (C₂₅H₁ₛBrS = 427.36) |
| Sub 1-52 | m/z = 426.01 (C₂₅H₁ₛBrS = 427.36) |
| Sub 1-53 | m/z = 444 (C₂₅H₁₄BrFS = 445.35) |
| Sub 1-54 | m/z = 459.97 (C₂₅H₁₄BrClS = 461.8) |
| Sub 1-55 | m/z = 459.97 (C₂₅H₁₄BrClS = 461.8) |
| Sub 1-56 | m/z = 459.97 (C₂₅H₁₄BrClS = 461.8) |
| Sub 1-57 | m/z = 534.12 (C₃₇H₂₃ClS = 535.1) |
| Sub 1-58 | m/z = 508.11 (C₃₅H₂₁ClS = 509.06) |
| Sub 1-59 | m/z = 552.05 (C₃₅H₂₁BrS = 553.52) |
| Sub 1-60 | m/z = 536 (C₃₁H₁₈BrClS = 537.9) |
| Sub 1-61 | m/z = 536 (C₃₁H₁₈BrClS = 537.9) |
| Sub 1-62 | m/z = 432.07 (C₂₉H₁₇ClS = 432.97) |
| Sub 1-63 | m/z = 476.02 (C₂₉H₁₇BrS = 477.42) |
| Sub 1-64 | m/z = 432.07 (C₂₉H₁₇ClS = 432.97) |
| Sub 1-65 | m/z = 591.07 (C₃₇H₂₂BrNS = 592.55) |
| Sub 1-66 | m/z = 472.07 (C₃₁H₁₇ClOS = 472.99) |
| Sub 1-67 | m/z = 441.13 (C₃₁H₂₀ClN = 441.96) |
| Sub 1-68 | m/z = 441.13 (C₃₁H₂₀ClN = 441.96) |
| Sub 1-69 | m/z = 441.13 (C₃₁H₂₀ClN = 441.96) |
| Sub 1-70 | m/z = 491.14 (C₃₅H₂₂ClN = 492.02) |
| Sub 1-71 | m/z = 596.1 8 (C₄₀H₂₅ClN₄ = 597.12) |
| Sub 1-72 | m/z = 517.16 (C₃₇H₂₄ClN = 518.06) |
| Sub 1-73 | m/z = 593.19 (C₄₃H₂₈ClN = 594.15) |
| Sub 1-74 | m/z = 595.07 (C₃₇H₂₃BrClN = 596.95) |
| Sub 1-75 | m/z = 51 9.04 (C₃₁H₁₉BrClN = 520.85) |
| Sub 1-76 | m/z = 519.04 (C₃₁H₁₉BrClN = 520.85) |
| Sub 1-77 | m/z = 51 9.04 (C₃₁H₁₉BrClN = 520.85) |
| Sub 1-78 | m/z = 569.05 (C₃₅H₆₄BrClN = 570.91) |
| Sub 1-79 | m/z = 595.07 (C₃₇H₂₃BrClN = 596.95) |
| Sub 1-80 | m/z = 491.14 (C₃₅H₂₂ClN = 492.02) |
| Sub 1-81 | m/z = 541.16 (C₃₉H₂₄ClN = 542.08) |
| Sub 1-82 | m/z = 491.14 (C₃₅H₂₂ClN = 492.02) |
| Sub 1-83 | m/z = 491.14 (C₃₅H₂₂ClN = 492.02) |
| Sub 1-84 | m/z = 491.14 (C₃₅H₂₂ClN = 492.02) |
| Sub 1-85 | m/z = 547.12 (C₃₇H₂₂ClNS = 548.1) |
| Sub 1-86 | m/z = 557.19 (C₄₀H₂₈ClN = 558.12) |

### II. Synthesis Example of Sub2

Sub 2 of Scheme 1 may be synthesized by the reaction pathway of Reaction Scheme 3, but is not limited thereto.

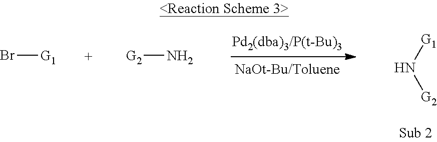

In Reaction Scheme 3, G₁ is Ar¹ or Ar³, G₂ is Ar³ or Ar⁴.

### Synthesis Example of Sub 2-1

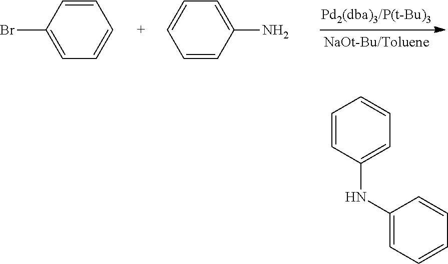 [SMILES: Brc1ccccc1 ; Nc1ccccc1 ; c1ccc(Nc2ccccc2)cc1]

After putting bromobenzene (37.1 g, 236.2 mmol) in a round bottom flask and dissolving it with toluene (2200 ml), aniline (20 g, 214.8 mmol), Pd₂(dba)₃ (9.83 g, 10.7 mmol), P(t-Bu)₃ (4.34 g, 21.5 mmol) and NaOt-Bu (62 g, 644.3 mmol) were added in that order and stirred at 100° C. After the reaction was completed, the mixture was extracted with ether and water, and the organic layer was dried over MgSO₄, concentrated, and the resulting compound was recrystallized using a silica gel column to obtain 28 g of Sub 2-1 (yield: 77%).

### Synthesis Example of Sub 2-37

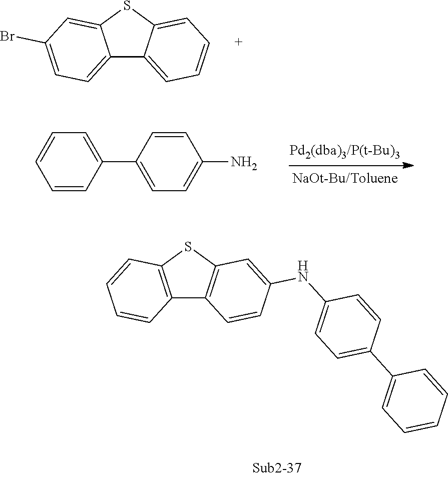 [SMILES: Brc1ccc2c(c1)sc1ccccc12 ; Nc1ccc(-c2ccccc2)cc1 ; c1ccc(-c2ccc(Nc3ccc4c(c3)sc3ccccc34)cc2)cc1]

3-bromodibenzo[b,d]thiophene (42.8 g, 162.5 mmol), toluene (1550 ml), [1,1′-biphenyl]-4-amine (25 g, 147.7 mmol), Pd₂(dba)₃ (6.76 g, 7.4 mmol), P(t-Bu)₃ (3 g, 14.8 mmol), NaOt-Bu (42.6 g, 443.2 mmol) were used to obtain 37.9 g of Sub 2-37 using the synthesis method of Sub 2-1. (Yield: 73%)

Compounds belonging to Sub 2 may be the following compounds, but are not limited thereto:

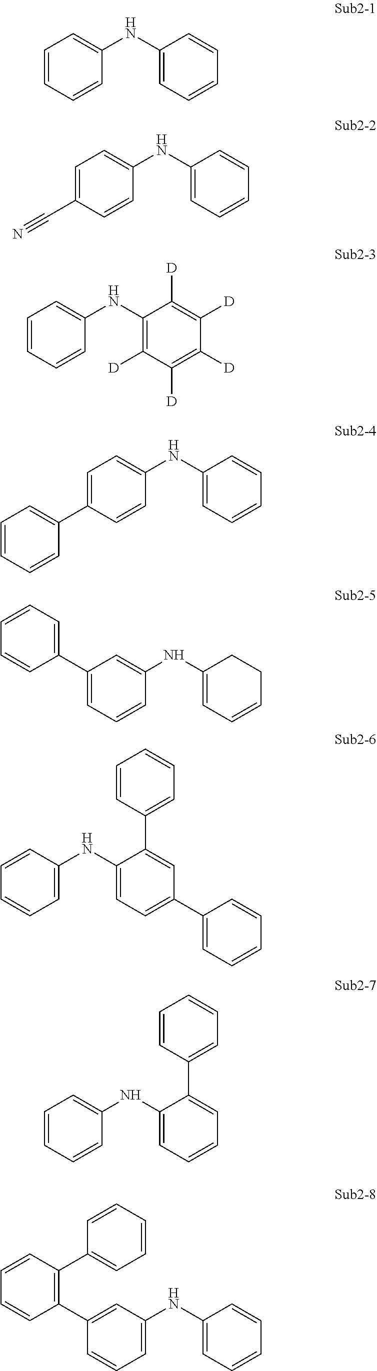

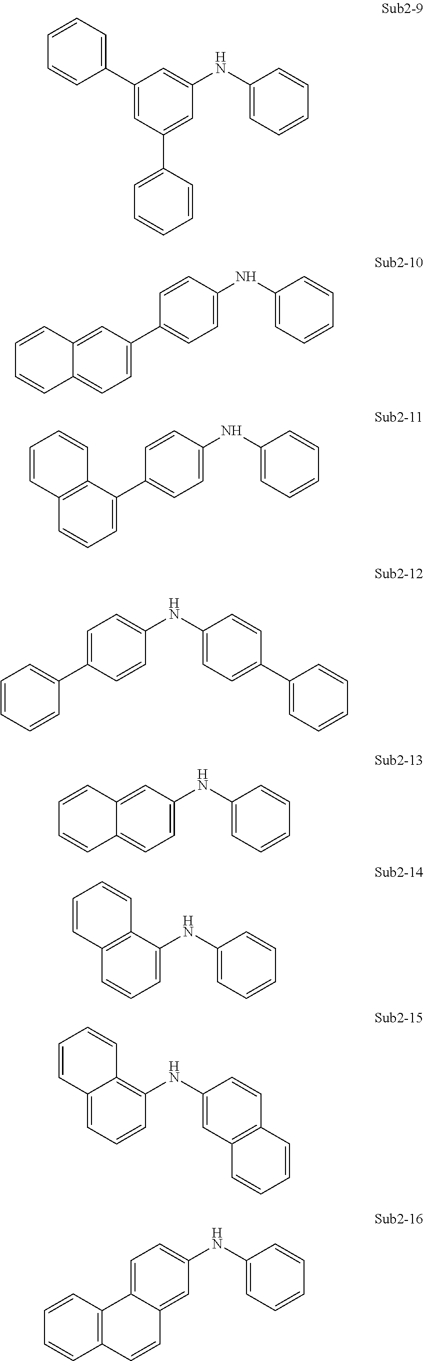

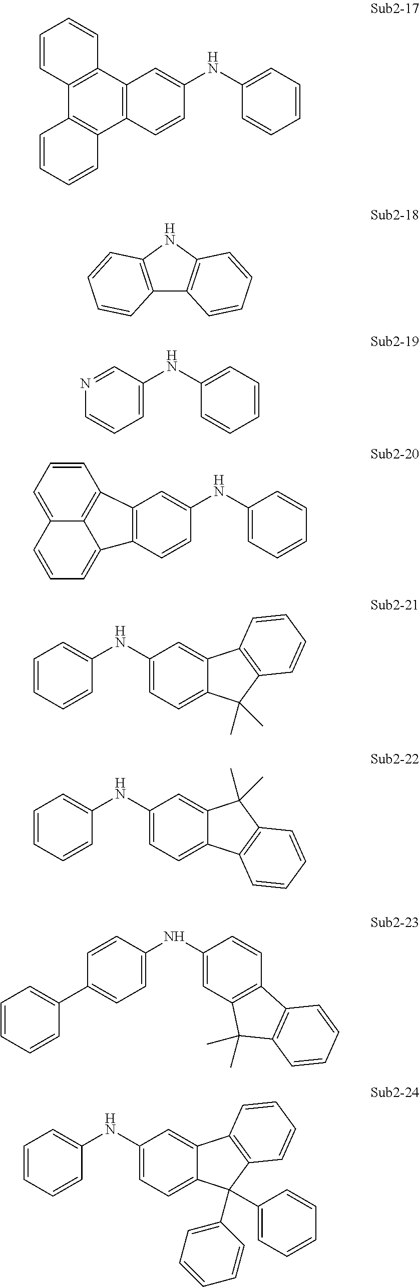

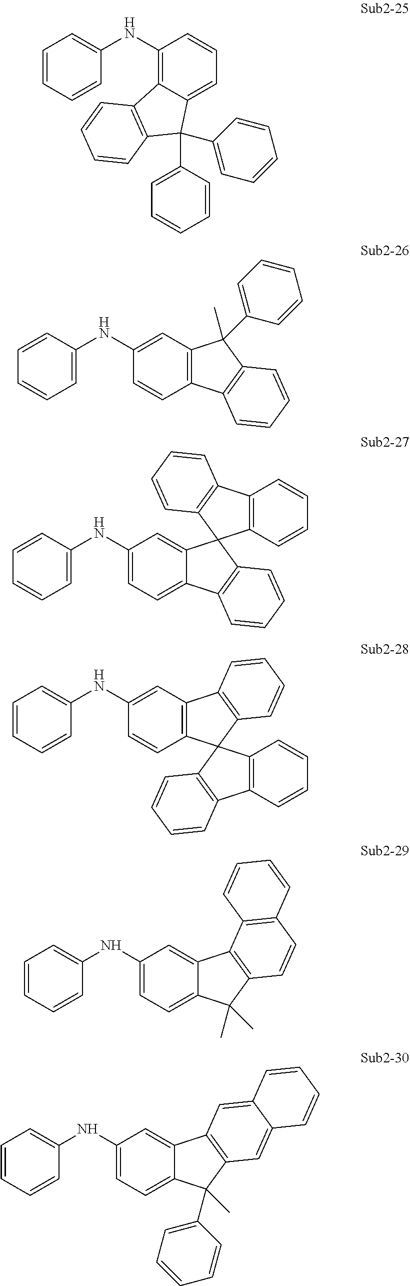

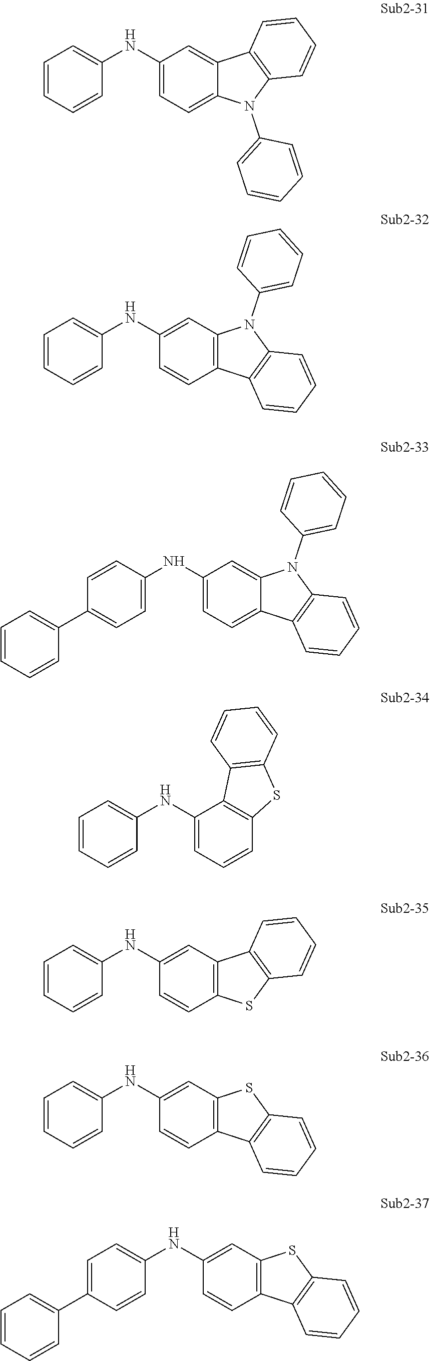

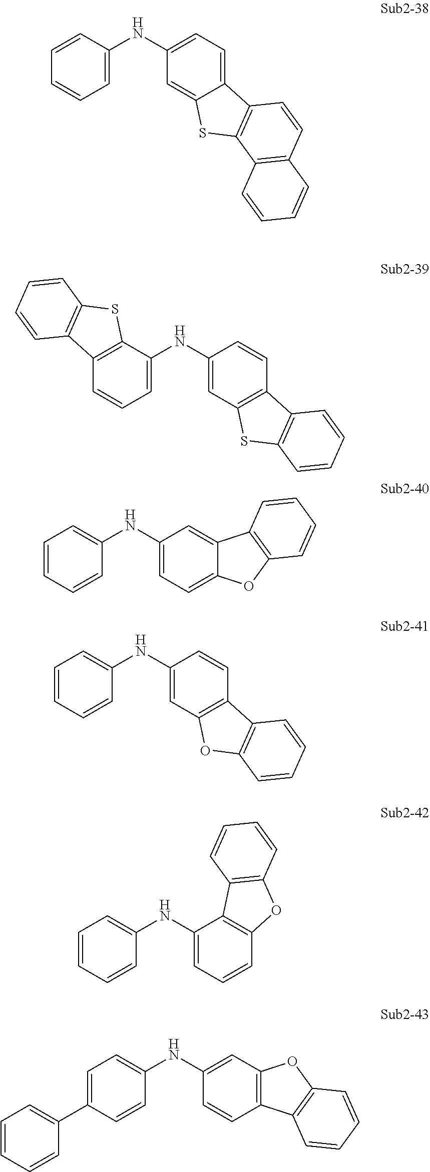

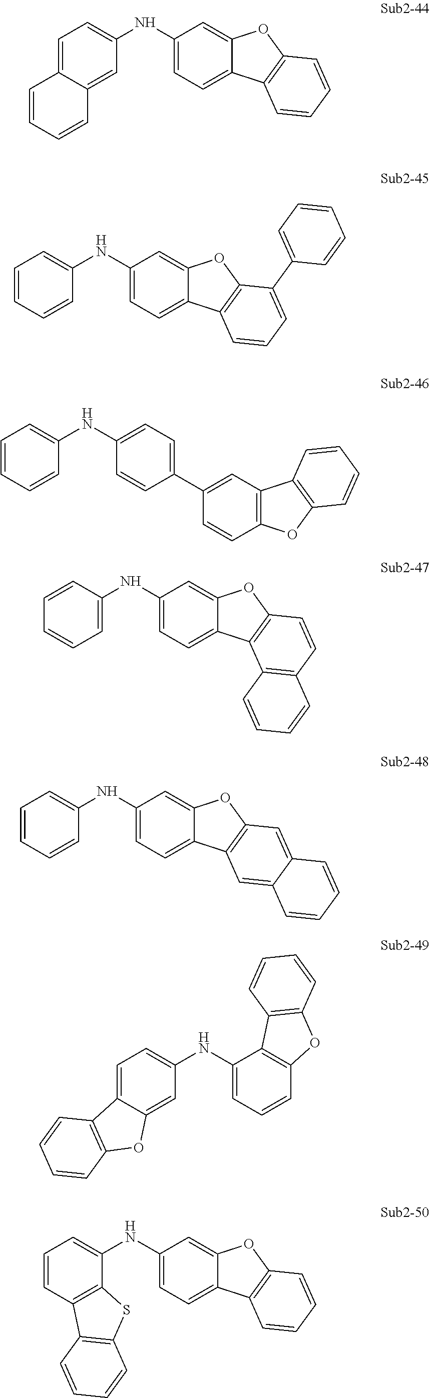

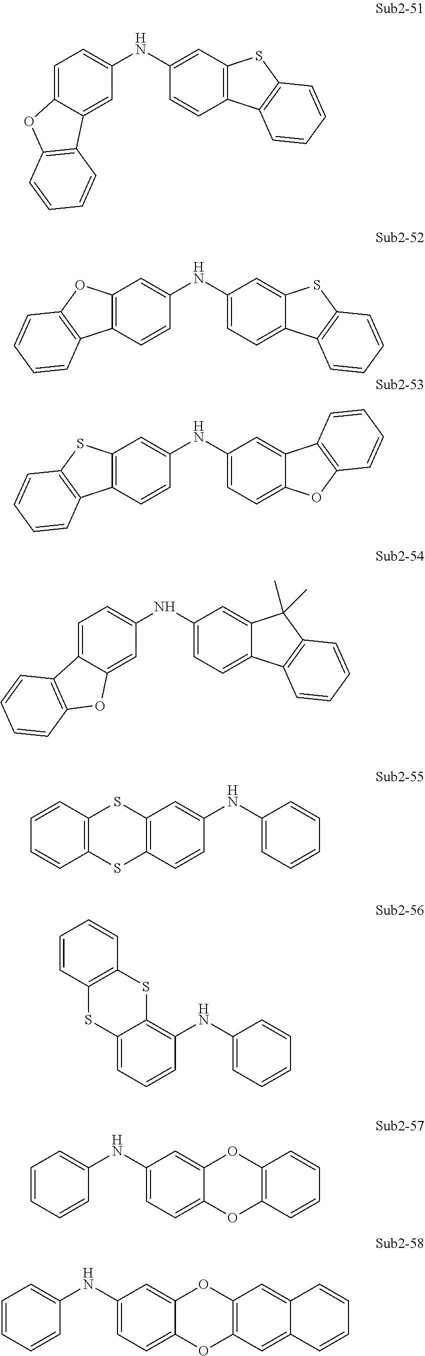

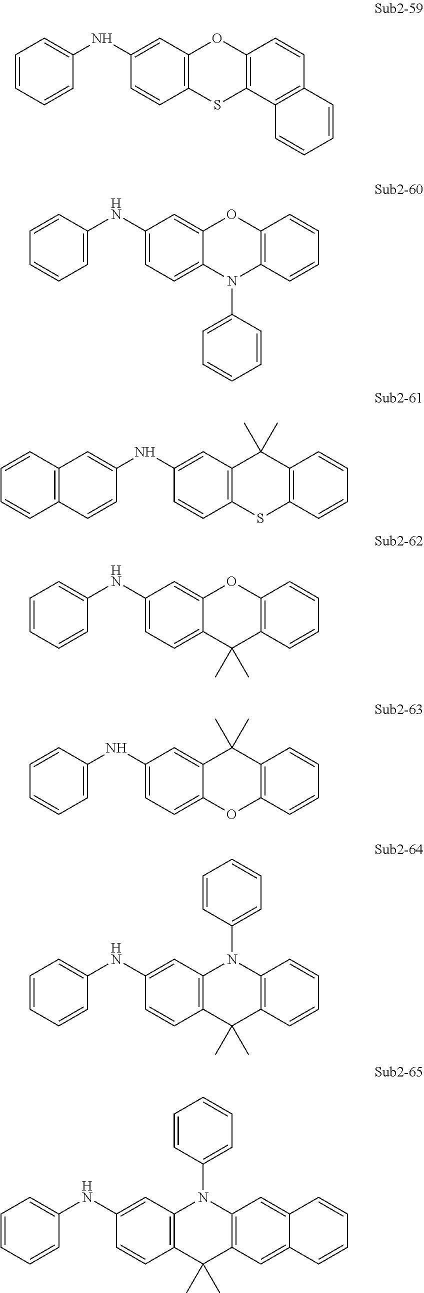

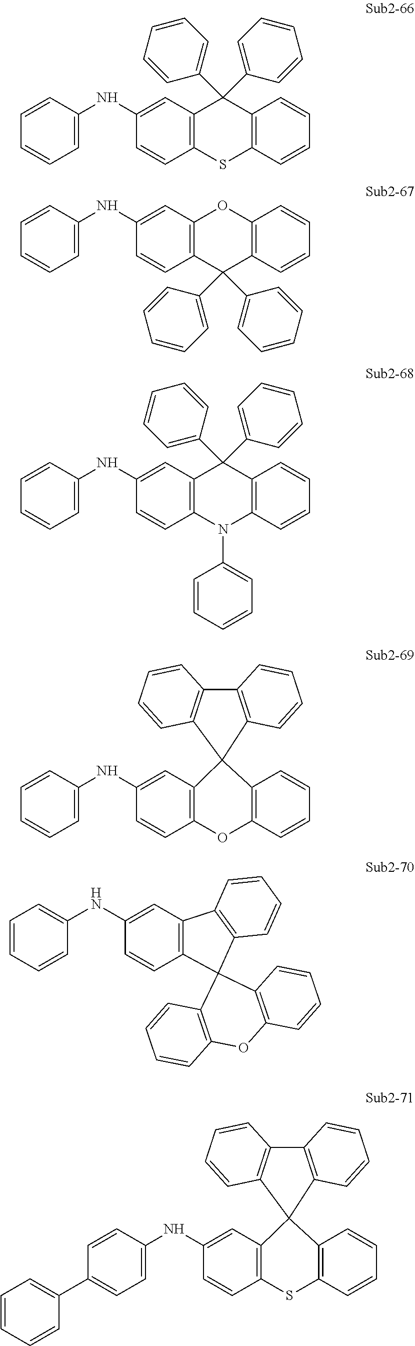

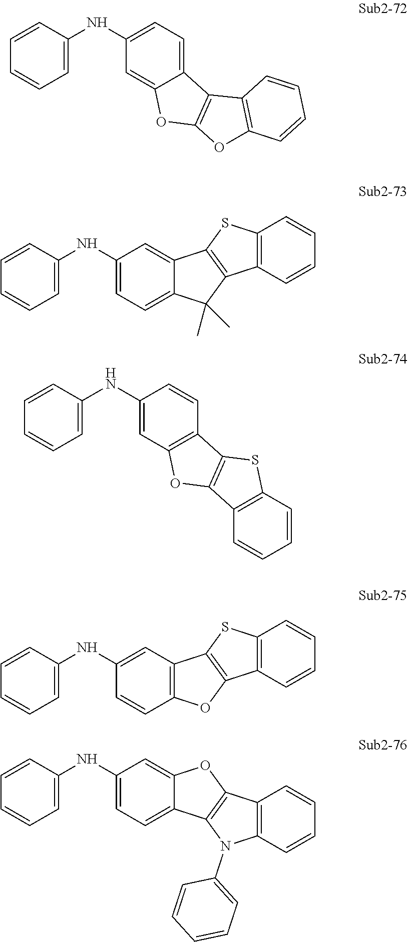

Table 2 shows FD-MS (Field Desorption-Mass Spectrometry) values of compounds belonging to Sub 2

TABLE 2

| Com- | |
|---|---|
| pound | FD-MS |
| Sub 2-1 | m/z = 169.09 (C₁₂H₁₁N = 169.23) |
| Sub 2-2 | m/z = 194.08 (C₁₃H₁₀N₂ = 194.24) |
| Sub 2-3 | m/z = 174.12 (C₁₂H₆D₅N = 174.26) |
| Sub 2-4 | m/z = 245.12 (C₁₂H₁₅N = 245.33) |
| Sub 2-5 | m/z = 245.12 (C₁₈H₁₅N = 245.33) |
| Sub 2-6 | m/z = 321.15 (C₂₄H₁₉N = 321.42) |
| Sub 2-7 | m/z = 245.12 (C₁₈H₁₅N = 245.33) |
| Sub 2-8 | m/z = 321.15 (C₂₄H₁₉N = 321.42) |
| Sub 2-9 | m/z = 321.15 (C₂₄H₁₉N = 321.42) |
| Sub 2-10 | m/z = 295.14 (C₂₂H₁₇N = 295.39) |
| Sub 2-11 | m/z = 295.14 (C₂₂H₁₇N = 295.39) |
| Sub 2-12 | m/z = 321.15 (C₂₄H₁₉N = 321.42) |
| Sub 2-13 | m/z = 219.1 (C₁₆H₁₃N = 219.29) |
| Sub 2-14 | m/z = 219.1 (C₁₆H₁₃N = 219.29) |
| Sub 2-15 | m/z = 269.12 (C₂₀H₁₅N = 269.35) |
| Sub 2-16 | m/z = 269.12 (C₂₀H₁₅N = 269.35) |
| Sub 2-17 | m/z = 319.14 (C₂₄H₁₇N = 319.41) |
| Sub 2-18 | m/z = 167.07 (C₁₂H₉N = 167.21) |
| Sub 2-19 | m/z = 170.08 (C₁₁H₁₀N₂ = 170.22) |
| Sub 2-20 | m/z = 293.12 (C₂₂H₁₅N = 293.37) |
| Sub 2-21 | m/z = 285.15 (C₂₁H₁₉N = 285.39) |
| Sub 2-22 | m/z = 285.15 (C₂₁H₁₉N = 285.39) |
| Sub 2-23 | m/z = 361.18 (C₂₇H₂₃N = 361.49) |
| Sub 2-24 | m/z = 409.18 (C₃₁H₂₃N = 409.53) |
| Sub 2-25 | m/z = 409.18 (C₃₁H₂₃N = 409.53) |
| Sub 2-26 | m/z = 347.17 (C₂₆H₂₁N = 347.46) |
| Sub 2-27 | m/z = 407.17 (C₃₁H₂₁ N = 407.52) |
| Sub 2-28 | m/z = 407.17 (C₃₁H₂₁N = 407.52) |
| Sub 2-29 | m/z = 335.1 7 (C₂₅H₂₁N = 335.45) |
| Sub 2-30 | m/z = 397.1 8 (C₃₀H₂₃N = 397.52) |
| Sub 2-31 | m/z = 334.15 (C₂₄H₁₈N2 = 334.42) |
| Sub 2-32 | m/z = 334.15 (C₂₄H₁₈N₂ = 334.42) |
| Sub 2-33 | m/z = 410.18 (C₃₀H₂₂N2 = 410.52) |
| Sub 2-34 | m/z = 275.08 (C₁₈H₁₃NS = 275.37) |
| Sub 2-35 | m/z = 275.08 (C₁₈H₁₃NS = 275.37) |
| Sub 2-36 | m/z = 275.08 (C₁₈H₁₃NS = 275.37) |
| Sub 2-37 | m/z = 351.11 (C₂₄H₁₇NS = 351.47) |
| Sub 2-38 | m/z = 325.09 (C₂₂H₁₅NS = 325.43) |
| Sub 2-39 | m/z = 381 .06 (C₂₄H₁₅NS₂ = 381.51) |
| Sub 2-40 | m/z = 259.1 (C₁₈H₁₃NO = 259.31) |
| Sub 2-41 | m/z = 259.1 (C₁₈H₁₃NO = 259.31) |
| Sub 2-42 | m/z = 259.1 (C₁₈H₁₃NO = 259.31) |
| Sub 2-43 | m/z = 335.13 (C₂₄H₁₇NO = 335.41) |
| Sub 2-44 | m/z = 309.12 (C₂₂H₁₅NO = 309.37) |
| Sub 2-45 | m/z = 335.13 (C₂₄H₁₇NO = 335.41) |
| Sub 2-46 | m/z = 335.13 (C₂₄H₁₇NO = 335.41) |
| Sub 2-47 | m/z = 309.1 2 (C₂₂H₁₅NO = 309.37) |
| Sub 2-48 | m/z = 309.12 (C₂₂H₁₅NO = 309.37) |
| Sub 2-49 | m/z = 349.11 (C₂₄H₁₅NO₂ = 349.39) |
| Sub 2-50 | m/z = 365.09 (C₂₄H₁₅NOS = 365.45) |
| Sub 2-51 | m/z = 365.09 (C₂₄H₁₅NOS = 365.45) |
| Sub 2-52 | m/z = 365.09 (C₂₄H₁₅NOS = 365.45) |
| Sub 2-53 | m/z = 365.09 (C₂₄H₁₅NOS = 365.45) |
| Sub 2-54 | m/z = 375.16 (C₂₇H₂₁NO = 375.47) |
| Sub 2-55 | m/z = 307.05 (C₁₈H₁₃NS₂ = 307.43) |
| Sub 2-56 | m/z = 307.05 (C₁₈H₁₃NS₂ = 307.43) |
| Sub 2-57 | m/z = 275.09 (C₁₈H₁₃NO₂ = 275.31) |
| Sub 2-58 | m/z = 325.11 (C₂₂H₁₅NO₂ = 325.37) |
| Sub 2-59 | m/z = 341.09 (C₂₂H₁₅NOS = 341.43) |
| Sub 2-60 | m/z = 350.14 (C₂₄H₁₈N₂O = 350.42) |
| Sub 2-61 | m/z = 367.14 (C₂₅H₂₁NS = 367.51) |
| Sub 2-62 | m/z = 301.15 (C₂₁H₁₉NO = 301.39) |
| Sub 2-63 | m/z = 301.15 (C₂₁H₁₉NO = 301.39) |
| Sub 2-64 | m/z = 376.19 (C₂₇H₂₄N₂ = 376.5) |
| Sub 2-65 | m/z = 426.21 (C₃₁H₂₆N₂ = 426.56) |
| Sub 2-66 | m/z = 441.16 (C₃₁H₂₃NS = 441.59) |
| Sub 2-67 | m/z = 425.18 (C₃₁H₂₃NO = 425.53) |
| Sub 2-68 | m/z = 500.23 (C₃₇H₂₈N₂ = 500.65) |
| Sub 2-69 | m/z = 423.16 (C₃₁H₂₁NO = 423.52) |
| Sub 2-70 | m/z = 423.16 (C₃₁H₂₁NO = 423.52) |
| Sub 2-71 | m/z = 515.17 (C₃₇H₂₅NS = 515.67) |
| Sub 2-72 | m/z = 299.09 (C₂₀H₁₃NO₂ = 299.33) |
| Sub 2-73 | m/z = 341.12 (C₂₃H₁₉NS = 341.47) |
| Sub 2-74 | m/z = 31 5.07 (C₂₀H₁₃NOS = 315.39) |
| Sub 2-75 | m/z = 31 5.07 (C₂₀H₁₃NOS = 315.39) |
| Sub 2-76 | m/z = 374.14 (C₂₆H₁₈N₂O = 374.44) |

### III. Synthesis Example of Final Product 1

### Synthesis Example of 1-1

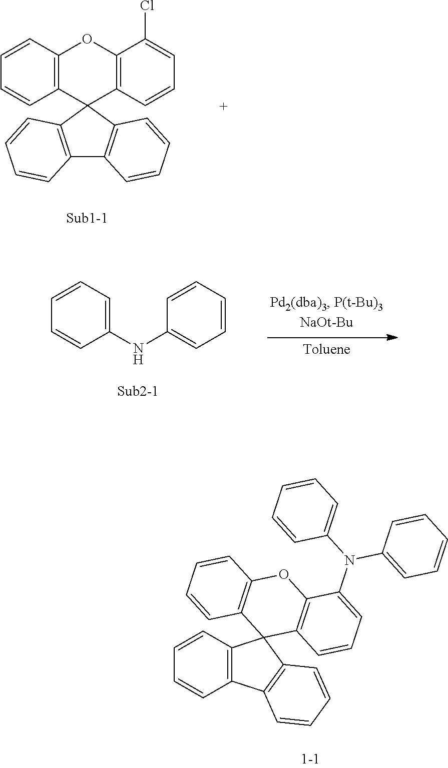 [SMILES: Clc1cccc2c1Oc1ccccc1C21c2ccccc2-c2ccccc21 ; c1ccc(Nc2ccccc2)cc1 ; c1ccc(N(c2ccccc2)c2cccc3c2Oc2ccccc2C32c3ccccc3-c3ccccc32)cc1]

After putting Sub 1-1 (10 g, 27.26 mmol) in a round bottom flask and dissolving it with Toluene (300 ml), Sub 2-1 (5.07 g, 29.99 mmol), Pd₂(dba)₃ (1.25 g, 1.36 mmol), P(t-Bu)₃ (0.55 g, 2.73 mmol) and NaOt-Bu (7.86 g, 81.78 mmol) were added and stirred at 100° C. After the reaction was completed, extraction was performed with CH₂Cl₂ and water, and the organic layer was dried with MgSO₄, concentrated, and the resulting compound was recrystallized using a silica gel column to obtain 11.7 g of product (yield: 86%).

### Synthesis Example of 1-15

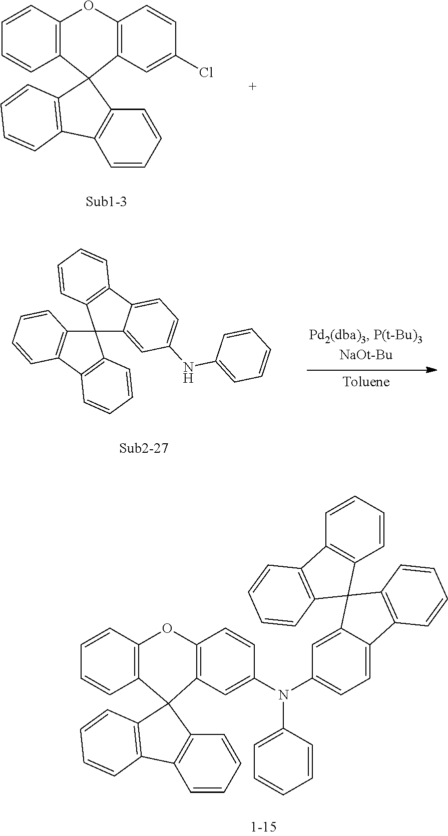 [SMILES: Clc1ccc2c(c1)C1(c3ccccc3O2)c2ccccc2-c2ccccc21 ; c1ccc(Nc2ccc3c(c2)C2(c4ccccc4-c4ccccc42)c2ccccc2-3)cc1 ; c1ccc(N(c2ccc3c(c2)C2(c4ccccc4O3)c3ccccc3-c3ccccc32)c2ccc3c(c2)C2(c4ccccc4-c4ccccc42)c2ccccc2-3)cc1]

Sub 1-3 (10 g, 27.26 mmol), Toluene (500 ml), Sub 2-27 (12.22 g, 29.99 mmol), Pd₂(dba)₃ (1.25 g, 1.36 mmol), P(t-Bu)₃ (0.55 g, 2.73 mmol), NaOt-Bu (7.86 g, 81.78 mmol) were used to obtain 15.89 g of the product using the synthesis method of 1-1. (Yield: 79%)

### Synthesis Example of 1-27

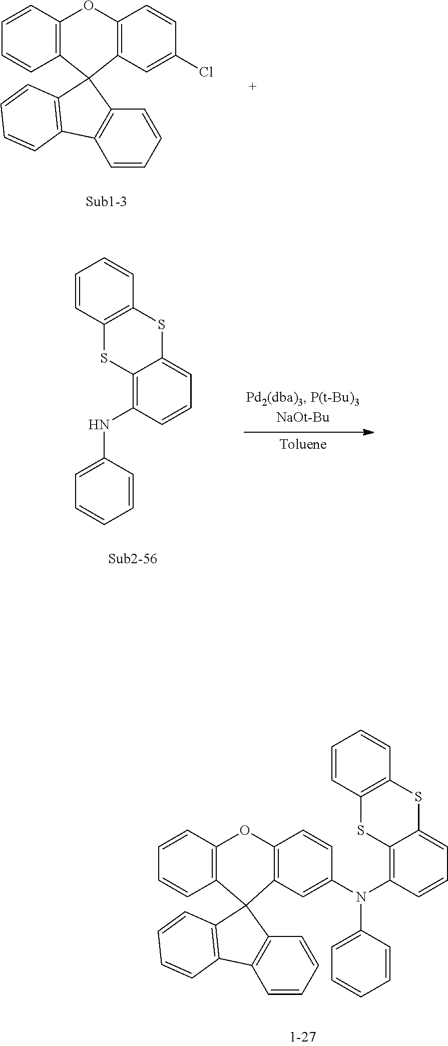 [SMILES: Clc1ccc2c(c1)C1(c3ccccc3O2)c2ccccc2-c2ccccc21 ; c1ccc(Nc2cccc3c2Sc2ccccc2S3)cc1 ; c1ccc(N(c2ccc3c(c2)C2(c4ccccc4O3)c3ccccc3-c3ccccc32)c2cccc3c2Sc2ccccc2S3)cc1]

Sub 1-3 (10 g, 27.26 mmol), Toluene (500 ml), Sub 2-56 (9.22 g, 29.99 mmol), Pd₂(dba)₃ (1.25 g, 1.36 mmol), P(t-Bu)₃ (0.55 g, 2.73 mmol), NaOt-Bu (7.86 g, 81.78 mmol) were used to obtain 14.6 g of the product using the synthesis method of 1-1. (Yield: 84%)

### Synthesis Example of 1-42

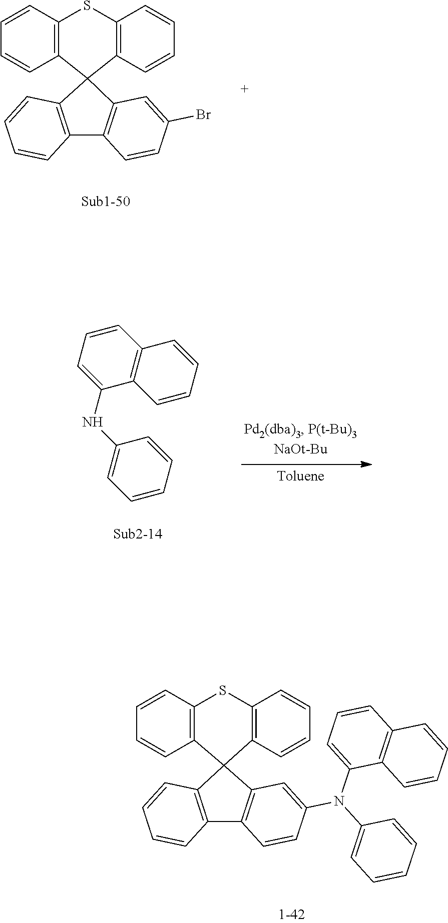 [SMILES: Brc1ccc2c(c1)C1(c3ccccc3Sc3ccccc31)c1ccccc1-2 ; c1ccc(Nc2cccc3ccccc23)cc1 ; c1ccc(N(c2ccc3c(c2)C2(c4ccccc4Sc4ccccc42)c2ccccc2-3)c2cccc3ccccc23)cc1]

Sub 1-50 (10 g, 23.40 mmol), Toluene (500 ml), Sub 2-14 (5.64 g, 25.74 mmol), Pd₂(dba)₃ (1.25 g, 1.36 mmol), P(t-Bu)₃ (0.55 g, 2.73 mmol), NaOt-Bu (7.86 g, 81.78 mmol) were used to obtain 10.85 g of the product using the synthesis method of 1-1. (Yield: 82%)

### Synthesis Example of 1-74

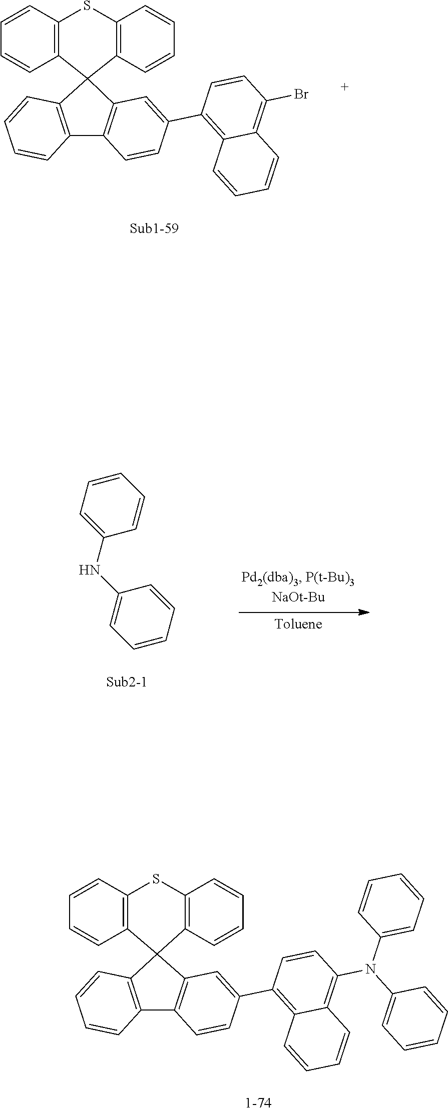 [SMILES: Brc1ccc(-c2ccc3c(c2)C2(c4ccccc4Sc4ccccc42)c2ccccc2-3)c2ccccc12 ; c1ccc(Nc2ccccc2)cc1 ; c1ccc(N(c2ccccc2)c2ccc(-c3ccc4c(c3)C3(c5ccccc5Sc5ccccc53)c3ccccc3-4)c3ccccc23)cc1]

Sub 1-59 (10 g, 18.07 mmol), Toluene (500 ml), Sub 2-1 (3.36 g, 19.87 mmol), Pd₂(dba)₃ (1.25 g, 1.36 mmol), P(t-Bu)₃ (0.55 g, 2.73 mmol), NaOt-Bu (7.86 g, 81.78 mmol) were used to obtain 9.9 g of the product using the synthesis method of 1-1. (Yield: 85%)

### Synthesis Example of 1-103

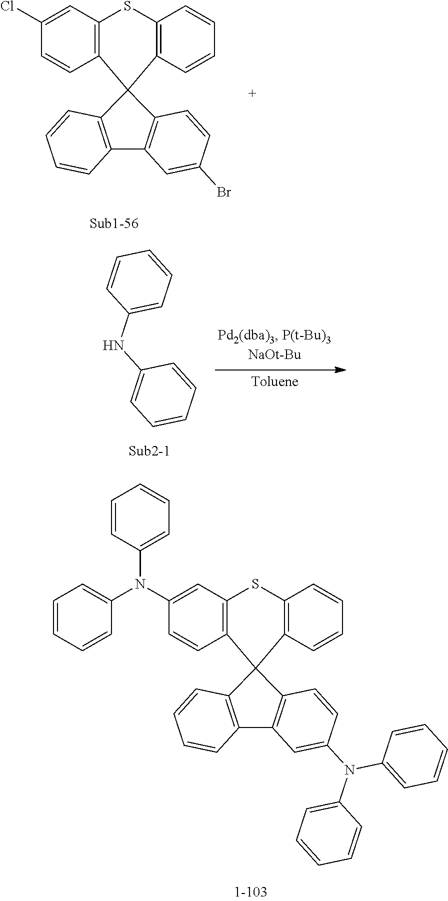 [SMILES: Clc1ccc2c(c1)Sc1ccccc1C21c2ccccc2-c2cc(Br)ccc21 ; c1ccc(Nc2ccccc2)cc1 ; c1ccc(N(c2ccccc2)c2ccc3c(c2)Sc2ccccc2C32c3ccccc3-c3cc(N(c4ccccc4)c4ccccc4)ccc32)cc1]

Sub 1-56 (10 g, 21.65 mmol), Toluene (500 ml), Sub 2-1 (4.03 g, 23.82 mmol), Pd₂(dba)₃ (1.25 g, 1.36 mmol), P(t-Bu)₃ (0.55 g, 2.73 mmol), NaOt-Bu (7.86 g, 81.78 mmol) were used to obtain 12.63 g of the product using the synthesis method of 1-1. (Yield: 90%)

### Synthesis Example of 1-126

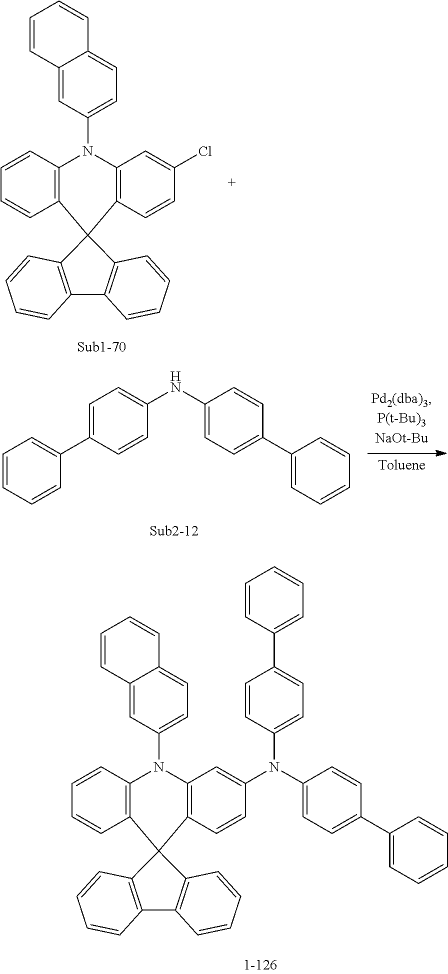 [SMILES: Clc1ccc2c(c1)N(c1ccc3ccccc3c1)c1ccccc1C21c2ccccc2-c2ccccc21 ; c1ccc(-c2ccc(Nc3ccc(-c4ccccc4)cc3)cc2)cc1 ; c1ccc(-c2ccc(N(c3ccc(-c4ccccc4)cc3)c3ccc4c(c3)N(c3ccc5ccccc5c3)c3ccccc3C43c4ccccc4-c4ccccc43)cc2)cc1]

Sub 1-70 (10 g, 20.32 mmol), Toluene (500 ml), Sub 2-12 (7.2 g, 22.36 mmol), Pd₂(dba)₃ (1.25 g, 1.36 mmol), P(t-Bu)₃ (0.55 g, 2.73 mmol), NaOt-Bu (7.86 g, 81.78 mmol) were used to obtain 14 g of the product using the synthesis method of 1-1. (Yield: 89%)

Table 3 shows the FD-MS (Field Desorption-Mass Spectrometry) values of the compounds belonging to Final product 1.

TABLE 3

| Com- | |
|---|---|
| pound | FD-MS |
| 1-1 | m/z = 499.19 (C₃₇H₂₅NO = 499.61) |
| 1-2 | m/z = 499.19 (C₃₇H₂₅NO = 499.61) |
| 1-3 | m/z = 499.19 (C₃₇H₂₅NO = 499.61) |
| 1-4 | m/z = 499.19 (C₃₇H₂₅NO = 499.61) |
| 1-5 | m/z = 575.22 (C₄₃H₂₉NO = 575.71) |
| 1-6 | m/z = 589.2 (C₄₃H₂₇NO₂ = 589.69) |
| 1-7 | m/z = 549.21 (C₄H₂₇NO = 549.67) |
| 1-8 | m/z = 651.26 (C₄₉H₃₃NO = 651.81) |
| 1-9 | m/z = 599.22 (C₄₅H₂₉NO = 599.73) |
| 1-10 | m/z = 605.18 (C₄₃H₂₇NOS = 605.76) |
| 1-11 | m/z = 615.26 (C₄₆H₃₃NO = 615.78) |
| 1-12 | m/z = 565.19 (C₄H₂₇NS = 565.73) |
| 1-13 | m/z = 695.19 (C₄₉H₂₉NO₂S = 695.84) |
| 1-14 | m/z = 755.26 (C₅₆H₃₇NS = 755.98) |
| 1-15 | m/z = 737.27 (C₅₆H₃₅NO = 737.9) |
| 1-16 | m/z = 671.17 (C₄₇H₂₉NS₂ = 671.88) |
| 1-17 | m/z = 639.22 (C₄₇H₂₉NO₂ = 639.75) |
| 1-18 | m/z = 681.25 (C₅H₃₅NS = 681 .9) |
| 1-19 | m/z = 589.2 (C₄₃H₂₇NO₂ = 589.69) |
| 1-20 | m/z = 664.25 (C₄₉H₃₂N₂O = 664.81) |
| 1-21 | m/z = 639.22 (C₄₇H₂₉NO₂ = 639.75) |
| 1-22 | m/z = 605.18 (C₄₃H₂₇NOS = 605.76) |
| 1-23 | m/z = 605.2 (C₄₃H₂₇NO₃ = 605.69) |
| 1-24 | m/z = 743.26 (C₅₅H₃₇NS = 743.97) |
| 1-25 | m/z = 720.22 (C₅₁H₃₂N₂OS = 720.89) |
| 1-26 | m/z = 687.21 (C₄H₃₃NS₂ = 687.92) |
| 1-27 | m/z = 637.15 (C₄₃H₂₇NOS₂ = 637.82) |
| 1-28 | m/z = 629.2 (C₄₅H₂₇NO₃ = 629.72) |
| 1-29 | m/z = 704.25 (C₅₁H₃₂N₂O₂ = 704.83) |
| 1-30 | m/z = 647.23 (C₄₆H₃₃NOS = 647.84) |
| 1-31 | m/z = 591.2 (C₄₃H₂₉NS = 591.77) |
| 1-32 | m/z = 575.22 (C₄₃H₂₉NO = 575.71) |
| 1-33 | m/z = 575.22 (C₄₃H₂₉NO = 575.71) |
| 1-34 | m/z = 641.22 (C₄₇H₃₁NS = 641.83) |
| 1-35 | m/z = 665.24 (C₄₉H₃₁NO₂ = 665.79) |
| 1-36 | m/z = 682.24 (C₄₉H₃₁FN₂O = 682.8) |
| 1-37 | m/z = 641.22 (C₄₇H₃₁NS = 641.83) |
| 1-38 | m/z = 681.21 (C₄₉H₃₁NOS = 681.85) |
| 1-39 | m/z = 665.24 (C₄₉H₃₁NO₂ = 665.79) |
| 1-40 | m/z = 651.26 (C₄₉H₃₃NO = 651 .81) |
| 1-41 | m/z = 499.19 (C₃₇H₂₅NO = 499.61) |
| 1-42 | m/z = 565.19 (C₄H₂₇NS = 565.73) |
| 1-43 | m/z = 575.22 (C₄₃H₂₉NO = 575.71) |
| 1-44 | m/z = 605.18 (C₄₃H₂₇NOS = 605.76) |
| 1-45 | m/z = 631.23 (C₄₆H₃₃NS = 631.84) |
| 1-46 | m/z = 667.23 (C₄₉H₃₃NS = 667.87) |
| 1-47 | m/z = 599.22 (C₄₅H₂₉NO = 599.73) |
| 1-48 | m/z = 695.19 (C₄₉H₂₉NO₂S = 695.84) |
| 1-49 | m/z = 639.22 (C₄₇H₂₉NO₂ = 639.75) |
| 1-50 | m/z = 641.22 (C₄₇H₃₁NS = 641.83) |
| 1-51 | m/z = 649.24 (C₄₉H₃₁NO = 649.79) |
| 1-52 | m/z = 639.2 (C₄₇H₂₉NS = 639.82) |
| 1-53 | m/z = 705.27 (C₅₂H₃₅NO₂ = 705.86) |
| 1-54 | m/z = 707.26 (C₅₂H₃₇NS =707.94) |
| 1-55 | m/z = 739.29 (C₅₆H₃₇NO =739.92) |
| 1-56 | m/z = 737.27 (C₅₆H₃₅NO = 737.9) |
| 1-57 | m/z = 740.28 (C₅₅H₃₆N₂O = 740.91) |
| 1-58 | m/z = 681.21 (C₄₉H₃₁NOS₌ 681.85₎ |
| 1-59 | m/z = 631.25 (C₄₆H₃₃NO₂ = 631 .78) |
| 1-60 | m/z = 753.27 (C₅₆H₃₅NO₂ = 753.9) |
| 1-61 | m/z = 845.28 (C₆₂H₃₉NOS = 846.06) |
| 1-62 | m/z = 753.27 (C₅₆H₃₅NO₂ = 753.9) |
| 1-63 | m/z = 605.2 (C₄₃H₂₇NO₃ = 605.69) |
| 1-64 | m/z = 706.3 (C₅₂H₃₈N₂O = 706.89) |
| 1-65 | m/z = 771.26 (C₅₆H₃₇NOS = 771 .98) |
| 1-66 | m/z = 755.28 (C₅₆H₃₇NO₂ = 755.92) |
| 1-67 | m/z = 655.21 (C₄₇H₂₉NO₃ = 655.75) |
| 1-68 | m/z = 772.29 (C₅₆H₄₀N₂S = 773.01) |
| 1-69 | m/z = 697.24 (C₅₀H₃₅NOS = 697.9) |
| 1-70 | m/z = 687.17 (C₄₇H₂₉NOS₂ = 687.88) |
| 1-71 | m/z = 680.25 (C₄₉H₃₂N₂O₂ = 680.81) |
| 1-72 | m/z = 645.18 (C₄₅H₂₇NO₂S = 645.78) |
| 1-73 | m/z = 830.33 (C₆₂H₄₂N₂O = 831.03) |
| 1-74 | m/z = 641.22 (C₄₇H₃₁NS = 641.83) |
| 1-75 | m/z = 665.24 (C₄₉H₃₁NO₂ = 665.79) |
| 1-76 | m/z = 681.21 (C₄₉H₃₁NOS = 681.85) |
| 1-77 | m/z = 665.24 (C₄₉H₃₁NO₂ = 665.79) |
| 1-78 | m/z = 625.24 (C₄₇H₃₁NO = 625.77) |
| 1-79 | m/z = 504.22 (C₃₇H₂₀D₅NO = 504.64) |
| 1-80 | m/z = 631.29 (C₄₇H₃₇NO = 631 .82) |
| 1-81 | m/z = 720.19 (C₅₀H₂₈N₂O₂S = 720.85) |
| 1-82 | m/z = 539.22 (C₄₀H₂₉NO = 539.68) |
| 1-83 | m/z = 529.2 (C₃₈H₂₇NO₂ = 529.64) |
| 1-84 | m/z = 787.2 (C₅₅H₃₃NOS₂ = 788) |
| 1-85 | m/z = 575.22 (C₄₃H₂₉NO = 575.71) |
| 1-86 | m/z = 651.26 (C₄₉H₃₃NO = 651 .81) |
| 1-87 | m/z = 576.22 (C₄₂H₂₈N₂O = 576.7) |
| 1-88 | m/z = 575.22 (C₄₃H₂₉NO = 575.71) |
| 1-89 | m/z = 503.22 (C₃₇H₂D₄NO = 503.64) |
| 1-90 | m/z = 742.3 (C₅₅H₃₈N₂O = 742.92) |
| 1-91 | m/z = 772.25 (C₅₅H₃₆N₂OS = 772.97) |
| 1-92 | m/z = 716.28 (C₅₃H₃₆N₂O = 716.88) |
| 1-93 | m/z = 766.31 (C₅₆H₃₈N₄ = 766.95) |
| 1-94 | m/z = 772.25 (C₅₅H₃₆N₂OS = 772.97) |
| 1-95 | m/z = 858.36 (C₆₄H₄₆N₂O = 859.09) |
| 1-96 | m/z = 862.27 (C₆₁H₃₈N₂O₂S = 863.05) |
| 1-97 | m/z = 847.3 (C₆₁H₄₁N₃S = 848.08) |
| 1-98 | m/z = 772.25 (C₅₅H₃₆N₂OS = 772.97) |
| 1-99 | m/z = 844.35 (C₆₃H₄₄N₂O = 845.06) |
| 1-100 | m/z = 1042.29 (C₇₃H₄₂N₂O₄S = 1043.21) |
| 1-101 | m/z = 920.32 (C₆₈H₄₄N₂S = 921.17) |
| 1-102 | m/z = 820.2 (C₅₅H₃₆N₂S₃ = 821.09) |
| 1-103 | m/z = 682.24 (C₄₉H₃₄N₂S = 682.89) |
| 1-104 | m/z = 848.29 (C_{61 H40}N₂OS = 849.06) |
| 1-105 | m/z = 766.3 (C₅₇H₃₈N₂O = 766.94) |
| 1-106 | m/z = 682.24 (C₄₉H₃₄N₂S = 682.89) |
| 1-107 | m/z = 834.31 (C₆₁H₄₂N₂S = 835.08) |
| 1-108 | m/z = 497.18 (C₃₇H₂₃NO = 497.6) |
| 1-109 | m/z = 667.23 (C₄₉H₃₃NS = 667.87) |
| 1-110 | m/z = 651.26 (C₄₉H₃₃NO = 651.81) |
| 1-111 | m/z = 727.29 (C₅₅H₃₇NO = 727.91) |
| 1-112 | m/z = 609.19 (C₄₃H₂₈FNS = 609.76) |
| 1-113 | m/z = 758.28 (C₅₅H₃₈N₂S = 758.98) |
| 1-114 | m/z = 742.3 (C₅₅H₃₈N₂O = 742.92) |
| 1-115 | m/z = 666.27 (C₄₉H₃₄N₂O = 666.82) |
| 1-116 | m/z = 792.31 (C₅₉H40N2O = 792.98) |
| 1-117 | m/z = 792.31 (C₅₉H40N2O = 792.98) |
| 1-118 | m/z = 808.29 (C₅₉H₄oN₂S = 809.04) |
| 1-119 | m/z = 682.24 (C₄₉H₃4N₂S = 682.89) |
| 1-120 | m/z = 864.26 (C₆₁H₄₀N₂S₂ =865.13) |
| 1-121 | m/z = 742.3 (C₅₅H₃₈N₂O = 742.92) |
| 1-122 | m/z = 858.36 (C₆₄H₄₆N₂O = 859.09) |
| 1-123 | m/z = 742.3 (C₅₅H₃₈N₂O = 742.92) |
| 1-124 | m/z = 756.28 (C₅₅H₃₆N₂O₂ = 756.91) |
| 1-125 | m/z = 575.22 (C₄₃H₂₉NO = 575.71) |
| 1-126 | m/z = 516.17 (C₃₆H₂₄N₂S = 516.66) |
| 1-127 | m/z = 515.17 (C₃₇H₂₅NS = 515.67) |
| 1-128 | m/z = 651.26 (C₄₉H₃₃NO = 651 .81) |
| 1-129 | m/z = 549.21 (C₄H₂₇NO = 549.67) |
| 1-130 | m/z = 549.21 (C₄H₂₇NO = 549.67) |
| 1-131 | m/z = 565.19 (C₄₁H₂₇NS = 565.73) |
| 1-132 | m/z = 641.22 (C₄₇H₃₁NS = 641.83) |
| 1-133 | m/z = 681.25 (C₅₀H₃₅NS = 681.9) |
| 1-134 | m/z = 599.22 (C₄₅H₂₉NO = 599.73) |
| 1-135 | m/z = 625.24 (C₄₇H₃₁NO = 625.77) |
| 1-136 | m/z = 599.22 (C₄₅H₂₉NO = 599.73) |
| 1-137 | m/z = 615.2 (C₄₅H₂₉NS = 615.79) |
| 1-138 | m/z = 565.19 (C₄H₂₇NS = 565.73) |
| 1-139 | m/z = 639.22 (C₄₇H₂₉NO₂ = 639.75) |
| 1-140 | m/z = 599.22 (C₄₅H₂₉NO = 599.73) |
| 1-141 | m/z = 605.18 (C₄₃H₂₇NOS = 605.76) |
| 1-142 | m/z = 621.16 (C₄₃H₂₇NS₂ = 621.82) |
| 1-143 | m/z = 695.19 (C₄₉H₂₉NO₂S = 695.84) |
| 1-144 | m/z = 664.25 (C₄₉H₃₂N₂O = 664.81) |
| 1-145 | m/z = 664.25 (C₄₉H₃₂N₂O = 664.81) |
| 1-146 | m/z = 615.26 (C₄₆H₃₃NO = 615.78) |
| 1-147 | m/z = 796.29 (C₅₈H₄₀N₂S = 797.03) |
| 1-148 | m/z = 589.2 (C₄₃H₂₇NO₂ = 589.69) |
| 1-149 | m/z = 766.3 (C₅₇H₃₈N₂O = 766.94) |
| 1-150 | m/z = 664.25 (C₄₉H₃₂N₂O = 664.81) |
| 1-151 | m/z = 772.25 (C₅₅H₃₆N₂OS = 772.97) |
| 1-152 | m/z = 831.32 (C₆₁H₄₁N₃O = 832.02) |
| 1-153 | m/z = 574.24 (C₄₃H₃₀N₂ = 574.73) |
| 1-154 | m/z = 739.3 (C₅₅H₃₇N₃ = 739.92) |
| 1-155 | m/z = 664.25 (C₄₉H₃₂N₂O = 664.81) |
| 1-156 | m/z = 720.22 (C₅₁H₃₂N₂OS = 720.89) |
| 1-157 | m/z = 650.27 (C₄₉H₃₄N₂ = 650.83) |
| 1-158 | m/z = 650.27 (C₄₉H₃₄N₂ = 650.83) |
| 1-159 | m/z = 726.3 (C₅₅H₃₈N₂ = 726.92) |
| 1-160 | m/z = 766.31 (C₅₆H₃₈N₄ = 766.95) |
| 1-161 | m/z = 831.32 (C₆₁H₄₁N₃O = 832.02) |
| 1-162 | m/z = 726.3 (C₅₅H₃₈N₂ = 726.92) |
| 1-163 | m/z = 624.26 (C₄₇H₃₂N₂ = 624.79) |
| 1-164 | m/z = 690.3 (C₅₂H₃₈N₂ = 690.89) |

### Synthesis Example 2

The compound represented by Formula 2 according to the present invention (final product 2) is synthesized as shown in Reaction Scheme 4, but is not limited thereto.

G¹ is L⁵ or L⁶, G² is Ar⁵ or Ar⁶, X¹ to X³, L⁴ to L⁶, and Ar⁴ to Ar⁶ are the same as defined in Formula 1, Hal³ and Hal⁴ are each independently I, Br or Cl.

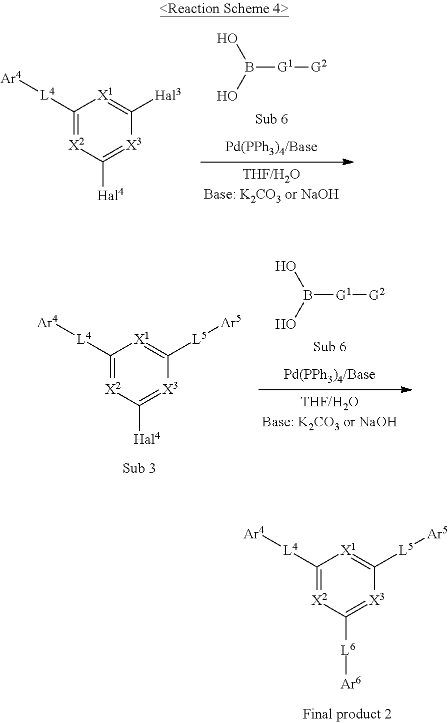

### I. Synthesis Example of Sub 3

### Synthesis Example of Sub 3-1

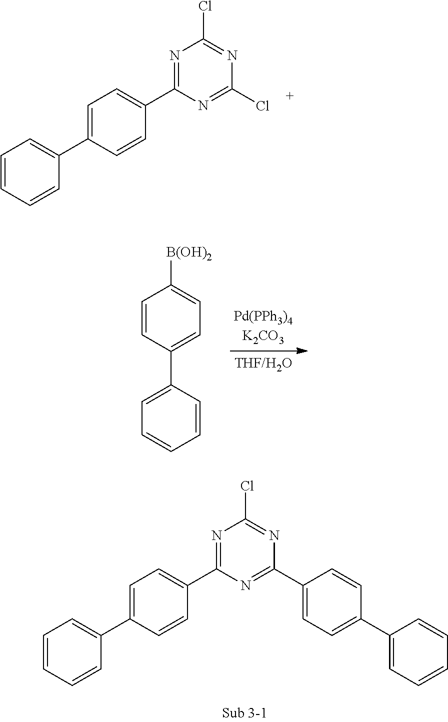 [SMILES: Clc1nc(Cl)nc(-c2ccc(-c3ccccc3)cc2)n1 ; OB(O)c1ccc(-c2ccccc2)cc1 ; Clc1nc(-c2ccc(-c3ccccc3)cc2)nc(-c2ccc(-c3ccccc3)cc2)n1]

2-([1,1′-biphenyl]-4-yl)-4,6-dichloro-1,3,5-triazine (CAS Registry Number: 10202-45-6) (20 g, 66.19 mmol), 4-Biphenylboronic acid (CAS Registry Number: 5122-94-1) (13.1 g, 66.19 mmol) were dissolved in THF (370 ml), Pd(PPh₃)₄ (3.8 g, 3.31 mmol), K₂CO₃ (27.4 g, 198.57 mmol) and water (165 ml) were added and stirred under reflux. When the reaction was completed, after extraction with ether and water, the organic layer is concentrated. The concentrated organic layer was dried over MgSO₄ and concentrated once more. The final concentrate was passed through a silica gel column and recrystallized to obtain 20.8 g of product. (75% yield)

### Synthesis Example of Sub 3-8

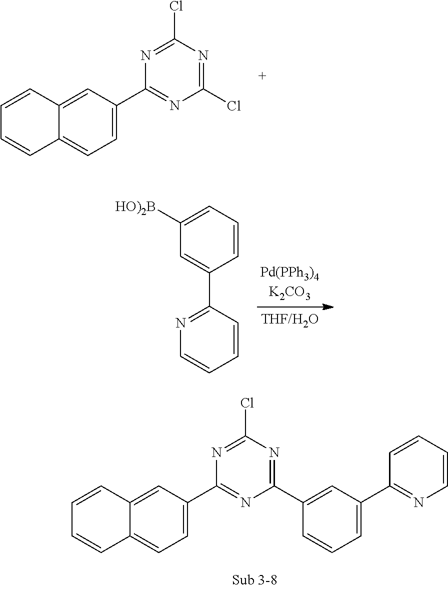

2,4-dichloro-6-(naphthalen-2-yl)-1,3,5-triazine (20 g, 72.43 mmol), (3-(pyridin-2-yl)phenyl)boronic acid (14.3 g, 72.43 mmol), Pd(PPh₃)₄ (0.05 eq.), K₂CO₃ (3 eq.), anhydrous THF and a small amount of water were added, and 20.3 g of the product was synthesized in the same manner as in the synthesis of Sub 3-1. (Yield 71%)

### Synthesis Example of Sub 3-19

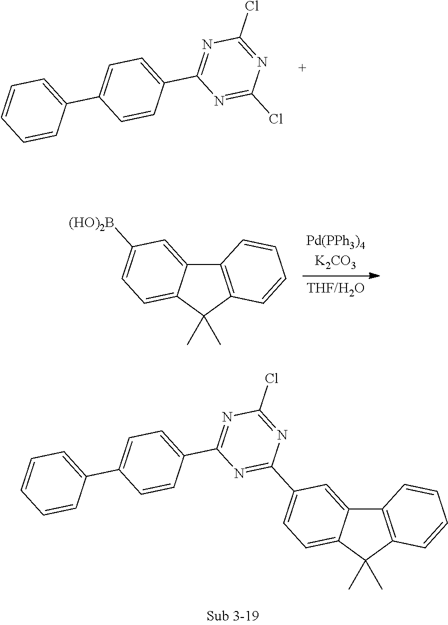

2-([1,1′-biphenyl]-4-yl)-4,6-dichloro-1,3,5-triazine (15 g, 49.64 mmol), (9,9-dimethyl-9H-fluoren-3-yl)boronic acid (11.8 g, 49.64 mmol), Pd(PPh₃)₄ (0.05 eq.), K₂CO₃ (3 eq.), Anhydrous THF and a small amount of water were added, and 15.7 g of the product was synthesized in the same manner as in the synthesis of Sub 3-1. (Yield 69%)

### Synthesis Example of Sub 3-35

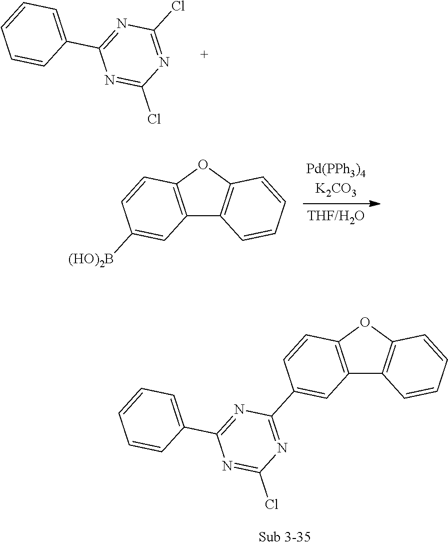 [SMILES: Clc1nc(Cl)nc(-c2ccccc2)n1 ; OB(O)c1ccc2oc3ccccc3c2c1 ; Clc1nc(-c2ccccc2)nc(-c2ccc3oc4ccccc4c3c2)n1]

2,4-dichloro-6-phenyl-1,3,5-triazine (30 g, 132.71 mmol), dibenzo[b,d]furan-2-ylboronic acid (28.1 g, 132.71 mmol), Pd(PPh₃)₄ (0.05 eq.), K₂CO₃ (3 eq.), anhydrous THF and a small amount of water were added, and 30.8 g of the product was synthesized in the same manner as in the synthesis of Sub 3-1. (Yield 65%)

Compounds belonging to Sub 3 may be the following compounds, but are not limited thereto, and Table 4 shows Field Desorption-Mass Spectrometry (FD-MS) values of some compounds belonging to Sub 3.

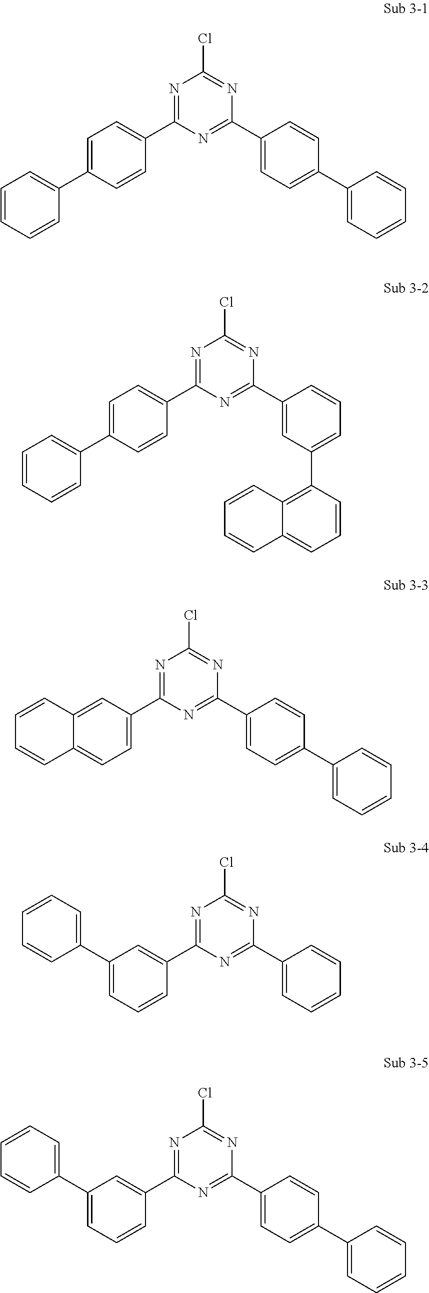 [SMILES: Clc1nc(-c2ccc(-c3ccccc3)cc2)nc(-c2ccc(-c3ccccc3)cc2)n1 ; Clc1nc(-c2ccc(-c3ccccc3)cc2)nc(-c2cccc(-c3cccc4ccccc34)c2)n1 ; Clc1nc(-c2ccc(-c3ccccc3)cc2)nc(-c2ccc3ccccc3c2)n1 ; Clc1nc(-c2ccccc2)nc(-c2cccc(-c3ccccc3)c2)n1 ; Clc1nc(-c2ccc(-c3ccccc3)cc2)nc(-c2cccc(-c3ccccc3)c2)n1]

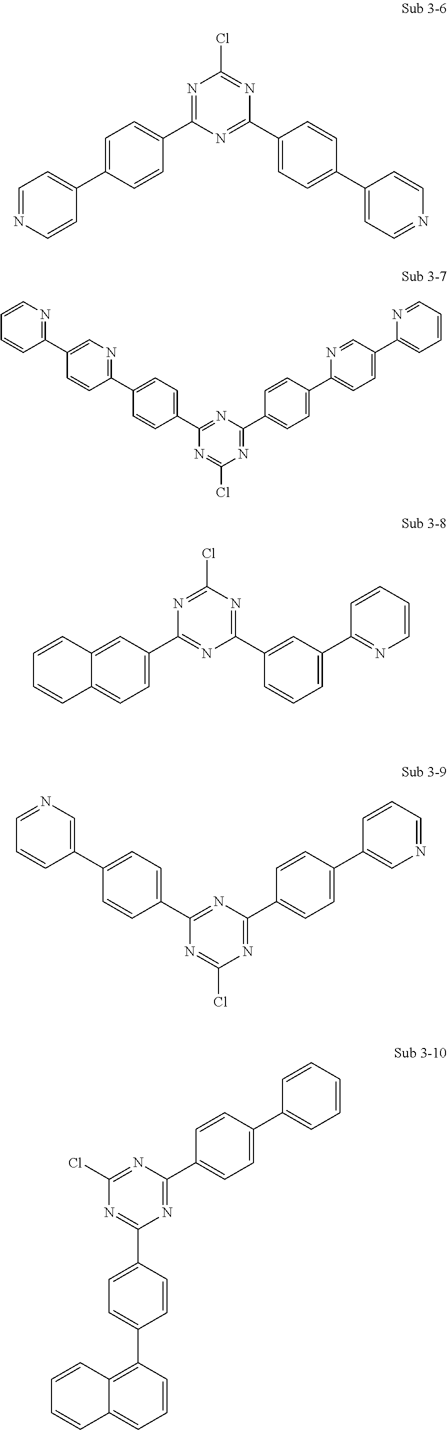 [SMILES: Clc1nc(-c2ccc(-c3ccncc3)cc2)nc(-c2ccc(-c3ccncc3)cc2)n1 ; Clc1nc(-c2ccc(-c3ccc(-c4ccccn4)cn3)cc2)nc(-c2ccc(-c3ccc(-c4ccccn4)cn3)cc2)n1 ; Clc1nc(-c2cccc(-c3ccccn3)c2)nc(-c2ccc3ccccc3c2)n1 ; Clc1nc(-c2ccc(-c3cccnc3)cc2)nc(-c2ccc(-c3cccnc3)cc2)n1 ; Clc1nc(-c2ccc(-c3ccccc3)cc2)nc(-c2ccc(-c3cccc4ccccc34)cc2)n1]

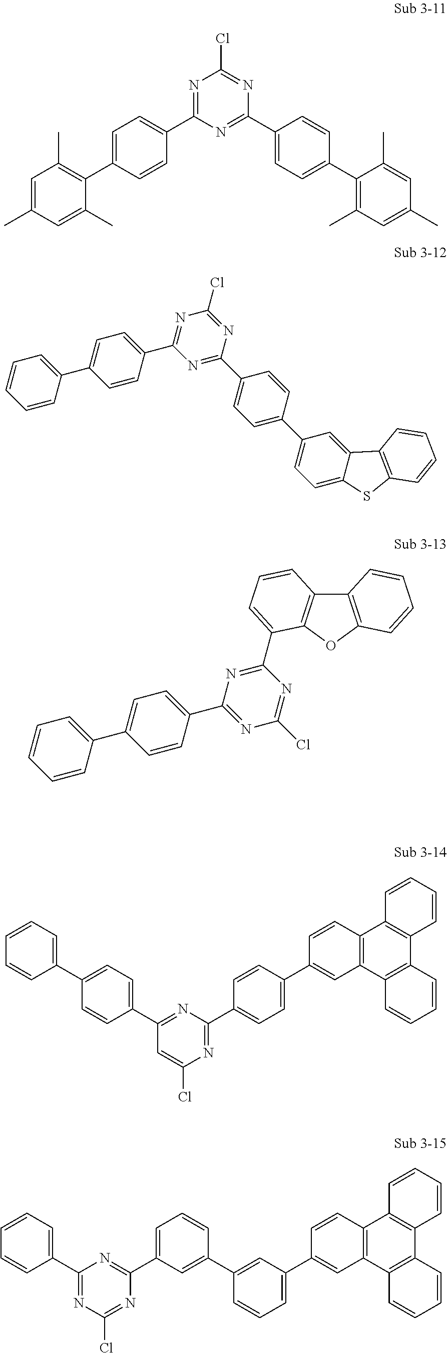

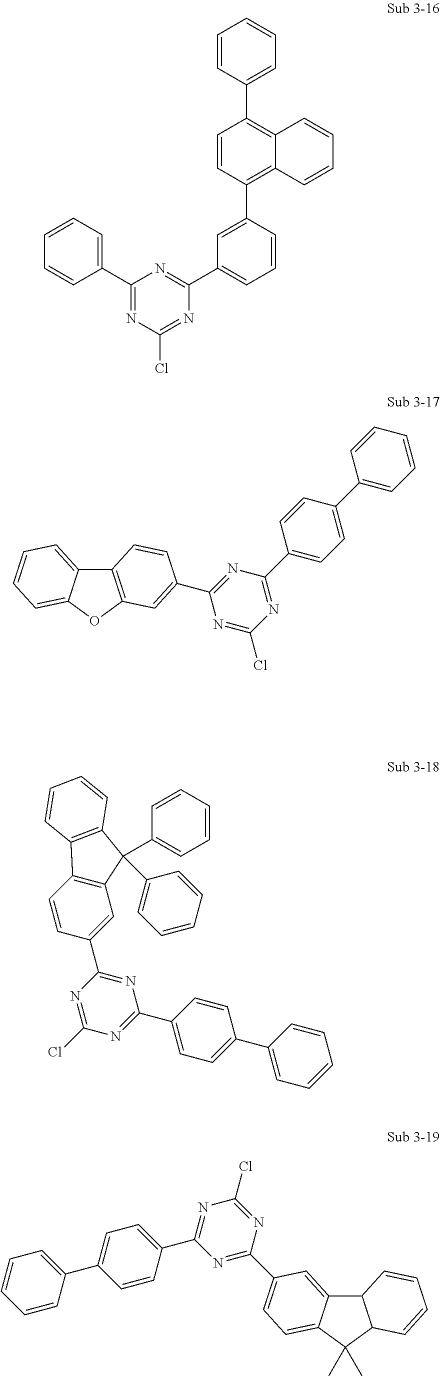 [SMILES: Clc1nc(-c2ccccc2)nc(-c2cccc(-c3ccc(-c4ccccc4)c4ccccc34)c2)n1 ; Clc1nc(-c2ccc(-c3ccccc3)cc2)nc(-c2ccc3c(c2)oc2ccccc23)n1 ; Clc1nc(-c2ccc(-c3ccccc3)cc2)nc(-c2ccc3c(c2)C(c2ccccc2)(c2ccccc2)c2ccccc2-3)n1 ; CC1(C)c2ccc(-c3nc(Cl)nc(-c4ccc(-c5ccccc5)cc4)n3)cc2C2C=CC=CC21]

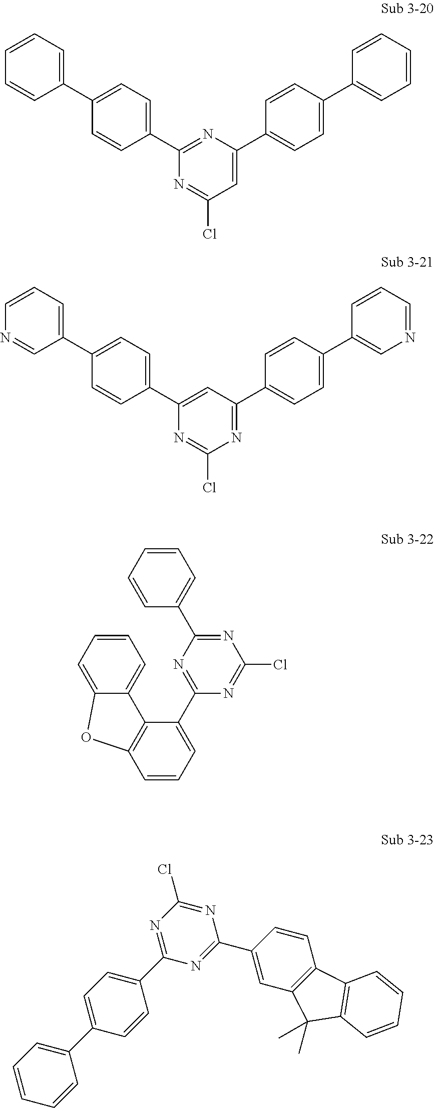 [SMILES: Clc1cc(-c2ccc(-c3ccccc3)cc2)nc(-c2ccc(-c3ccccc3)cc2)n1 ; Clc1nc(-c2ccc(-c3cccnc3)cc2)cc(-c2ccc(-c3cccnc3)cc2)n1 ; Clc1nc(-c2ccccc2)nc(-c2cccc3oc4ccccc4c23)n1 ; CC1(C)c2ccccc2-c2ccc(-c3nc(Cl)nc(-c4ccc(-c5ccccc5)cc4)n3)cc21]

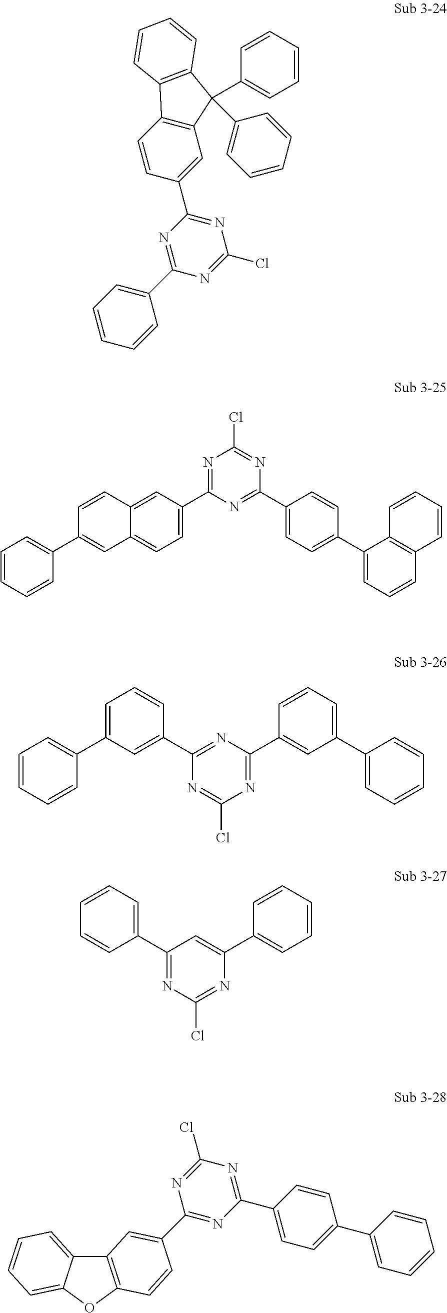 [SMILES: Clc1nc(-c2ccccc2)nc(-c2ccc3c(c2)C(c2ccccc2)(c2ccccc2)c2ccccc2-3)n1 ; Clc1nc(-c2ccc(-c3cccc4ccccc34)cc2)nc(-c2ccc3cc(-c4ccccc4)ccc3c2)n1 ; Clc1nc(-c2cccc(-c3ccccc3)c2)nc(-c2cccc(-c3ccccc3)c2)n1 ; Clc1nc(-c2ccccc2)cc(-c2ccccc2)n1 ; Clc1nc(-c2ccc(-c3ccccc3)cc2)nc(-c2ccc3oc4ccccc4c3c2)n1]

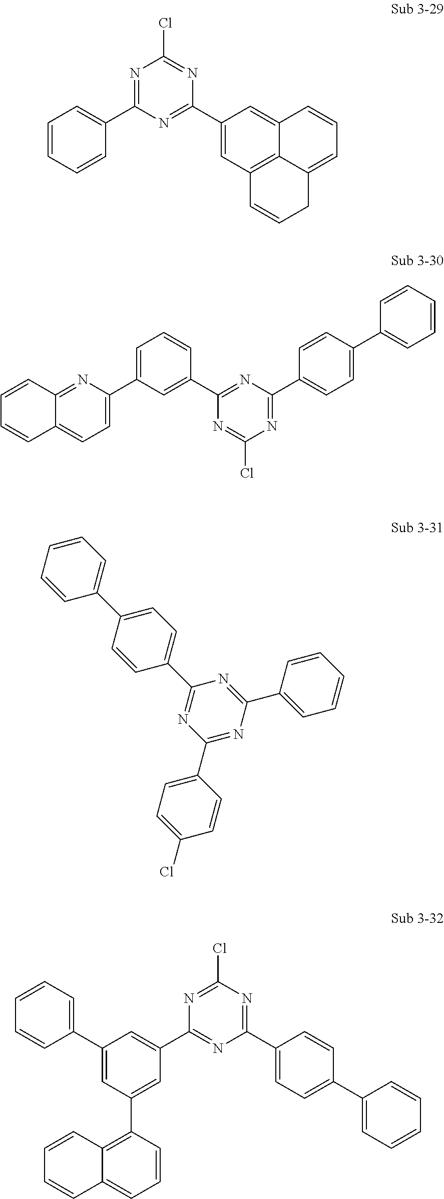 [SMILES: Clc1nc(-c2ccccc2)nc(-c2cc3c4c(cccc4c2)CC=C3)n1 ; Clc1nc(-c2ccc(-c3ccccc3)cc2)nc(-c2cccc(-c3ccc4ccccc4n3)c2)n1 ; Clc1ccc(-c2nc(-c3ccccc3)nc(-c3ccc(-c4ccccc4)cc3)n2)cc1 ; Clc1nc(-c2ccc(-c3ccccc3)cc2)nc(-c2cc(-c3ccccc3)cc(-c3cccc4ccccc34)c2)n1]

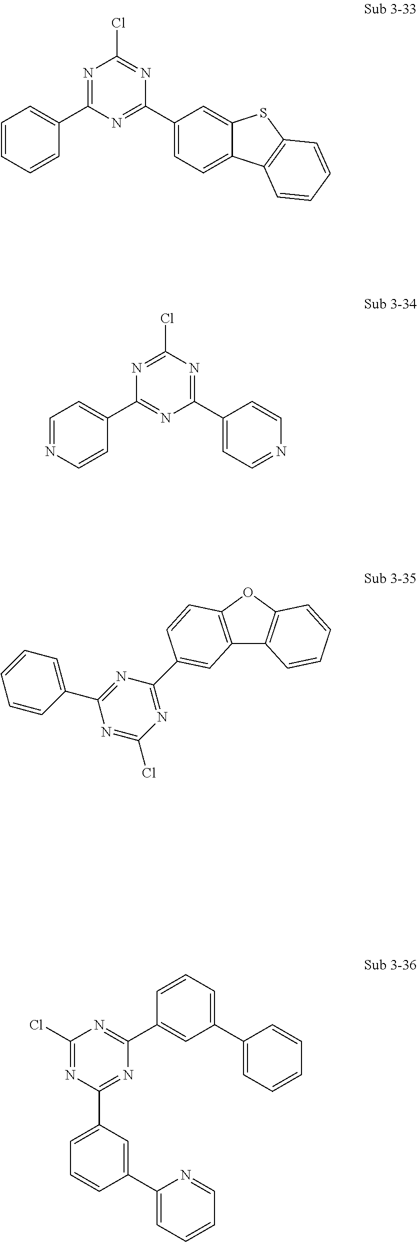 [SMILES: Clc1nc(-c2ccccc2)nc(-c2ccc3c(c2)sc2ccccc23)n1 ; Clc1nc(-c2ccncc2)nc(-c2ccncc2)n1 ; Clc1nc(-c2ccccc2)nc(-c2ccc3oc4ccccc4c3c2)n1 ; Clc1nc(-c2cccc(-c3ccccc3)c2)nc(-c2cccc(-c3ccccn3)c2)n1]

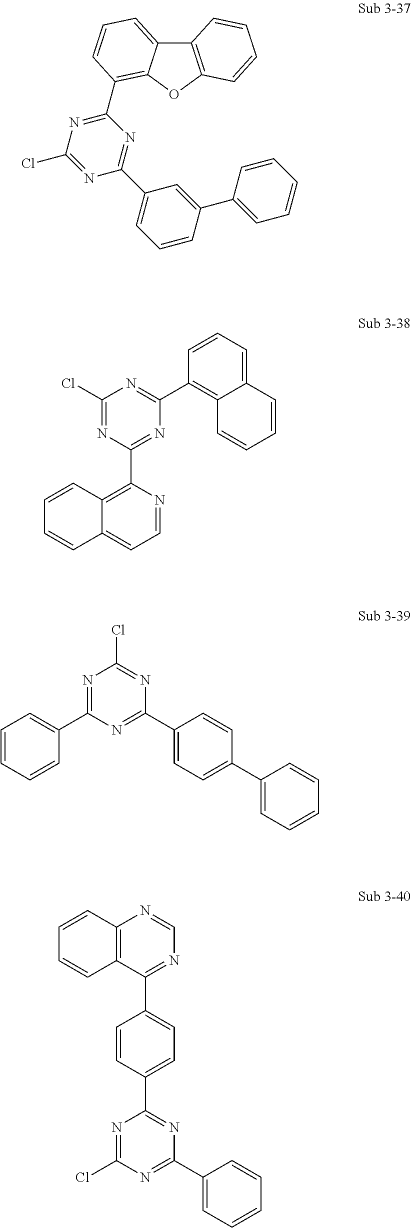 [SMILES: Clc1nc(-c2cccc(-c3ccccc3)c2)nc(-c2cccc3c2oc2ccccc23)n1 ; Clc1nc(-c2cccc3ccccc23)nc(-c2nccc3ccccc23)n1 ; Clc1nc(-c2ccccc2)nc(-c2ccc(-c3ccccc3)cc2)n1 ; Clc1nc(-c2ccccc2)nc(-c2ccc(-c3ncnc4ccccc34)cc2)n1]

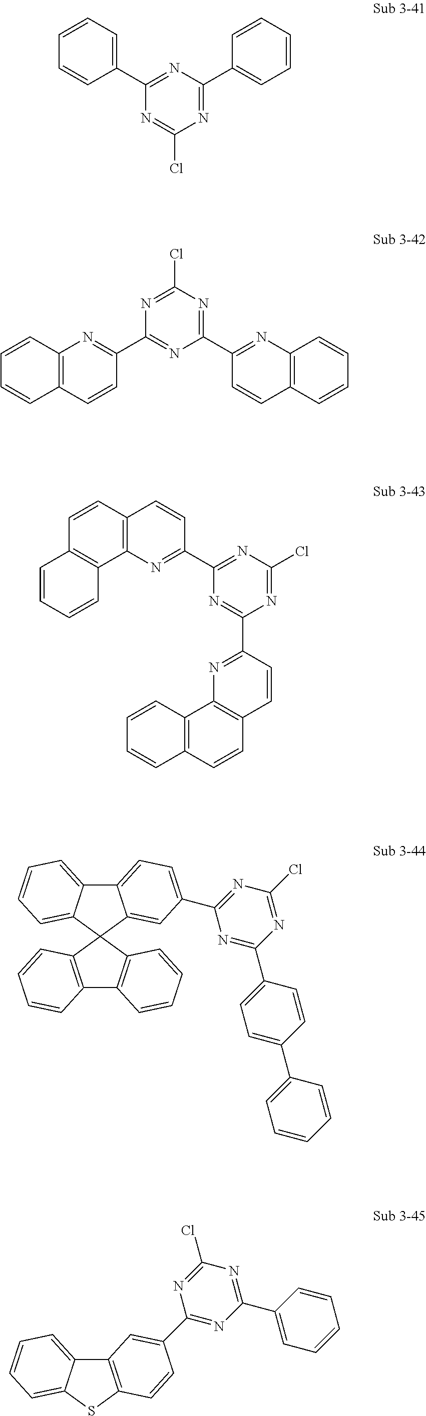 [SMILES: Clc1nc(-c2ccccc2)nc(-c2ccccc2)n1 ; Clc1nc(-c2ccc3ccccc3n2)nc(-c2ccc3ccccc3n2)n1 ; Clc1nc(-c2ccc3ccc4ccccc4c3n2)nc(-c2ccc3ccc4ccccc4c3n2)n1 ; Clc1nc(-c2ccc(-c3ccccc3)cc2)nc(-c2ccc3c(c2)C2(c4ccccc4-c4ccccc42)c2ccccc2-3)n1 ; Clc1nc(-c2ccccc2)nc(-c2ccc3sc4ccccc4c3c2)n1]

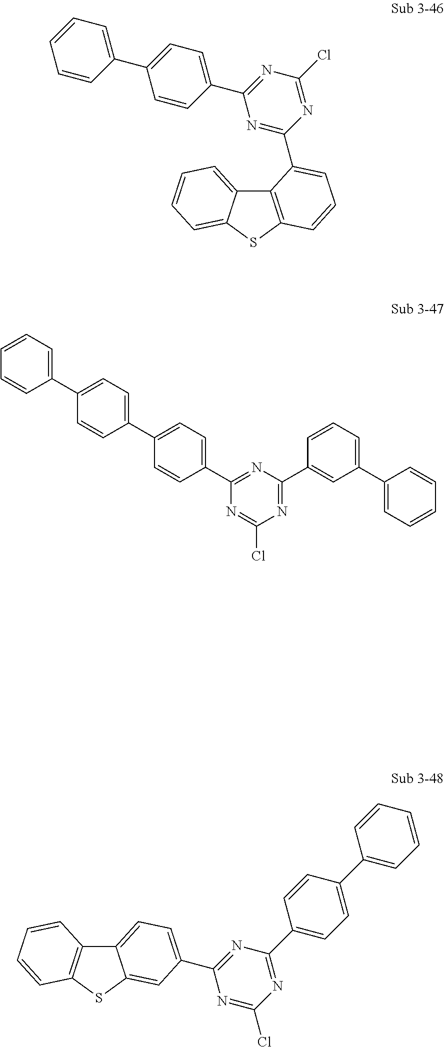 [SMILES: Clc1nc(-c2ccc(-c3ccccc3)cc2)nc(-c2cccc3sc4ccccc4c23)n1 ; Clc1nc(-c2ccc(-c3ccc(-c4ccccc4)cc3)cc2)nc(-c2cccc(-c3ccccc3)c2)n1 ; Clc1nc(-c2ccc(-c3ccccc3)cc2)nc(-c2ccc3c(c2)sc2ccccc23)n1]

TABLE 4

| com- | |
|---|---|
| pound | FD-MS |
| Sub 3-1 | m/z = 419.12 (C₂₇H₁₈ClN₃ = 419.91) |
| Sub 3-2 | m/z = 469.13 (C₃₁H₂₀ClN₃ = 469.97) |
| Sub 3-3 | m/z = 393.1 (C₂₅H₁₆ClN₃ = 393.87) |
| Sub 3-4 | m/z = 343.09 (C₂₁H₁₄ClN₃ = 343.81) |
| Sub 3-5 | m/z = 419.12 (C₂₇H₁₈ClN₃ = 419.91) |
| Sub 3-6 | m/z = 421.11 (C₂₅H₁₆ClN₅ = 421.89) |
| Sub 3-7 | m/z = 575.16 (C₃₅H₂₂ClN₇ = 576.06) |
| Sub 3-8 | m/z = 394.1 (C₂₄H₁₅ClN₄ = 394.86) |
| Sub 3-9 | m/z = 421.11 (C₂₅H₁₆ClN₅ = 421.89) |
| Sub 3-10 | m/z = 469.13 (C₃₁H₂₀ClN₃ = 469.97) |
| Sub 3-11 | m/z = 503.21 (C₃₃H₃₀ClN₃ = 504.07) |
| Sub 3-12 | m/z = 525.11 (C₃₃H₂₀ClN₃S = 526.05) |
| Sub 3-13 | m/z = 433.1 (C₂₇H₁₆ClN₃O = 433.9) |
| Sub 3-14 | m/z = 568.17 (C₄₀H₂₅ClN₂ = 569.1) |
| Sub 3-15 | m/z = 569.17 (C₃₉H₂₄ClN₃ = 570.09) |
| Sub 3-16 | m/z = 469.13 (C₃₁H₂₀ClN₃ = 469.97) |
| Sub 3-17 | m/z = 433.1 (C₂₇H₁₆ClN₃O = 433.9) |
| Sub 3-18 | m/z = 583.18 (C₄₀H₂₆ClN₃ = 584.12) |
| Sub 3-19 | m/z = 461.17 (C₃₀H₂₄ClN₃ = 461.99) |
| Sub 3-20 | m/z = 418.12 (C₁₈H₁₉ClN₂ = 418.92) |
| Sub 3-21 | m/z = 420.11 (C₂₆H₁₇ClN₄ = 420.9) |
| Sub 3-22 | m/z = 357.07 (C₂₁H₁₂ClN₃O = 357.8) |
| Sub 3-23 | m/z = 459.15 (C₃₀H₂₂ClN₃ = 459.98) |
| Sub 3-24 | m/z = 507.15 (C₃₄H₂₂ClN₃ = 508.02) |
| Sub 3-25 | m/z = 519.15 (C₃₅H₂₂ClN₃ = 520.03) |
| Sub 3-26 | m/z = 419.12 (C₂₇H₁₈ClN₃ = 419.91) |
| Sub 3-27 | m/z = 266.06 (C₁₆H₁₁ClN₂ = 266.73) |
| Sub 3-28 | m/z = 433.1 (C₂₇H₁₆ClN₃O = 433.9) |
| Sub 3-29 | m/z = 355.09 (C₂₂H₁₄ClN₃ = 355.83) |
| Sub 3-30 | m/z = 470.13 (C₃₀H₁₉ClN₄ = 470.96) |
| Sub 3-31 | m/z = 419.12 (C₂₇H₁₈ClN₃ = 419.91) |
| Sub 3-32 | m/z = 545.17 (C₃₇H₂₄ClN₃ = 546.07) |
| Sub 3-33 | m/z = 373.04 (C₂₁H₁₂ClN₃S = 373.86) |
| Sub 3-34 | m/z = 269.05 (C₁₃H₈ClN₅ = 269.69) |
| Sub 3-35 | m/z = 357.07 (C₂₁ H₁₂ClN₃O = 357.8) |
| Sub 3-36 | m/z = 420.11 (C₂₆H₁₇ClN₄ = 420.9) |
| Sub 3-37 | m/z = 433.1 (C₂₇H₁₆ClN₃O = 433.9) |
| Sub 3-38 | m/z = 368.08 (C₂₂H₁₃ClN₄ = 368.82) |
| Sub 3-39 | m/z = 343.09 (C₂₁H₁₄ClN₃ = 343.81) |
| Sub 3-40 | m/z = 395.09 (C₂₃H₁₄ClN₅ = 395.85) |
| Sub 3-41 | m/z = 267.06 (C₁₅H₁₀ClN₃ = 267.72) |
| Sub 3-42 | m/z = 369.08 (C₂₁H₁₂ClN₅ = 369.81) |
| Sub 3-43 | m/z = 469.11 (C₂₉H₁₆ClN₅ = 469.93) |
| Sub 3-44 | m/z = 581.17 (C₄₀H₂₄ClN₃ = 582.1) |
| Sub 3-45 | m/z = 373.04 (C₂₁H₁₂ClN₃S = 373.86) |
| Sub 3-46 | m/z = 449.08 (C₂₇H₁₆ClN₃S = 449.96) |
| Sub 3-47 | m/z = 495.15 (C₃₃H₂₂ClN₃ = 496.01) |
| Sub 3-48 | m/z = 449.08 (C₂₇H₁₆ClN₃S = 449.96) |

### II. Synthesis Example of Sub 4

Sub 4 of Reaction Scheme 1 may be synthesized by the reaction pathway of Reaction Scheme 5, but is not limited thereto. Hal⁵ is I, Br or Cl.

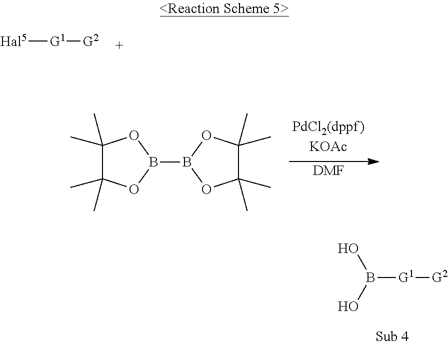

### Synthesis Example of Sub 4-2

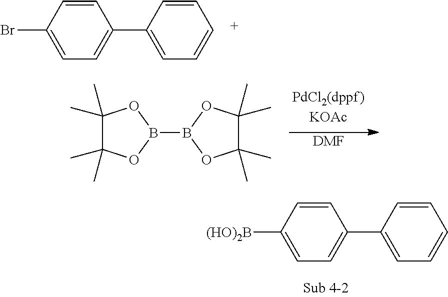

4-bromo-1,1′-biphenyl (5 g, 21.45 mmol), bis(pinacolato)diboron (7.1 g, 27.89 mmol), PdCl₂(dppf), (0.78 g, 1.07 mmol), KOAc (6.3 g, 64.35 mmol), DMF (270 ml) were added and stirred and refluxed at 120° C. When the reaction is completed, the reactant is cooled to room temperature, extracted with MC, and washed with water. After drying the organic layer with MgSO₄ and concentrating, the resulting organic material was separated using a silica gel column to obtain 3.4 g of Sub 4-2 (yield: 80%).

### Synthesis Example of Sub 4-37

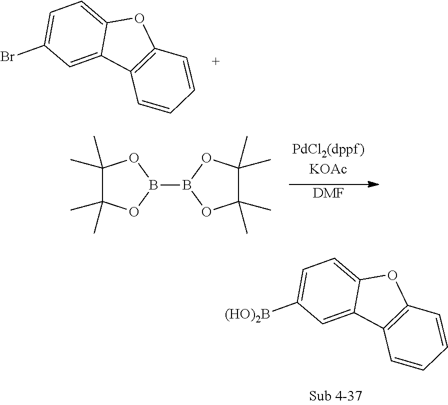

2-bromodibenzo[b,d]furan (10 g, 40.47 mmol) bis(pinacolato)diboron (13.3 g, 52.61 mmol), PdCl₂(dppf), (0.05 eq.), KOAc (3 eq.) and anhydrous DMF were added, and 7 g of the product was synthesized in the same manner as in the synthesis of Sub 4-2.(yield: 82%)

Compounds belonging to Sub 4 may be the following compounds, but are not limited thereto, and Table 5 shows Field Desorption-Mass Spectrometry (FD-MS) values of some compounds belonging to Sub 4.

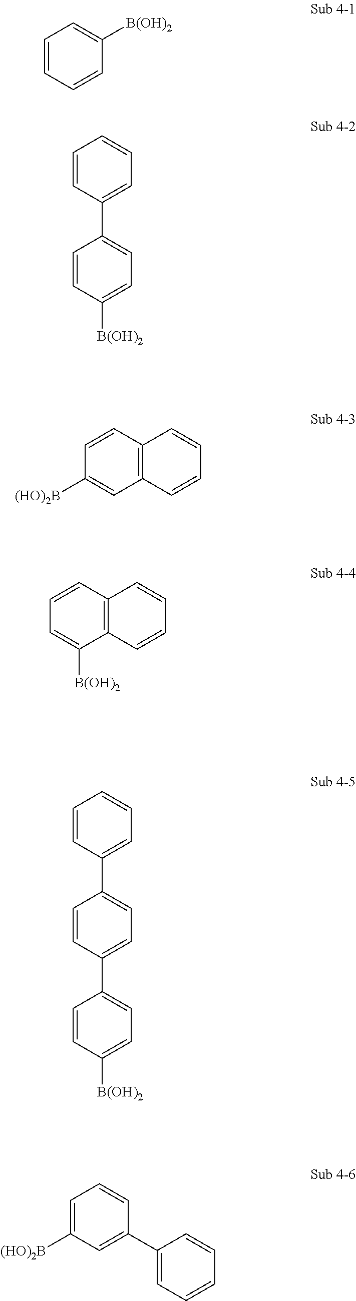 [SMILES: OB(O)c1ccccc1 ; OB(O)c1ccc(-c2ccccc2)cc1 ; OB(O)c1ccc2ccccc2c1 ; OB(O)c1cccc2ccccc12 ; OB(O)c1ccc(-c2ccc(-c3ccccc3)cc2)cc1 ; OB(O)c1cccc(-c2ccccc2)c1]

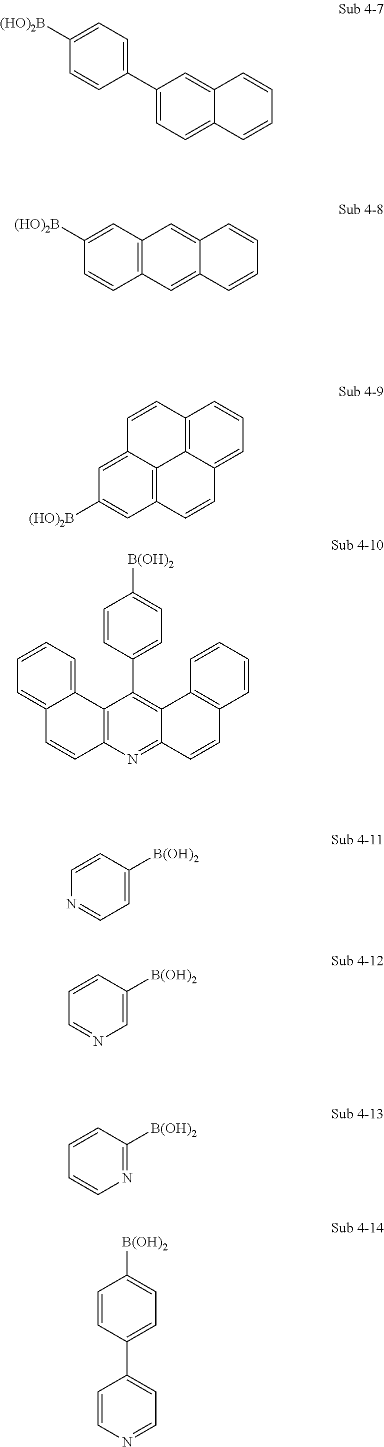

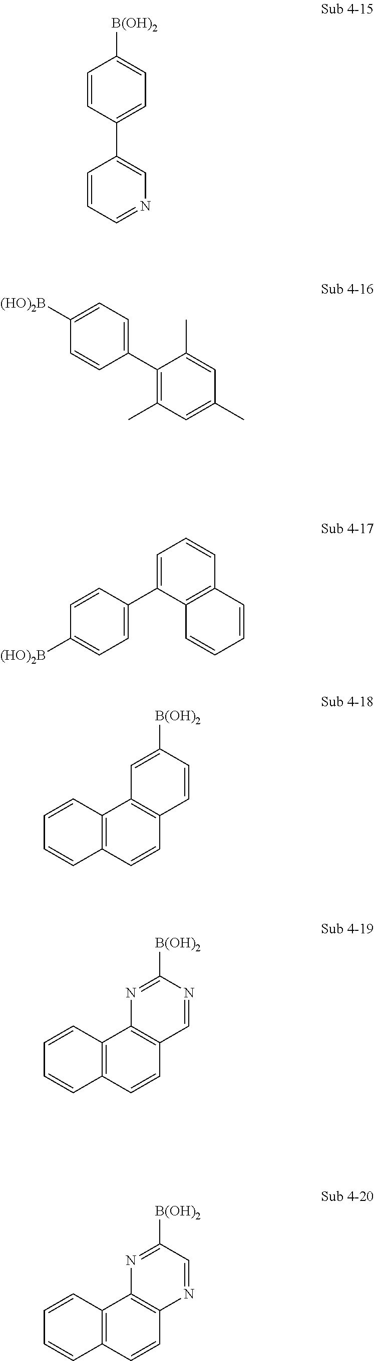 [SMILES: OB(O)c1ccc(-c2cccnc2)cc1 ; Cc1cc(C)c(-c2ccc(B(O)O)cc2)c(C)c1 ; OB(O)c1ccc(-c2cccc3ccccc23)cc1 ; OB(O)c1ccc2ccc3ccccc3c2c1 ; OB(O)c1ncc2ccc3ccccc3c2n1 ; OB(O)c1cnc2ccc3ccccc3c2n1]

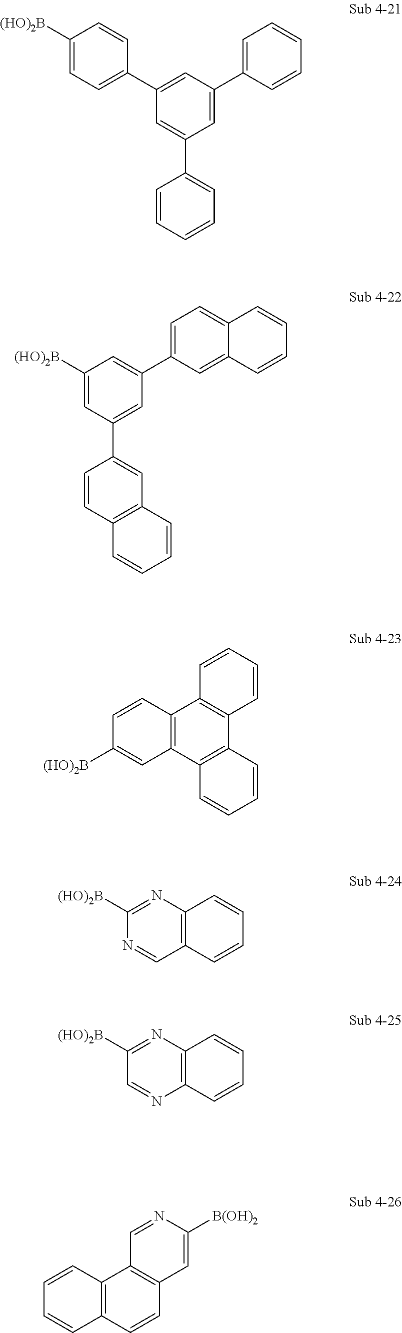 [SMILES: OB(O)c1ccc(-c2cc(-c3ccccc3)cc(-c3ccccc3)c2)cc1 ; OB(O)c1cc(-c2ccc3ccccc3c2)cc(-c2ccc3ccccc3c2)c1 ; OB(O)c1ccc2c3ccccc3c3ccccc3c2c1 ; OB(O)c1ncc2ccccc2n1 ; OB(O)c1cnc2ccccc2n1 ; OB(O)c1cc2ccc3ccccc3c2cn1]

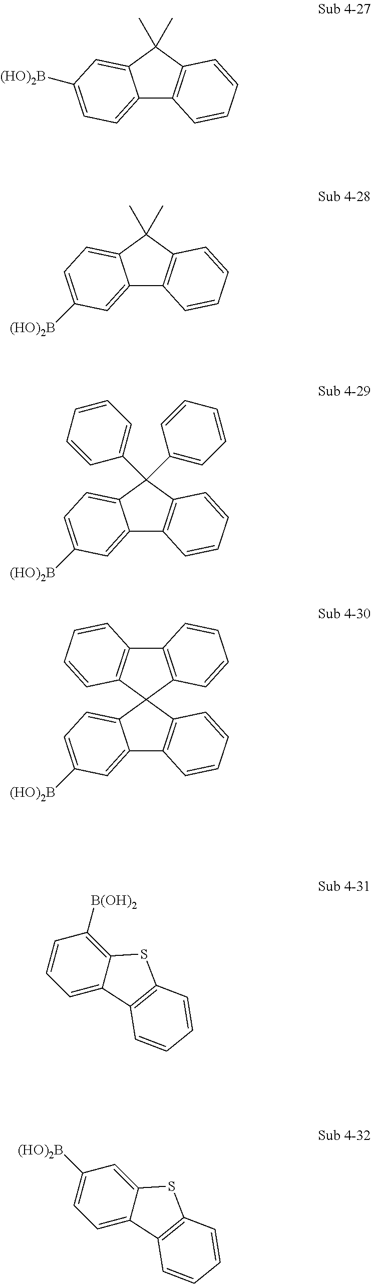 [SMILES: CC1(C)c2ccccc2-c2ccc(B(O)O)cc21 ; CC1(C)c2ccccc2-c2cc(B(O)O)ccc21 ; OB(O)c1ccc2c(c1)-c1ccccc1C2(c1ccccc1)c1ccccc1 ; OB(O)c1ccc2c(c1)-c1ccccc1C21c2ccccc2-c2ccccc21 ; OB(O)c1cccc2c1sc1ccccc12 ; OB(O)c1ccc2c(c1)sc1ccccc12]

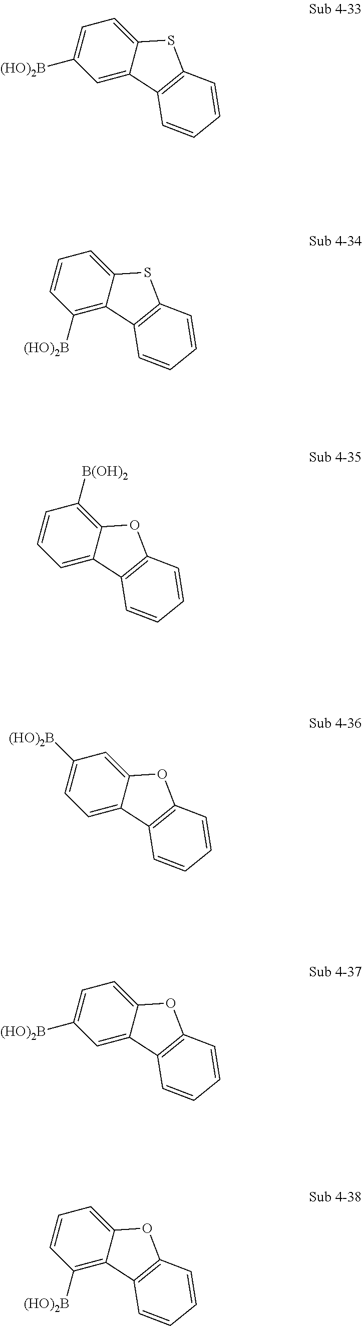 [SMILES: OB(O)c1ccc2sc3ccccc3c2c1 ; OB(O)c1cccc2sc3ccccc3c12 ; OB(O)c1cccc2c1oc1ccccc12 ; OB(O)c1ccc2c(c1)oc1ccccc12 ; OB(O)c1ccc2oc3ccccc3c2c1 ; OB(O)c1cccc2oc3ccccc3c12]

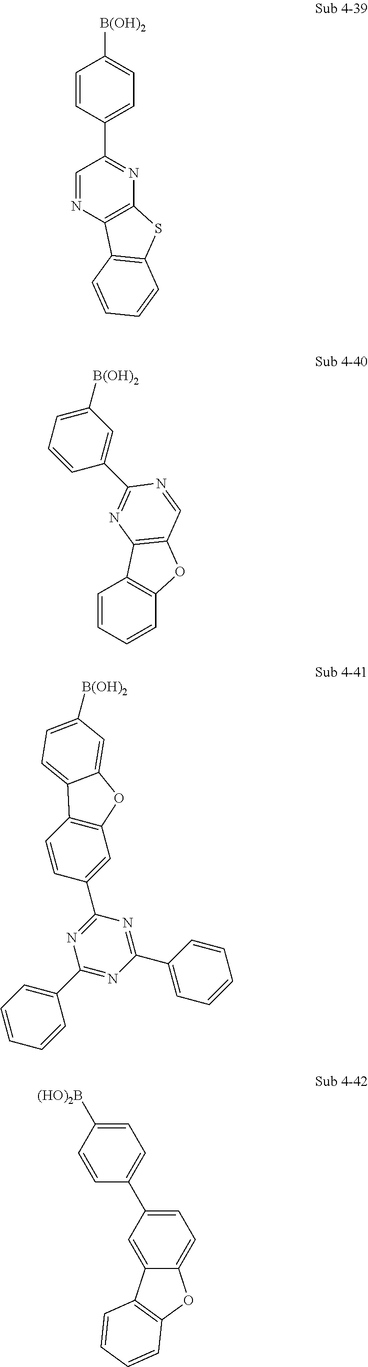 [SMILES: OB(O)c1ccc(-c2cnc3c(n2)sc2ccccc23)cc1 ; OB(O)c1cccc(-c2ncc3oc4ccccc4c3n2)c1 ; OB(O)c1ccc2c(c1)oc1cc(-c3nc(-c4ccccc4)nc(-c4ccccc4)n3)ccc12 ; OB(O)c1ccc(-c2ccc3oc4ccccc4c3c2)cc1]

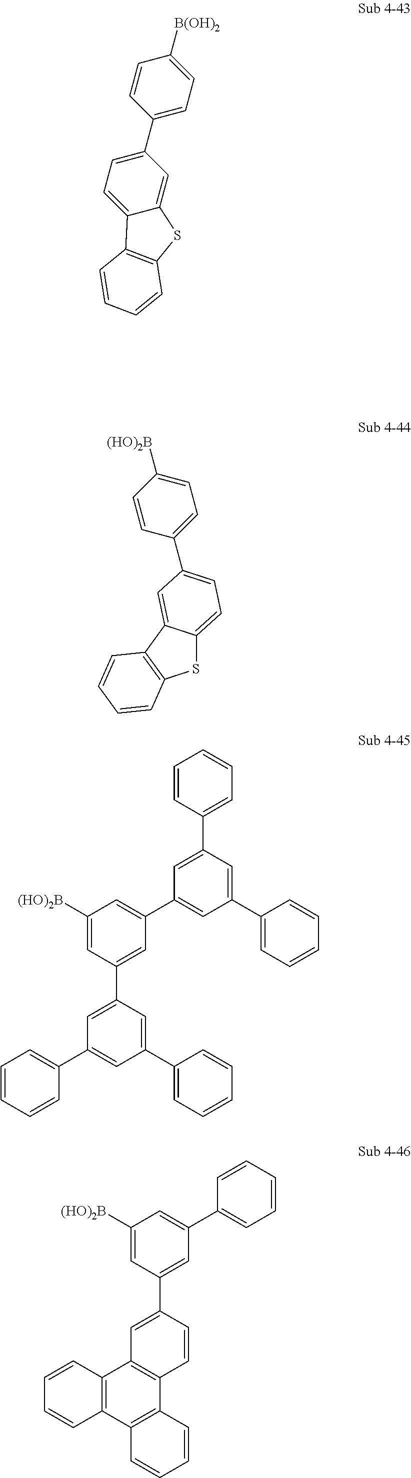 [SMILES: OB(O)c1ccc(-c2ccc3c(c2)sc2ccccc23)cc1 ; OB(O)c1ccc(-c2ccc3sc4ccccc4c3c2)cc1 ; OB(O)c1cc(-c2cc(-c3ccccc3)cc(-c3ccccc3)c2)cc(-c2cc(-c3ccccc3)cc(-c3ccccc3)c2)c1 ; OB(O)c1cc(-c2ccccc2)cc(-c2ccc3c4ccccc4c4ccccc4c3c2)c1]

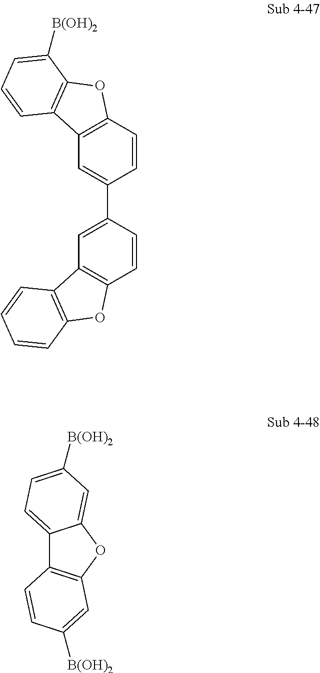 [SMILES: OB(O)c1cccc2c1oc1ccc(-c3ccc4oc5ccccc5c4c3)cc12 ; OB(O)c1ccc2c(c1)oc1cc(B(O)O)ccc12]

TABLE 5

| com- | |
|---|---|
| pound | FD-MS |
| Sub 4-1 | m/z = 122.05 (C₆H₇BO₂ = 121.93) |
| Sub 4-2 | m/z = 198.09 (C₁₂H₁₁BO₂ = 198.03) |
| Sub 4-3 | m/z = 172.07 (C₁₀H₉BO₂ = 171.99) |
| Sub 4-4 | m/z = 172.07 (C₁₀H₉BO₂ = 171.99) |
| Sub 4-5 | m/z = 274.12 (C₁₈H₁₅BO₂ = 274.13) |
| Sub 4-6 | m/z = 198.09 (C₁₂H₁₁BO₂ = 198.03) |
| Sub 4-7 | m/z = 248.1 (C₁₆H₁₃BO₂ = 248.09) |
| Sub 4-8 | m/z = 222.09 (C₁₄H₁₁BO₂ = 222.05) |
| Sub 4-9 | m/z = 246.09 (C₁₆H₁₁BO₂ = 246.07) |
| Sub 4-10 | m/z = 399.14 (C₂₇H₁₈BNO₂ = 399.26) |
| Sub 4-11 | m/z = 123.05 (C₅H₆BNO₂ = 122.92) |
| Sub 4-12 | m/z = 123.05 (C₅H₆BNO₂ = 122.92) |
| Sub 4-13 | m/z = 123.05 (C₅H₆BNO₂ = 122.92) |
| Sub 4-14 | m/z = 199.08 (C₁₁H₁₀BNO₂ = 199.02) |
| Sub 4-15 | m/z = 199.08 (C₁₁H₁₀BNO₂ = 199.02) |
| Sub 4-16 | m/z = 240.13 (C₁₅H₁₇BO₂ = 240.11) |
| Sub 4-17 | m/z = 248.1 (C₁₆H₁₃BO₂ = 248.09) |
| Sub 4-18 | m/z = 222.09 (C₁₄H₁₁BO₂ = 222.05) |
| Sub 4-19 | m/z = 224.08 (C₁₂H₉BN₂O₂ = 224.03) |
| Sub 4-20 | m/z = 224.08 (C₁₂H₉BN₂O₂ = 224.03) |
| Sub 4-21 | m/z = 350.15 (C₂₄H₁₉BO₂ = 350.22) |
| Sub 4-22 | m/z = 374.15 (C₂₆H₁₉BO₂ = 374.25) |
| Sub 4-23 | m/z = 272.1 (C₁₈H₁₃BO₂ = 272.11) |
| Sub 4-24 | m/z = 174.06 (C₈H₇BN₂O₂ = 173.97) |
| Sub 4-25 | m/z = 174.06 (C₈H₇BN₂O₂ = 173.97) |
| Sub 4-26 | m/z = 223.08 (C₁₃H₁₀BNO₂ = 223.04) |
| Sub 4-27 | m/z = 238.12 (C₁₅H₁₅BO₂ = 238.09) |
| Sub 4-28 | m/z = 238.12 (C₁₅H₁₅BO₂ = 238.09) |
| Sub 4-29 | m/z = 362.15 (C₂₅H₁₉BO₂ = 362.24) |
| Sub 4-30 | m/z = 360.13 (C₂₅H₁₇BO₂ = 360.22) |
| Sub 4-31 | m/z = 228.04 (C₁₂H₉BO₂S = 228.07) |
| Sub 4-32 | m/z = 228.04 (C₁₂H₉BO₂S = 228.07) |
| Sub 4-33 | m/z = 228.04 (C₁₂H₉BO₂S = 228.07) |
| Sub 4-34 | m/z = 228.04 (C₁₂H₉BO₂S = 228.07) |
| Sub 4-35 | m/z = 212.06 (C₁₂H₉BO₃ = 212.01) |
| Sub 4-36 | m/z = 212.06 (C₁₂H₉BO₃ = 212.01) |
| Sub 4-37 | m/z = 212.06 (C₁₂H₉BO₃ = 212.01) |
| Sub 4-38 | m/z = 212.06 (C₁₂H₉BO₃ = 212.01) |
| Sub 4-39 | m/z = 306.06 (C₁₆H₁₁BN₂O₂S = 306.15) |
| Sub 4-40 | m/z = 290.09 (C₁₆H₁₁BN₂O₃ = 290.09) |
| Sub 4-41 | m/z = 443.14 (C₂₇H₁₈BN₃O₃ = 443.27) |
| Sub 4-42 | m/z = 288.1 (C₁₈H₁₃BO₃ = 288.11) |
| Sub 4-43 | m/z = 304.07 (C₁₈H₁₃BO₂S = 304.17) |
| Sub 4-44 | m/z = 304.07 (C₁₈H₁₃BO₂S = 304.17) |
| Sub 4-45 | m/z = 578.24 (C₄₂H₃₁BO₂ = 578.52) |
| Sub 4-46 | m/z = 424.16 (C₃₀H₂₁BO₂ = 424.31) |
| Sub 4-47 | m/z = 378.11 (C₂₄H₁₅BO₄ = 378.19) |
| Sub 4-48 | m/z = 256.07 (C₁₂H₁₀B₂O₅ = 255.83) |

### III. Synthesis Example of Final Product 2

Sub 5 (1 equiv.) and Sub 6 (1-2 equiv.) were put in a round flask, dissolved in THF, and Pd(PPh₃)₄ (0.05 equiv.), K₂CO₃ (3 equiv.) and water were added and stirred and refluxed. When the reaction was completed, after extraction with ether and water, the organic layer was dried with MgSO₄, concentrated, and the resulting compound was recrystallized using a silica gel column to obtain Final product 2.

### Synthesis Example of 2-9

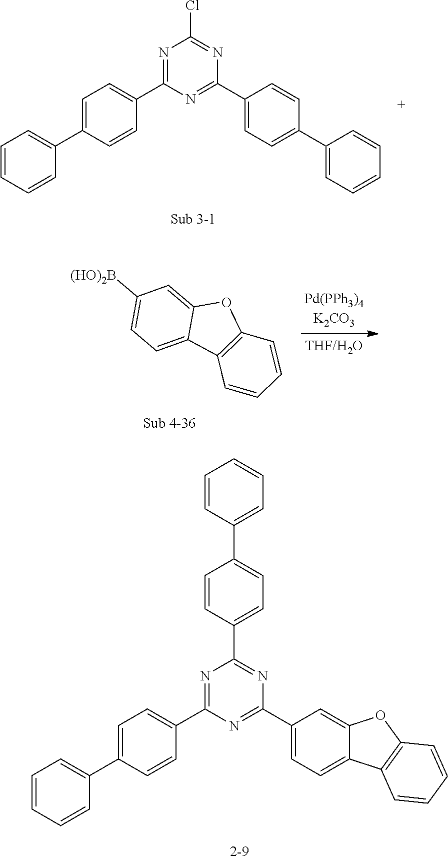 [SMILES: Clc1nc(-c2ccc(-c3ccccc3)cc2)nc(-c2ccc(-c3ccccc3)cc2)n1 ; OB(O)c1ccc2c(c1)oc1ccccc12 ; c1ccc(-c2ccc(-c3nc(-c4ccc(-c5ccccc5)cc4)nc(-c4ccc5c(c4)oc4ccccc45)n3)cc2)cc1]

Sub 3-1 (5 g, 11.91 mmol) was dissolved in Sub 4-36 (2.8 g, 13.1 mmol) in THF (70 ml), Pd(PPh₃)₄ (0.7 g, 0.6 mmol), K₂CO₃ (5 g, 35.73 mmol) and water (30 ml) were added and stirred at reflux. When the reaction was completed, after extraction with ether and water, the organic layer is concentrated. The concentrated organic layer was dried with MgSO₄, and concentrated once more. The final concentrate was passed through a silica gel column and recrystallized to obtain 5.4 g of product. (Yield: 71%)

### Synthesis Example of 2-29

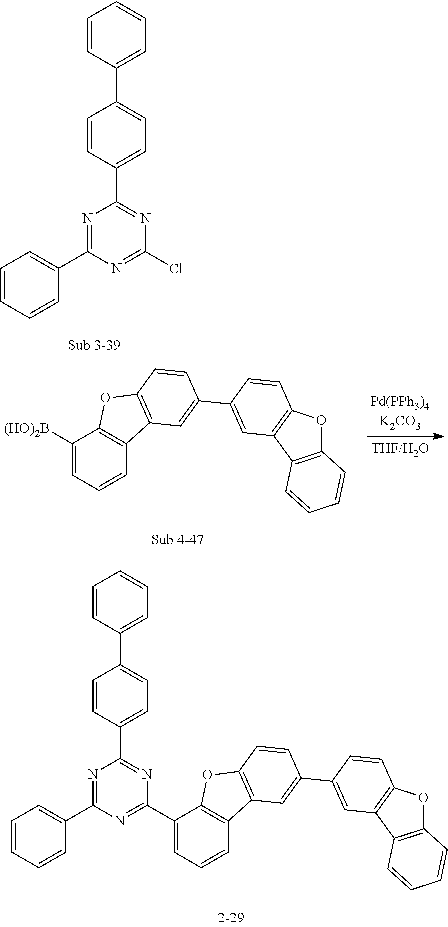 [SMILES: Clc1nc(-c2ccccc2)nc(-c2ccc(-c3ccccc3)cc2)n1 ; OB(O)c1cccc2c1oc1ccc(-c3ccc4oc5ccccc5c4c3)cc12 ; c1ccc(-c2ccc(-c3nc(-c4ccccc4)nc(-c4cccc5c4oc4ccc(-c6ccc7oc8ccccc8c7c6)cc45)n3)cc2)cc1]

Sub 3-39 (4 g, 14.94 mmol), Sub 4-47 (6.2 g, 16.43 mmol), Pd(PPh₃)₄ (0.05 equiv.), K₂CO₃ (3 equiv.), anhydrous THF and a small amount of water were added, and 8.1 g of the product was synthesized in the same manner as in the above synthesis method 2-9. (yield 84%)

### Synthesis Example of 2-62

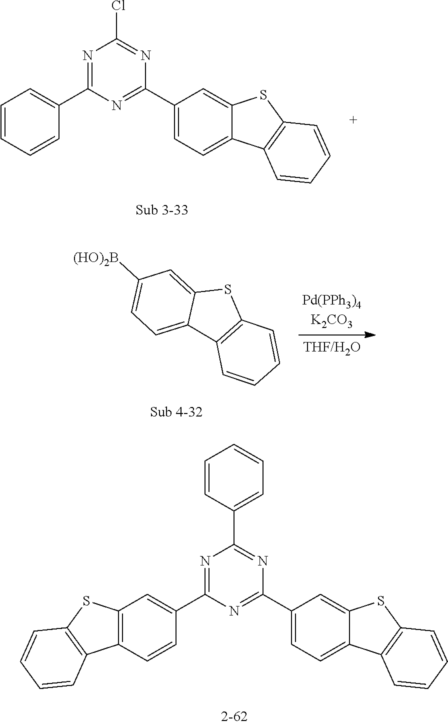 [SMILES: Clc1nc(-c2ccccc2)nc(-c2ccc3c(c2)sc2ccccc23)n1 ; OB(O)c1ccc2c(c1)sc1ccccc12 ; c1ccc(-c2nc(-c3ccc4c(c3)sc3ccccc34)nc(-c3ccc4c(c3)sc3ccccc34)n2)cc1]

Sub 3-33 (4 g, 10.7 mmol), Sub 4-32 (2.7 g, 11.8 mmol), Pd(PPh₃)₄ (0.05 equiv.), K₂CO₃ (3 equiv.), anhydrous THF and a small amount of water were added, and 4.4 g of the product was synthesized in the same manner as in the above synthesis method 2-9. (yield 80%)

### Synthesis Example of 2-115

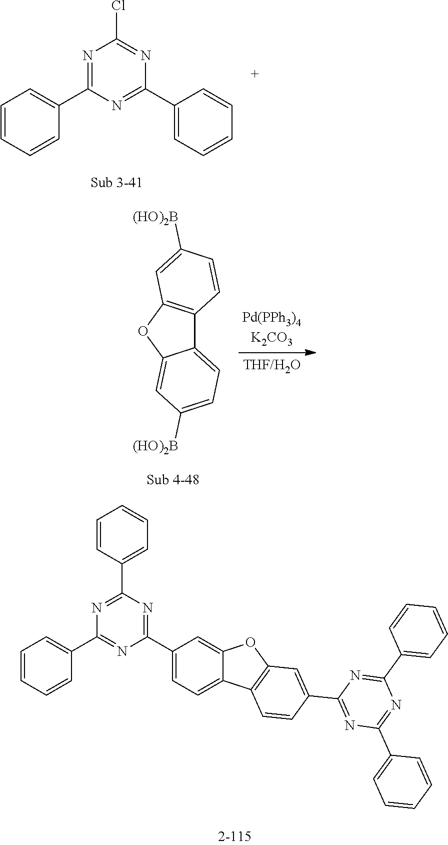 [SMILES: Clc1nc(-c2ccccc2)nc(-c2ccccc2)n1 ; OB(O)c1ccc2c(c1)oc1cc(B(O)O)ccc12 ; c1ccc(-c2nc(-c3ccccc3)nc(-c3ccc4c(c3)oc3cc(-c5nc(-c6ccccc6)nc(-c6ccccc6)n5)ccc34)n2)cc1]

Sub 3-41 (10 g, 37.35 mmol), Sub 4-48 (4.7 g, 18.5 mmol), Pd(PPh₃)₄ (0.1 equiv.), K₂CO₃ (6 equiv.), anhydrous THF and a small amount of water were added, and 9.1 g of the product was synthesized in the same manner as in the above synthesis method 2-9. (yield 78%)

Moreover, the FD-MS values of the compounds 2-1 to 2-118 of the present invention prepared according to the Synthesis Example as described above are shown in Table 6.

TABLE 6

| com- | |
|---|---|
| pound | FD-MS |
| 2-1 | m/z = 631.17 (C₄₃H₂₅N₃OS = 631.75) |
| 2-2 | m/z = 665.21 (C₄₇H₂₇N₃O₂ = 665.75) |
| 2-3 | m/z = 615.19 (C₄₃H₂₅N₃O₂ = 615.69) |
| 2-4 | m/z = 605.16 (C₄₁H₂₃N₃OS = 605.72) |
| 2-5 | m/z = 681.19 (C₄₇H₂₇N₃OS = 681.81) |
| 2-6 | m/z = 691.23 (C₄₉H₂N₃O₂ = 691.79) |
| 2-7 | m/z = 589.18 (C₄₁H₂₃N₃O₂ = 589.65) |
| 2-8 | m/z = 681.19 (C₄₇H₂₇N₃OS = 681.81) |
| 2-9 | m/z = 551.2 (C₃₉H₂₅N₃O = 551.65) |
| 2-10 | m/z = 567.18 (C₃₉H₂₅N₃S = 567.71) |
| 2-11 | m/z = 702.28 (C₅₁H₃₄N₄ = 702.86) |
| 2-12 | m/z = 657.22 (C₄₆H₃₁N₃S = 657.84) |
| 2-13 | m/z = 551.2 (C₃₉H₂₅N₃O = 551.65) |
| 2-14 | m/z = 541.16 (C₃₇H₂₃N₃S = 541.67) |
| 2-15 | m/z = 700.26 (C₅₁ H₃₂N₄ = 700.85) |
| 2-16 | m/z = 703.21 (C₅₀H₂₉N₃S = 703.86) |
| 2-17 | m/z = 525.18 (C₃₇H₂₃N₃O = 525.61) |
| 2-18 | m/z = 591.18 (C₄₁H₂₅N₃S = 591.73) |
| 2-19 | m/z = 627.24 (C₄₄H₂₉N₅ = 627.75) |
| 2-20 | m/z = 524.2 (C₃₇H₂₄N₄ = 524.63) |
| 2-21 | m/z = 551.2 (C₃₉H₂₅N₃O = 551.65) |
| 2-22 | m/z = 567.18 (C₃₉H₂₅N₃S = 567.71) |
| 2-23 | m/z = 702.28 (C₅₁H₃₄N₄ = 702.86) |
| 2-24 | m/z = 562.17 (C₄₀H₂₂N₂O₂ = 562.63) |
| 2-25 | m/z = 779.29 (C₅₇H₃₇N₃O = 779.94) |
| 2-26 | m/z = 731.24 (C₅₂H₃₃N₃S = 731.92) |
| 2-27 | m/z = 601.23 (C₄₂H₂₇N₅ = 601.71) |
| 2-28 | m/z = 475.17 (C₃₃H₂₁N₃O = 475.55) |
| 2-29 | m/z = 641.21 (C₄₅H₂₇N₃O₂ = 641.73) |
| 2-30 | m/z = 783.23 (C₅₅H₃₃N₃OS = 783.95) |
| 2-31 | m/z = 766.27 (C₅₅H₃₄N₄O = 766.9) |
| 2-32 | m/z = 681.19 (C₄₇H₂₇N₃OS = 681.81) |
| 2-33 | m/z = 601.22 (C₄₃H₂₇N₃O = 601 .71) |
| 2-34 | m/z = 693.22 (C₄₉H₃₁N₃S = 693.87) |
| 2-35 | m/z = 550.22 (C₃₉H₂₆N₄ = 550.67) |
| 2-36 | m/z = 525.18 (C₃₇H₂₃N₃O = 525.61) |
| 2-37 | m/z = 703.26 (C₅₁H₃₃N₃O = 703.85) |
| 2-38 | m/z = 693.22 (C₄₉H₃₁N₃S = 693.87) |
| 2-39 | m/z = 636.31 (C₄₅H₂₀D₁₀N₄ = 636.82) |
| 2-40 | m/z = 591.18 (C₄₁H₂₅N₃S = 591.73) |
| 2-41 | m/z = 551.2 (C₃₉H₂₅N₃O = 551.65) |
| 2-42 | m/z = 541.16 (C₃₇H₂₃N₃S = 541.67) |
| 2-43 | m/z = 611.24 (C₄₅H₂₉N₃ = 611.75) |
| 2-44 | m/z = 676.26 (C₄₉H₃₂N₄ = 676.82) |
| 2-45 | m/z = 551.2 (C₃₉H₂₅N₃O = 551.65) |
| 2-46 | m/z = 567.18 (C₃₉H₂₅N₃S = 567.71) |
| 2-47 | m/z = 614.25 (C₄₄H₃₀N₄ = 614.75) |
| 2-48 | m/z = 575.17 (C₃₉H₂₁N₅O = 575.63) |
| 2-49 | m/z = 525.18 (C₃₇H₂₃N₃O = 525.61) |
| 2-50 | m/z = 541.16 (C₃₇H₂₃N₃S = 541.67) |
| 2-51 | m/z = 600.23 (C₄₃H₂₈N₄ = 600.73) |
| 2-52 | m/z = 625.22 (C₄₅H₂₇N₃O = 625.73) |
| 2-53 | m/z = 525.18 (C₃₇H₂₃N₃O = 525.61) |
| 2-54 | m/z = 591.18 (C₄₁H₂₅N₃S = 591.73) |
| 2-55 | m/z = 651.23 (C₄₇H₂₉N₃O = 651.77) |
| 2-56 | m/z = 693.22 (C₄₉H₃₁N₃S = 693.87) |
| 2-57 | m/z = 505.12 (C_{33H19}N₃OS = 505.6) |
| 2-58 | m/z = 641.21 (C₄₅H₂₇N₃O₂ = 641.73) |
| 2-59 | m/z = 571.12 (C₃₇H₂₁N₃S₂ = 571.72) |
| 2-60 | m/z = 564.2 (C₃₉H₂₄N₄O = 564.65) |
| 2-61 | m/z = 681.19 (C₄₇H₂₇N₃OS = 681.81) |
| 2-62 | m/z = 521.1 (C₃₃H₁₉N₃S₂ = 521.66) |
| 2-63 | m/z = 575.14 (C₃₇H₁₉F₂N₃O₂ = 575.57) |
| 2-64 | m/z = 640.23 (C₄₅H₂₈N₄O = 640.75) |
| 2-65 | m/z = 489.15 (C₃₃H₁₉N₃O₂ = 489.53) |
| 2-66 | m/z = 505.12 (C₃₃H₁₉N₃OS = 505.6) |
| 2-67 | m/z = 580.17 (C₃₉H₂₄N₄S = 580.71) |
| 2-68 | m/z = 564.2 (C₃₉H₂₄N₄O = 564.65) |
| 2-69 | m/z = 489.15 (C₃₃H₁₉N₃O₂ = 489.53) |
| 2-70 | m/z = 505.12 (C₃₃H₁₉N₃OS = 505.6) |
| 2-71 | m/z = 505.1 2 (C₃₃H₁₉N₃OS = 505.6) |
| 2-72 | m/z = 639.24 (C₄₅H₂₉N₅ = 639.76) |
| 2-73 | m/z = 657.22 (C₄₆H₃₁N₃S = 657.84) |
| 2-74 | m/z = 689.25 (C₅₀H₃₁N₃O = 689.82) |
| 2-75 | m/z = 690.24 (C₄₉H₃₀N₄₀ = 690.81) |
| 2-76 | m/z = 707.2 (C₄₉H₂₉N₃OS = 707.85) |
| 2-77 | m/z = 591.23 (C₄₂H₂₉N₃O = 591.71) |
| 2-78 | m/z = 617.28 (C₄₅H₃₅N₃ = 6 17.8) |
| 2-79 | m/z = 653.25 (C₄₇H₃₁N₃O = 653.79) |
| 2-80 | m/z = 733.22 (C₅₁H₃₁N₃OS = 733.89) |
| 2-81 | m/z = 615.19 (C₄₃H₂₅N₃O₂ = 615.69) |
| 2-82 | m/z = 681.19 (C₄₇H₂₇N₃OS = 681.81) |
| 2-83 | m/z = 716.29 (C₅₂H₃₆N₄ = 716.89) |
| 2-84 | m/z = 690.24 (C₄₉H₃₀N₄O = 690.81) |
| 2-85 | m/z = 641.25 (C₄₆H₃₁N₃O = 641.77) |
| 2-86 | m/z = 693.22 (C₄₉H₃₁N₃S = 693.87) |
| 2-87 | m/z = 690.24 (C₄₉H₃₀N₄O = 690.81) |
| 2-88 | m/z = 631.17 (C₄₃H₂₅N₃OS = 631.75) |
| 2-89 | m/z = 595.14 (C₃₉H₂₁N₃O₂S = 595.68) |
| 2-90 | m/z = 659.24 (C₄₅H₂₁D₅N₄O₂ = 659.76) |
| 2-91 | m/z = 637.16 (C₄₂H₂₇N₃S₂ = 637.82) |
| 2-92 | m/z = 729.25 (C₅₁H₃₁N₅O = 729.84) |
| 2-93 | m/z = 743.22 (C₅₂H₂₉N₃O₃ = 743.82) |
| 2-94 | m/z = 812.22 (C₅₅H₃₂N₄O₂S = 812.95) |
| 2-95 | m/z = 711.2 (C₄₈H₂₉N₃O₂S = 711.84) |
| 2-96 | m/z = 762.19 (C₅ H₃₀N₄S₂ = 762.95) |
| 2-97 | m/z = 436.17 (C₃₀H₂₀N₄ = 436.52) |
| 2-98 | m/z = 437.16 (C₂₉H₁₉N₅ = 437.51) |
| 2-99 | m/z = 513.2 (C₃₅H₂₃N₅ = 513.6) |
| 2-100 | m/z = 589.23 (C₄₁H₂₇N₅ = 589.7) |
| 2-101 | m/z = 486.18 (C₃₄H₂₂N₄ = 486.58) |
| 2-102 | m/z = 527.17 (C₃₅H₂₁N₅O = 527.59) |
| 2-103 | m/z = 589.23 (C₄₁H₂₇N₅ = 589.7) |
| 2-104 | m/z = 502.18 (C₃₄H₂₂N₄O = 502.58) |
| 2-105 | m/z = 511.2 (C₃₇H₂₅N₃ = 511.63) |
| 2-106 | m/z = 563.21 (C₃₉H₂₅N₅ = 563.66) |
| 2-107 | m/z = 511.2 (C₃₇H₂₅N₃ = 511.63) |
| 2-108 | m/z = 589.23 (C₄₁H₂₇N₅ = 589.7) |
| 2-109 | m/z = 513.2 (C₃₅H₂₃N₅ = 513.6) |
| 2-110 | m/z = 462.16 (C₃₀H₁₈N₆ = 462.52) |
| 2-111 | m/z = 612.21 (C₄₂H₂₄N₆ = 612.7) |
| 2-112 | m/z = 499.2 (C₃₆H₂₅N₃ = 499.62) |
| 2-113 | m/z = 569.17 (C₃₇H₂₃N₅S = 569.69) |
| 2-114 | m/z = 629.22 (C₄₃H₂₇N₅O = 629.72) |
| 2-115 | m/z = 629.22 (C₄₃H₂₇N₅O = 629.72) |
| 2-116 | m/z = 563.21 (C₃₉H₂₅N₅ = 563.66) |
| 2-117 | m/z = 565.2 (C₃₇H₂₃N₇ = 565.64) |
| 2-118 | m/z = 630.22 (C₄₂H₂₆N₆O = 630.71) |

### Manufacturing Evaluation of Organic Electronic Elements

### [Example 1] Red Organic Light Emitting Device

First, N1-(naphthalen-2-yl)-N4, N4-bis(4-(naphthalen yl(phenyl)amino)phenyl)-N1-phenylbenzene-1,4-diamine (hereinafter abbreviated as 2-TNATA) film was vacuum-deposited as a hole injection layer on the ITO layer (anode) formed on the glass substrate to have a thickness of 60 nm. Subsequently, 4,4-bis[N-(1-naphthyl)-N-phenylamino]biphenyl (hereinafter abbreviated as -NPD) as a hole transport compound was vacuum deposited on the film to a thickness of 60 nm to form a hole transport layer. Subsequently, as a material for the emitting auxiliary layer, compounds 1-3 of the present invention were vacuum deposited to a thickness of 30 nm to form an emitting auxiliary layer. Then, the compound 2-36 of the present invention as a host on the emitting auxiliary layer, an emitting layer having a thickness of 30 nm was deposited by doping (piq)2lr(acac)[bis-(1-phenylisoquinolyl)iridium(III)acetylacetonate] as a dopant in a weight ratio of 95:5. After that, (1,1′bisphenyl)-4-oleato)bis(2-methyl-8-quinolinolato)aluminum (hereinafter abbreviated as BAlq) was vacuum deposited to a thickness of 10 nm on the emitting layer to form a hole blocking layer, and an electron transport layer was formed by depositing tris(8-quinolinol) aluminum (hereinafter abbreviated as Alq3) to a thickness of 40 nm on the hole blocking layer. Thereafter, LiF, an alkali metal halide, was deposited to a thickness of 0.2 nm as an electron injection layer on the electron transport layer, and then Al was deposited to a thickness of 150 nm and used as a cathode to manufacture an organic light emitting device.

### [Example 2] to [Example 16]

An organic electroluminescent device was manufactured in the same manner as in Example 1, except that the compounds of the present invention shown in Table 7 were used for the emitting auxiliary layer and the emitting layer.

### [Example 17] and [Example 18]

An organic electroluminescent device was manufactured in the same manner as in Example 1, except that Compound 1-54 of the present invention is used for the hole transport layer, and Compound 1-61 of the present invention is used for the emitting auxiliary layer, and for the emitting layer, as shown in Table 7, the compounds of the present invention were used in a ratio of 5:5.

### [Comparative Example 1] and [Comparative Example 2]

An organic light emitting device was manufactured in the same manner as in Example 1, except that the host material was used as shown in Table 7 without using the emitting auxiliary layer.

### [Comparative Example 3] to [Comparative Example 7]

As shown in Table 7, an organic light emitting device was manufactured in the same manner as in Example 1, except that the emitting auxiliary layer material and the host material were used.

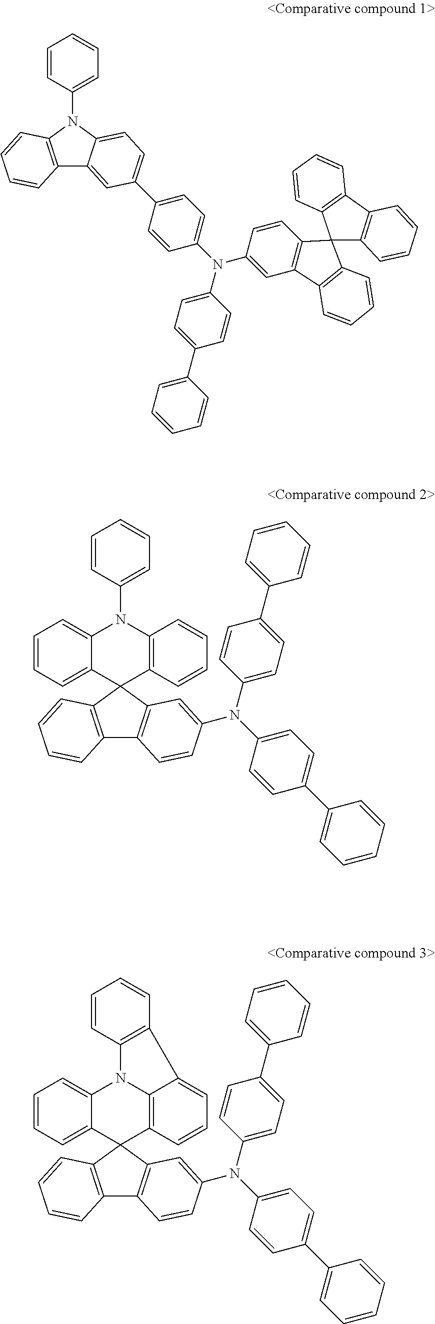 [SMILES: c1ccc(-c2ccc(N(c3ccc(-c4ccc5c(c4)c4ccccc4n5-c4ccccc4)cc3)c3ccc4c(c3)-c3ccccc3C43c4ccccc4-c4ccccc43)cc2)cc1 ; c1ccc(-c2ccc(N(c3ccc(-c4ccccc4)cc3)c3ccc4c(c3)C3(c5ccccc5-4)c4ccccc4N(c4ccccc4)c4ccccc43)cc2)cc1 ; c1ccc(-c2ccc(N(c3ccc(-c4ccccc4)cc3)c3ccc4c(c3)C3(c5ccccc5-4)c4ccccc4-n4c5ccccc5c5cccc3c54)cc2)cc1]

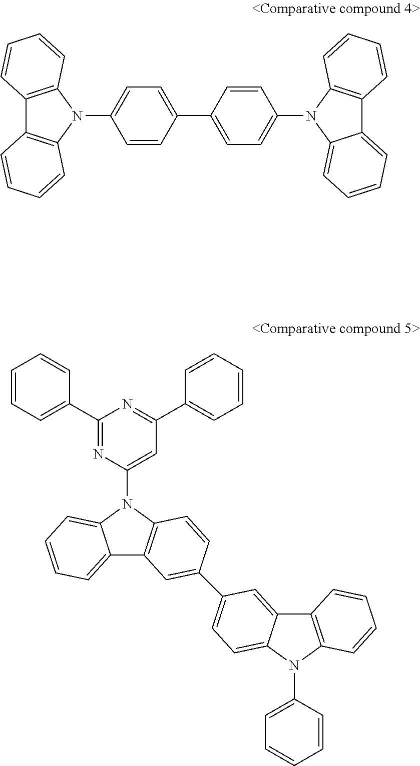 [SMILES: c1ccc2c(c1)c1ccccc1n2-c1ccc(-c2ccc(-n3c4ccccc4c4ccccc43)cc2)cc1 ; c1ccc(-c2cc(-n3c4ccccc4c4cc(-c5ccc6c(c5)c5ccccc5n6-c5ccccc5)ccc43)nc(-c3ccccc3)n2)cc1]

By applying a forward bias DC voltage to the organic electroluminescent devices prepared by Example 1 to Example 18 and Comparative Example 1 to Comparative Example 7 of the present invention, Electroluminescence (EL) characteristics were measured with PR-650 from Photoresearch, and the T95 lifespan was measured using a lifespan measuring device manufactured by McScience at 2500 cd/m² standard luminance. The measurement results are shown in Table 7.

TABLE 7

| | Emitting | | | Current | | | |
|---|---|---|---|---|---|---|---|
| | auxiliary | Emitting | | Density | Brightness | Efficiency | |
| | layer | layer | Voltage | (mA/cm²) | (cd/m²) | (cd/A) | T(95) |
| Comparative | — | Comparative | 6.7 | 34.2 | 2500.0 | 7.3 | 75.6 |
| example (1) | | compound 4 | | | | | |
| Comparative | — | Comparative | 6.4 | 17.4 | 2500.0 | 14.4 | 78.5 |
| example (2) | | compound 5 | | | | | |
| Comparative | Comparative | Comparative | 6.2 | 15.8 | 2500.0 | 15.8 | 81.8 |
| example (3) | compound1 | compound 5 | | | | | |
| Comparative | Comparative | Comparative | 6 | 14.4 | 2500.0 | 17.4 | 85.7 |
| example (4) | compound 2 | compound 5 | | | | | |
| Comparative | Comparative | Comparative | 6.1 | 15.4 | 2500.0 | 16.2 | 83.5 |
| example (5) | compound 3 | compound 5 | | | | | |
| Comparative | Comparative | 2-32 | 5.9 | 12.1 | 2500.0 | 20.7 | 91.1 |
| example (6) | compound 2 | | | | | | |
| comparative | 1-1 | Comparative | 6.1 | 12.4 | 2500.0 | 20.2 | 88.7 |
| example (7) | | compound 5 | | | | | |
| Example (1) | 1-3 | 2-36 | 5.5 | 8.2 | 2500.0 | 30.5 | 112.8 |
| Example (2) | 1-3 | 2-40 | 5.5 | 8.2 | 2500.0 | 30.6 | 110.7 |
| Example (3) | 1-3 | 2-76 | 5.5 | 8.6 | 2500.0 | 28.9 | 111.9 |
| Example (4) | 1-3 | 2-89 | 5.6 | 8.8 | 2500.0 | 28.4 | 106.1 |
| Example (5) | 1-10 | 2-36 | 5.5 | 8.0 | 2500.0 | 31.3 | 112.0 |
| Example (6) | 1-10 | 2-40 | 5.5 | 8.1 | 2500.0 | 30.8 | 112.5 |
| Example (7) | 1-10 | 2-76 | 5.5 | 8.6 | 2500.0 | 29.1 | 108.0 |
| Example (8) | 1-10 | 2-89 | 5.6 | 9.0 | 2500.0 | 27.9 | 105.0 |
| Example (9) | 1-43 | 2-36 | 5.6 | 8.5 | 2500.0 | 29.4 | 108.5 |
| Example (10) | 1-43 | 2-40 | 5.7 | 8.6 | 2500.0 | 29.1 | 110.4 |
| Example (11) | 1-43 | 2-76 | 5.6 | 9.0 | 2500.0 | 27.7 | 108.3 |
| Example (12) | 1-43 | 2-89 | 5.8 | 9.1 | 2500.0 | 27.4 | 101.3 |
| Example (13) | 1-50 | 2-36 | 5.6 | 8.8 | 2500.0 | 28.4 | 105.8 |
| Example (14) | 1-50 | 2-40 | 5.6 | 9.0 | 2500.0 | 27.7 | 103.1 |
| Example (15) | 1-50 | 2-76 | 5.6 | 9.4 | 2500.0 | 26.5 | 102.1 |
| Example (16) | 1-154 | 2-89 | 5.8 | 9.9 | 2500.0 | 25.2 | 97.2 |
| Example (17) | 1-15 | 2-76, 3-36 | 5.7 | 8.0 | 2500.0 | 31.3 | 107.9 |
| Example (18) | 1-15 | 2-89, 3-132 | 5.5 | 8.1 | 2500.0 | 30.7 | 108.8 |

From the results of Table 7, when the material for an organic light emitting device of the present invention represented by Formula 1 is used for the emitting auxiliary layer and the material for an organic light emitting device of the present invention represented by Formula 2 is used as a phosphorescent host, it can be seen that the driving voltage is lowered and the efficiency and lifespan are improved compared to Comparative Examples 1 to 7.

Driving voltage, efficiency and lifespan of Comparative Examples 3 to 6 using one of Comparative Compounds 1 to 3 in the emitting auxiliary layer was improved than Comparative Examples 1 and 2 using Comparative Compounds 4 or 5 without forming the emitting auxiliary layer as a host. Also, Examples 1 to 18 in which an emitting auxiliary layer was formed with the compound represented by Formula 1 of the present invention and the material represented by Formula 2 was used as a host were significantly improved compared to Comparative Examples 1 to 7.

It is presumed that this is because the compounds of the present invention represented by Formula 1 have a deep HOMO energy level, so when used as an emitting auxiliary layer, holes and electrons achieve charge balance and light is emitted inside the emitting layer rather than at the hole transport layer interface to maximize efficiency. Also, by using the compound of the present invention represented by Formula 2 as a phosphorescent host, it is determined that the combination of the devices has a synergistic effect electrochemically to improve the performance of the device as a whole.

Therefore, when an organic electronic element is prepared by appropriately combining the compounds represented by Formulas 1 and 2, more holes move quickly and easily to the emitting layer, and accordingly, the charge balance of holes and electrons in the emitting layer is increased, so that light is emitted well inside the emitting layer, not at the interface of the hole transport layer, as a result, deterioration at the interface between ITO and HTL is also reduced, driving voltage of the entire device is lowered, and efficiency and lifespan can be improved. That is, when the compounds represented by Formulas 1 and 2 are appropriately combined, synergistic electrochemical action appears to improve overall performance of the device.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### INDUSTRIAL APPLICABILITY

According to the present invention, it is possible to manufacture an organic device having excellent device characteristics of high luminance, high light emission and long lifespan, and thus there is industrial applicability.

1. An organic electronic element comprising an anode, a cathode, and an organic material layer formed between the anode and the cathode, wherein the organic material layer comprises an emitting layer, and a hole transport band layer formed between the emitting layer and the anode,
wherein the hole transport band layer comprises a compound represented by Formula 1, and the emitting layer comprises a compound represented by Formula 2: 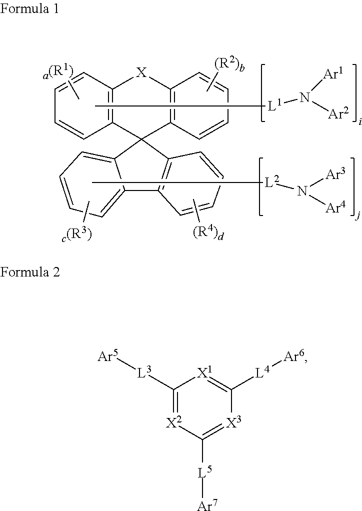
wherein:
1) X is O, S or NR, provided that where X is NR, i is 0 and j is 1,
2) X¹, X² and X³ are each independently CR′ or N, provided that at least two of X¹, X² and X³ are N,
3) R¹, R², R³, R⁴, R and R′ are each independently the same as or different from each other, and are each independently selected from the group consisting of hydrogen;
deuterium; halogen; a C₁-C₆₀ alkyl group; a C₂-C₆₀ alkenyl group; a C₂-C₆₀ alkynyl group;
a C₁-C₆₀ alkoxyl group; a C₆-C₆₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and -L′-NR^{a}R^{b}; or in case a, b, c and d are 2 or more, a plurality of adjacent R¹s, R²s, R³s, or R⁴s may be bonded to each other to form a ring,
4) L′, L¹, L², L³, L⁴ and L⁵ are each independently selected from the group consisting of a single bond; a C₆-C₆₀ arylene group; fluorenylene group; a fused ring group of a C₃-C₆₀ aliphatic ring and a C₆-C₆₀ aromatic ring; a C₂-C₆₀ heterocyclic group;
5) R^{a} and R^{b} are each independently selected from the group consisting of a C₆-C₆₀ aryl group; fluorenyl group; a fused ring group of a C₃-C₆₀ aliphatic ring and a C₆-C₆₀ aromatic ring; a C₂-C₆₀ heterocyclic group including at least one heteroatom of O, N, S, Si or P;
6) a, b, c and d are each independently an integer of 0 to 4,
7) i and j are independently an integer of 0 to 2, provided that i+j is an integer of 1 or more;
8) Ar¹, Ar², Ar³, Ar⁴, Ar⁵, Ar⁶ and Ar⁷ are each independently selected from the group consisting of a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a C₂-C₆₀ heterocyclic group including at least one heteroatom of O, N, S, Si or P; and a fused ring group of a C₃-C₆₀ aliphatic ring and a C₆-C₆₀ aromatic ring; alternatively, Ar¹ and Ar² or Ar³ and Ar⁴ may be bonded to each other to form a ring;
9) wherein the aryl group, arylene group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and -L′-NR^{a}R^{b}; and the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

2. The organic electronic element of claim 1, wherein the compound represented by Formula 1 is represented by any one of Formulas 1-1 to 1-7: 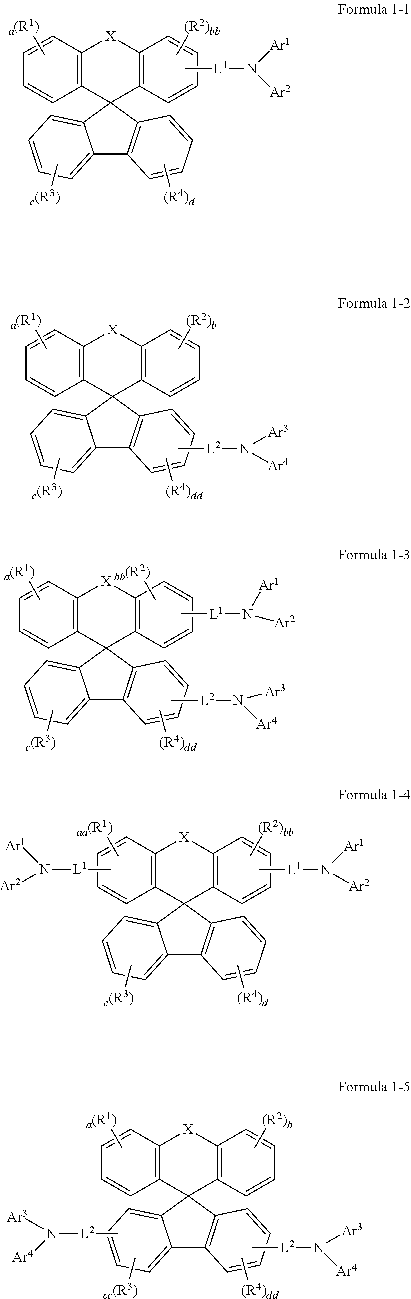 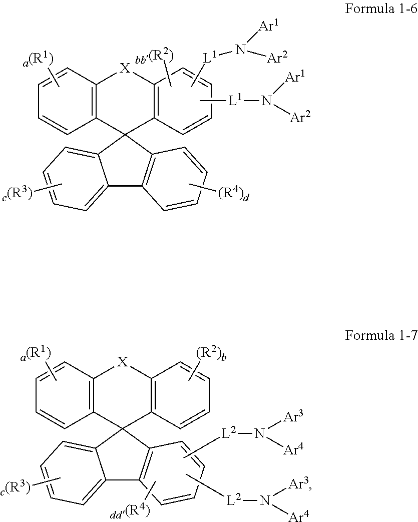
wherein:
1) X, R¹, R², R³, R⁴, a, b, c, d, L¹, L², Ar¹, Ar², Ar³ and Ar⁴ are the same as defined in claim 1,
2) aa, bb, cc and dd are each independently an integer of 0 to 3, and
3) bb′ and dd′ are each independently an integer of 0 to 2.

3. The organic electronic element of claim 1, wherein the compound represented by Formula 1 is represented by any one of Formulas 1-8 to 1-9: 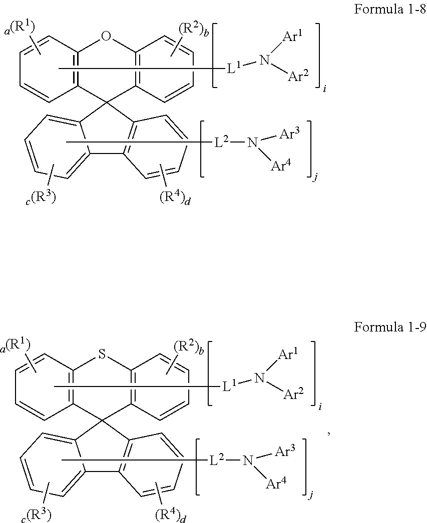
wherein R¹, R², R³, R⁴, a, b, c, d, L¹, L², Ar¹, Ar², Ar³, Ar⁴, i and j are the same as defined in claim 1.

4. The organic electronic element of claim 1, wherein at least one of Ar¹ to Ar⁴ in Formula 1 is represented by Formula B-1: 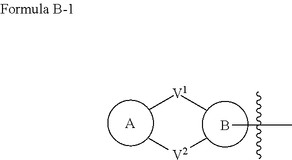
wherein:
1) V¹ and V² are each independently a single bond, NR⁵, CR⁶R⁷, O or S,
2) R⁵, R⁶ and R⁷ are the same as the definition of R¹ in Formula 1, and R⁶ and R⁷ may be bonded to each other to form a ring, and
3) Ring A and ring B are each independently a substituted or unsubstituted C₆-C₂₀ aryl group; or a substituted or unsubstituted C₄˜C₂₀ heterocyclic group.

5. The organic electronic element of claim 1, wherein the compound represented by Formula 1 is selected from the group consisting of the following compounds:

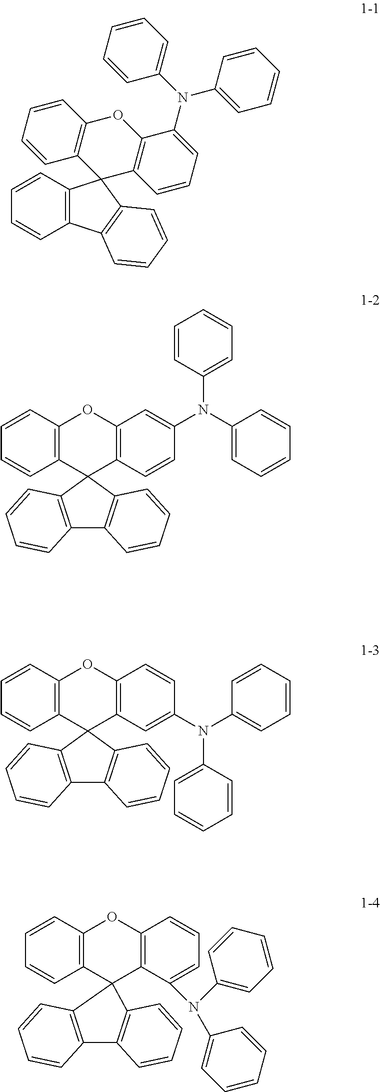 [SMILES: c1ccc(N(c2ccccc2)c2cccc3c2Oc2ccccc2C32c3ccccc3-c3ccccc32)cc1 ; c1ccc(N(c2ccccc2)c2ccc3c(c2)Oc2ccccc2C32c3ccccc3-c3ccccc32)cc1 ; c1ccc(N(c2ccccc2)c2ccc3c(c2)C2(c4ccccc4O3)c3ccccc3-c3ccccc32)cc1 ; c1ccc(N(c2ccccc2)c2cccc3c2C2(c4ccccc4O3)c3ccccc3-c3ccccc32)cc1]

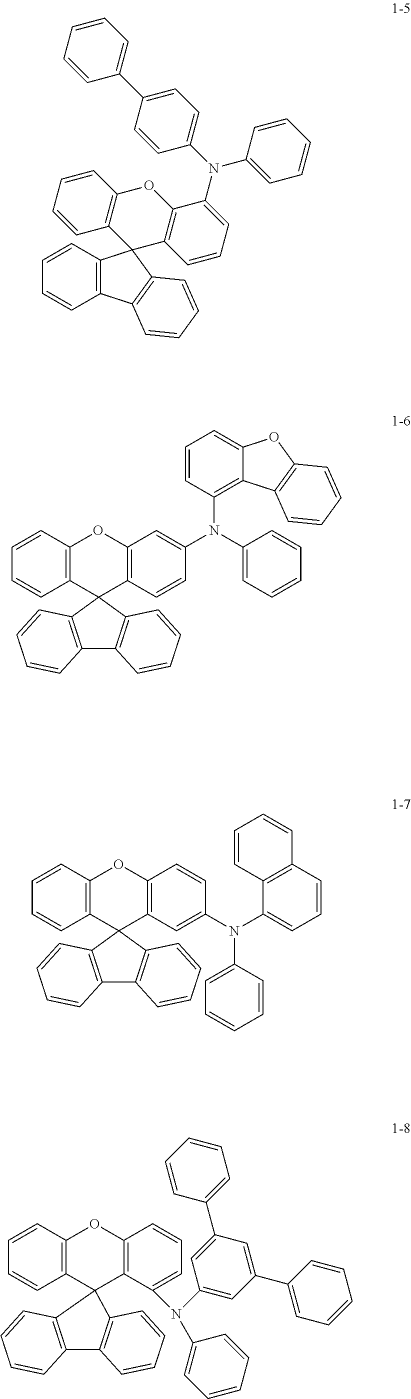 [SMILES: c1ccc(-c2ccc(N(c3ccccc3)c3cccc4c3Oc3ccccc3C43c4ccccc4-c4ccccc43)cc2)cc1 ; c1ccc(N(c2ccc3c(c2)Oc2ccccc2C32c3ccccc3-c3ccccc32)c2cccc3oc4ccccc4c23)cc1 ; c1ccc(N(c2ccc3c(c2)C2(c4ccccc4O3)c3ccccc3-c3ccccc32)c2cccc3ccccc23)cc1 ; c1ccc(-c2cc(-c3ccccc3)cc(N(c3ccccc3)c3cccc4c3C3(c5ccccc5O4)c4ccccc4-c4ccccc43)c2)cc1]

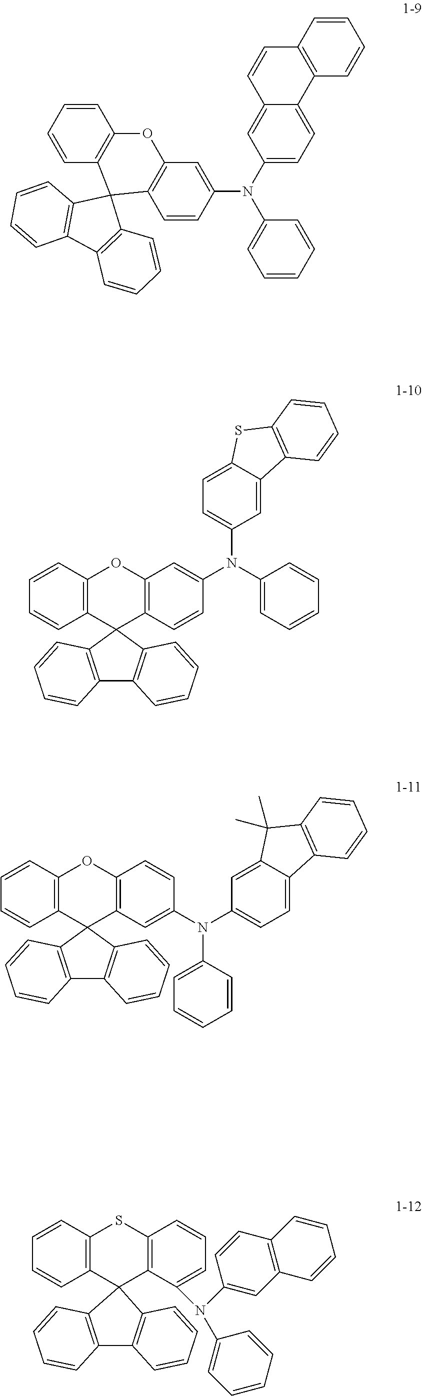 [SMILES: c1ccc(N(c2ccc3c(c2)Oc2ccccc2C32c3ccccc3-c3ccccc32)c2ccc3c(ccc4ccccc43)c2)cc1 ; c1ccc(N(c2ccc3c(c2)Oc2ccccc2C32c3ccccc3-c3ccccc32)c2ccc3sc4ccccc4c3c2)cc1 ; CC1(C)c2ccccc2-c2ccc(N(c3ccccc3)c3ccc4c(c3)C3(c5ccccc5O4)c4ccccc4-c4ccccc43)cc21 ; c1ccc(N(c2ccc3ccccc3c2)c2cccc3c2C2(c4ccccc4S3)c3ccccc3-c3ccccc32)cc1]

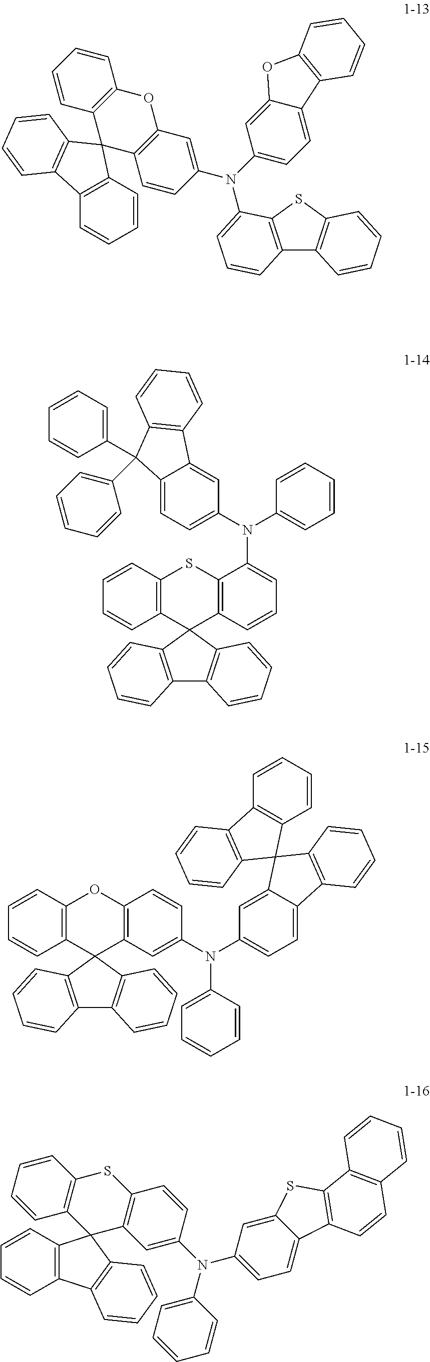 [SMILES: c1ccc2c(c1)Oc1cc(N(c3ccc4c(c3)oc3ccccc34)c3cccc4c3sc3ccccc34)ccc1C21c2ccccc2-c2ccccc21 ; c1ccc(N(c2ccc3c(c2)-c2ccccc2C3(c2ccccc2)c2ccccc2)c2cccc3c2Sc2ccccc2C32c3ccccc3-c3ccccc32)cc1 ; c1ccc(N(c2ccc3c(c2)C2(c4ccccc4O3)c3ccccc3-c3ccccc32)c2ccc3c(c2)C2(c4ccccc4-c4ccccc42)c2ccccc2-3)cc1 ; c1ccc(N(c2ccc3c(c2)C2(c4ccccc4S3)c3ccccc3-c3ccccc32)c2ccc3c(c2)sc2c4ccccc4ccc32)cc1]

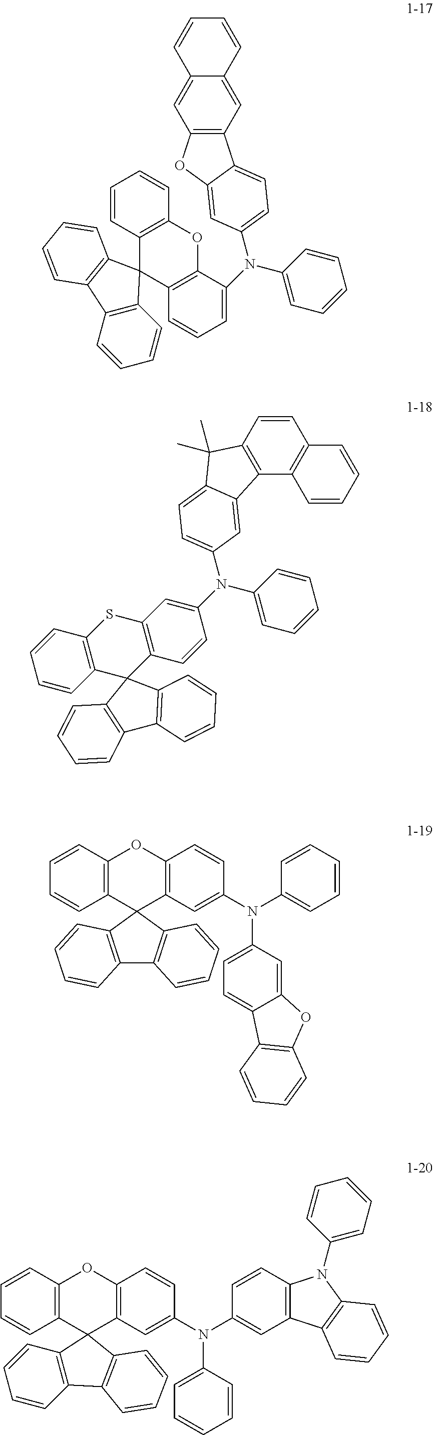 [SMILES: c1ccc(N(c2ccc3c(c2)oc2cc4ccccc4cc23)c2cccc3c2Oc2ccccc2C32c3ccccc3-c3ccccc32)cc1 ; CC1(C)c2ccc(N(c3ccccc3)c3ccc4c(c3)Sc3ccccc3C43c4ccccc4-c4ccccc43)cc2-c2c1ccc1ccccc21 ; c1ccc(N(c2ccc3c(c2)C2(c4ccccc4O3)c3ccccc3-c3ccccc32)c2ccc3c(c2)oc2ccccc23)cc1 ; c1ccc(N(c2ccc3c(c2)C2(c4ccccc4O3)c3ccccc3-c3ccccc32)c2ccc3c(c2)c2ccccc2n3-c2ccccc2)cc1]

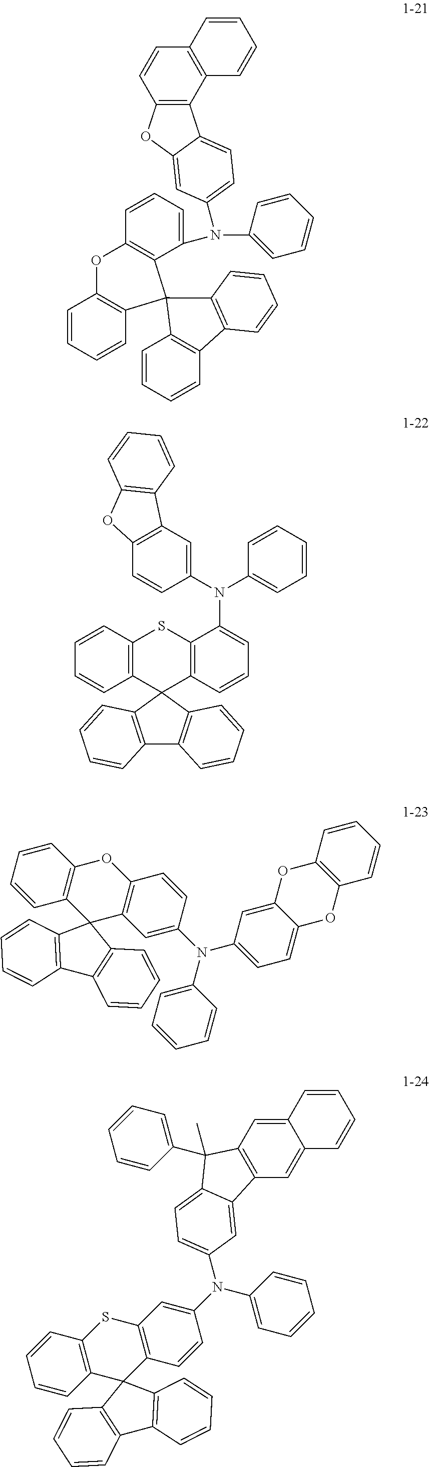

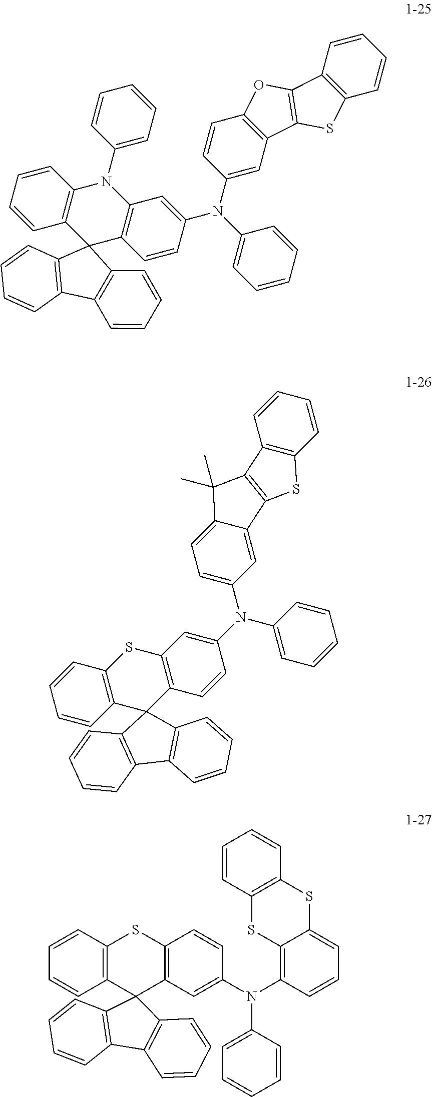 [SMILES: c1ccc(N(c2ccc3c(c2)N(c2ccccc2)c2ccccc2C32c3ccccc3-c3ccccc32)c2ccc3oc4c5ccccc5sc4c3c2)cc1 ; CC1(C)c2ccc(N(c3ccccc3)c3ccc4c(c3)Sc3ccccc3C43c4ccccc4-c4ccccc43)cc2-c2sc3ccccc3c21 ; c1ccc(N(c2ccc3c(c2)C2(c4ccccc4S3)c3ccccc3-c3ccccc32)c2cccc3c2Sc2ccccc2S3)cc1]

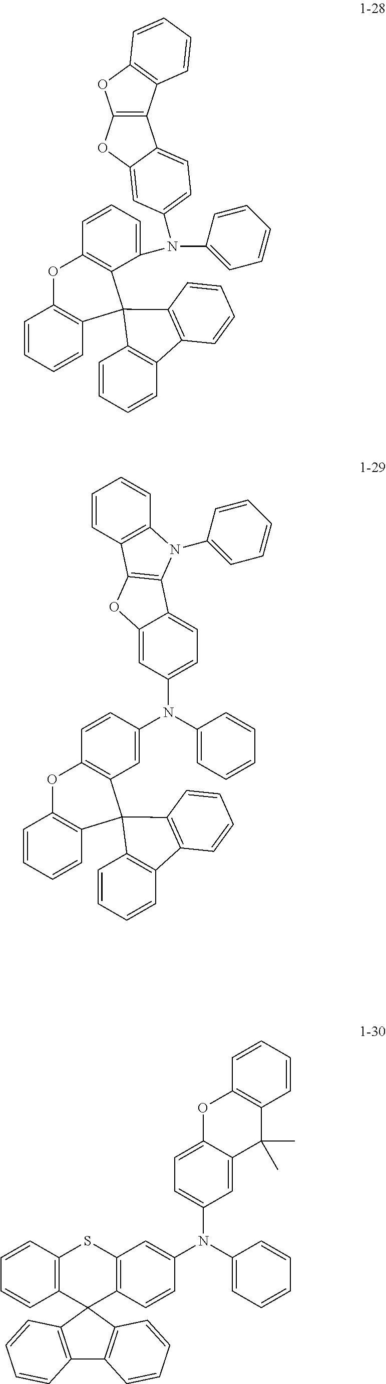 [SMILES: c1ccc(N(c2ccc3c(c2)oc2oc4ccccc4c23)c2cccc3c2C2(c4ccccc4O3)c3ccccc3-c3ccccc32)cc1 ; c1ccc(N(c2ccc3c(c2)C2(c4ccccc4O3)c3ccccc3-c3ccccc32)c2ccc3c(c2)oc2c4ccccc4n(-c4ccccc4)c32)cc1 ; CC1(C)c2ccccc2Oc2ccc(N(c3ccccc3)c3ccc4c(c3)Sc3ccccc3C43c4ccccc4-c4ccccc43)cc21]

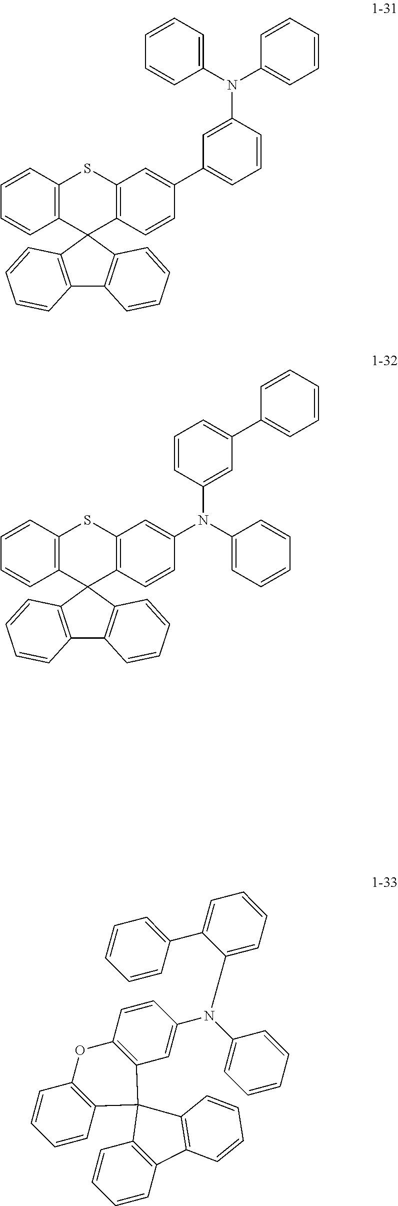 [SMILES: c1ccc(N(c2ccccc2)c2cccc(-c3ccc4c(c3)Sc3ccccc3C43c4ccccc4-c4ccccc43)c2)cc1 ; c1ccc(-c2cccc(N(c3ccccc3)c3ccc4c(c3)Sc3ccccc3C43c4ccccc4-c4ccccc43)c2)cc1 ; c1ccc(-c2ccccc2N(c2ccccc2)c2ccc3c(c2)C2(c4ccccc4O3)c3ccccc3-c3ccccc32)cc1]

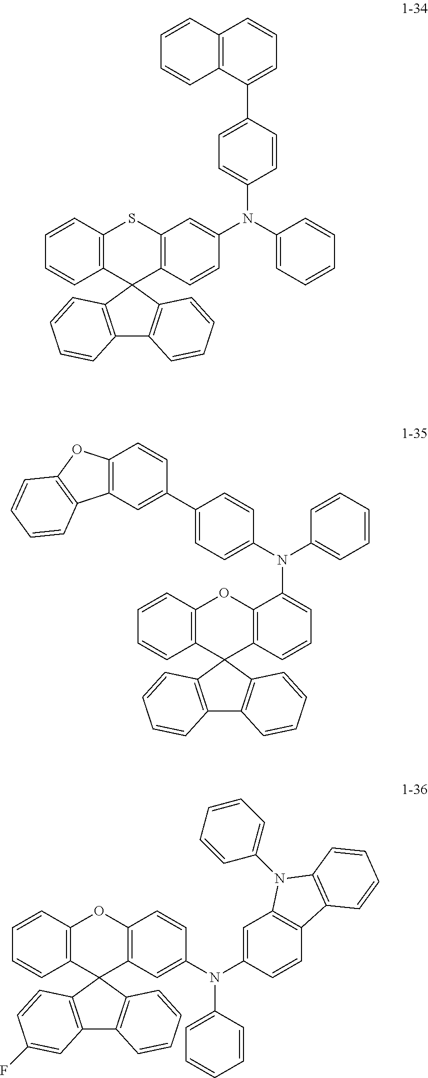 [SMILES: c1ccc(N(c2ccc(-c3cccc4ccccc34)cc2)c2ccc3c(c2)Sc2ccccc2C32c3ccccc3-c3ccccc32)cc1 ; c1ccc(N(c2ccc(-c3ccc4oc5ccccc5c4c3)cc2)c2cccc3c2Oc2ccccc2C32c3ccccc3-c3ccccc32)cc1 ; Fc1ccc2c(c1)-c1ccccc1C21c2ccccc2Oc2ccc(N(c3ccccc3)c3ccc4c5ccccc5n(-c5ccccc5)c4c3)cc21]

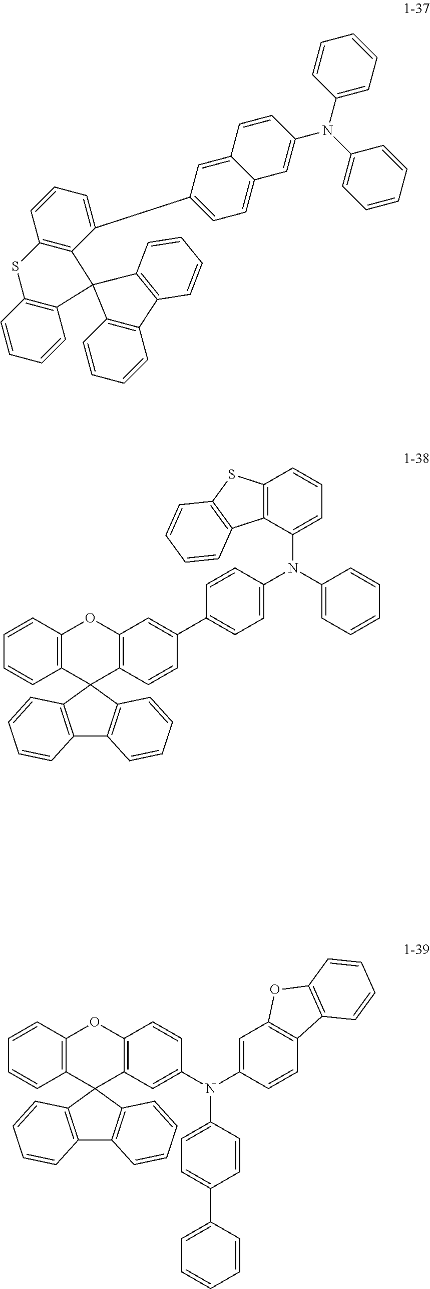 [SMILES: c1ccc(N(c2ccccc2)c2ccc3cc(-c4cccc5c4C4(c6ccccc6S5)c5ccccc5-c5ccccc54)ccc3c2)cc1 ; c1ccc(N(c2ccc(-c3ccc4c(c3)Oc3ccccc3C43c4ccccc4-c4ccccc43)cc2)c2cccc3sc4ccccc4c23)cc1 ; c1ccc(-c2ccc(N(c3ccc4c(c3)C3(c5ccccc5O4)c4ccccc4-c4ccccc43)c3ccc4c(c3)oc3ccccc34)cc2)cc1]

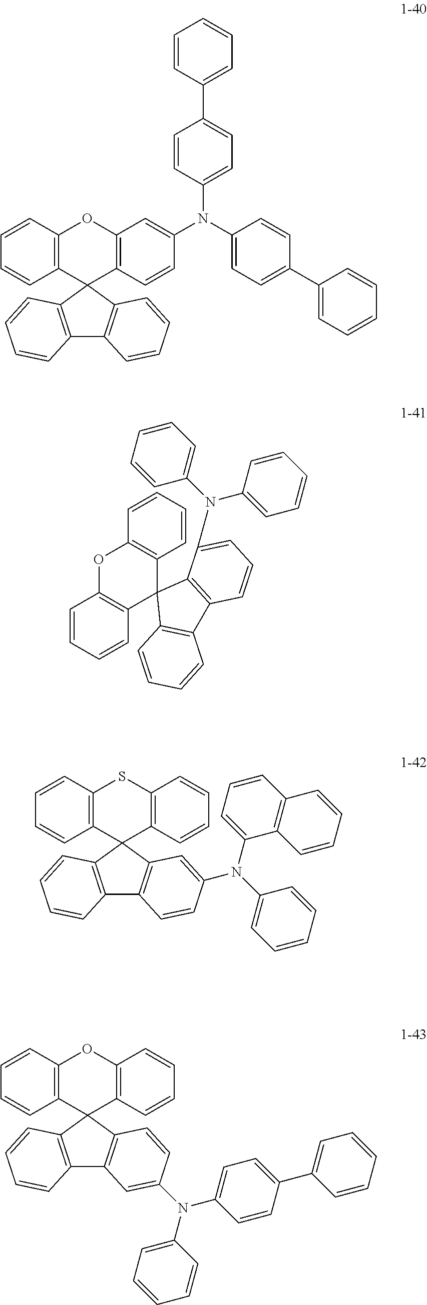 [SMILES: c1ccc(-c2ccc(N(c3ccc(-c4ccccc4)cc3)c3ccc4c(c3)Oc3ccccc3C43c4ccccc4-c4ccccc43)cc2)cc1 ; c1ccc(N(c2ccccc2)c2cccc3c2C2(c4ccccc4Oc4ccccc42)c2ccccc2-3)cc1 ; c1ccc(N(c2ccc3c(c2)C2(c4ccccc4Sc4ccccc42)c2ccccc2-3)c2cccc3ccccc23)cc1 ; c1ccc(-c2ccc(N(c3ccccc3)c3ccc4c(c3)-c3ccccc3C43c4ccccc4Oc4ccccc43)cc2)cc1]

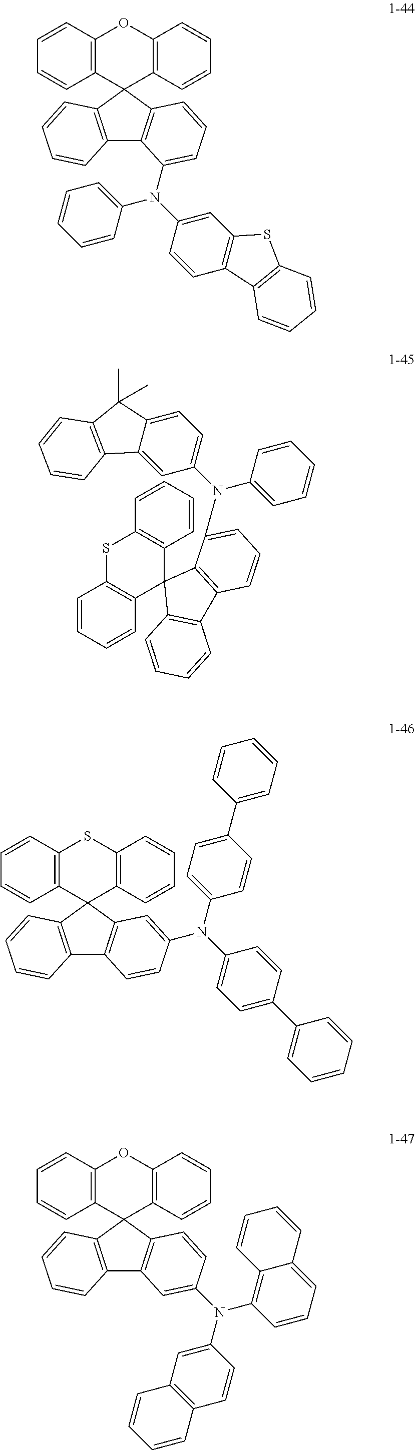 [SMILES: c1ccc(N(c2ccc3c(c2)sc2ccccc23)c2cccc3c2-c2ccccc2C32c3ccccc3Oc3ccccc32)cc1 ; CC1(C)c2ccccc2-c2cc(N(c3ccccc3)c3cccc4c3C3(c5ccccc5Sc5ccccc53)c3ccccc3-4)ccc21 ; c1ccc(-c2ccc(N(c3ccc(-c4ccccc4)cc3)c3ccc4c(c3)C3(c5ccccc5Sc5ccccc53)c3ccccc3-4)cc2)cc1 ; c1ccc2c(c1)Oc1ccccc1C21c2ccccc2-c2cc(N(c3ccc4ccccc4c3)c3cccc4ccccc34)ccc21]

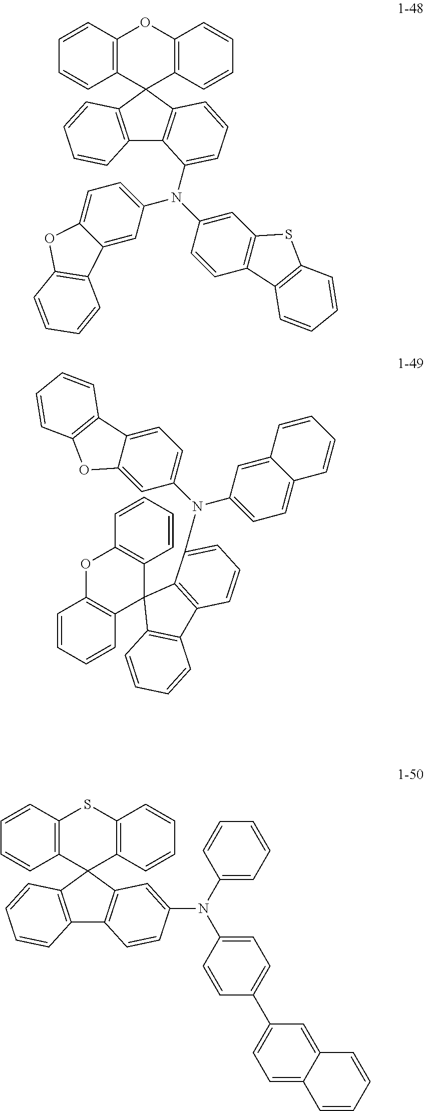

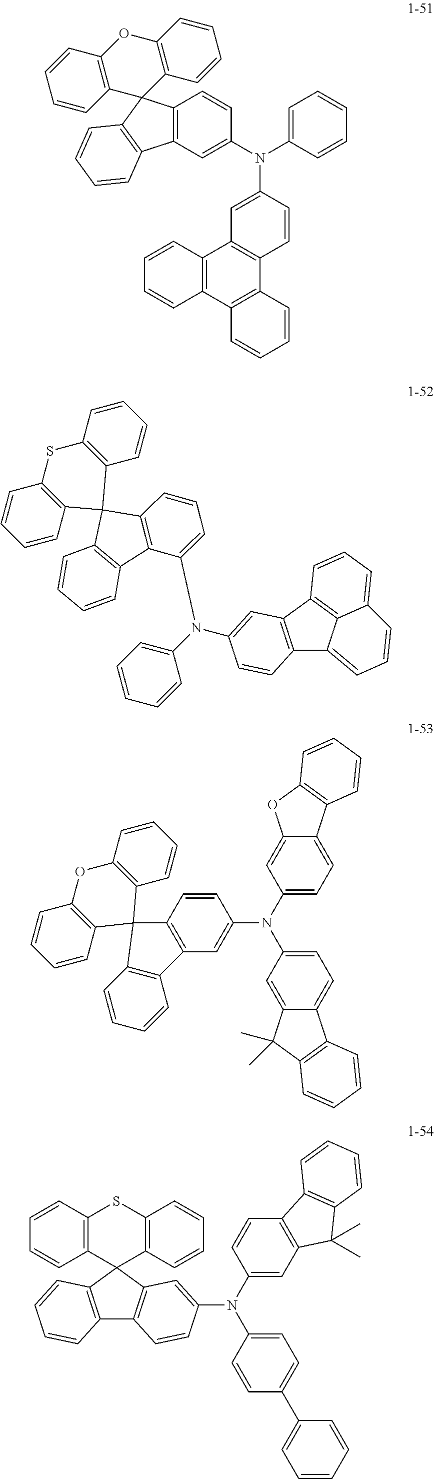

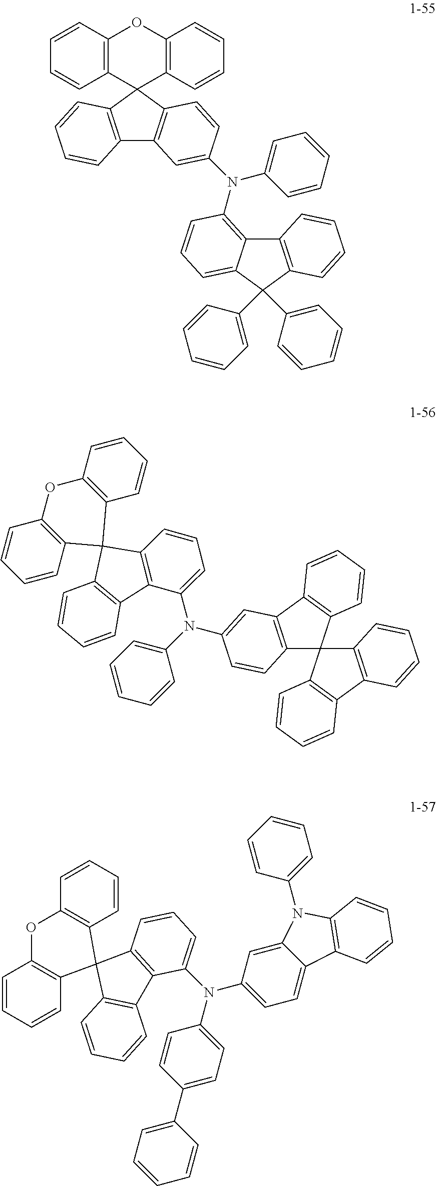 [SMILES: c1ccc(N(c2ccc3c(c2)-c2ccccc2C32c3ccccc3Oc3ccccc32)c2cccc3c2-c2ccccc2C3(c2ccccc2)c2ccccc2)cc1 ; c1ccc(N(c2ccc3c(c2)-c2ccccc2C32c3ccccc3-c3ccccc32)c2cccc3c2-c2ccccc2C32c3ccccc3Oc3ccccc32)cc1 ; c1ccc(-c2ccc(N(c3ccc4c5ccccc5n(-c5ccccc5)c4c3)c3cccc4c3-c3ccccc3C43c4ccccc4Oc4ccccc43)cc2)cc1]

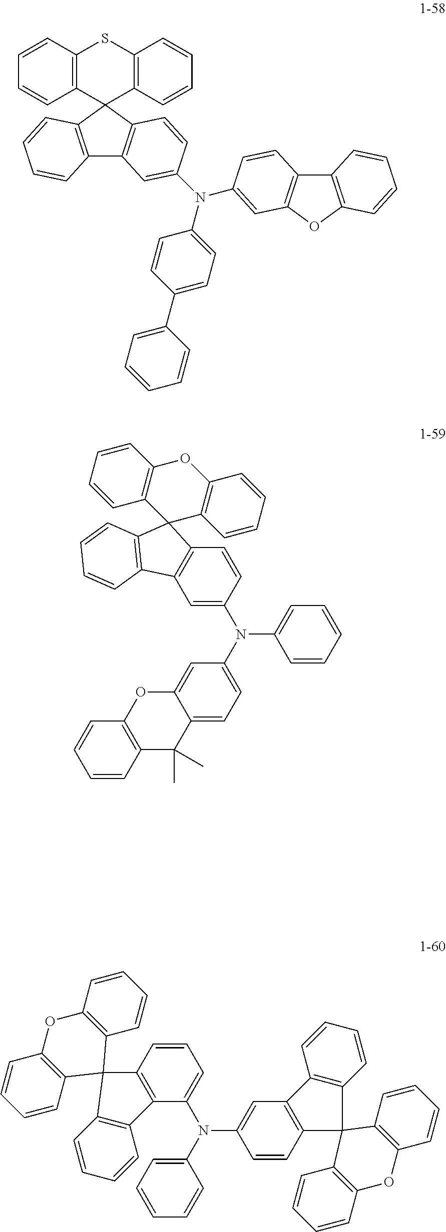 [SMILES: c1ccc(-c2ccc(N(c3ccc4c(c3)-c3ccccc3C43c4ccccc4Sc4ccccc43)c3ccc4c(c3)oc3ccccc34)cc2)cc1 ; CC1(C)c2ccccc2Oc2cc(N(c3ccccc3)c3ccc4c(c3)-c3ccccc3C43c4ccccc4Oc4ccccc43)ccc21 ; c1ccc(N(c2ccc3c(c2)-c2ccccc2C32c3ccccc3Oc3ccccc32)c2cccc3c2-c2ccccc2C32c3ccccc3Oc3ccccc32)cc1]

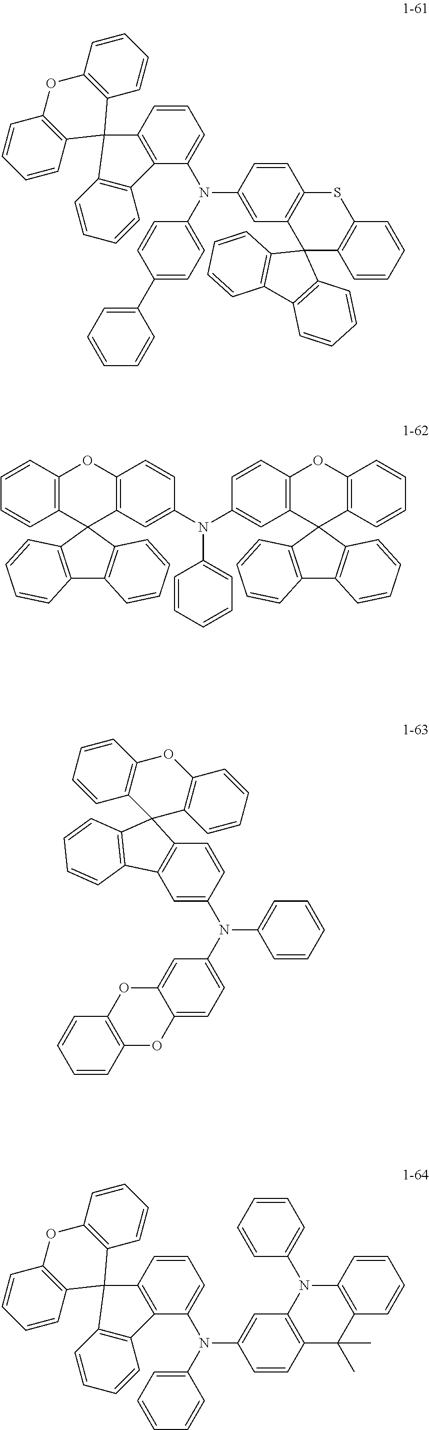 [SMILES: c1ccc(-c2ccc(N(c3ccc4c(c3)C3(c5ccccc5S4)c4ccccc4-c4ccccc43)c3cccc4c3-c3ccccc3C43c4ccccc4Oc4ccccc43)cc2)cc1 ; c1ccc(N(c2ccc3c(c2)C2(c4ccccc4O3)c3ccccc3-c3ccccc32)c2ccc3c(c2)C2(c4ccccc4O3)c3ccccc3-c3ccccc32)cc1 ; c1ccc(N(c2ccc3c(c2)Oc2ccccc2O3)c2ccc3c(c2)-c2ccccc2C32c3ccccc3Oc3ccccc32)cc1 ; CC1(C)c2ccccc2N(c2ccccc2)c2cc(N(c3ccccc3)c3cccc4c3-c3ccccc3C43c4ccccc4Oc4ccccc43)ccc21]

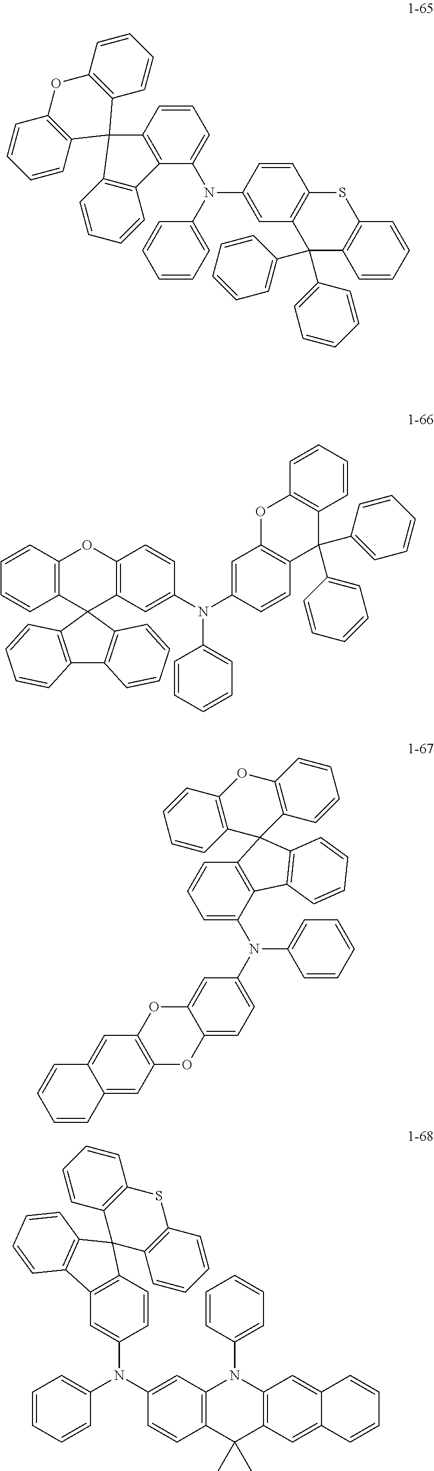

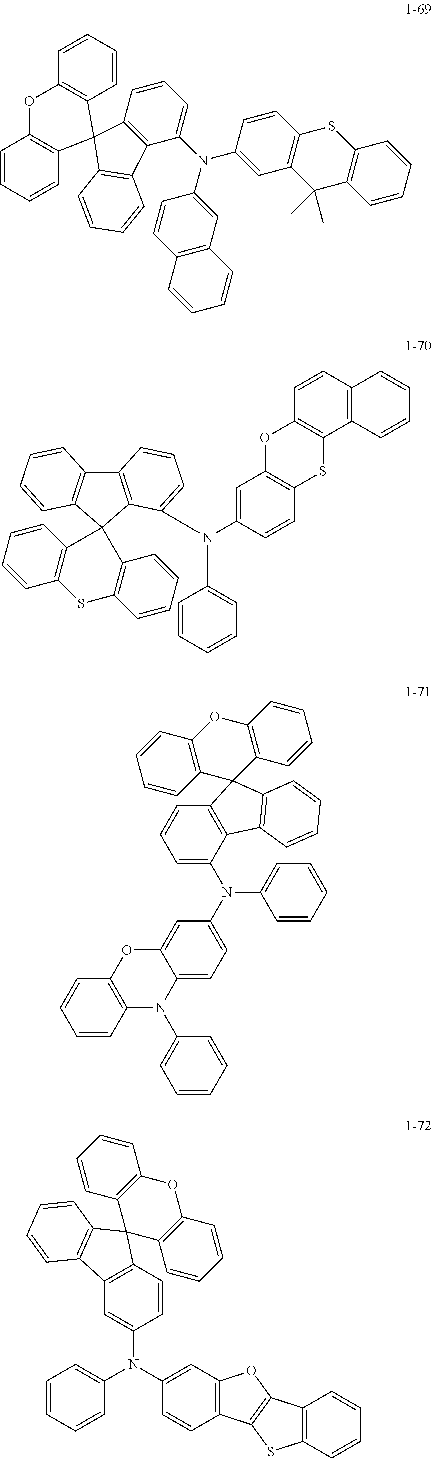 [SMILES: CC1(C)c2ccccc2Sc2ccc(N(c3ccc4ccccc4c3)c3cccc4c3-c3ccccc3C43c4ccccc4Oc4ccccc43)cc21 ; c1ccc(N(c2ccc3c(c2)Oc2ccc4ccccc4c2S3)c2cccc3c2C2(c4ccccc4Sc4ccccc42)c2ccccc2-3)cc1 ; c1ccc(N(c2ccc3c(c2)Oc2ccccc2N3c2ccccc2)c2cccc3c2-c2ccccc2C32c3ccccc3Oc3ccccc32)cc1 ; c1ccc(N(c2ccc3c(c2)-c2ccccc2C32c3ccccc3Oc3ccccc32)c2ccc3c(c2)oc2c4ccccc4sc32)cc1]

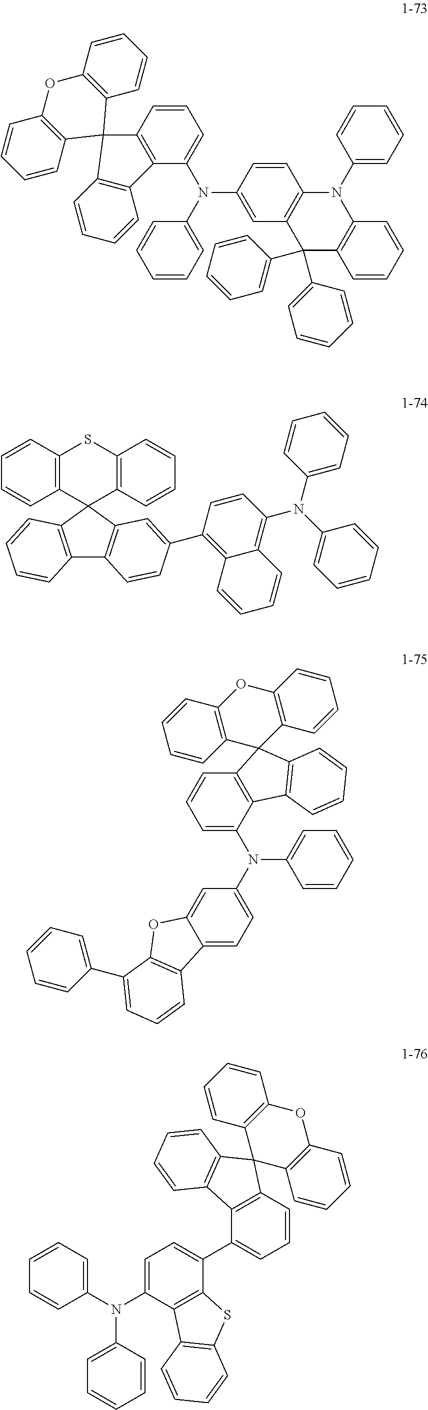 [SMILES: c1ccc(N(c2ccc3c(c2)C(c2ccccc2)(c2ccccc2)c2ccccc2N3c2ccccc2)c2cccc3c2-c2ccccc2C32c3ccccc3Oc3ccccc32)cc1 ; c1ccc(N(c2ccccc2)c2ccc(-c3ccc4c(c3)C3(c5ccccc5Sc5ccccc53)c3ccccc3-4)c3ccccc23)cc1 ; c1ccc(-c2cccc3c2oc2cc(N(c4ccccc4)c4cccc5c4-c4ccccc4C54c5ccccc5Oc5ccccc54)ccc23)cc1 ; c1ccc(N(c2ccccc2)c2ccc(-c3cccc4c3-c3ccccc3C43c4ccccc4Oc4ccccc43)c3sc4ccccc4c23)cc1]

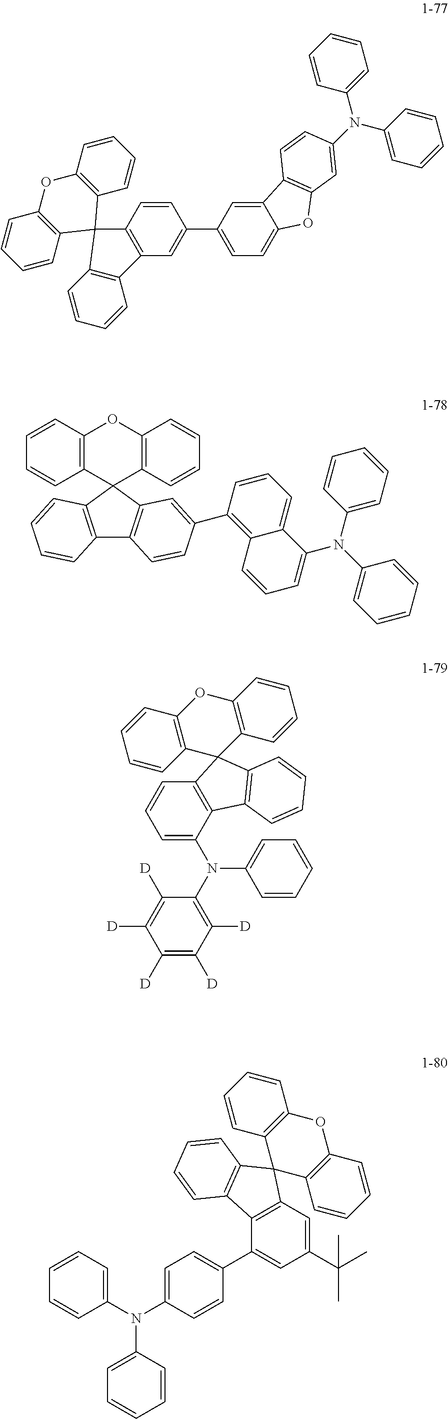 [SMILES: c1ccc(N(c2ccccc2)c2ccc3c(c2)oc2ccc(-c4ccc5c(c4)-c4ccccc4C54c5ccccc5Oc5ccccc54)cc23)cc1 ; c1ccc(N(c2ccccc2)c2cccc3c(-c4ccc5c(c4)C4(c6ccccc6Oc6ccccc64)c4ccccc4-5)cccc23)cc1 ; [2H]c1c([2H])c([2H])c(N(c2ccccc2)c2cccc3c2-c2ccccc2C32c3ccccc3Oc3ccccc32)c([2H])c1[2H] ; CC(C)(C)c1cc(-c2ccc(N(c3ccccc3)c3ccccc3)cc2)c2c(c1)C1(c3ccccc3Oc3ccccc31)c1ccccc1-2]

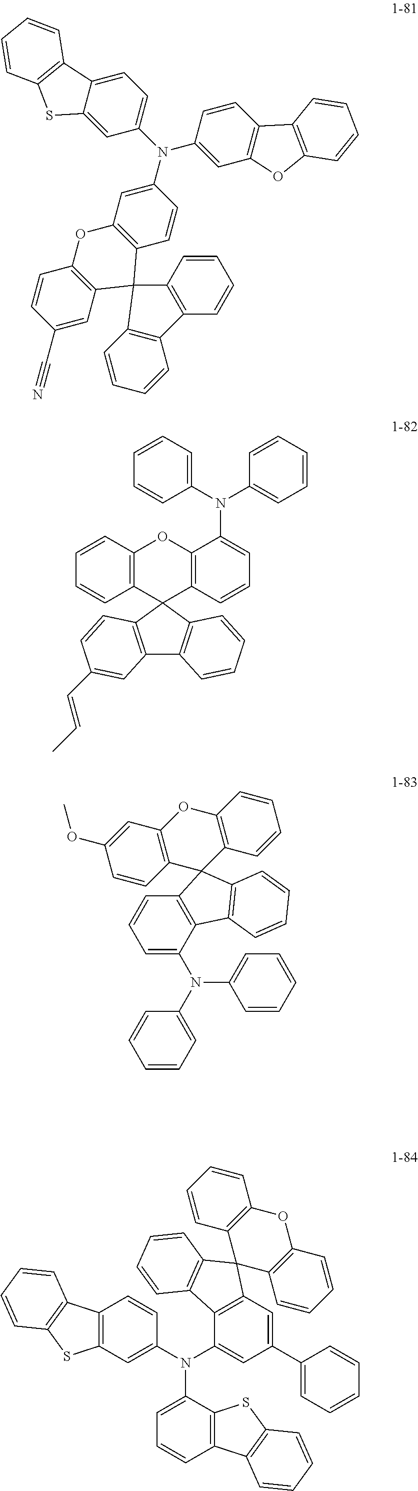

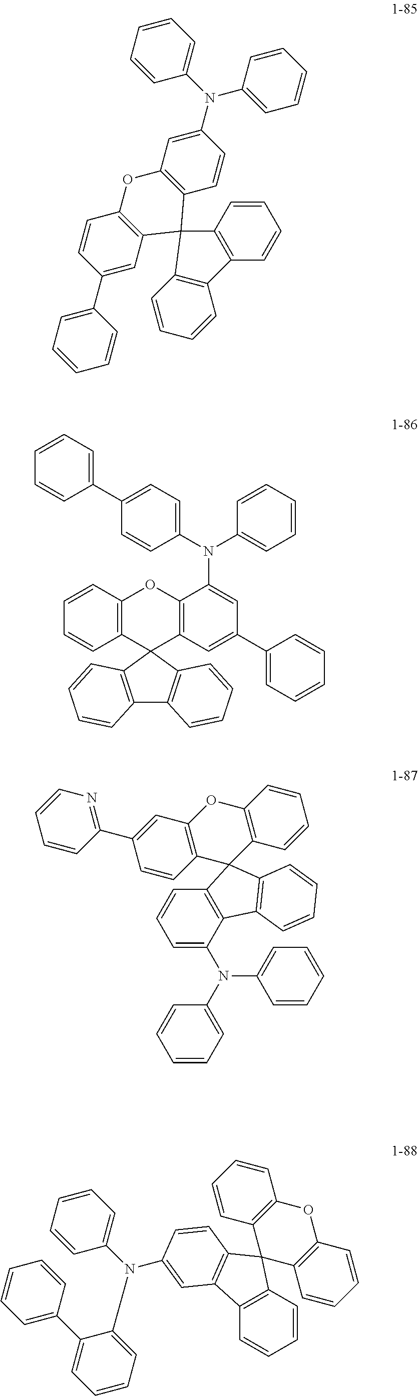 [SMILES: c1ccc(-c2ccc3c(c2)C2(c4ccc(N(c5ccccc5)c5ccccc5)cc4O3)c3ccccc3-c3ccccc32)cc1 ; c1ccc(-c2ccc(N(c3ccccc3)c3cc(-c4ccccc4)cc4c3Oc3ccccc3C43c4ccccc4-c4ccccc43)cc2)cc1 ; c1ccc(N(c2ccccc2)c2cccc3c2-c2ccccc2C32c3ccccc3Oc3cc(-c4ccccn4)ccc32)cc1 ; c1ccc(-c2ccccc2N(c2ccccc2)c2ccc3c(c2)-c2ccccc2C32c3ccccc3Oc3ccccc32)cc1]

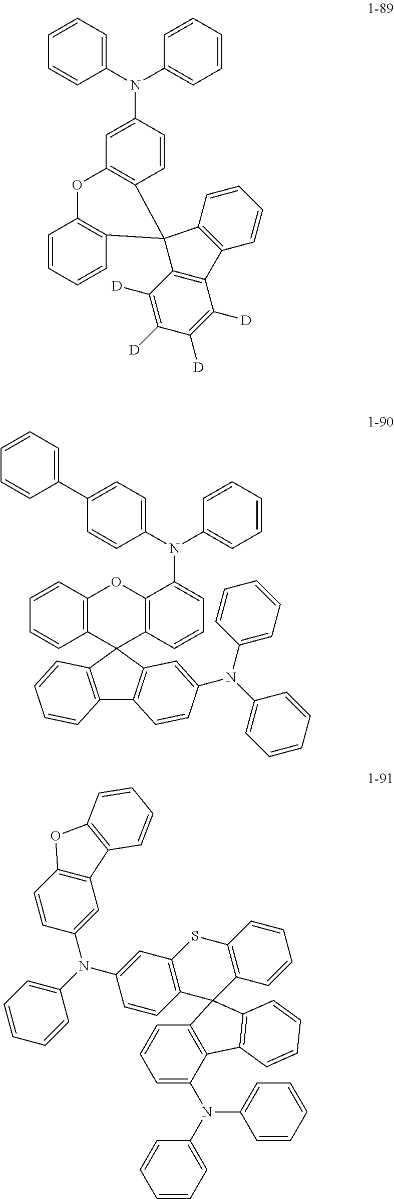 [SMILES: [2H]c1c([2H])c([2H])c2c(c1[2H])-c1ccccc1C21c2ccccc2Oc2cc(N(c3ccccc3)c3ccccc3)ccc21 ; c1ccc(-c2ccc(N(c3ccccc3)c3cccc4c3Oc3ccccc3C43c4ccccc4-c4ccc(N(c5ccccc5)c5ccccc5)cc43)cc2)cc1 ; c1ccc(N(c2ccc3c(c2)Sc2ccccc2C32c3ccccc3-c3c(N(c4ccccc4)c4ccccc4)cccc32)c2ccc3oc4ccccc4c3c2)cc1]

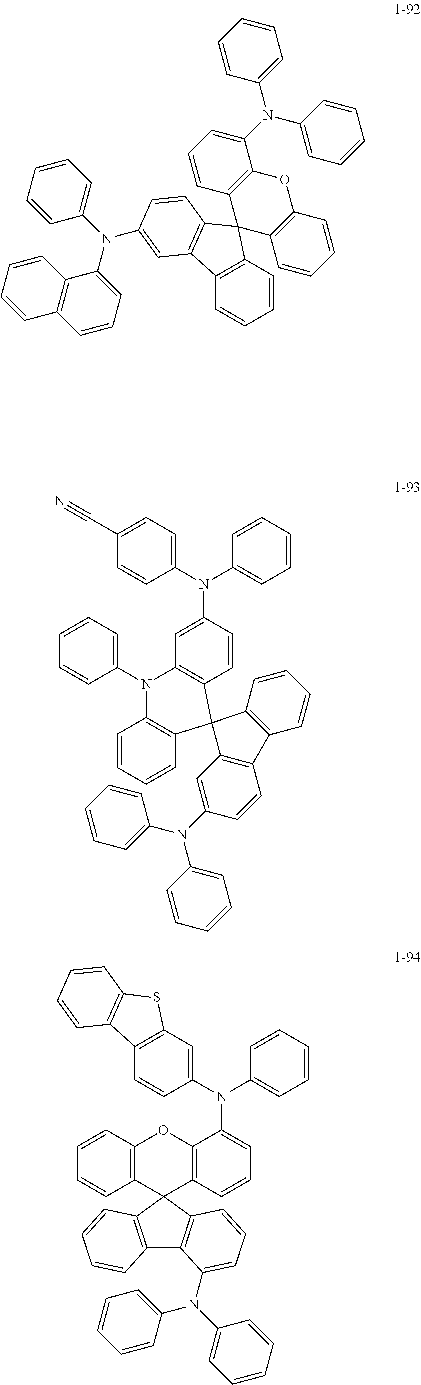 [SMILES: c1ccc(N(c2ccccc2)c2cccc3c2Oc2ccccc2C32c3ccccc3-c3cc(N(c4ccccc4)c4cccc5ccccc45)ccc32)cc1 ; N#Cc1ccc(N(c2ccccc2)c2ccc3c(c2)N(c2ccccc2)c2ccccc2C32c3ccccc3-c3ccc(N(c4ccccc4)c4ccccc4)cc32)cc1 ; c1ccc(N(c2ccc3c(c2)sc2ccccc23)c2cccc3c2Oc2ccccc2C32c3ccccc3-c3c(N(c4ccccc4)c4ccccc4)cccc32)cc1]

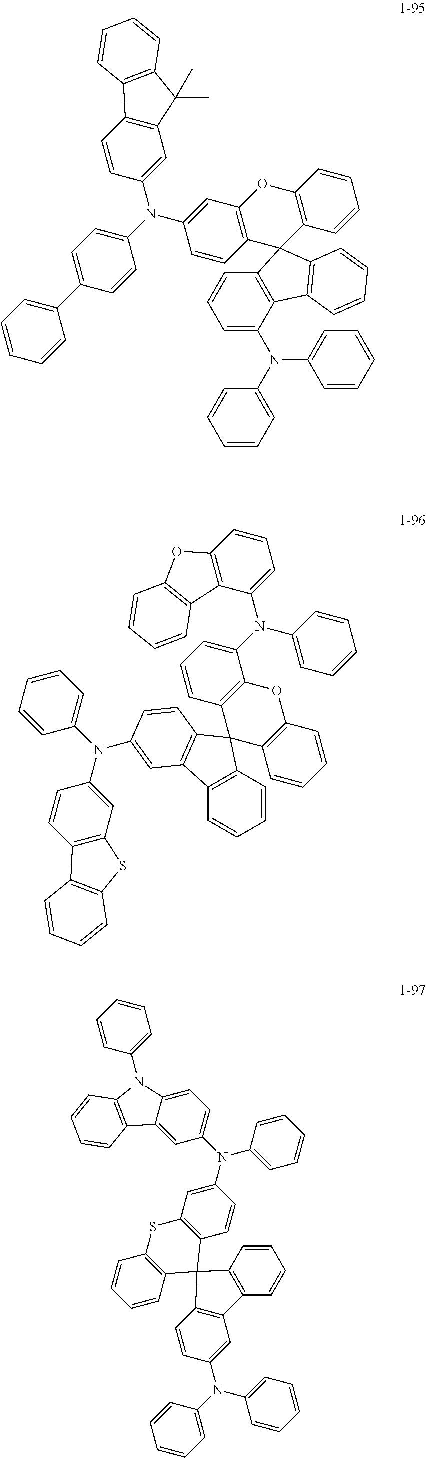 [SMILES: CC1(C)c2ccccc2-c2ccc(N(c3ccc(-c4ccccc4)cc3)c3ccc4c(c3)Oc3ccccc3C43c4ccccc4-c4c(N(c5ccccc5)c5ccccc5)cccc43)cc21 ; c1ccc(N(c2ccc3c(c2)-c2ccccc2C32c3ccccc3Oc3c(N(c4ccccc4)c4cccc5oc6ccccc6c45)cccc32)c2ccc3c(c2)sc2ccccc23)cc1 ; c1ccc(N(c2ccccc2)c2ccc3c(c2)-c2ccccc2C32c3ccccc3Sc3cc(N(c4ccccc4)c4ccc5c(c4)c4ccccc4n5-c4ccccc4)ccc32)cc1]

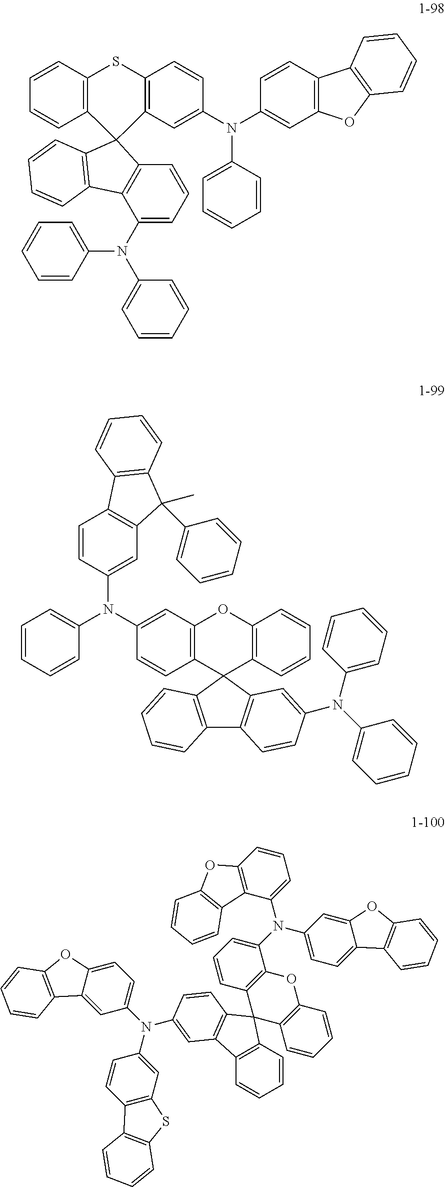 [SMILES: c1ccc(N(c2ccc3c(c2)C2(c4ccccc4S3)c3ccccc3-c3c(N(c4ccccc4)c4ccccc4)cccc32)c2ccc3c(c2)oc2ccccc23)cc1 ; CC1(c2ccccc2)c2ccccc2-c2ccc(N(c3ccccc3)c3ccc4c(c3)Oc3ccccc3C43c4ccccc4-c4ccc(N(c5ccccc5)c5ccccc5)cc43)cc21 ; c1ccc2c(c1)Oc1c(N(c3ccc4c(c3)oc3ccccc34)c3cccc4oc5ccccc5c34)cccc1C21c2ccccc2-c2cc(N(c3ccc4c(c3)sc3ccccc34)c3ccc4oc5ccccc5c4c3)ccc21]

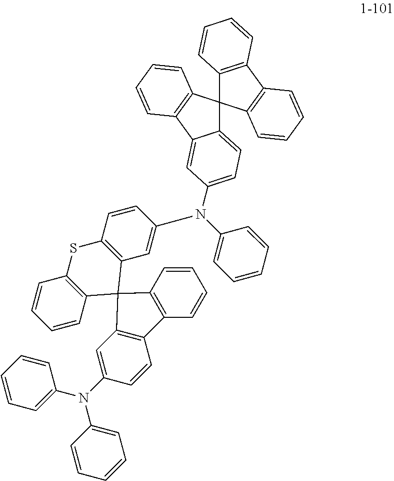 [SMILES: c1ccc(N(c2ccc3c(c2)-c2ccccc2C32c3ccccc3-c3ccccc32)c2ccc3c(c2)C2(c4ccccc4S3)c3ccccc3-c3ccc(N(c4ccccc4)c4ccccc4)cc32)cc1]

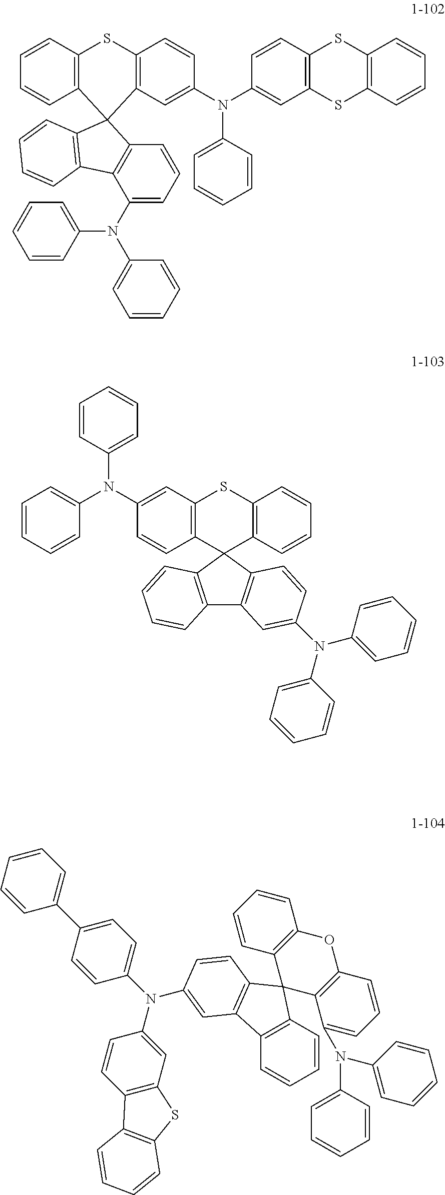 [SMILES: c1ccc(N(c2ccc3c(c2)Sc2ccccc2S3)c2ccc3c(c2)C2(c4ccccc4S3)c3ccccc3-c3c(N(c4ccccc4)c4ccccc4)cccc32)cc1 ; c1ccc(N(c2ccccc2)c2ccc3c(c2)Sc2ccccc2C32c3ccccc3-c3cc(N(c4ccccc4)c4ccccc4)ccc32)cc1 ; c1ccc(-c2ccc(N(c3ccc4c(c3)-c3ccccc3C43c4ccccc4Oc4cccc(N(c5ccccc5)c5ccccc5)c43)c3ccc4c(c3)sc3ccccc34)cc2)cc1]

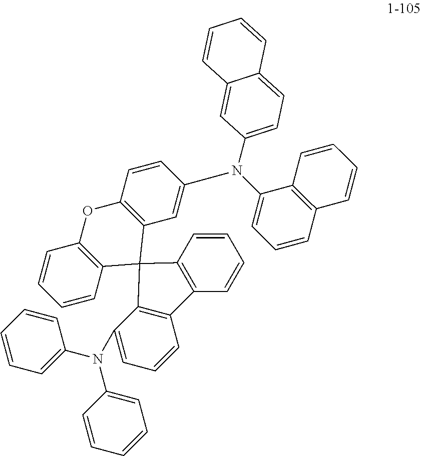 [SMILES: c1ccc(N(c2ccccc2)c2cccc3c2C2(c4ccccc4Oc4ccc(N(c5ccc6ccccc6c5)c5cccc6ccccc56)cc42)c2ccccc2-3)cc1]

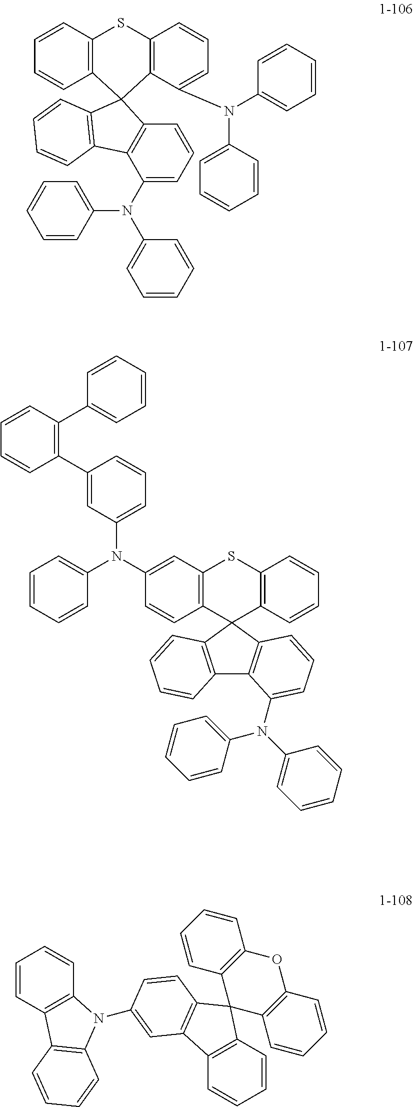 [SMILES: c1ccc(N(c2ccccc2)c2cccc3c2-c2ccccc2C32c3ccccc3Sc3cccc(N(c4ccccc4)c4ccccc4)c32)cc1 ; c1ccc(-c2ccccc2-c2cccc(N(c3ccccc3)c3ccc4c(c3)Sc3ccccc3C43c4ccccc4-c4c(N(c5ccccc5)c5ccccc5)cccc43)c2)cc1 ; c1ccc2c(c1)Oc1ccccc1C21c2ccccc2-c2cc(-n3c4ccccc4c4ccccc43)ccc21]

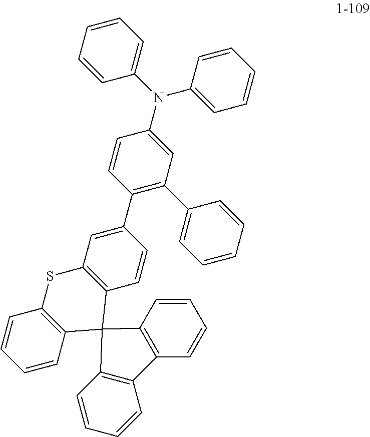 [SMILES: c1ccc(-c2cc(N(c3ccccc3)c3ccccc3)ccc2-c2ccc3c(c2)Sc2ccccc2C32c3ccccc3-c3ccccc32)cc1]

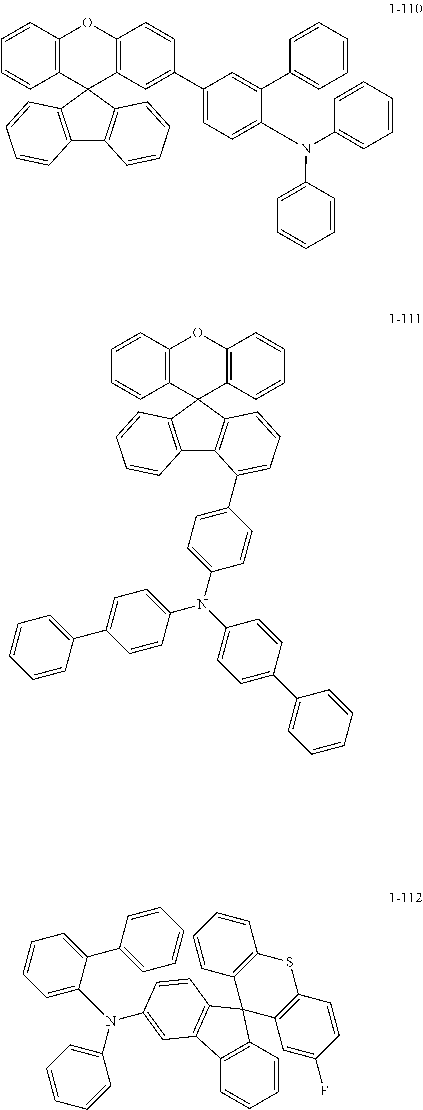 [SMILES: c1ccc(-c2cc(-c3ccc4c(c3)C3(c5ccccc5O4)c4ccccc4-c4ccccc43)ccc2N(c2ccccc2)c2ccccc2)cc1 ; c1ccc(-c2ccc(N(c3ccc(-c4ccccc4)cc3)c3ccc(-c4cccc5c4-c4ccccc4C54c5ccccc5Oc5ccccc54)cc3)cc2)cc1 ; Fc1ccc2c(c1)C1(c3ccccc3S2)c2ccccc2-c2cc(N(c3ccccc3)c3ccccc3-c3ccccc3)ccc21]

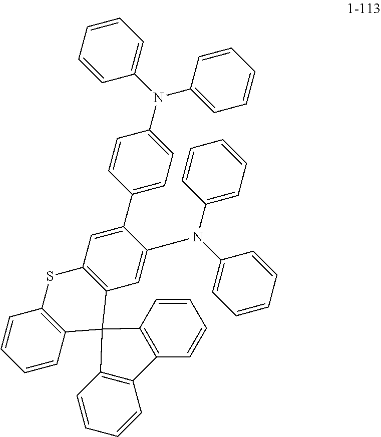 [SMILES: c1ccc(N(c2ccccc2)c2ccc(-c3cc4c(cc3N(c3ccccc3)c3ccccc3)C3(c5ccccc5S4)c4ccccc4-c4ccccc43)cc2)cc1]

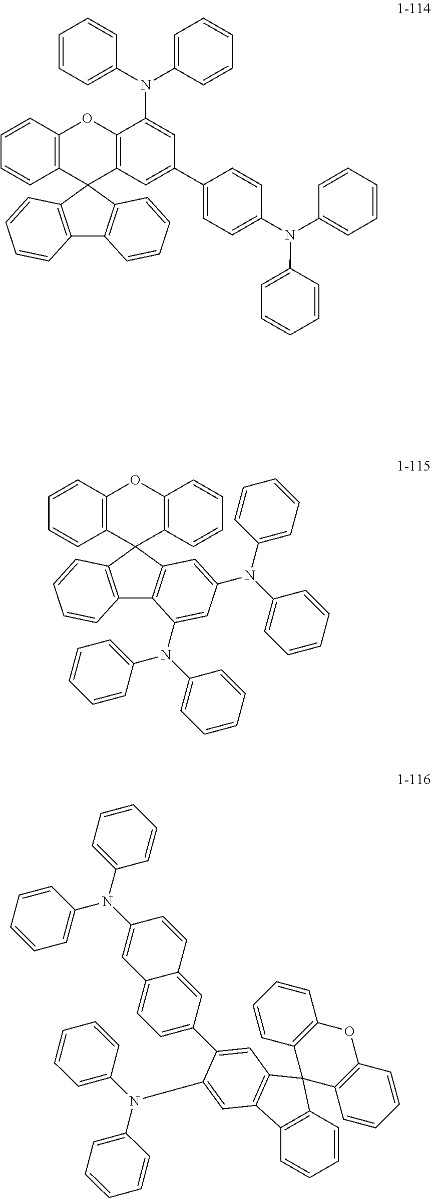 [SMILES: c1ccc(N(c2ccccc2)c2ccc(-c3cc(N(c4ccccc4)c4ccccc4)c4c(c3)C3(c5ccccc5O4)c4ccccc4-c4ccccc43)cc2)cc1 ; c1ccc(N(c2ccccc2)c2cc(N(c3ccccc3)c3ccccc3)c3c(c2)C2(c4ccccc4Oc4ccccc42)c2ccccc2-3)cc1 ; c1ccc(N(c2ccccc2)c2ccc3cc(-c4cc5c(cc4N(c4ccccc4)c4ccccc4)-c4ccccc4C54c5ccccc5Oc5ccccc54)ccc3c2)cc1]

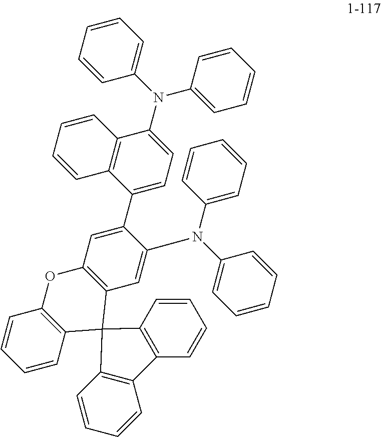 [SMILES: c1ccc(N(c2ccccc2)c2cc3c(cc2-c2ccc(N(c4ccccc4)c4ccccc4)c4ccccc24)Oc2ccccc2C32c3ccccc3-c3ccccc32)cc1]

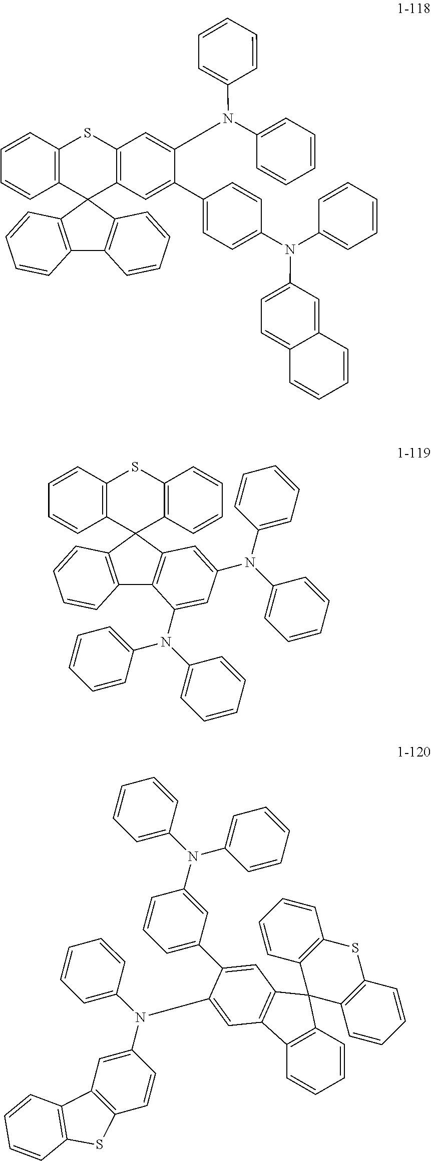 [SMILES: c1ccc(N(c2ccc(-c3cc4c(cc3N(c3ccccc3)c3ccccc3)Sc3ccccc3C43c4ccccc4-c4ccccc43)cc2)c2ccc3ccccc3c2)cc1 ; c1ccc(N(c2ccccc2)c2cc(N(c3ccccc3)c3ccccc3)c3c(c2)C2(c4ccccc4Sc4ccccc42)c2ccccc2-3)cc1 ; c1ccc(N(c2ccccc2)c2cccc(-c3cc4c(cc3N(c3ccccc3)c3ccc5sc6ccccc6c5c3)-c3ccccc3C43c4ccccc4Sc4ccccc43)c2)cc1]

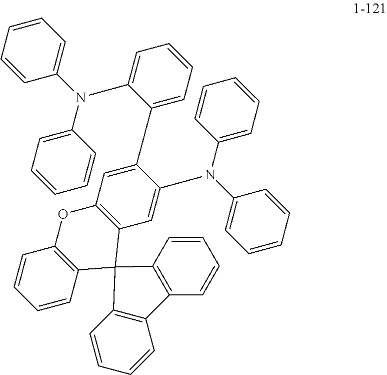 [SMILES: c1ccc(N(c2ccccc2)c2ccccc2-c2cc3c(cc2N(c2ccccc2)c2ccccc2)C2(c4ccccc4O3)c3ccccc3-c3ccccc32)cc1]

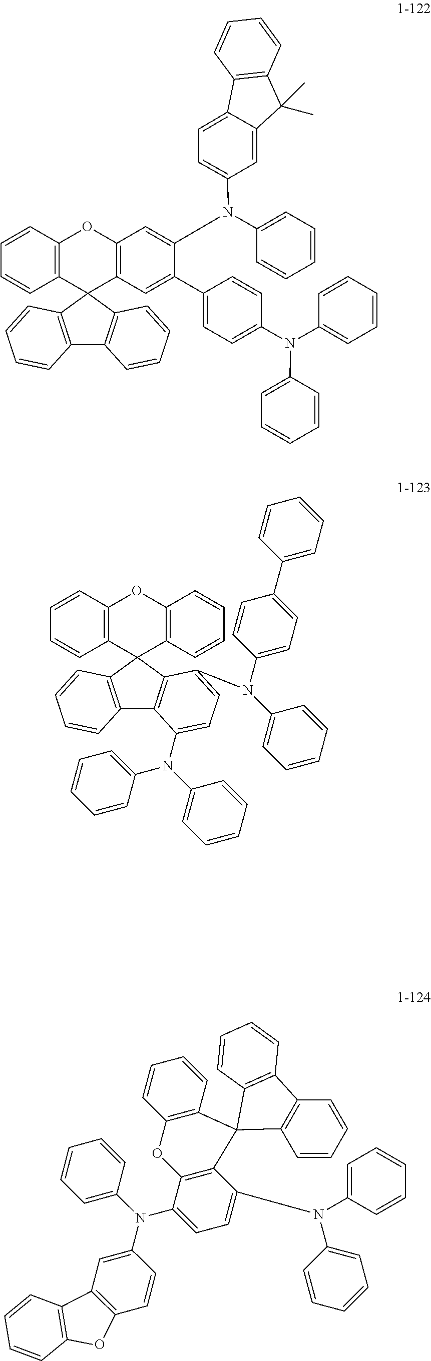 [SMILES: CC1(C)c2ccccc2-c2ccc(N(c3ccccc3)c3cc4c(cc3-c3ccc(N(c5ccccc5)c5ccccc5)cc3)C3(c5ccccc5O4)c4ccccc4-c4ccccc43)cc21 ; c1ccc(-c2ccc(N(c3ccccc3)c3ccc(N(c4ccccc4)c4ccccc4)c4c3C3(c5ccccc5Oc5ccccc53)c3ccccc3-4)cc2)cc1 ; c1ccc(N(c2ccc3oc4ccccc4c3c2)c2ccc(N(c3ccccc3)c3ccccc3)c3c2Oc2ccccc2C32c3ccccc3-c3ccccc32)cc1]

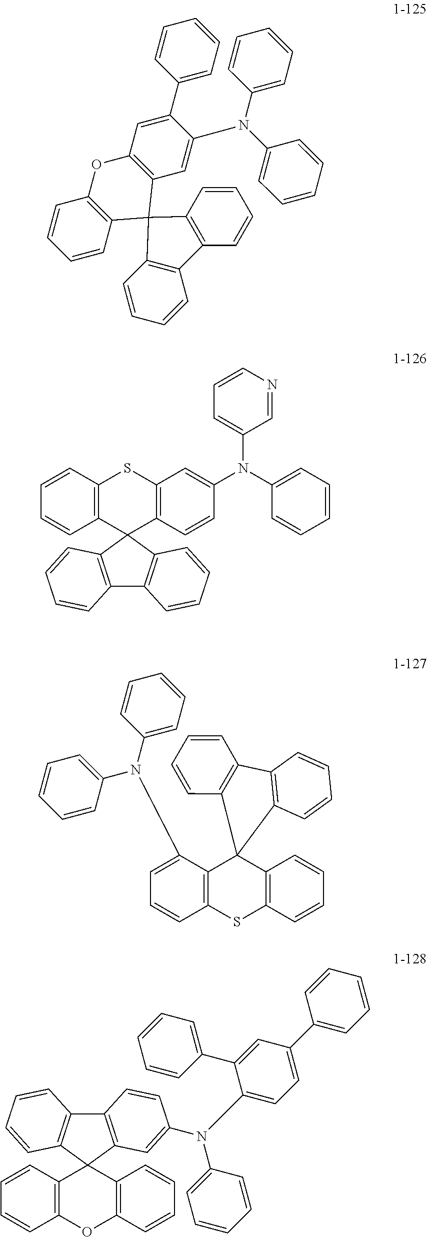 [SMILES: c1ccc(-c2cc3c(cc2N(c2ccccc2)c2ccccc2)C2(c4ccccc4O3)c3ccccc3-c3ccccc32)cc1 ; c1ccc(N(c2cccnc2)c2ccc3c(c2)Sc2ccccc2C32c3ccccc3-c3ccccc32)cc1 ; c1ccc(N(c2ccccc2)c2cccc3c2C2(c4ccccc4S3)c3ccccc3-c3ccccc32)cc1 ; c1ccc(-c2ccc(N(c3ccccc3)c3ccc4c(c3)C3(c5ccccc5Oc5ccccc53)c3ccccc3-4)c(-c3ccccc3)c2)cc1]

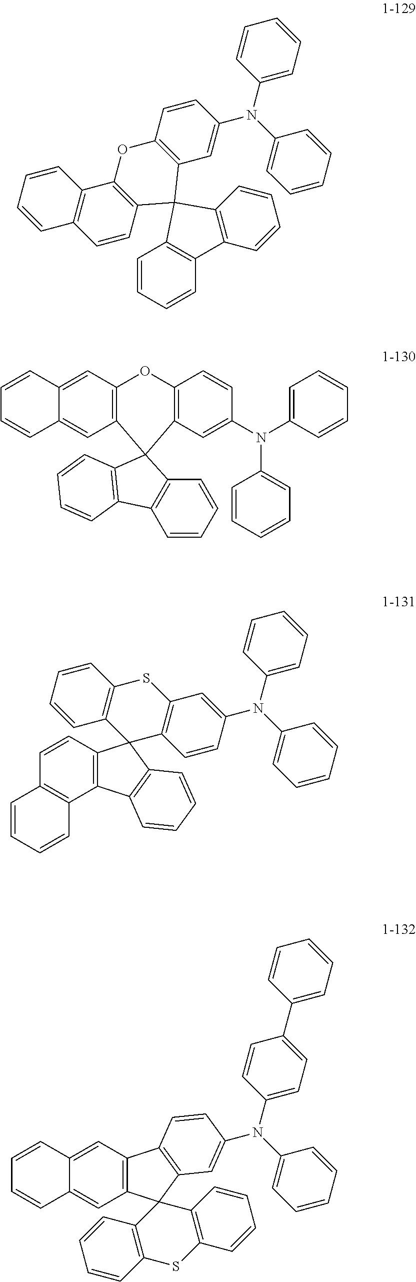 [SMILES: c1ccc(N(c2ccccc2)c2ccc3c(c2)C2(c4ccccc4-c4ccccc42)c2ccc4ccccc4c2O3)cc1 ; c1ccc(N(c2ccccc2)c2ccc3c(c2)C2(c4cc5ccccc5cc4O3)c3ccccc3-c3ccccc32)cc1 ; c1ccc(N(c2ccccc2)c2ccc3c(c2)Sc2ccccc2C32c3ccccc3-c3c2ccc2ccccc32)cc1 ; c1ccc(-c2ccc(N(c3ccccc3)c3ccc4c(c3)C3(c5ccccc5Sc5ccccc53)c3cc5ccccc5cc3-4)cc2)cc1]

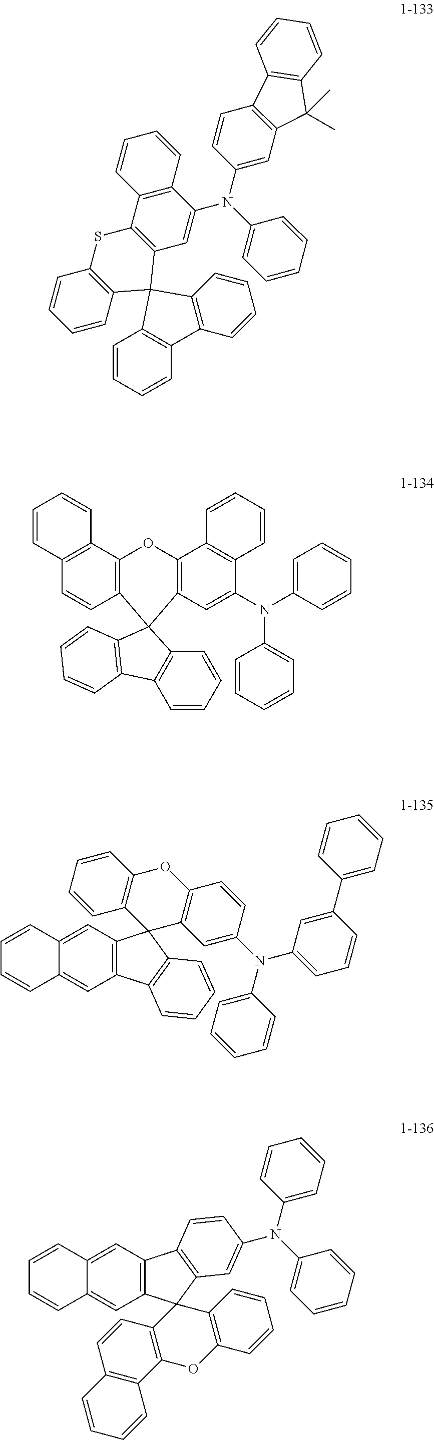 [SMILES: CC1(C)c2ccccc2-c2ccc(N(c3ccccc3)c3cc4c(c5ccccc35)Sc3ccccc3C43c4ccccc4-c4ccccc43)cc21 ; c1ccc(N(c2ccccc2)c2cc3c(c4ccccc24)Oc2c(ccc4ccccc24)C32c3ccccc3-c3ccccc32)cc1 ; c1ccc(-c2cccc(N(c3ccccc3)c3ccc4c(c3)C3(c5ccccc5O4)c4ccccc4-c4cc5ccccc5cc43)c2)cc1 ; c1ccc(N(c2ccccc2)c2ccc3c(c2)C2(c4ccccc4Oc4c2ccc2ccccc42)c2cc4ccccc4cc2-3)cc1]

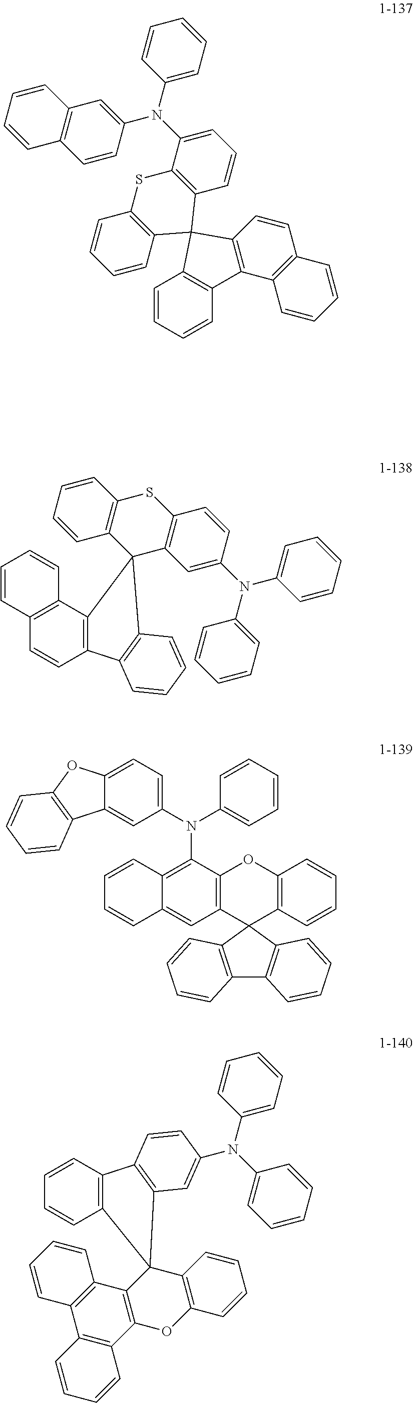 [SMILES: c1ccc(N(c2ccc3ccccc3c2)c2cccc3c2Sc2ccccc2C32c3ccccc3-c3c2ccc2ccccc32)cc1 ; c1ccc(N(c2ccccc2)c2ccc3c(c2)C2(c4ccccc4S3)c3ccccc3-c3ccc4ccccc4c32)cc1 ; c1ccc(N(c2ccc3oc4ccccc4c3c2)c2c3c(cc4ccccc24)C2(c4ccccc4O3)c3ccccc3-c3ccccc32)cc1 ; c1ccc(N(c2ccccc2)c2ccc3c(c2)C2(c4ccccc4Oc4c2c2ccccc2c2ccccc42)c2ccccc2-3)cc1]

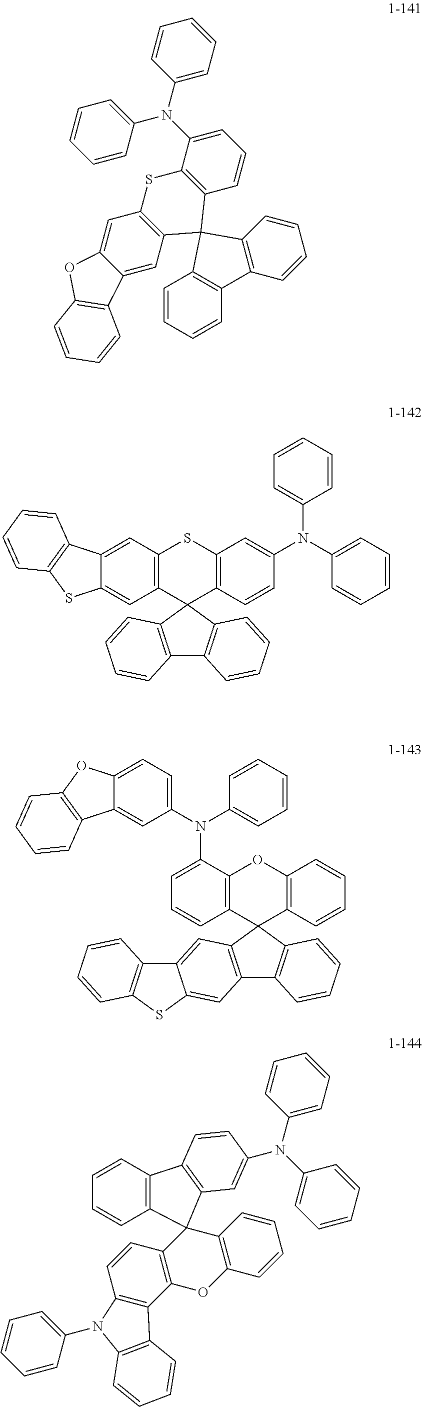 [SMILES: c1ccc(N(c2ccccc2)c2cccc3c2Sc2cc4oc5ccccc5c4cc2C32c3ccccc3-c3ccccc32)cc1 ; c1ccc(N(c2ccccc2)c2ccc3c(c2)Sc2cc4c(cc2C32c3ccccc3-c3ccccc32)sc2ccccc24)cc1 ; c1ccc(N(c2ccc3oc4ccccc4c3c2)c2cccc3c2Oc2ccccc2C32c3ccccc3-c3cc4sc5ccccc5c4cc32)cc1 ; c1ccc(N(c2ccccc2)c2ccc3c(c2)C2(c4ccccc4Oc4c2ccc2c4c4ccccc4n2-c2ccccc2)c2ccccc2-3)cc1]

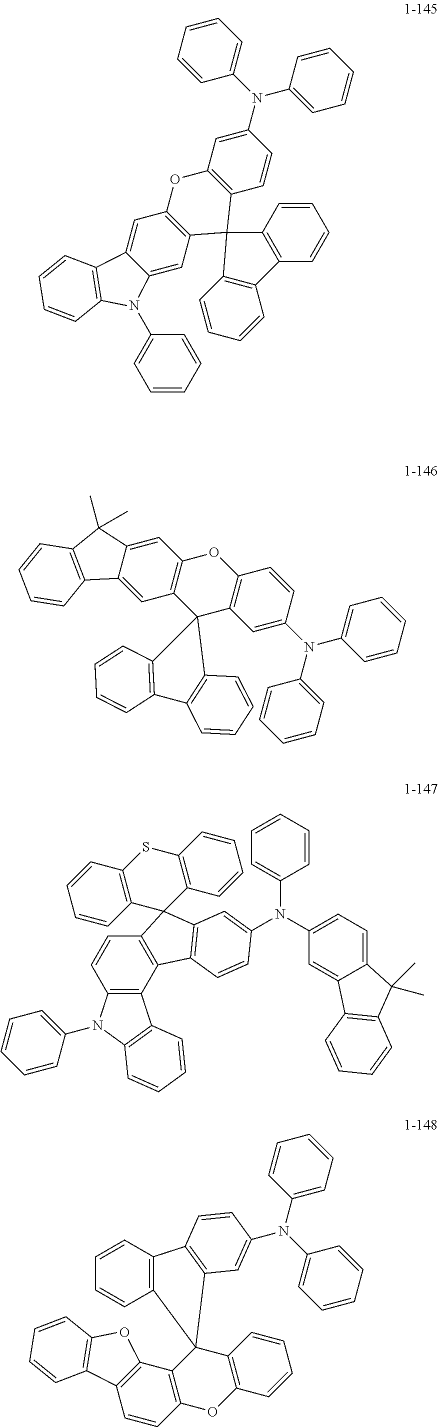 [SMILES: c1ccc(N(c2ccccc2)c2ccc3c(c2)Oc2cc4c5ccccc5n(-c5ccccc5)c4cc2C32c3ccccc3-c3ccccc32)cc1 ; CC1(C)c2ccccc2-c2cc3c(cc21)Oc1ccc(N(c2ccccc2)c2ccccc2)cc1C31c2ccccc2-c2ccccc21 ; CC1(C)c2ccccc2-c2cc(N(c3ccccc3)c3ccc4c(c3)C3(c5ccccc5Sc5ccccc53)c3ccc5c(c3-4)c3ccccc3n5-c3ccccc3)ccc21 ; c1ccc(N(c2ccccc2)c2ccc3c(c2)C2(c4ccccc4Oc4ccc5c(oc6ccccc65)c42)c2ccccc2-3)cc1]

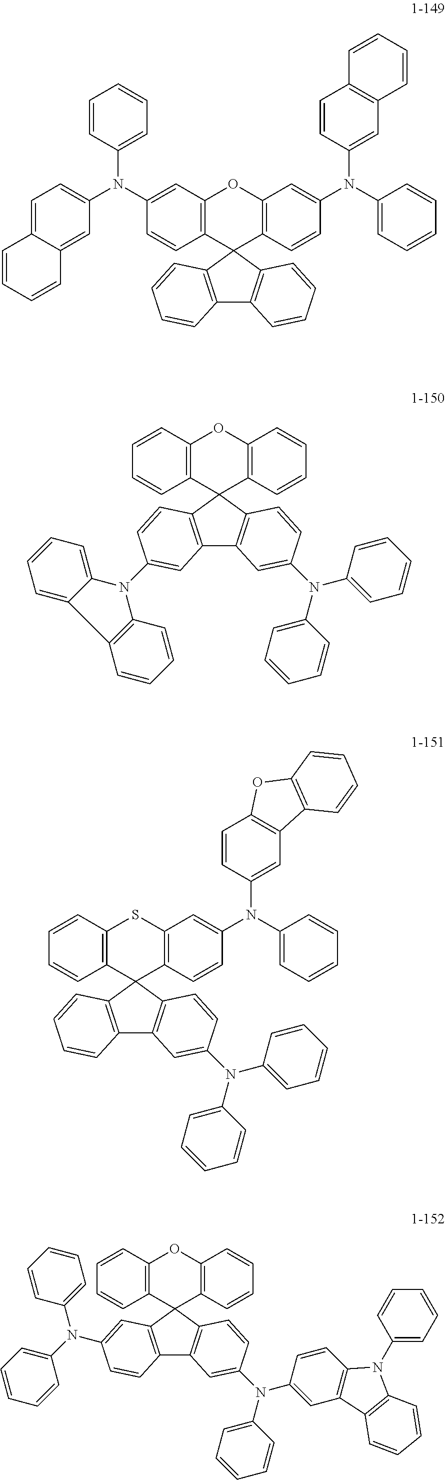 [SMILES: c1ccc(N(c2ccc3c(c2)Oc2cc(N(c4ccccc4)c4ccc5ccccc5c4)ccc2C32c3ccccc3-c3ccccc32)c2ccc3ccccc3c2)cc1 ; c1ccc(N(c2ccccc2)c2ccc3c(c2)-c2cc(-n4c5ccccc5c5ccccc54)ccc2C32c3ccccc3Oc3ccccc32)cc1 ; c1ccc(N(c2ccccc2)c2ccc3c(c2)-c2ccccc2C32c3ccccc3Sc3cc(N(c4ccccc4)c4ccc5oc6ccccc6c5c4)ccc32)cc1 ; c1ccc(N(c2ccccc2)c2ccc3c(c2)C2(c4ccccc4Oc4ccccc42)c2ccc(N(c4ccccc4)c4ccc5c(c4)c4ccccc4n5-c4ccccc4)cc2-3)cc1]

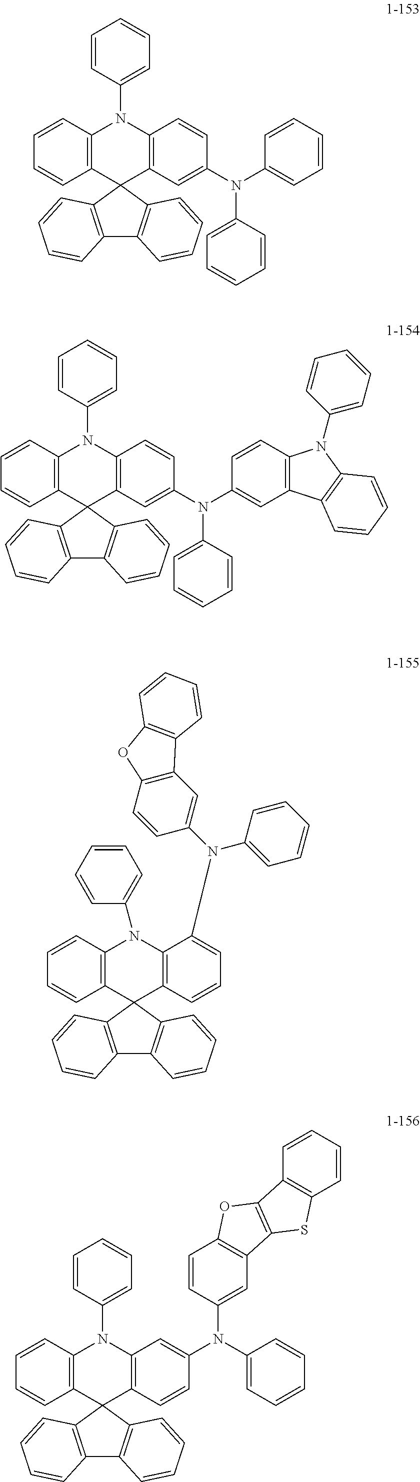 [SMILES: c1ccc(N(c2ccccc2)c2ccc3c(c2)C2(c4ccccc4-c4ccccc42)c2ccccc2N3c2ccccc2)cc1 ; c1ccc(N(c2ccc3c(c2)C2(c4ccccc4-c4ccccc42)c2ccccc2N3c2ccccc2)c2ccc3c(c2)c2ccccc2n3-c2ccccc2)cc1 ; c1ccc(N(c2ccc3oc4ccccc4c3c2)c2cccc3c2N(c2ccccc2)c2ccccc2C32c3ccccc3-c3ccccc32)cc1 ; c1ccc(N(c2ccc3c(c2)N(c2ccccc2)c2ccccc2C32c3ccccc3-c3ccccc32)c2ccc3oc4c5ccccc5sc4c3c2)cc1]

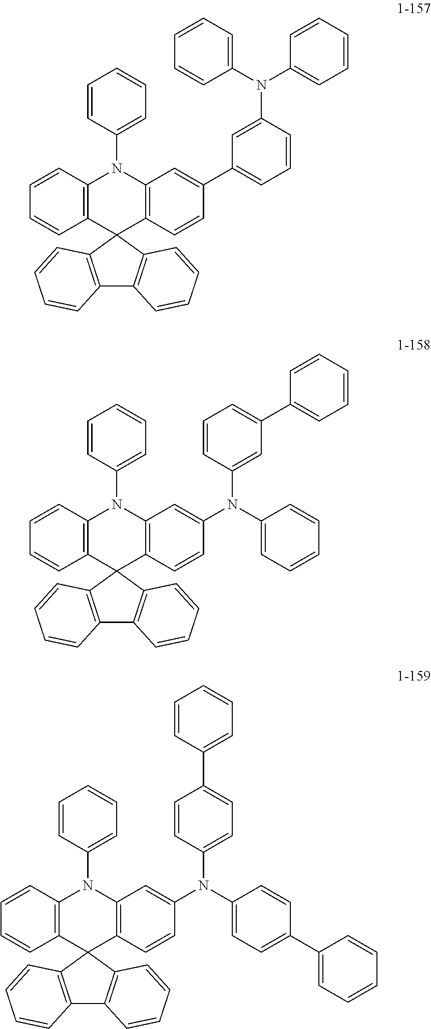 [SMILES: c1ccc(N(c2ccccc2)c2cccc(-c3ccc4c(c3)N(c3ccccc3)c3ccccc3C43c4ccccc4-c4ccccc43)c2)cc1 ; c1ccc(-c2cccc(N(c3ccccc3)c3ccc4c(c3)N(c3ccccc3)c3ccccc3C43c4ccccc4-c4ccccc43)c2)cc1 ; c1ccc(-c2ccc(N(c3ccc(-c4ccccc4)cc3)c3ccc4c(c3)N(c3ccccc3)c3ccccc3C43c4ccccc4-c4ccccc43)cc2)cc1]

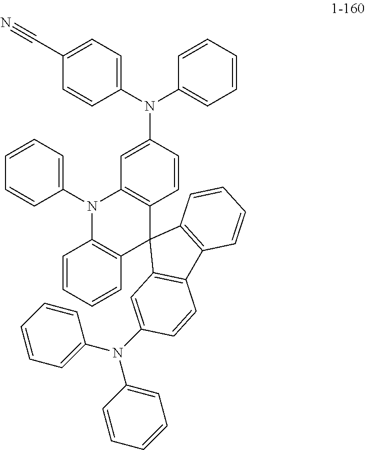 [SMILES: N#Cc1ccc(N(c2ccccc2)c2ccc3c(c2)N(c2ccccc2)c2ccccc2C32c3ccccc3-c3ccc(N(c4ccccc4)c4ccccc4)cc32)cc1]

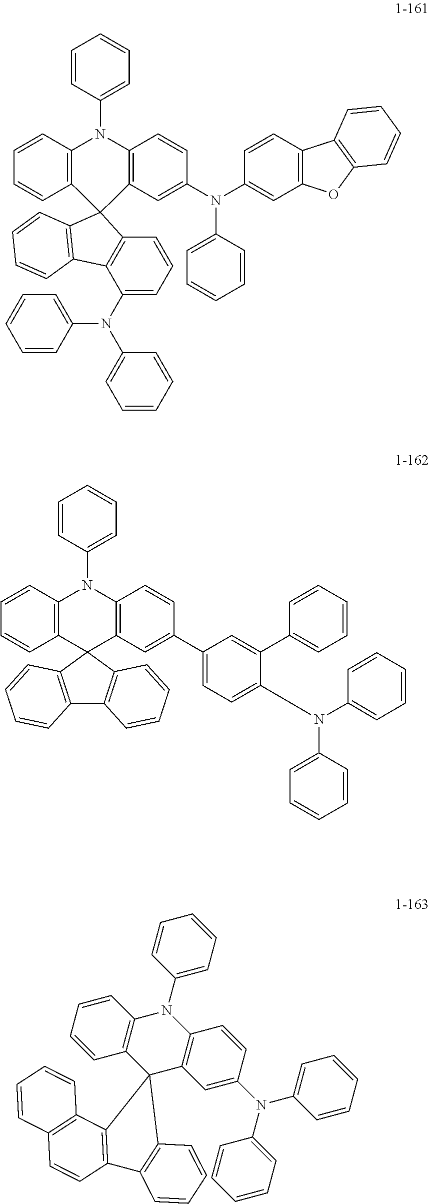 [SMILES: c1ccc(N(c2ccc3c(c2)C2(c4ccccc4-c4c(N(c5ccccc5)c5ccccc5)cccc42)c2ccccc2N3c2ccccc2)c2ccc3c(c2)oc2ccccc23)cc1 ; c1ccc(-c2cc(-c3ccc4c(c3)C3(c5ccccc5-c5ccccc53)c3ccccc3N4c3ccccc3)ccc2N(c2ccccc2)c2ccccc2)cc1 ; c1ccc(N(c2ccccc2)c2ccc3c(c2)C2(c4ccccc4-c4ccc5ccccc5c42)c2ccccc2N3c2ccccc2)cc1]

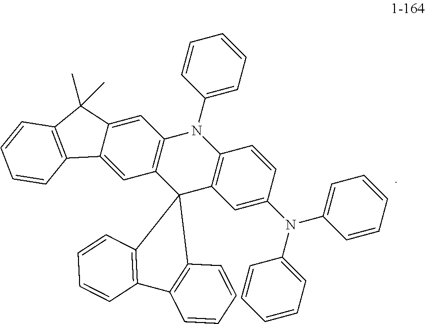 [SMILES: CC1(C)c2ccccc2-c2cc3c(cc21)N(c1ccccc1)c1ccc(N(c2ccccc2)c2ccccc2)cc1C31c2ccccc2-c2ccccc21]

6. The organic electronic element of claim 1, wherein at least one of Ar⁵ to Ar⁷ in Formula 2 is represented by any one of Formulas 2-1 to 2-6: 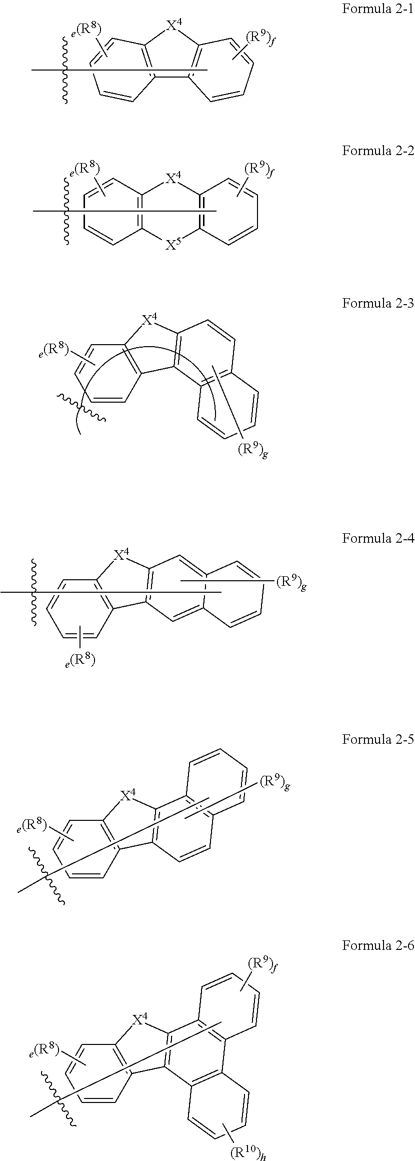
wherein:
1) X⁴ and X⁵ are each independently NAr⁸, O, S or CR^{c}R^{d},
2) Ar⁸ is the same as the definition of Ar¹ in Formula 1,
3) R⁸, R⁹, R¹⁰, R^{c} and R^{d} are each independently selected from the group consisting of hydrogen; deuterium; halogen; a silane group unsubstituted or substituted with a C₁-C₂₀ alkyl group or a C₆-C₂₀ aryl group; cyano group; nitro group; C₁-C₂₀ alkoxy group; C₆-C₂₀ aryloxy group; C₁-C₂₀ alkyl group; C₂-C₂₀ alkenyl group; C₂-C₂₀ alkynyl group; C₆-C₂₀ aryl group; fluorenyl group; a C₂-C₂₀ heterocyclic group including at least one heteroatom of O, N, S, Si or P; and a C₃-C₂₀ aliphatic ring; alternatively, adjacent groups may be bonded to each other to form a ring, and adjacent substituents may be bonded to form a ring, and
4) e, f and h are integers from 0 to 4, and g is an integer from 0 to 6.

7. The organic electronic element of claim 1, wherein the compound represented by Formula 2 is represented by any one of Formulas 2-7 to 2-9: 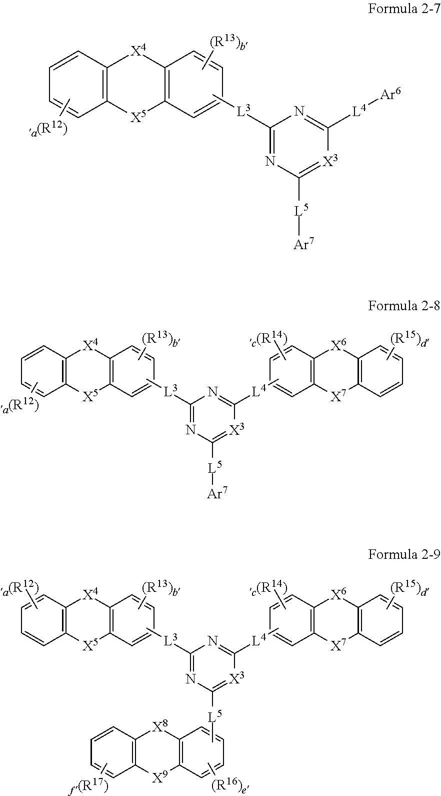
wherein:
1) X³, L³, L⁴, L⁵, Ar⁶ and Ar⁷ are the same as defined in claim 1,
2) X⁴, X⁶ and X⁸ are each independently O, S, NAr⁹ or CR^{c}R^{d},
3) X⁵, X⁷ and X⁹ are each independently O, S, NAr¹⁰, CR^{e}R^{f} or single bond, 4) a′, d′ and f′ are an integer of 0 to 4, b′, c′ and e′ are an integer of 0 to 3,
5) Ar⁹ and Ar¹⁰ are the same as the definition of Ar¹ in claim 1,
6) R^{c}, R^{d}, R^{e}, R^{f}, R¹², R¹³, R¹⁴, R¹⁵, R¹⁶ and R¹⁷ are each independently selected from the group consisting of hydrogen; deuterium; halogen; a silane group unsubstituted or substituted with a C₁-C₂₀ alkyl group or a C₆-C₂₀ aryl group; cyano group; nitro group; C₁-C₂₀ alkoxy group; C₆-C₂₀ aryloxy group; C₁-C₂₀ alkyl group; C₂-C₂₀ alkenyl group; C₂-C₂₀ alkynyl group; C₆-C₂₀ aryl group; fluorenyl group; a C₂-C₂₀ heterocyclic group including at least one heteroatom of O, N, S, Si or P; and a C₃-C₂₀ aliphatic ring, and alternatively adjacent groups may be bonded to each other to form a ring, and adjacent substituents may be bonded to form a ring.

8. The organic electronic element of claim 1, wherein at least one of L¹ to L⁵ in Formulas 1 to 2 is represented by one of the following Formulas b-1 to Formula b-13: 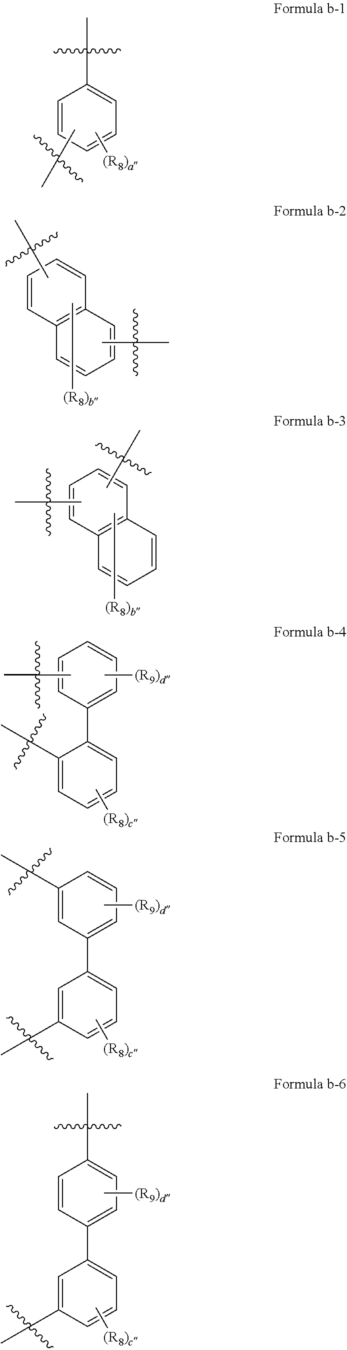 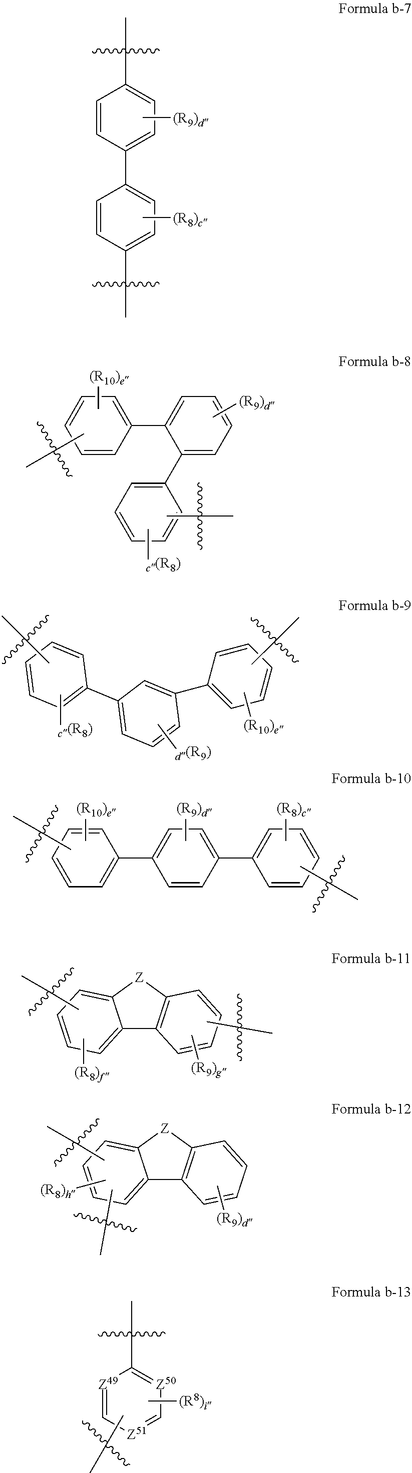
wherein:
1) Z is O, S, N-L⁶-Ar¹¹ or CR₆R₇,
2) L⁶ is the same as the definition of L¹ in claim 1,
3) Ar¹¹ is the same as the definition of Ar¹ in claim 1,
4) R⁶, R⁷, R⁸, R⁹ and R¹⁰ are the same or different from each other, and each independently selected from the group consisting of hydrogen; deuterium; C₆-C₂₀ aryl group; fluorenyl group; a C₂-C₂₀ heterocyclic group including at least one heteroatom of O, N, S, Si or P; and a C₃-C₂₀ aliphatic ring; and adjacent groups may combine with each other to form a ring,
5) a″, c″, d″ and e″ are each independently an integer of 0 to 4, b″ is an integer of to 6, f″ and g″ are each independently an integer of 0 to 3, h″ is an integer of 0 to 2, i″ is or 1,
6) Z⁴⁹, Z⁵⁰ and Z⁵¹ are each independently CR^{g} or N, and at least one of Z⁴⁹, Z⁵⁰ and Z⁵¹ is N,
7) R^{g} is are selected from the group consisting of hydrogen; deuterium; halogen; a silane group unsubstituted or substituted with a C₁-C₂₀ alkyl group or a C₆-C₂₀ aryl group; cyano group; nitro group; C₁˜C₂₀ alkylthio group; C₁-C₂₀ alkoxy group; C₆-C₂₀ aryloxy group; C₁-C₂₀ alkyl group; C₂-C₂₀ alkenyl group; C₂-C₂₀ alkynyl group; C₆-C₂₀ aryl group; fluorenyl group; a C₂-C₂₀ heterocyclic group including at least one heteroatom of O, N, S, Si or P; and a C₃-C₂₀ aliphatic ring; C₇˜C₂₀ arylalkyl group; and a C₈˜C₂₀ arylalkenyl group.

9. The organic electronic element of claim 1, wherein the compound represented by Formula 2 is selected from the following compounds:

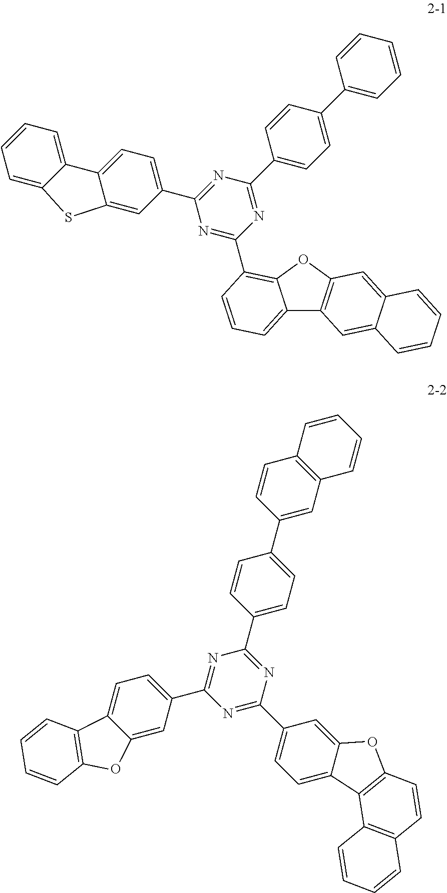 [SMILES: c1ccc(-c2ccc(-c3nc(-c4ccc5c(c4)sc4ccccc45)nc(-c4cccc5c4oc4cc6ccccc6cc45)n3)cc2)cc1 ; c1ccc2cc(-c3ccc(-c4nc(-c5ccc6c(c5)oc5ccccc56)nc(-c5ccc6c(c5)oc5ccc7ccccc7c56)n4)cc3)ccc2c1]

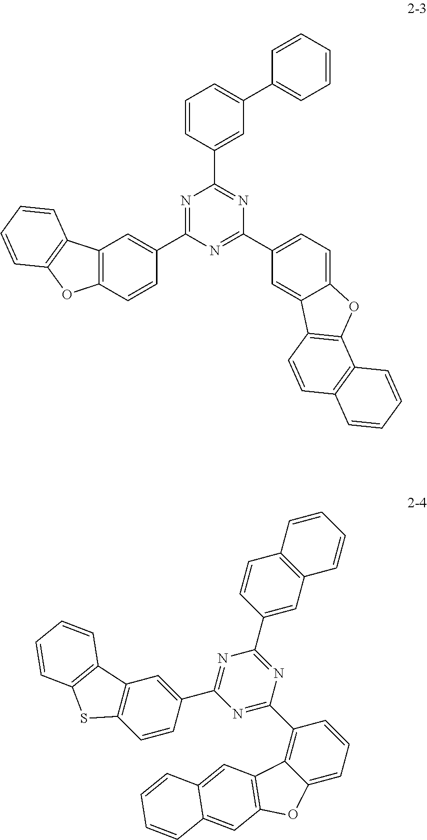 [SMILES: c1ccc(-c2cccc(-c3nc(-c4ccc5oc6ccccc6c5c4)nc(-c4ccc5oc6c7ccccc7ccc6c5c4)n3)c2)cc1 ; c1ccc2cc(-c3nc(-c4ccc5sc6ccccc6c5c4)nc(-c4cccc5oc6cc7ccccc7cc6c45)n3)ccc2c1]

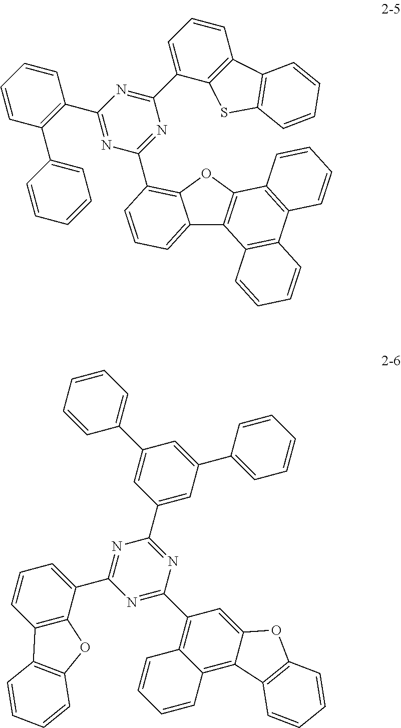 [SMILES: c1ccc(-c2ccccc2-c2nc(-c3cccc4c3oc3c5ccccc5c5ccccc5c43)nc(-c3cccc4c3sc3ccccc34)n2)cc1 ; c1ccc(-c2cc(-c3ccccc3)cc(-c3nc(-c4cc5oc6ccccc6c5c5ccccc45)nc(-c4cccc5c4oc4ccccc45)n3)c2)cc1]

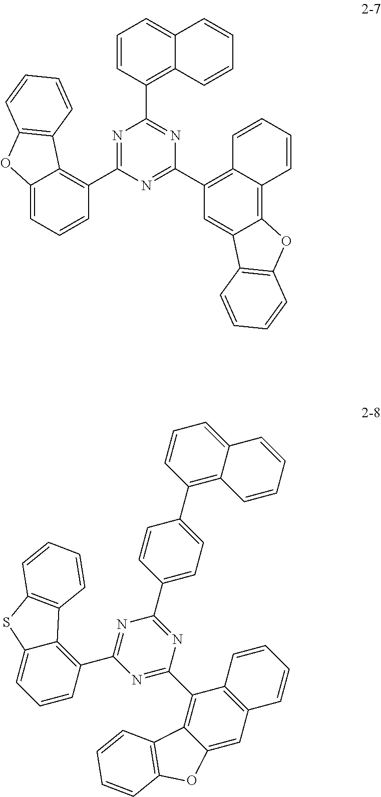 [SMILES: c1ccc2c(-c3nc(-c4cc5c6ccccc6oc5c5ccccc45)nc(-c4cccc5oc6ccccc6c45)n3)cccc2c1 ; c1ccc2c(-c3ccc(-c4nc(-c5c6ccccc6cc6oc7ccccc7c56)nc(-c5cccc6sc7ccccc7c56)n4)cc3)cccc2c1]

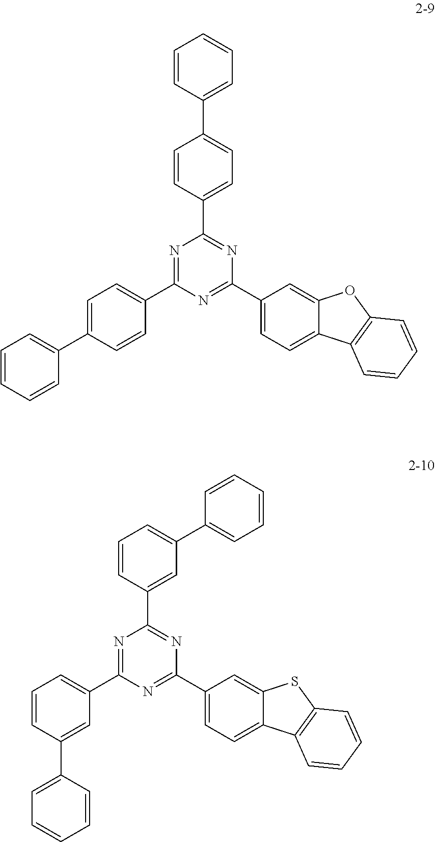 [SMILES: c1ccc(-c2ccc(-c3nc(-c4ccc(-c5ccccc5)cc4)nc(-c4ccc5c(c4)oc4ccccc45)n3)cc2)cc1 ; c1ccc(-c2cccc(-c3nc(-c4cccc(-c5ccccc5)c4)nc(-c4ccc5c(c4)sc4ccccc45)n3)c2)cc1]

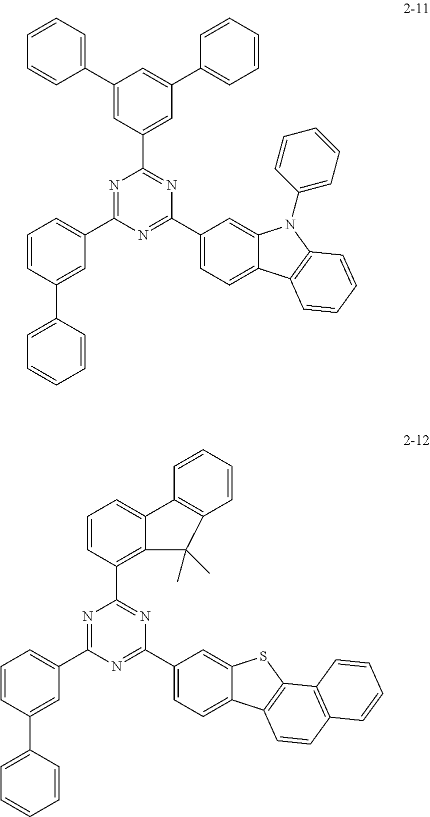 [SMILES: c1ccc(-c2cccc(-c3nc(-c4cc(-c5ccccc5)cc(-c5ccccc5)c4)nc(-c4ccc5c6ccccc6n(-c6ccccc6)c5c4)n3)c2)cc1 ; CC1(C)c2ccccc2-c2cccc(-c3nc(-c4cccc(-c5ccccc5)c4)nc(-c4ccc5c(c4)sc4c6ccccc6ccc54)n3)c21]

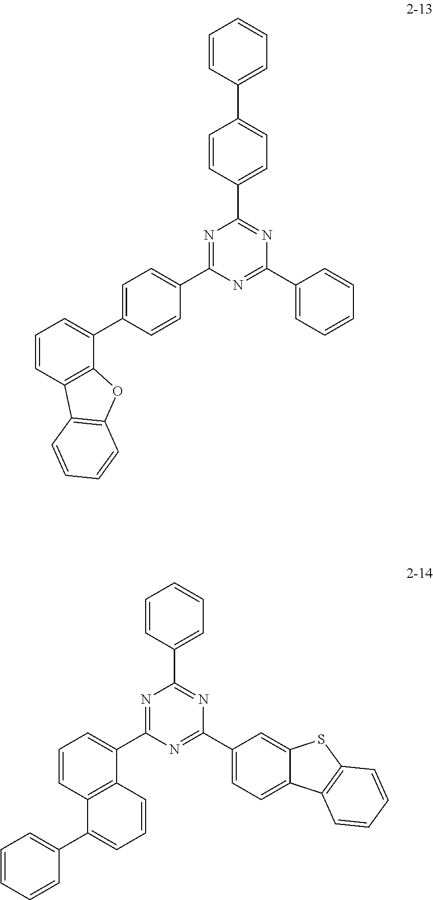 [SMILES: c1ccc(-c2ccc(-c3nc(-c4ccccc4)nc(-c4ccc(-c5cccc6c5oc5ccccc56)cc4)n3)cc2)cc1 ; c1ccc(-c2nc(-c3ccc4c(c3)sc3ccccc34)nc(-c3cccc4c(-c5ccccc5)cccc34)n2)cc1]

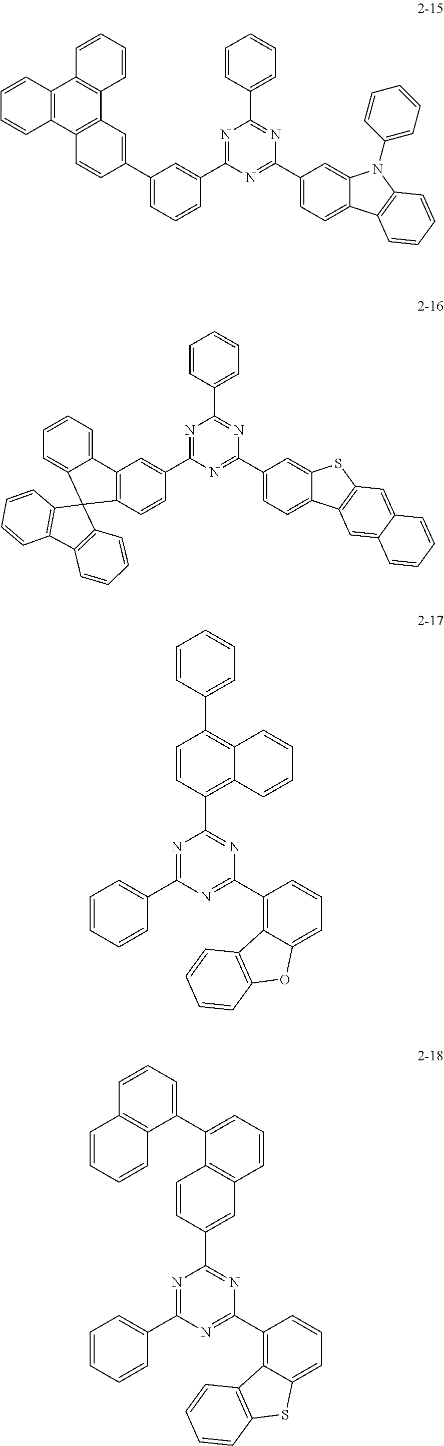 [SMILES: c1ccc(-c2nc(-c3cccc(-c4ccc5c6ccccc6c6ccccc6c5c4)c3)nc(-c3ccc4c5ccccc5n(-c5ccccc5)c4c3)n2)cc1 ; c1ccc(-c2nc(-c3ccc4c(c3)-c3ccccc3C43c4ccccc4-c4ccccc43)nc(-c3ccc4c(c3)sc3cc5ccccc5cc34)n2)cc1 ; c1ccc(-c2nc(-c3ccc(-c4ccccc4)c4ccccc34)nc(-c3cccc4oc5ccccc5c34)n2)cc1 ; c1ccc(-c2nc(-c3ccc4c(-c5cccc6ccccc56)cccc4c3)nc(-c3cccc4sc5ccccc5c34)n2)cc1]

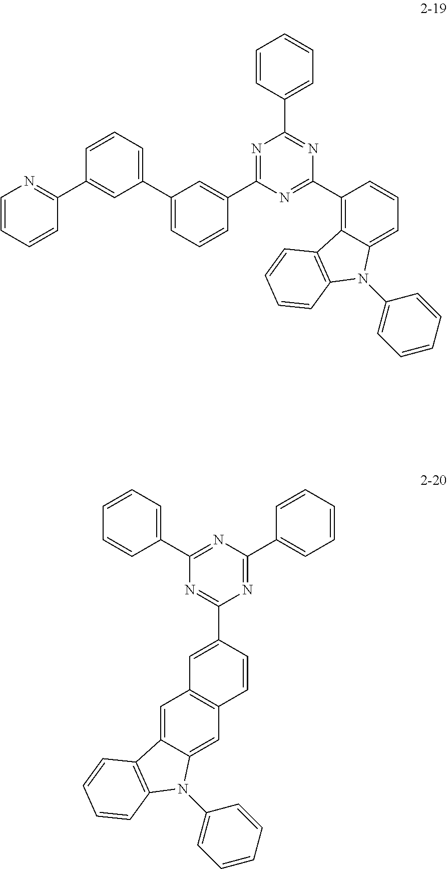 [SMILES: c1ccc(-c2nc(-c3cccc(-c4cccc(-c5ccccn5)c4)c3)nc(-c3cccc4c3c3ccccc3n4-c3ccccc3)n2)cc1 ; c1ccc(-c2nc(-c3ccccc3)nc(-c3ccc4cc5c(cc4c3)c3ccccc3n5-c3ccccc3)n2)cc1]

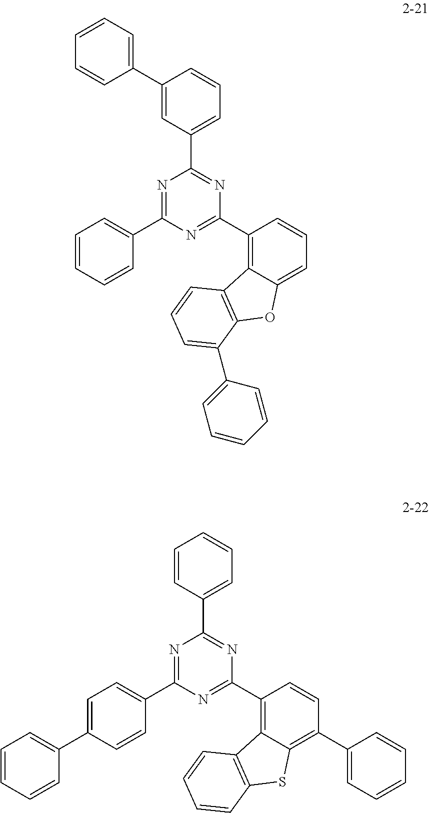 [SMILES: c1ccc(-c2cccc(-c3nc(-c4ccccc4)nc(-c4cccc5oc6c(-c7ccccc7)cccc6c45)n3)c2)cc1 ; c1ccc(-c2ccc(-c3nc(-c4ccccc4)nc(-c4ccc(-c5ccccc5)c5sc6ccccc6c45)n3)cc2)cc1]

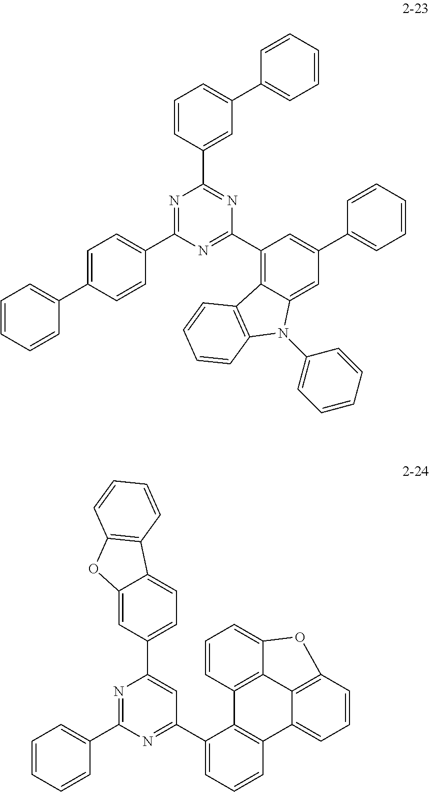 [SMILES: c1ccc(-c2ccc(-c3nc(-c4cccc(-c5ccccc5)c4)nc(-c4cc(-c5ccccc5)cc5c4c4ccccc4n5-c4ccccc4)n3)cc2)cc1 ; c1ccc(-c2nc(-c3ccc4c(c3)oc3ccccc34)cc(-c3cccc4c5cccc6oc7cccc(c34)c7c65)n2)cc1]

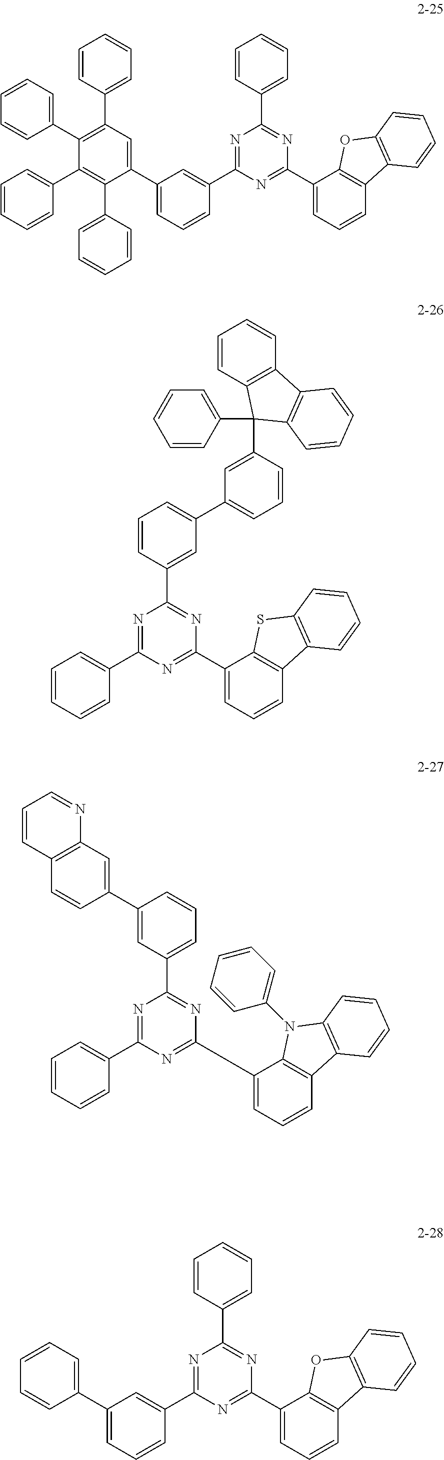 [SMILES: c1ccc(-c2nc(-c3cccc(-c4cc(-c5ccccc5)c(-c5ccccc5)c(-c5ccccc5)c4-c4ccccc4)c3)nc(-c3cccc4c3oc3ccccc34)n2)cc1 ; c1ccc(-c2nc(-c3cccc(-c4cccc(C5(c6ccccc6)c6ccccc6-c6ccccc65)c4)c3)nc(-c3cccc4c3sc3ccccc34)n2)cc1 ; c1ccc(-c2nc(-c3cccc(-c4ccc5cccnc5c4)c3)nc(-c3cccc4c5ccccc5n(-c5ccccc5)c34)n2)cc1 ; c1ccc(-c2cccc(-c3nc(-c4ccccc4)nc(-c4cccc5c4oc4ccccc45)n3)c2)cc1]

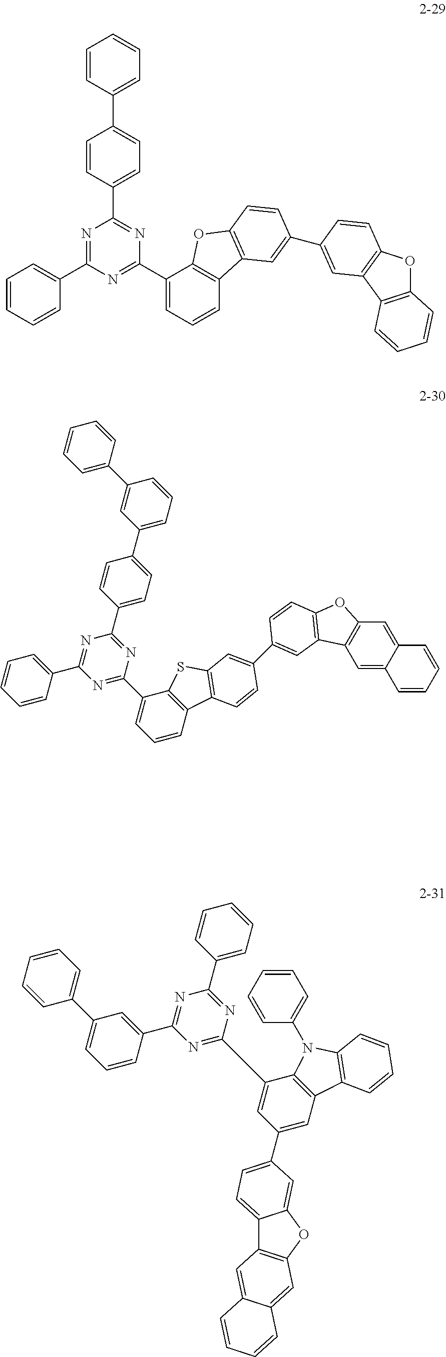 [SMILES: c1ccc(-c2ccc(-c3nc(-c4ccccc4)nc(-c4cccc5c4oc4ccc(-c6ccc7oc8ccccc8c7c6)cc45)n3)cc2)cc1 ; c1ccc(-c2cccc(-c3ccc(-c4nc(-c5ccccc5)nc(-c5cccc6c5sc5cc(-c7ccc8oc9cc%10ccccc%10cc9c8c7)ccc56)n4)cc3)c2)cc1 ; c1ccc(-c2cccc(-c3nc(-c4ccccc4)nc(-c4cc(-c5ccc6c(c5)oc5cc7ccccc7cc56)cc5c6ccccc6n(-c6ccccc6)c45)n3)c2)cc1]

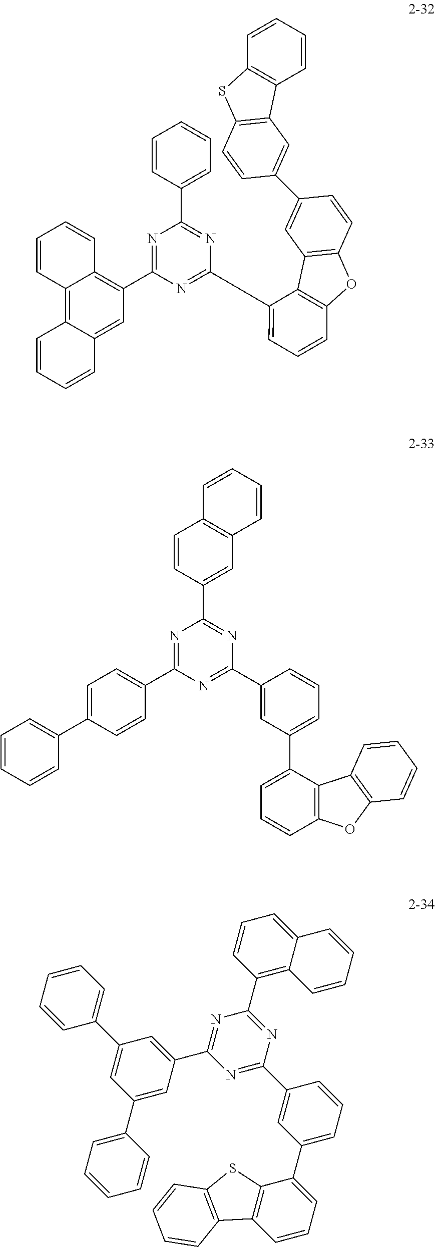 [SMILES: c1ccc(-c2nc(-c3cc4ccccc4c4ccccc34)nc(-c3cccc4oc5ccc(-c6ccc7sc8ccccc8c7c6)cc5c34)n2)cc1 ; c1ccc(-c2ccc(-c3nc(-c4cccc(-c5cccc6oc7ccccc7c56)c4)nc(-c4ccc5ccccc5c4)n3)cc2)cc1 ; c1ccc(-c2cc(-c3ccccc3)cc(-c3nc(-c4cccc(-c5cccc6c5sc5ccccc56)c4)nc(-c4cccc5ccccc45)n3)c2)cc1]

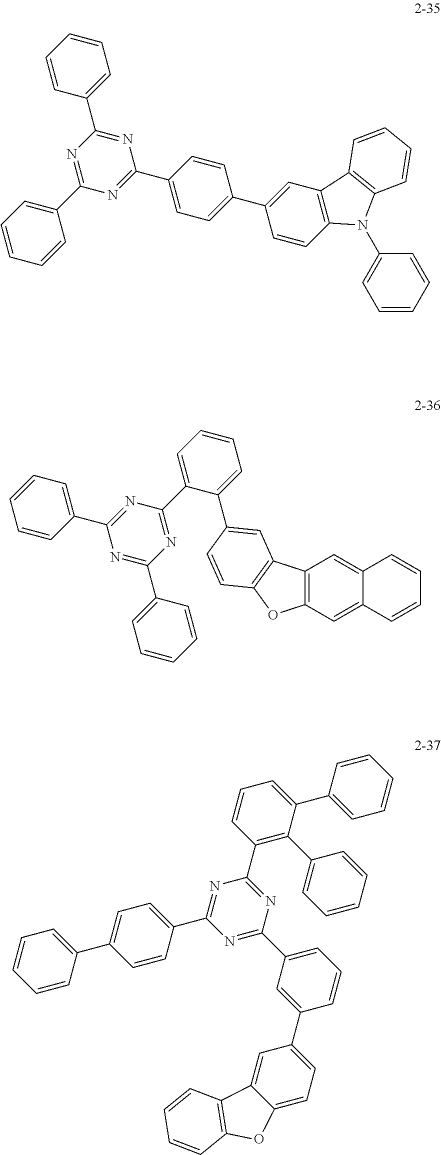 [SMILES: c1ccc(-c2nc(-c3ccccc3)nc(-c3ccc(-c4ccc5c(c4)c4ccccc4n5-c4ccccc4)cc3)n2)cc1 ; c1ccc(-c2nc(-c3ccccc3)nc(-c3ccccc3-c3ccc4oc5cc6ccccc6cc5c4c3)n2)cc1 ; c1ccc(-c2ccc(-c3nc(-c4cccc(-c5ccc6oc7ccccc7c6c5)c4)nc(-c4cccc(-c5ccccc5)c4-c4ccccc4)n3)cc2)cc1]

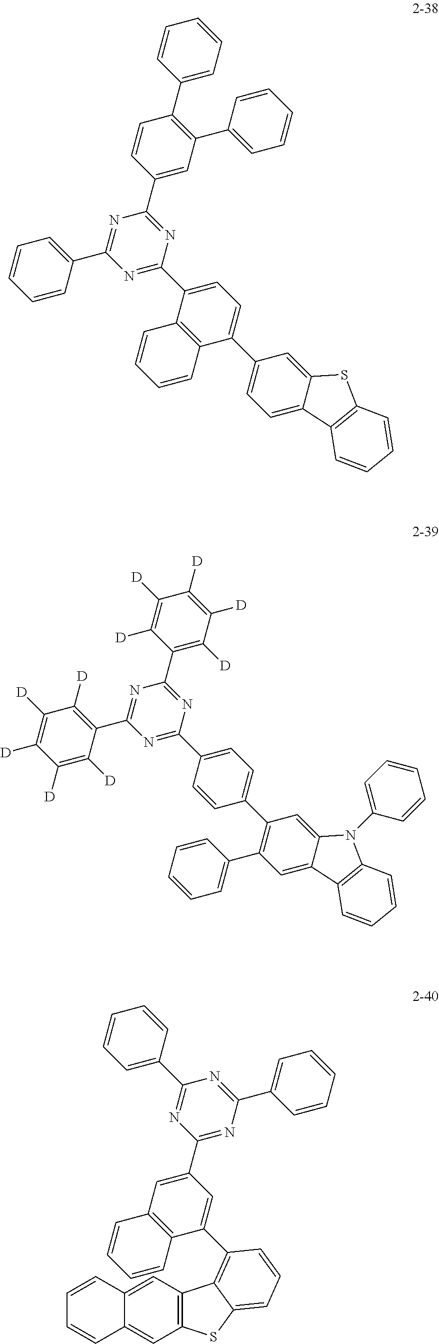 [SMILES: c1ccc(-c2nc(-c3ccc(-c4ccccc4)c(-c4ccccc4)c3)nc(-c3ccc(-c4ccc5c(c4)sc4ccccc45)c4ccccc34)n2)cc1 ; [2H]c1c([2H])c([2H])c(-c2nc(-c3ccc(-c4cc5c(cc4-c4ccccc4)c4ccccc4n5-c4ccccc4)cc3)nc(-c3c([2H])c([2H])c([2H])c([2H])c3[2H])n2)c([2H])c1[2H] ; c1ccc(-c2nc(-c3ccccc3)nc(-c3cc(-c4cccc5sc6cc7ccccc7cc6c45)c4ccccc4c3)n2)cc1]

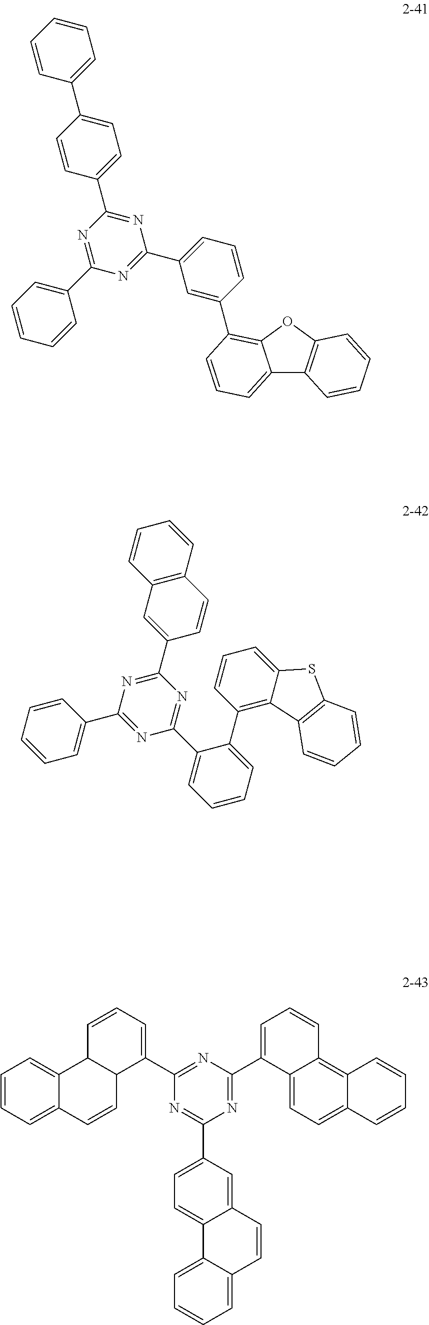 [SMILES: c1ccc(-c2ccc(-c3nc(-c4ccccc4)nc(-c4cccc(-c5cccc6c5oc5ccccc56)c4)n3)cc2)cc1 ; c1ccc(-c2nc(-c3ccc4ccccc4c3)nc(-c3ccccc3-c3cccc4sc5ccccc5c34)n2)cc1 ; C1=CC2c3ccccc3C=CC2C(c2nc(-c3ccc4c(ccc5ccccc54)c3)nc(-c3cccc4c3ccc3ccccc34)n2)=C1]

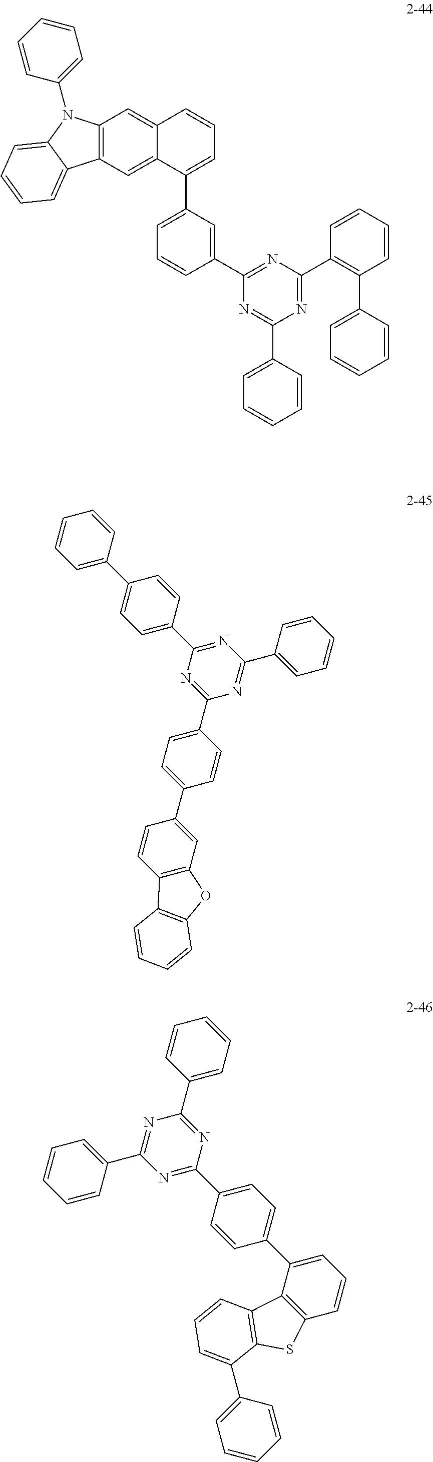 [SMILES: c1ccc(-c2nc(-c3cccc(-c4cccc5cc6c(cc45)c4ccccc4n6-c4ccccc4)c3)nc(-c3ccccc3-c3ccccc3)n2)cc1 ; c1ccc(-c2ccc(-c3nc(-c4ccccc4)nc(-c4ccc(-c5ccc6c(c5)oc5ccccc56)cc4)n3)cc2)cc1 ; c1ccc(-c2nc(-c3ccccc3)nc(-c3ccc(-c4cccc5sc6c(-c7ccccc7)cccc6c45)cc3)n2)cc1]

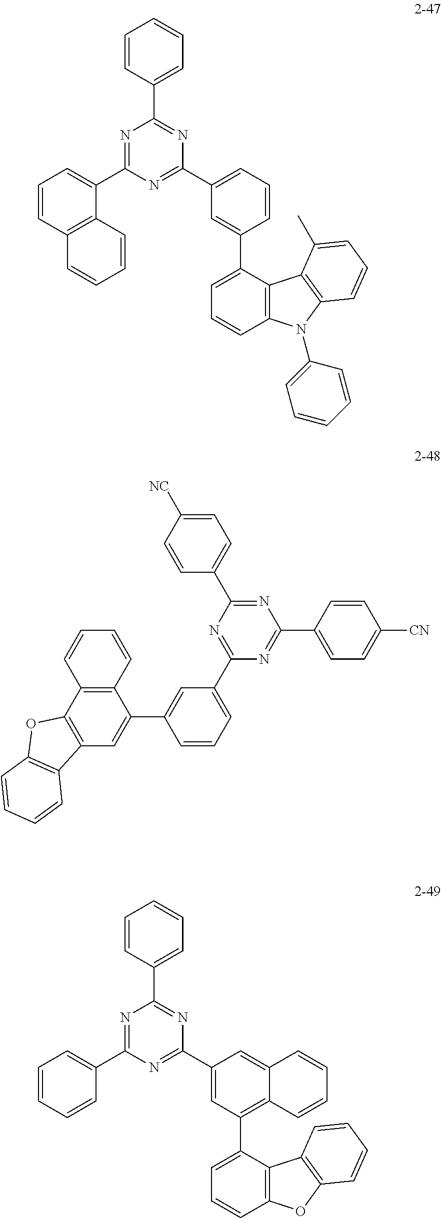 [SMILES: Cc1cccc2c1c1c(-c3cccc(-c4nc(-c5ccccc5)nc(-c5cccc6ccccc56)n4)c3)cccc1n2-c1ccccc1 ; N#Cc1ccc(-c2nc(-c3ccc(C#N)cc3)nc(-c3cccc(-c4cc5c6ccccc6oc5c5ccccc45)c3)n2)cc1 ; c1ccc(-c2nc(-c3ccccc3)nc(-c3cc(-c4cccc5oc6ccccc6c45)c4ccccc4c3)n2)cc1]

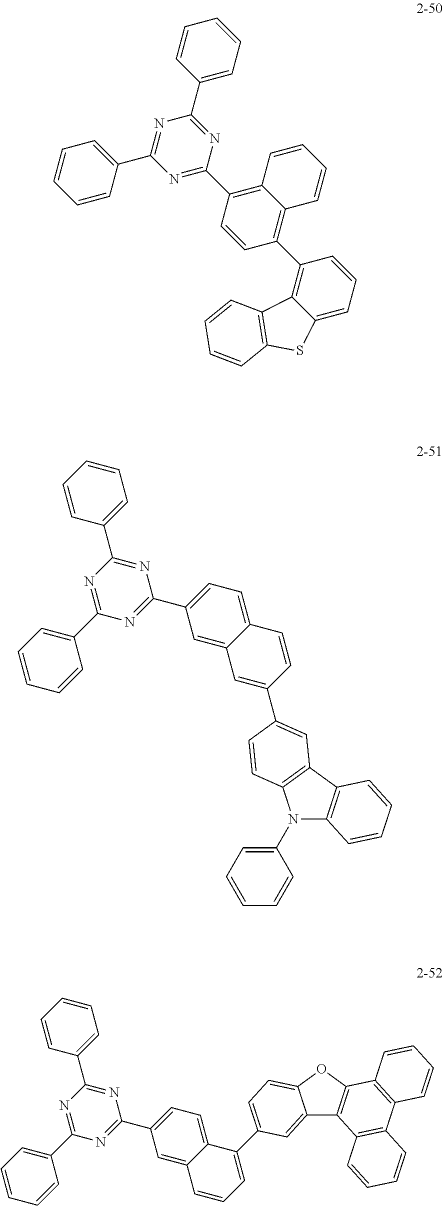 [SMILES: c1ccc(-c2nc(-c3ccccc3)nc(-c3ccc(-c4cccc5sc6ccccc6c45)c4ccccc34)n2)cc1 ; c1ccc(-c2nc(-c3ccccc3)nc(-c3ccc4ccc(-c5ccc6c(c5)c5ccccc5n6-c5ccccc5)cc4c3)n2)cc1 ; c1ccc(-c2nc(-c3ccccc3)nc(-c3ccc4c(-c5ccc6oc7c8ccccc8c8ccccc8c7c6c5)cccc4c3)n2)cc1]

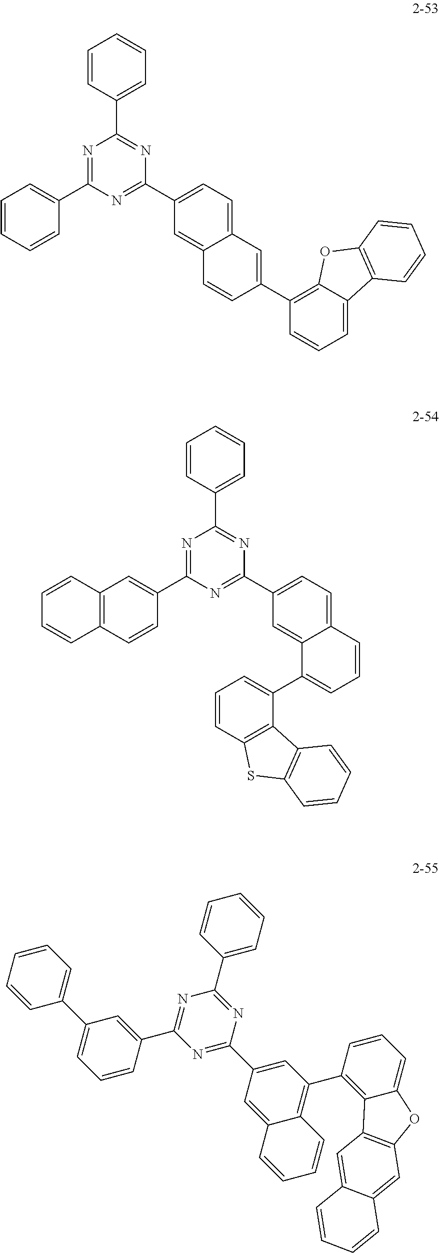 [SMILES: c1ccc(-c2nc(-c3ccccc3)nc(-c3ccc4cc(-c5cccc6c5oc5ccccc56)ccc4c3)n2)cc1 ; c1ccc(-c2nc(-c3ccc4ccccc4c3)nc(-c3ccc4cccc(-c5cccc6sc7ccccc7c56)c4c3)n2)cc1 ; c1ccc(-c2cccc(-c3nc(-c4ccccc4)nc(-c4cc(-c5cccc6oc7cc8ccccc8cc7c56)c5ccccc5c4)n3)c2)cc1]

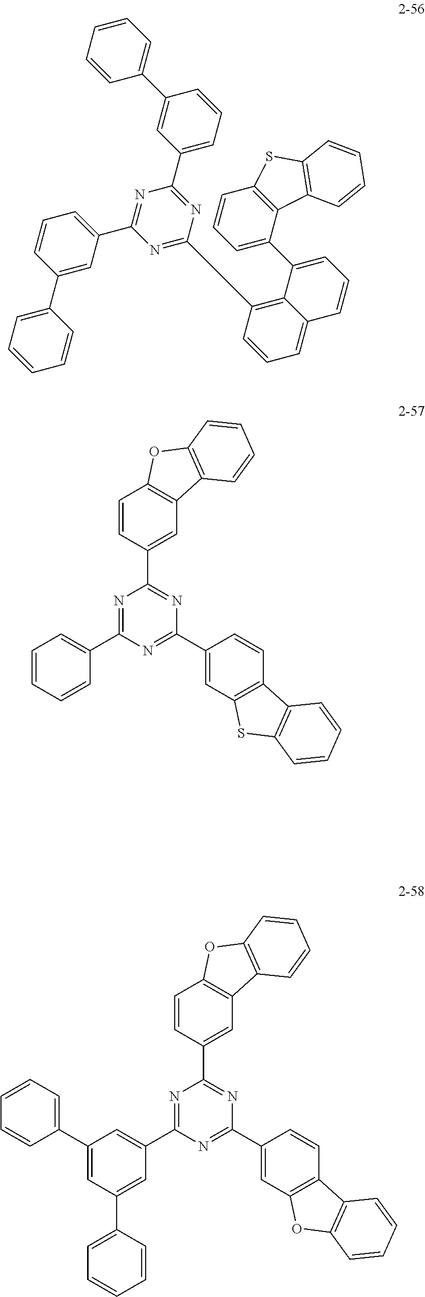 [SMILES: c1ccc(-c2cccc(-c3nc(-c4cccc(-c5ccccc5)c4)nc(-c4cccc5cccc(-c6cccc7sc8ccccc8c67)c45)n3)c2)cc1 ; c1ccc(-c2nc(-c3ccc4c(c3)sc3ccccc34)nc(-c3ccc4oc5ccccc5c4c3)n2)cc1 ; c1ccc(-c2cc(-c3ccccc3)cc(-c3nc(-c4ccc5c(c4)oc4ccccc45)nc(-c4ccc5oc6ccccc6c5c4)n3)c2)cc1]

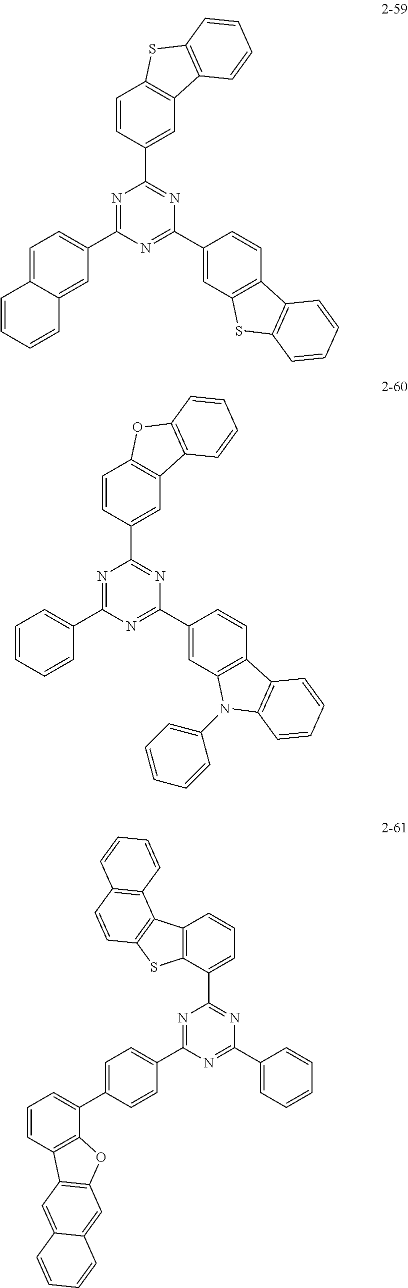 [SMILES: c1ccc2cc(-c3nc(-c4ccc5c(c4)sc4ccccc45)nc(-c4ccc5sc6ccccc6c5c4)n3)ccc2c1 ; c1ccc(-c2nc(-c3ccc4oc5ccccc5c4c3)nc(-c3ccc4c5ccccc5n(-c5ccccc5)c4c3)n2)cc1 ; c1ccc(-c2nc(-c3ccc(-c4cccc5c4oc4cc6ccccc6cc45)cc3)nc(-c3cccc4c3sc3ccc5ccccc5c34)n2)cc1]

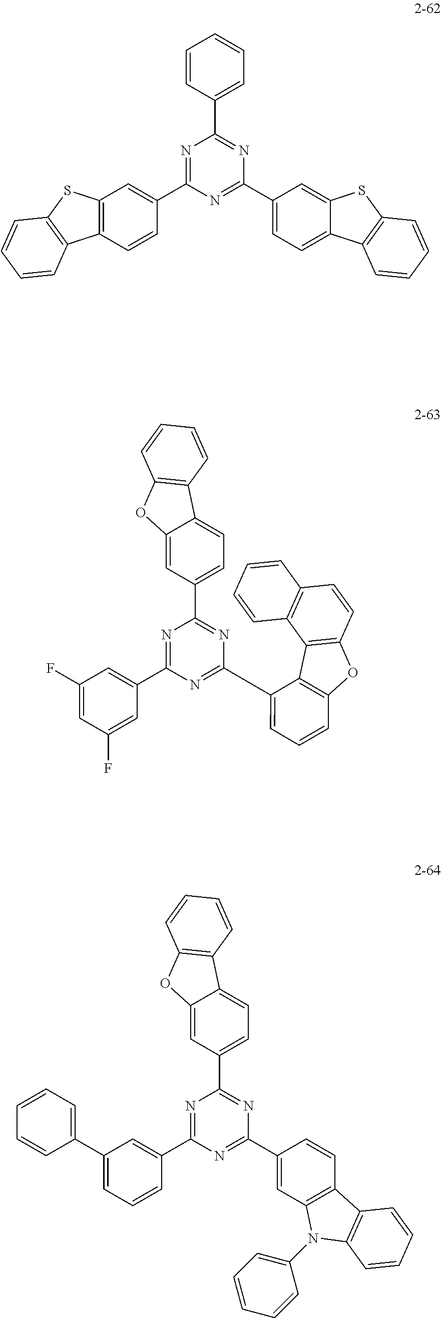 [SMILES: c1ccc(-c2nc(-c3ccc4c(c3)sc3ccccc34)nc(-c3ccc4c(c3)sc3ccccc34)n2)cc1 ; Fc1cc(F)cc(-c2nc(-c3ccc4c(c3)oc3ccccc34)nc(-c3cccc4oc5ccc6ccccc6c5c34)n2)c1 ; c1ccc(-c2cccc(-c3nc(-c4ccc5c(c4)oc4ccccc45)nc(-c4ccc5c6ccccc6n(-c6ccccc6)c5c4)n3)c2)cc1]

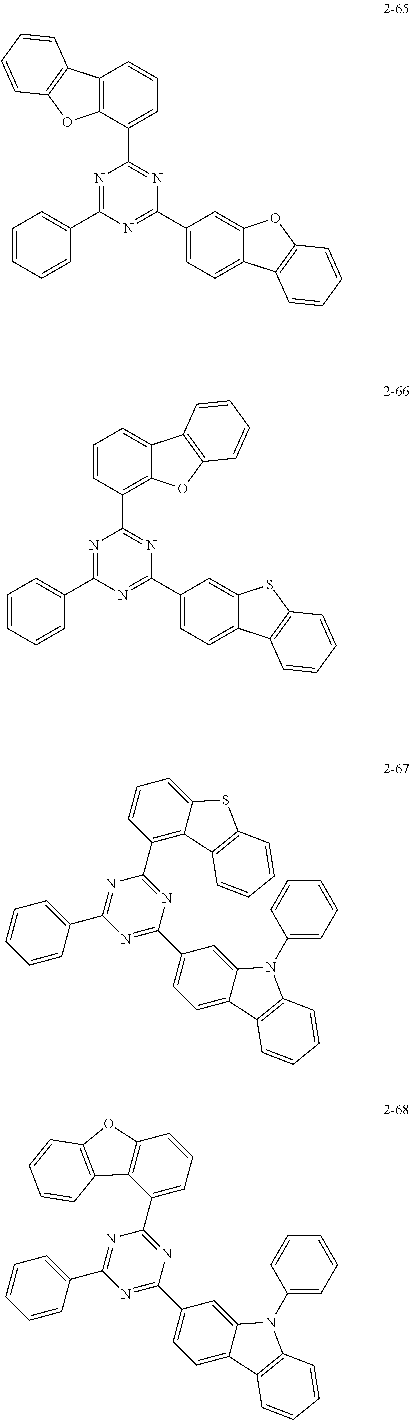 [SMILES: c1ccc(-c2nc(-c3ccc4c(c3)oc3ccccc34)nc(-c3cccc4c3oc3ccccc34)n2)cc1 ; c1ccc(-c2nc(-c3ccc4c(c3)sc3ccccc34)nc(-c3cccc4c3oc3ccccc34)n2)cc1 ; c1ccc(-c2nc(-c3ccc4c5ccccc5n(-c5ccccc5)c4c3)nc(-c3cccc4sc5ccccc5c34)n2)cc1 ; c1ccc(-c2nc(-c3ccc4c5ccccc5n(-c5ccccc5)c4c3)nc(-c3cccc4oc5ccccc5c34)n2)cc1]

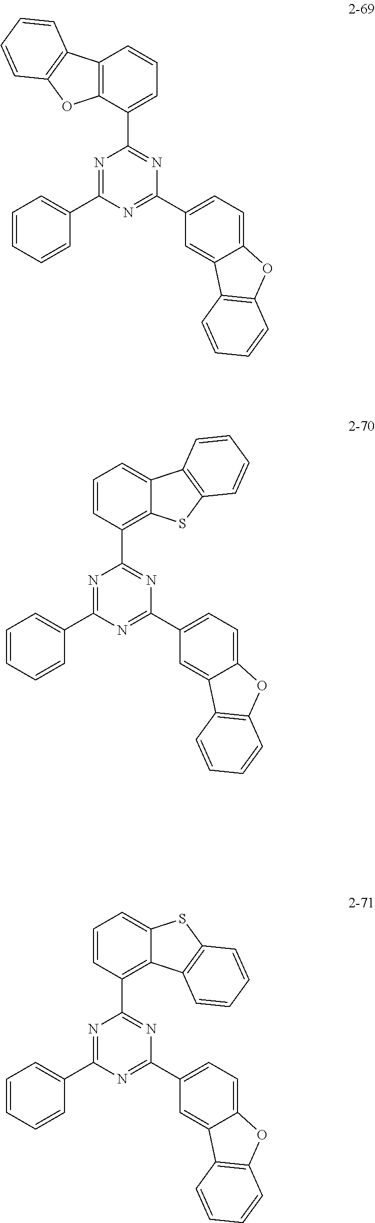 [SMILES: c1ccc(-c2nc(-c3ccc4oc5ccccc5c4c3)nc(-c3cccc4c3oc3ccccc34)n2)cc1 ; c1ccc(-c2nc(-c3ccc4oc5ccccc5c4c3)nc(-c3cccc4c3sc3ccccc34)n2)cc1 ; c1ccc(-c2nc(-c3ccc4oc5ccccc5c4c3)nc(-c3cccc4sc5ccccc5c34)n2)cc1]

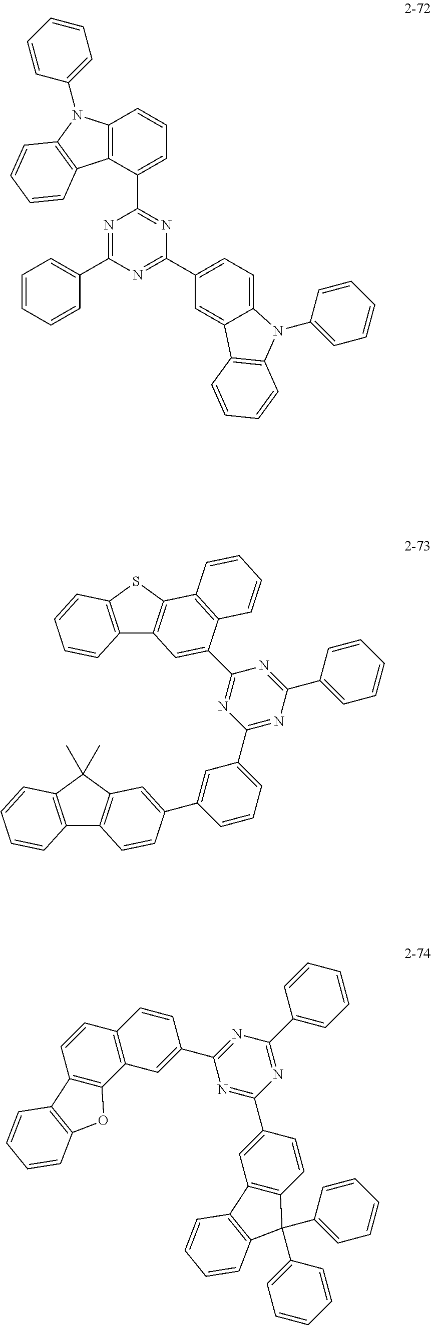 [SMILES: c1ccc(-c2nc(-c3ccc4c(c3)c3ccccc3n4-c3ccccc3)nc(-c3cccc4c3c3ccccc3n4-c3ccccc3)n2)cc1 ; CC1(C)c2ccccc2-c2ccc(-c3cccc(-c4nc(-c5ccccc5)nc(-c5cc6c7ccccc7sc6c6ccccc56)n4)c3)cc21 ; c1ccc(-c2nc(-c3ccc4c(c3)-c3ccccc3C4(c3ccccc3)c3ccccc3)nc(-c3ccc4ccc5c6ccccc6oc5c4c3)n2)cc1]

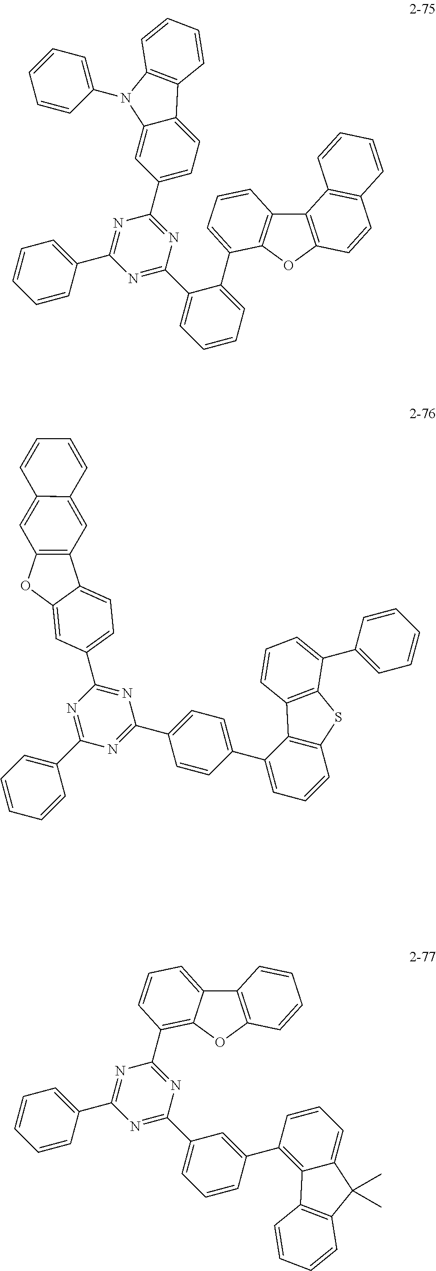 [SMILES: c1ccc(-c2nc(-c3ccc4c5ccccc5n(-c5ccccc5)c4c3)nc(-c3ccccc3-c3cccc4c3oc3ccc5ccccc5c34)n2)cc1 ; c1ccc(-c2nc(-c3ccc(-c4cccc5sc6c(-c7ccccc7)cccc6c45)cc3)nc(-c3ccc4c(c3)oc3cc5ccccc5cc34)n2)cc1 ; CC1(C)c2ccccc2-c2c(-c3cccc(-c4nc(-c5ccccc5)nc(-c5cccc6c5oc5ccccc56)n4)c3)cccc21]

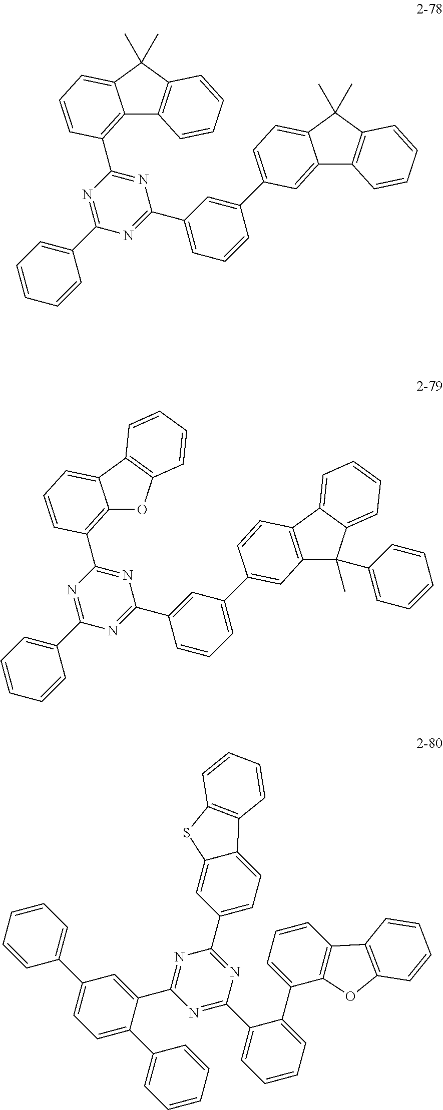 [SMILES: CC1(C)c2ccccc2-c2cc(-c3cccc(-c4nc(-c5ccccc5)nc(-c5cccc6c5-c5ccccc5C6(C)C)n4)c3)ccc21 ; CC1(c2ccccc2)c2ccccc2-c2ccc(-c3cccc(-c4nc(-c5ccccc5)nc(-c5cccc6c5oc5ccccc56)n4)c3)cc21 ; c1ccc(-c2ccc(-c3ccccc3)c(-c3nc(-c4ccc5c(c4)sc4ccccc45)nc(-c4ccccc4-c4cccc5c4oc4ccccc45)n3)c2)cc1]

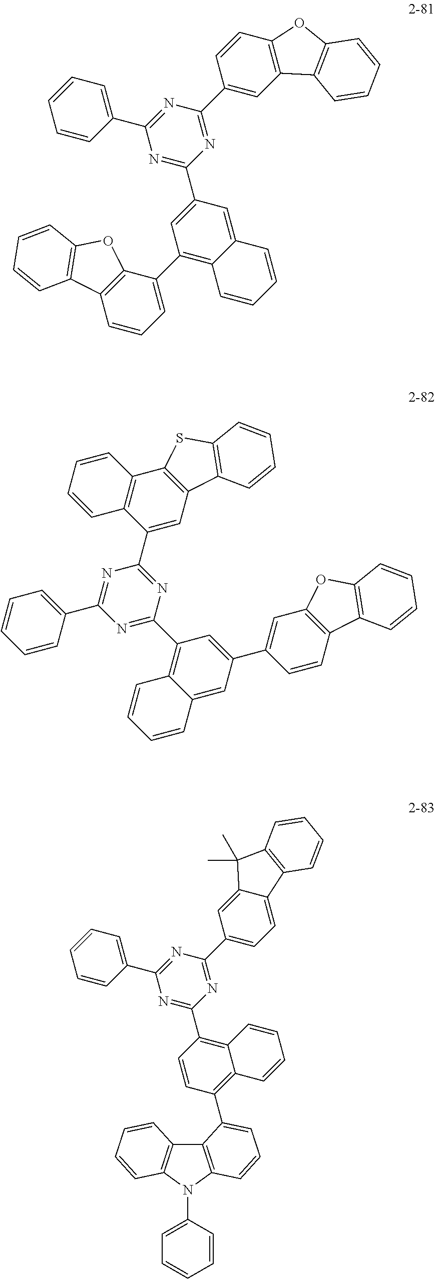 [SMILES: c1ccc(-c2nc(-c3cc(-c4cccc5c4oc4ccccc45)c4ccccc4c3)nc(-c3ccc4oc5ccccc5c4c3)n2)cc1 ; c1ccc(-c2nc(-c3cc(-c4ccc5c(c4)oc4ccccc45)cc4ccccc34)nc(-c3cc4c5ccccc5sc4c4ccccc34)n2)cc1 ; CC1(C)c2ccccc2-c2ccc(-c3nc(-c4ccccc4)nc(-c4ccc(-c5cccc6c5c5ccccc5n6-c5ccccc5)c5ccccc45)n3)cc21]

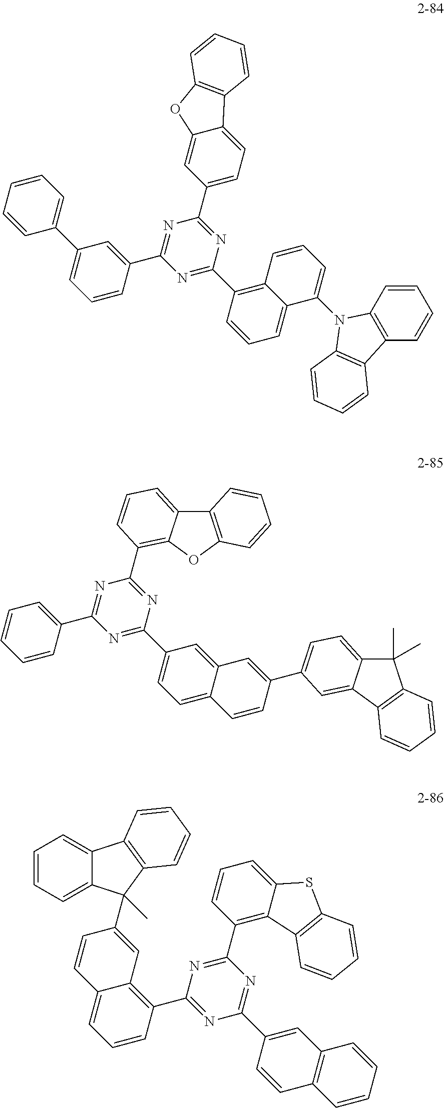 [SMILES: c1ccc(-c2cccc(-c3nc(-c4ccc5c(c4)oc4ccccc45)nc(-c4cccc5c(-n6c7ccccc7c7ccccc76)cccc45)n3)c2)cc1 ; CC1(C)c2ccccc2-c2cc(-c3ccc4ccc(-c5nc(-c6ccccc6)nc(-c6cccc7c6oc6ccccc67)n5)cc4c3)ccc21 ; CC1(c2ccc3cccc(-c4nc(-c5ccc6ccccc6c5)nc(-c5cccc6sc7ccccc7c56)n4)c3c2)c2ccccc2-c2ccccc21]

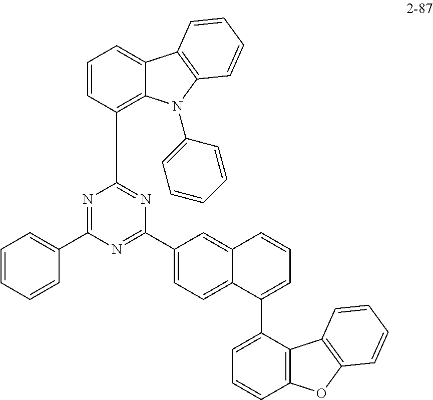 [SMILES: c1ccc(-c2nc(-c3ccc4c(-c5cccc6oc7ccccc7c56)cccc4c3)nc(-c3cccc4c5ccccc5n(-c5ccccc5)c34)n2)cc1]

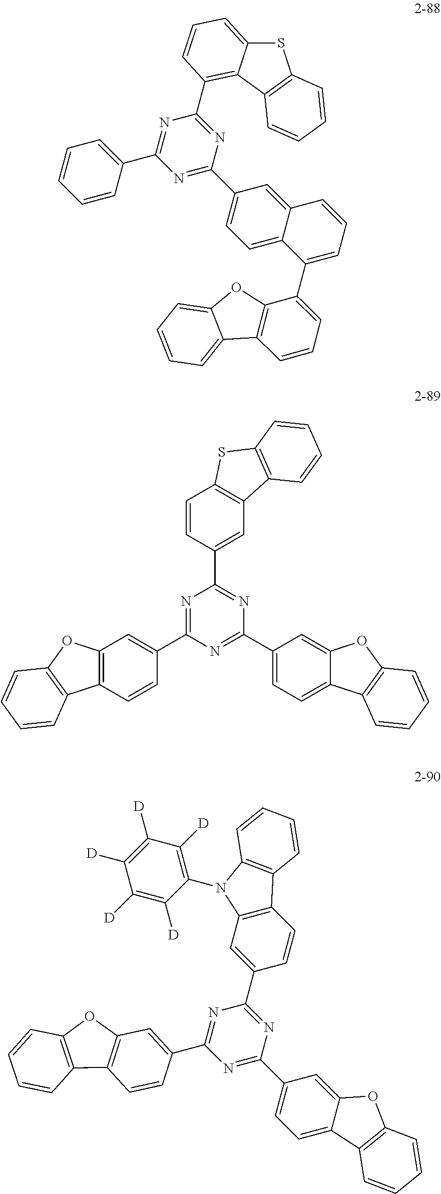 [SMILES: c1ccc(-c2nc(-c3ccc4c(-c5cccc6c5oc5ccccc56)cccc4c3)nc(-c3cccc4sc5ccccc5c34)n2)cc1 ; c1ccc2c(c1)oc1cc(-c3nc(-c4ccc5c(c4)oc4ccccc45)nc(-c4ccc5sc6ccccc6c5c4)n3)ccc12 ; [2H]c1c([2H])c([2H])c(-n2c3ccccc3c3ccc(-c4nc(-c5ccc6c(c5)oc5ccccc56)nc(-c5ccc6c(c5)oc5ccccc56)n4)cc32)c([2H])c1[2H]]

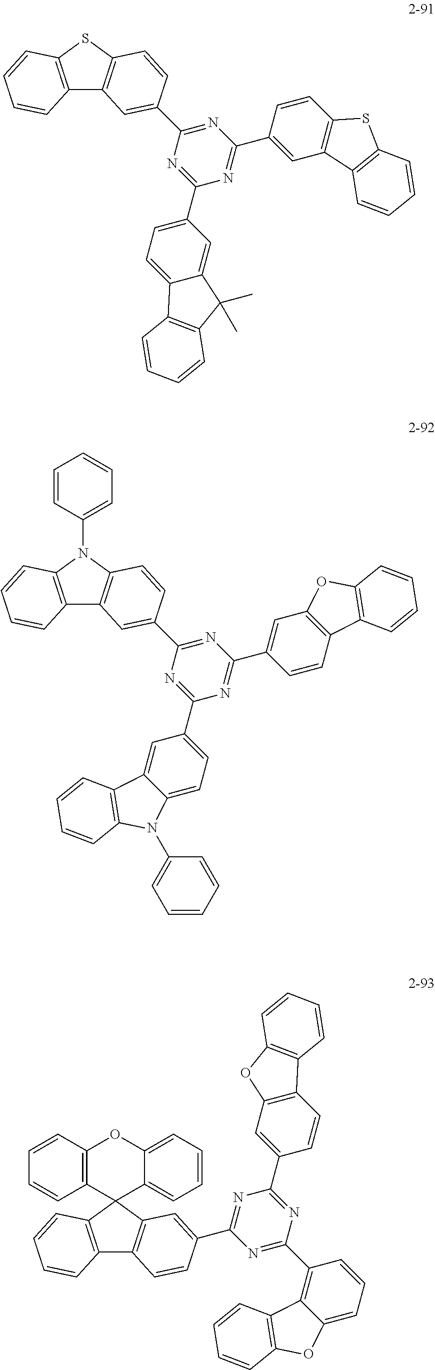 [SMILES: CC1(C)c2ccccc2-c2ccc(-c3nc(-c4ccc5sc6ccccc6c5c4)nc(-c4ccc5sc6ccccc6c5c4)n3)cc21 ; c1ccc(-n2c3ccccc3c3cc(-c4nc(-c5ccc6c(c5)oc5ccccc56)nc(-c5ccc6c(c5)c5ccccc5n6-c5ccccc5)n4)ccc32)cc1 ; c1ccc2c(c1)Oc1ccccc1C21c2ccccc2-c2ccc(-c3nc(-c4ccc5c(c4)oc4ccccc45)nc(-c4cccc5oc6ccccc6c45)n3)cc21]

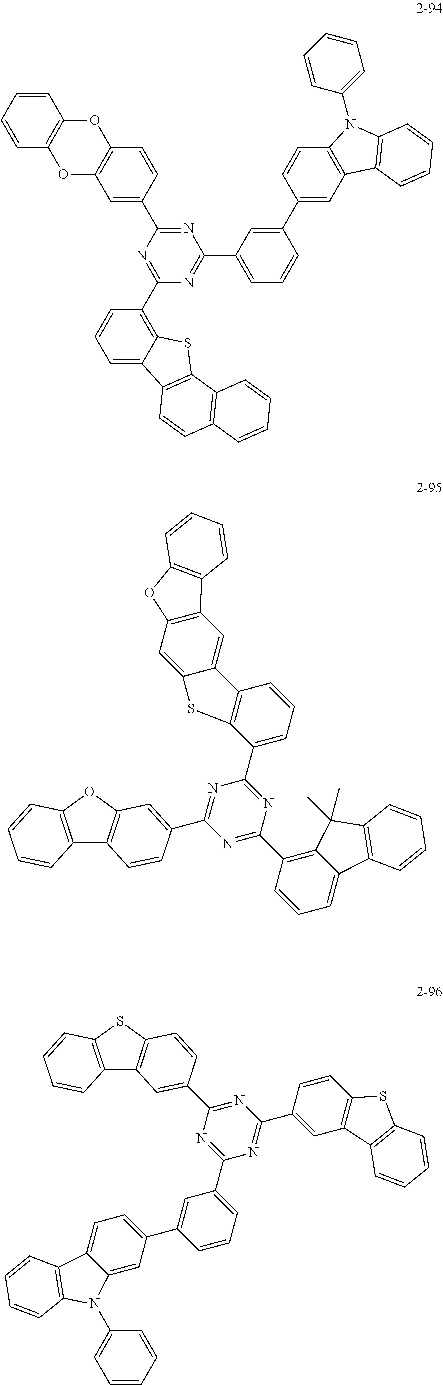 [SMILES: c1ccc(-n2c3ccccc3c3cc(-c4cccc(-c5nc(-c6ccc7c(c6)Oc6ccccc6O7)nc(-c6cccc7c6sc6c8ccccc8ccc76)n5)c4)ccc32)cc1 ; CC1(C)c2ccccc2-c2cccc(-c3nc(-c4ccc5c(c4)oc4ccccc45)nc(-c4cccc5c4sc4cc6oc7ccccc7c6cc45)n3)c21 ; c1ccc(-n2c3ccccc3c3ccc(-c4cccc(-c5nc(-c6ccc7sc8ccccc8c7c6)nc(-c6ccc7sc8ccccc8c7c6)n5)c4)cc32)cc1]

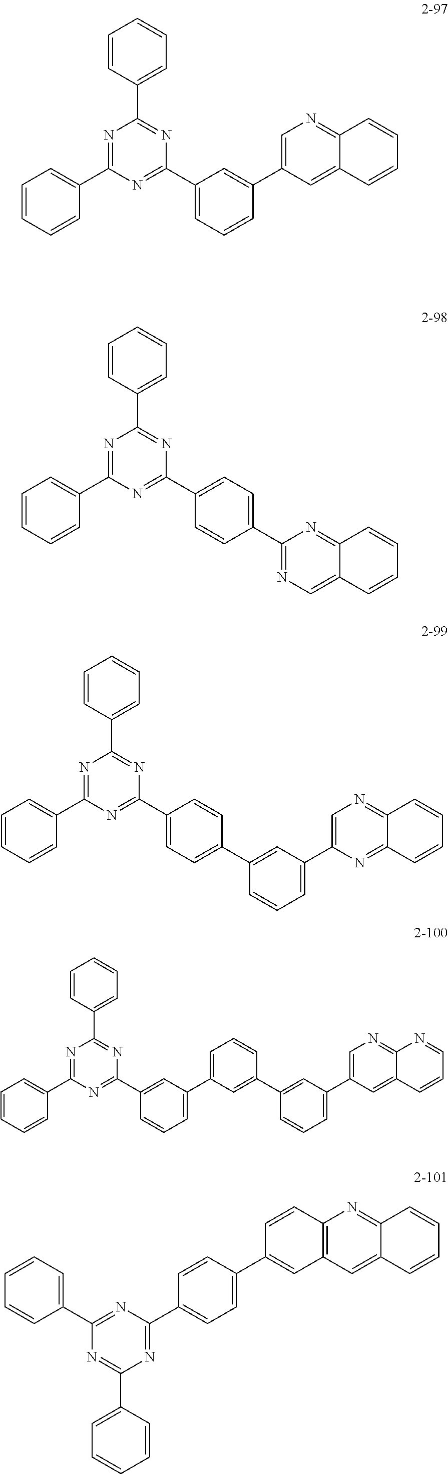 [SMILES: c1ccc(-c2nc(-c3ccccc3)nc(-c3cccc(-c4cnc5ccccc5c4)c3)n2)cc1 ; c1ccc(-c2nc(-c3ccccc3)nc(-c3ccc(-c4ncc5ccccc5n4)cc3)n2)cc1 ; c1ccc(-c2nc(-c3ccccc3)nc(-c3ccc(-c4cccc(-c5cnc6ccccc6n5)c4)cc3)n2)cc1 ; c1ccc(-c2nc(-c3ccccc3)nc(-c3cccc(-c4cccc(-c5cccc(-c6cnc7ncccc7c6)c5)c4)c3)n2)cc1 ; c1ccc(-c2nc(-c3ccccc3)nc(-c3ccc(-c4ccc5nc6ccccc6cc5c4)cc3)n2)cc1]

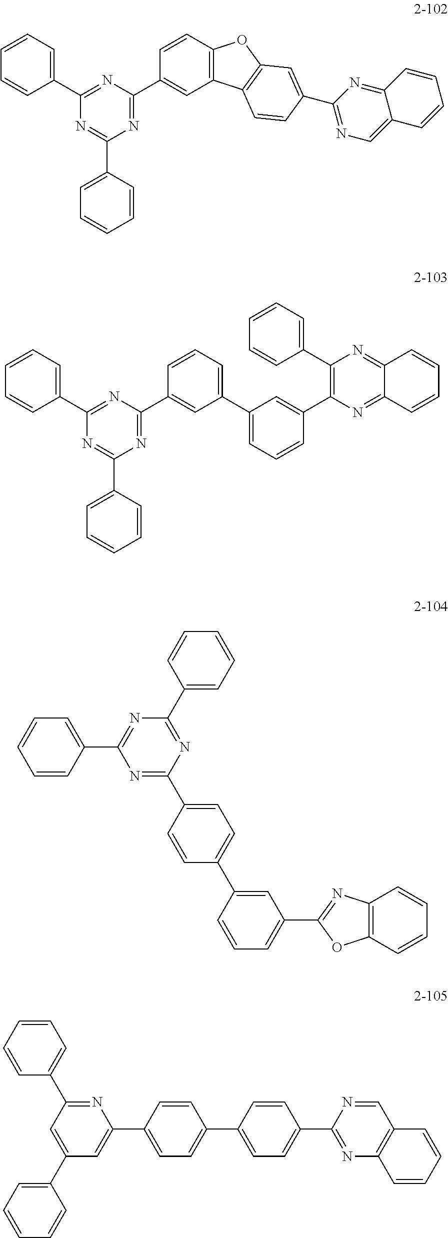 [SMILES: c1ccc(-c2nc(-c3ccccc3)nc(-c3ccc4oc5cc(-c6ncc7ccccc7n6)ccc5c4c3)n2)cc1 ; c1ccc(-c2nc(-c3ccccc3)nc(-c3cccc(-c4cccc(-c5nc6ccccc6nc5-c5ccccc5)c4)c3)n2)cc1 ; c1ccc(-c2nc(-c3ccccc3)nc(-c3ccc(-c4cccc(-c5nc6ccccc6o5)c4)cc3)n2)cc1 ; c1ccc(-c2cc(-c3ccccc3)nc(-c3ccc(-c4ccc(-c5ncc6ccccc6n5)cc4)cc3)c2)cc1]

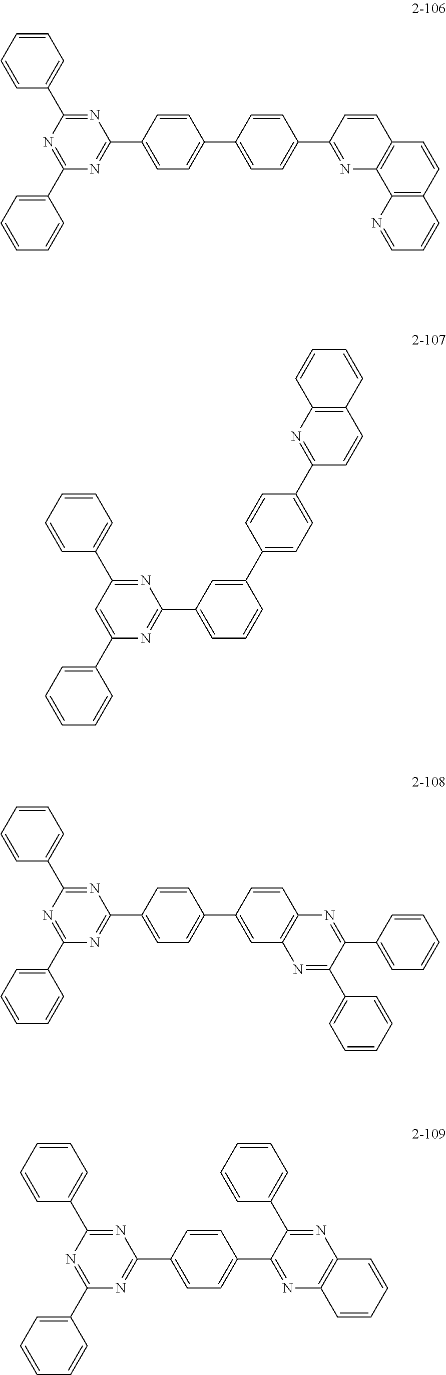 [SMILES: c1ccc(-c2nc(-c3ccccc3)nc(-c3ccc(-c4ccc(-c5ccc6ccc7cccnc7c6n5)cc4)cc3)n2)cc1 ; c1ccc(-c2cc(-c3ccccc3)nc(-c3cccc(-c4ccc(-c5ccc6ccccc6n5)cc4)c3)n2)cc1 ; c1ccc(-c2nc(-c3ccccc3)nc(-c3ccc(-c4ccc5nc(-c6ccccc6)c(-c6ccccc6)nc5c4)cc3)n2)cc1 ; c1ccc(-c2nc(-c3ccccc3)nc(-c3ccc(-c4nc5ccccc5nc4-c4ccccc4)cc3)n2)cc1]

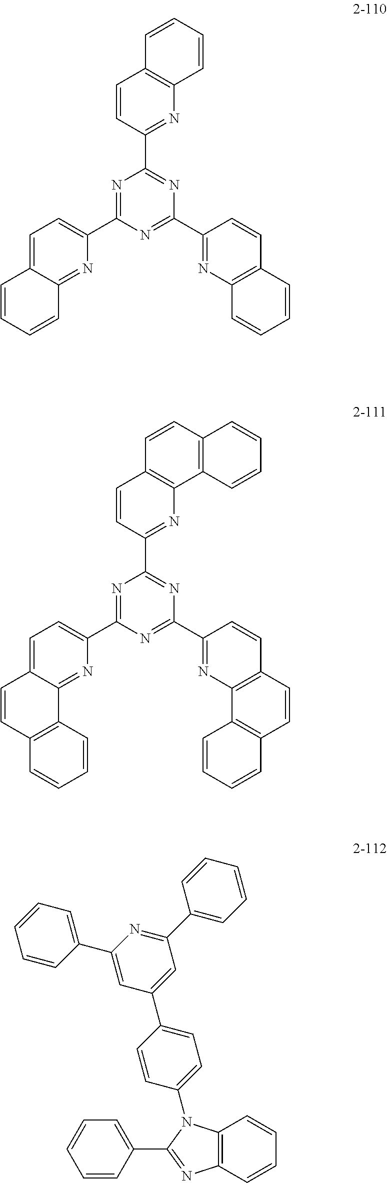 [SMILES: c1ccc2nc(-c3nc(-c4ccc5ccccc5n4)nc(-c4ccc5ccccc5n4)n3)ccc2c1 ; c1ccc2c(c1)ccc1ccc(-c3nc(-c4ccc5ccc6ccccc6c5n4)nc(-c4ccc5ccc6ccccc6c5n4)n3)nc12 ; c1ccc(-c2cc(-c3ccc(-n4c(-c5ccccc5)nc5ccccc54)cc3)cc(-c3ccccc3)n2)cc1]

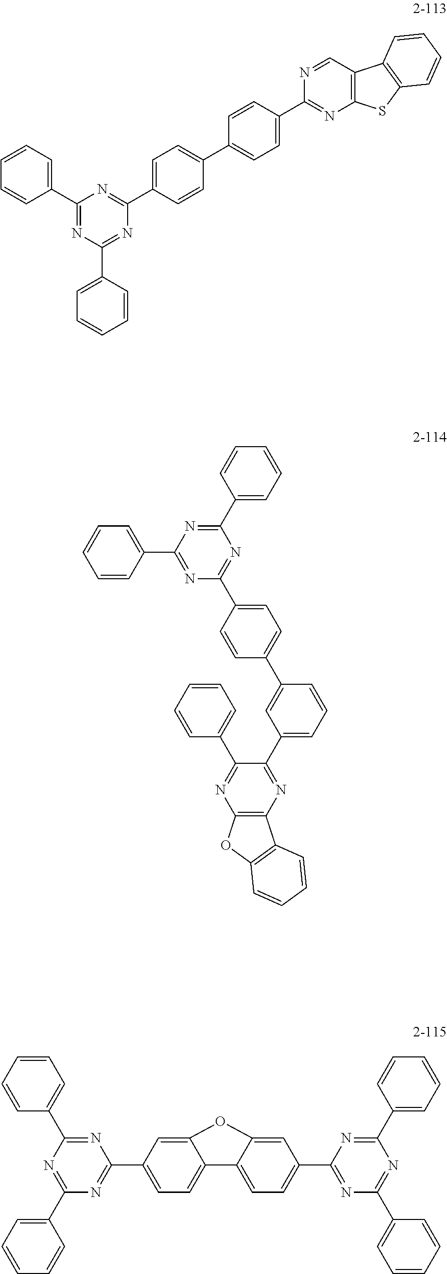 [SMILES: c1ccc(-c2nc(-c3ccccc3)nc(-c3ccc(-c4ccc(-c5ncc6c(n5)sc5ccccc56)cc4)cc3)n2)cc1 ; c1ccc(-c2nc(-c3ccccc3)nc(-c3ccc(-c4cccc(-c5nc6c(nc5-c5ccccc5)oc5ccccc56)c4)cc3)n2)cc1 ; c1ccc(-c2nc(-c3ccccc3)nc(-c3ccc4c(c3)oc3cc(-c5nc(-c6ccccc6)nc(-c6ccccc6)n5)ccc34)n2)cc1]

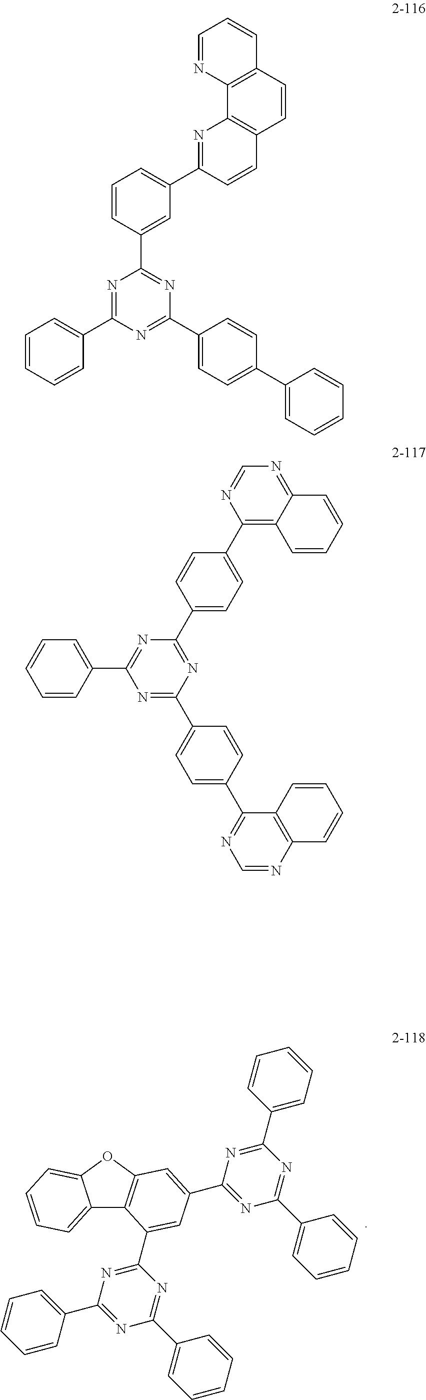 [SMILES: c1ccc(-c2ccc(-c3nc(-c4ccccc4)nc(-c4cccc(-c5ccc6ccc7cccnc7c6n5)c4)n3)cc2)cc1 ; c1ccc(-c2nc(-c3ccc(-c4ncnc5ccccc45)cc3)nc(-c3ccc(-c4ncnc5ccccc45)cc3)n2)cc1 ; c1ccc(-c2nc(-c3ccccc3)nc(-c3cc(-c4nc(-c5ccccc5)nc(-c5ccccc5)n4)c4c(c3)oc3ccccc34)n2)cc1]

10. The organic electronic element of claim 1, comprising at least one hole transport band layer between the anode and the emitting layer, wherein the hole transport band layer comprises a hole transport layer, an emitting auxiliary layer, or both, wherein the hole transport band layer comprises a compound represented by Formula 1.

11. The organic electronic element of claim 1, further comprising a light efficiency enhancing layer formed on at least one surface of the anode and the cathode, the surface being opposite to the organic material layer.

12. The organic electronic element of claim 1, wherein the organic material layer comprises 2 or more stacks, each stack comprising a hole transport layer, an emitting layer, and an electron transport layer sequentially formed on the anode.

13. The organic electronic element of claim 1, wherein the organic material layer further comprises a charge generating layer formed between 2 or more stacks.

14. The electronic device comprising: a display device comprising the organic electronic element of claim 1; and a control unit for driving the display device.

15. The electronic device of claim 14, wherein the organic electronic element is at least one of an OLED, an organic solar cell, an organic photo conductor (OPC), an organic transistor (organic TFT), and an element for monochromic or white illumination.
